(19) United States
(12) Patent Application Publication
(10) Pub. No.: US 2026/0224691 A1
(43) Pub. Date: Aug. 6, 2026

(54) EPSTEIN-BARR VIRUS MRNA VACCINES

(71) Applicant: ModernaTX, Inc., Cambridge, MA (US)

(72) Inventors: Brooke Bollman, Brookline, MA (US); Andrea Carfi, Cambridge, MA (US); Sumana Chandramouli, Cambridge, MA (US); Yen-Ting Lai, Cambridge, MA (US); Emilia Vanni, Cambridge, MA (US)

(21) Appl. No.: 19/149,954

(22) PCT Filed: Jan. 30, 2024
(86) PCT No.: PCT/US2024/013539

§ 371 Date: Jul. 22, 2025

Related U.S. Application Data
(60) Provisional application No. 63/482,272, filed on Jan. 30, 2023.

(51) Int. Cl. A61K 39/245; A61K 39/00; A61P 37/04

(52) U.S. Cl. CPC A61K 39/245; A61P 37/04; A61K 2039/53; A61K 2039/572

(57) ABSTRACT

The disclosure provides mRNA vaccines for treating or preventing Epstein-Barr virus infection. The mRNA vaccines encode lytic and/or latent EBV antigens.

### RELATED APPLICATION

This application claims the benefit under 35 U.S.C. § 119(e) to U.S. Provisional Application No. 63/482,272, entitled “EPSTEIN-BARR VIRUS MRNA VACCINES,” filed on Jan. 30, 2023, the entire contents of which are incorporated herein by reference.

### REFERENCE TO AN ELECTRONIC SEQUENCE LISTING

The contents of the electronic sequence listing (M137870226WO00-VLJ-SEQ.xml; Size: 133,651 bytes; and Date of Creation: Jan. 29, 2024) is herein incorporated by reference in its entirety.

### BACKGROUND

Epstein-Barr virus (EBV) is a complex, large, double-stranded gamma-herpesvirus with a worldwide prevalence of nearly 94% in adulthood. It is linked to several serious acute and chronic medical conditions, including infectious mononucleosis, nasopharyngeal carcinoma, Burkitt and Hodgkin's lymphomas, gastric cancers, posttransplant lymphoproliferative disorders (PTLDs) and, more recently, systemic lupus erythematosus and multiple sclerosis. Infectious mononucleosis is a clinical syndrome that presents with fever, fatigue, sore throat, and lymphadenopathy, and can result in prolonged symptoms as well as hospitalization and splenic rupture.

Posttransplant lymphoproliferative disorders are a group of clinical diseases that occur following transplantation and are characterized by abnormal proliferation of lymphoid cells in the setting of iatrogenic immunosuppression. These disorders have been associated with both solid organ transplantation as well as hematopoietic stem-cell transplantation and occur in both EBV-seropositive and EBV-seronegative transplant recipients. Indeed, in the first year following solid organ transplantation, >90% of B cell PTLDs have evidence of EBV genome likely reflecting primary EBV infection in a seronegative transplant recipient. A bimodal distribution in the incidence of PTLDs has been described with an initial increase in the first year following transplantation involving EBV-positive recipients and a second spike, 5 to 15 years post-transplantation, representing EBV-negative transplant recipients. The cumulative incidence of PTLDs per 100 patient-years continues to increase steadily for approximately 20 years post transplantation.

Multiple sclerosis is an autoimmune disease of the central nervous system that predominantly affects young women and is associated with significant neurological impairment and shortened life-expectancy when untreated. It has been known for some time that a past history of clinical infectious mononucleosis and a later age of onset of infectious mononucleosis are associated with multiple sclerosis. Evidence of past infection with EBV, the cause of infectious mononucleosis, is essentially universal in people with multiple sclerosis, compared to being seen in around 90% of the general adult population. More recent studies have shown that multiple sclerosis only occurs in people who acquire EBV prior to the onset of their disease. Thus, there is now compelling evidence that EBV is “essential, but not sufficient” to cause multiple sclerosis. This has led to the conclusion that EBV is the primary driver of multiple sclerosis pathology and disease activity. Whilst a number of effective disease modifying therapies for the relapsing forms of multiple sclerosis (where attacks of symptoms recur sporadically) have been developed over the past 30 years, these therapies are not a “cure” and are not appropriate or available for all people with multiple sclerosis. Consequently, many people with multiple sclerosis continue to have poor outcomes.

A licensed EBV vaccine is currently unavailable.

### SUMMARY

Epstein-Barr Virus (EBV) is a complex, large, double-stranded DNA gamma-herpesvirus with high rates of seroprevalence (>90%) in adults worldwide. It is responsible for 90% of Infectious mononucleosis cases worldwide, most commonly in adolescents and young adults contracting a primary infection. Infectious mononucleosis is a clinical syndrome that presents with fever, fatigue, sore throat, and lymphadenopathy and can result in prolonged symptoms as well as hospitalization and splenic rupture. EBV has also been associated with other serious diseases including infectious mononucleosis, nasopharyngeal carcinoma, Burkitt lymphoma, Hodgkin's lymphoma, gastric cancer, posttransplant lymphoproliferative disorders (PTLDs) and, more recently, systemic lupus erythematosus and multiple sclerosis.

With a genome approximately 175 kb in size, EBV utilizes several surface glycoproteins to mediate viral entry into B cells and epithelial cells. These include the principal glycoproteins gp350, gHgL, gp42, and gB6. Like other herpesviruses, EBV then establishes lifelong latency in the host, periodically cycling between lytic and latent replication7 (Kempkes and Robertson, 2015). EBV latency in B cells is maintained by a set of latent viral proteins, most notably the Epstein Barr nuclear antigens (EBNAs) and latent membrane proteins (LMPs) expressed at various stages of latency. While the entry glycoproteins are the primary targets of neutralizing antibodies, the latent antigens induce potent T cell responses following natural infection. As such, a vaccine composition that combines glycoproteins with latent antigens could significantly impact serious outcomes of EBV infection in EBV seronegative and seropositive individuals by inducing or boosting humoral and cell-mediated immunity against EBV and establishing immune control over latently infected B cells.

The messenger ribonucleic acid (mRNA) vaccines provided herein safely direct the body's cellular machinery to produce both lytic and latent EBV antigens designed to have therapeutically immunogenic activity inside and outside of cells. These EBV mRNA vaccines herein comprise multiple mRNA polynucleotides, each of which encodes a different EBV lytic antigen (e.g., glycoprotein) or latent antigen protein that collectively elicit improved neutralizing antibody and CD8⁺ and CD4⁺ T cell responses that can clear cells in which the EBV has re-emerged (re-activated) from latency and/or is actively replicating and expressing the wild-type protein counterparts. For example, vaccines of the present disclosure that include mRNA encoding a modified EBV latent nuclear antigen (e.g., EBNA3A) induced a CD4⁺ T cell response with a Th1-like signature, while those vaccine that included both an mRNA encoding the EBV latent nuclear antigen (e.g., EBNA3A) and an mRNA encoding an EBV latent membrane protein (e.g., LMP2B) induced both a CD4⁺ T cell response and a CD8+ T cell response characterized by upregulation of cytotoxicity markers, such as CD107a, IFN-γ, TNF-α, and IL-2. The data provided herein also demonstrates that there is no significant interference when mRNAs encoding multiple EBV latent antigens are co-administered. In fact, the CD8+ T cell response to LMP2B, for example, increased in the presence of EBNA3A. T cell responses were also detected against glycoprotein antigens: EBV gH elicited both CD8⁺ and CD4⁺ T cell responses, whereas soluble EBV gp42 and the EBV gp220 isoform elicited CD4⁺ T cell responses. The mRNA vaccines provided herein, containing both glycoproteins and latent antigens, may be useful, for example, in both EBV-seronegative and EBV seropositive populations at risk for EBV-associated diseases such as PTLD, systemic lupus erythematosus, multiple sclerosis, and/or cancer. Further, the EBV mRNA vaccines, as provided herein may be used to induce a balanced immune response, comprising both cellular and humoral immunity, without many of the risks associated with DNA vaccination.

Some aspects of the present disclosure relate to an Epstein-Barr virus (EBV) messenger ribonucleic acid (mRNA) vaccine comprising: (a) one or more mRNAs, each comprising an open reading frame (ORF) encoding an EBV lytic antigen; (b) one or more mRNAs, each comprising an ORF encoding an EBV latent antigen; and (c) a lipid nanoparticle, wherein at least 50% of the mass of the mRNAs in the vaccine are mRNAs encoding the EBV lytic antigen.

Some aspects of the present disclosure relate to a therapeutic Epstein-Barr virus (EBV) messenger ribonucleic acid (mRNA) vaccine comprising: one or more mRNAs encoding at least two EBV latent antigens; and a lipid nanoparticle, wherein the EBV latent antigens are selected from EBV nuclear antigen 1 (EBNA1), EBV nuclear antigen 2 (EBNA2), EBV nuclear antigen 3A (EBNA3A), EBV nuclear antigen 3B (EBNA3B), EBV nuclear antigen 3C (EBNA3C), EBV latent membrane protein 1 (LMP1), EBV latent membrane protein 2A (LMP2A), and EBV latent membrane protein 2B (LMP2B).

Some aspects of the present disclosure relate to a therapeutic Epstein-Barr virus (EBV) messenger ribonucleic acid (mRNA) vaccine comprising: one or more mRNAs encoding a EBNA3A antigen; and a lipid nanoparticle, wherein the EBNA3A antigen has one or more of the following features: a. lacks a C-terminal transcriptional regulator domain, b. has a length of about 523 amino acids, c. comprises amino acid residues corresponding to amino acid residues 1-523 of the naturally occurring EBNA3A, d. does not include amino acid residues corresponding to amino acid residues 524-944 of the naturally occurring EBNA3A, e. the N-terminus of the EBNA3A is truncated relative to the naturally occurring EBNA3A, wherein the C-terminus of the EBNA3A is truncated relative to the naturally occurring EBNA3A, f. has a length of about 455 amino acids, g. comprises amino acid residues corresponding to amino acid residues 68-523 of the naturally occurring EBNA3A, h. does not include amino acid residues corresponding to amino acid residues 1-67 and 524-944 of the naturally occurring EBNA3A, i. one or more nuclear localization signals (NLS) in the EBNA3A are mutated relative to the naturally occurring EBNA3A, j. 1-4, 2-4, 3-4, or 4 of the NLS in the EBNA3A are mutated relative to the naturally occurring EBNA3A, optionally wherein the mutated NLS are at amino acid positions selected from: 63-66, 146-155, 375-381, and 394-398, relative to the naturally occurring EBNA3A, and/or, k. has a C-terminus that is truncated at amino acid residue 68, the N-terminus of the EBNA3A is truncated at amino acid residue 523, and the EBNA3 comprises NLS mutations at amino acid residues 146-155, 375-378, and 394-398, relative to the naturally occurring EBNA3A.

Some aspects of the present disclosure relate to a method for treating an EBV infection, comprising administering to a subject an EBV mRNA vaccine comprising one or more mRNAs encoding at least two latent EBV antigens to induce a therapeutically effective cytotoxic CD8 T cell response against EBV infected cells.

Some aspects of the present disclosure relate to an Epstein-Barr virus (EBV) messenger ribonucleic acid (mRNA) vaccine comprising: (a) one or more mRNAs, each encoding an EBV lytic antigen; (b) one or more mRNAs, each encoding EBV latent antigens; and (c) a lipid nanoparticle, wherein at least 50% (e.g., at least 55%, at least 60%, at least 65%, at least 70%, at least 75%, or at least 80%) of the mRNAs of the vaccine encodes the EBV lytic antigens.

In some embodiments, about 50% of the mRNAs of the vaccine encodes the EBV lytic antigens.

In some embodiments, about 50% to about 80% of the mRNAs of the vaccine encodes the EBV lytic antigens. In some embodiments, about 50% to about 70% of the mRNAs of the vaccine encodes the EBV lytic antigens. In some embodiments, about 50% to about 60% of the mRNAs of the vaccine encodes the EBV lytic antigens. In some embodiments, about 60% to about 80% of the mRNAs of the vaccine encodes the EBV lytic antigens. In some embodiments, about 60% to about 70% of the mRNAs of the vaccine encodes the EBV lytic antigens.

In some embodiments, about 20% to about 50% of the mRNAs of the vaccine encodes the EBV latent antigens. In some embodiments, about 20% to about 40% of the mRNAs of the vaccine encodes the EBV latent antigens. In some embodiments, about 20% to about 30% of the mRNAs of the vaccine encodes the EBV latent antigens. In some embodiments, about 30% to about 50% of the mRNAs of the vaccine encodes the EBV latent antigens. In some embodiments, about 30% to about 40% of the mRNAs of the vaccine encodes the EBV latent antigens.

In some embodiments, the EBV lytic antigens are EBV glycoproteins.

In some embodiments, the EBV glycoproteins are selected from EBV glycoprotein 350 (gp330), EBV glycoprotein 220 (gp220), EBV glycoprotein H (gH), EBV glycoprotein L (gL), EBV glycoprotein 42 (gp42), and EBV glycoprotein B (gB).

In some embodiments, the EBV glycoproteins comprise EBV gp220, EBV gH, EBV gL, and EBV gp42.

In some embodiments, the EBV latent antigens are selected from EBV nuclear antigen 1 (EBNA1), EBV nuclear antigen 2 (EBNA2), EBV nuclear antigen 3A (EBNA3A), EBV nuclear antigen 3B (EBNA3B), EBV nuclear antigen 3C (EBNA3C), EBV latent membrane protein 1 (LMP1), EBV latent membrane protein 2A (LMP2A), and EBV latent membrane protein 2B (LMP2B).

In some embodiments, the EBV latent antigens comprise EBNA3A and LMP2B.

In some embodiments: about 25% to about 45% of the mRNAs encode EBV gp220; about 10% to about 20% of the mRNAs encode EBV gH; about 5% to about 15% of the mRNAs encode EBV gL; about 5% to about 15% of the mRNAs encode EBV gp42; about 10% to about 25% of the mRNAs encode EBBA3A; and about 10% to about 25% of the mRNAs encode EBV LMP2B.

In some embodiments: about 40% to about 45% of the mRNAs encode EBV gp220; about 15% to about 20% of the mRNAs encode EBV gH; about 10% to about 15% of the mRNAs encode EBV gL; about 10% to about 15% of the mRNAs encode EBV gp42; about 10% to about 15% of the mRNAs encode EBBA3A; and about 10% to about 15% of the mRNAs encode EBV LMP2B.

In some embodiments: about 42% of the mRNAs encode EBV gp220; about 16% of the mRNAs encode EBV gH; about 10.5% of the mRNAs encode EBV gL; about 10.5% of the mRNAs encode EBV gp42; about 10.5% of the mRNAs encode EBBA3A; and about 10.5% of the mRNAs encode EBV LMP2B.

In some embodiments: about 25% to about 30% of the mRNAs encode EBV gp220; about 10% to about 15% of the mRNAs encode EBV gH; about 5% to about 10% of the mRNAs encode EBV gL; about 5% to about 10% of the mRNAs encode EBV gp42; about 20% to about 25% of the mRNAs encode EBBA3A; and about 20% to about 25% of the mRNAs encode EBV LMP2B.

In some embodiments: about 27% of the mRNAs encode EBV gp220; about 10% of the mRNAs encode EBV gH; about 6.5% of the mRNAs encode EBV gL; about 6.5% of the mRNAs encode EBV gp42; about 25% of the mRNAs encode EBBA3A; and about 25% of the mRNAs encode EBV LMP2B.

In some embodiments, the C-terminus of the EBNA3A is truncated relative to the naturally occurring EBNA3A.

In some embodiments, the EBNA3A lacks the C-terminal transcriptional regulator domain.

In some embodiments, the EBNA3A has a length of about 523 amino acids.

In some embodiments, the EBNA3A comprises amino acid residues corresponding to amino acid residues 1-523 of the naturally occurring EBNA3A.

In some embodiments, the EBNA3A does not include amino acid residues corresponding to amino acid residues 524-944 of the naturally occurring EBNA3A.

In some embodiments, the N-terminus of the EBNA3A is truncated relative to the naturally occurring EBNA3A.

In some embodiments, the EBNA3A has a length of about 455 amino acids.

In some embodiments, the EBNA3A comprises amino acid residues corresponding to amino acid residues 68-523 of the naturally occurring EBNA3A.

In some embodiments, the EBNA3A does not include amino acid residues corresponding to amino acid residues 1-67 and 524-944 of the naturally occurring EBNA3A.

In some embodiments, one or more nuclear localization signals (NLS) in the EBNA3A are mutated relative to the naturally occurring EBNA3A.

In some embodiments, 1-4, 2-4, 3-4, or 4 of the NLS in the EBNA3A are mutated relative to the naturally occurring EBNA3A.

In some embodiments, the mutated NLS are at amino acid positions selected from: 63-66, 146-155, 375-381, and 394-398, relative to the naturally occurring EBNA3A.

In some embodiments, the C-terminus of the EBNA3A is truncated at amino acid residue 68, the N-terminus of the EBNA3A is truncated at amino acid residue 523, and the EBNA3 comprises NLS mutations at amino acid residues 146-155, 375-378, and 394-398, relative to the naturally occurring EBNA3A.

In some embodiments, the EBV gp220 comprises an amino acid sequence having at least 80%, at least 85%, at least 90%, at least 95%, or 100% identity to the amino acid sequence of SEQ ID NO: 1.

In some embodiments, the mRNA encoding EBV gp220 comprises an open reading frame comprising a nucleic acid sequence having at least 80%, at least 85%, at least 90%, at least 95%, or 100% identity to the nucleic acid sequence of SEQ ID NO: 7.

In some embodiments, the mRNA encoding EBV gp220 comprises a nucleic acid sequence having at least 80%, at least 85%, at least 90%, at least 95%, or 100% identity to the nucleic acid sequence of SEQ ID NO: 13.

In some embodiments, the EBV gH comprises an amino acid sequence having at least 80%, at least 85%, at least 90%, at least 95%, or 100% identity to the amino acid sequence of SEQ ID NO: 2.

In some embodiments, the mRNA encoding EBV gH comprises an open reading frame comprising a nucleic acid sequence having at least 80%, at least 85%, at least 90%, at least 95%, or 100% identity to the nucleic acid sequence of SEQ ID NO: 8.

In some embodiments, the mRNA encoding EBV gH comprises a nucleic acid sequence having at least 80%, at least 85%, at least 90%, at least 95%, or 100% identity to the nucleic acid sequence of SEQ ID NO: 14.

In some embodiments, the EBV gL comprises an amino acid sequence having at least 80%, at least 85%, at least 90%, at least 95%, or 100% identity to the amino acid sequence of SEQ ID NO: 3.

In some embodiments, the mRNA encoding EBV gL comprises an open reading frame comprising a nucleic acid sequence having at least 80%, at least 85%, at least 90%, at least 95%, or 100% identity to the nucleic acid sequence of SEQ ID NO: 9.

In some embodiments, the mRNA encoding EBV gL comprises a nucleic acid sequence having at least 80%, at least 85%, at least 90%, at least 95%, or 100% identity to the nucleic acid sequence of SEQ ID NO: 15.

In some embodiments, the EBV gp42 is soluble.

In some embodiments, the EBV gp42 comprises an amino acid sequence having at least 80%, at least 85%, at least 90%, at least 95%, or 100% identity to the amino acid sequence of any one of SEQ ID NOs: 4 or 21.

In some embodiments, the mRNA encoding EBV gp42 comprises an open reading frame comprising a nucleic acid sequence having at least 80%, at least 85%, at least 90%, at least 95%, or 100% identity to the nucleic acid sequence of SEQ ID NO: 10.

In some embodiments, the mRNA encoding EBV gp42 comprises a nucleic acid sequence having at least 80%, at least 85%, at least 90%, at least 95%, or 100% identity to the nucleic acid sequence of SEQ ID NO: 16.

In some embodiments, the EBNA3A comprises an amino acid sequence having at least 80%, at least 85%, at least 90%, at least 95%, or 100% identity to the amino acid sequence of any one of SEQ ID NOs: 5 or 22; or having at least 80%, at least 85%, at least 90%, at least 95%, or 100% identity to the amino acid sequence of any one of SEQ ID NOs: 28-31.

In some embodiments, the mRNA encoding EBNA3A comprises an open reading frame comprising a nucleic acid sequence having at least 80%, at least 85%, at least 90%, at least 95%, or 100% identity to the nucleic acid sequence of SEQ ID NO: 11; or having at least 80%, at least 85%, at least 90%, at least 95%, or 100% identity to the nucleic acid sequence of any one of SEQ ID NOs: 24-27.

In some embodiments, the mRNA encoding EBNA3A comprises a nucleic acid sequence having at least 80%, at least 85%, at least 90%, at least 95%, or 100% identity to the nucleic acid sequence of SEQ ID NO: 17; or having at least 80%, at least 85%, at least 90%, at least 95%, or 100% identity to the nucleic acid sequence of any one of SEQ ID NOs: 32-35.

In some embodiments, the EBV LMP2B comprises an amino acid sequence having at least 80%, at least 85%, at least 90%, at least 95%, or 100% identity to the amino acid sequence of any one of SEQ ID NOs: 6 or 23.

In some embodiments, the mRNA encoding EBV LMP2B comprises an open reading frame comprising a nucleic acid sequence having at least 80%, at least 85%, at least 90%, at least 95%, or 100% identity to the nucleic acid sequence of SEQ ID NO: 12.

In some embodiments, the mRNA encoding EBV LMP2B comprises a nucleic acid sequence having at least 80%, at least 85%, at least 90%, at least 95%, or 100% identity to the nucleic acid sequence of SEQ ID NO: 18.

In some embodiments, one or more of the mRNAs comprise a chemical modification.

In some embodiments, 100% of the uracil nucleotides of the one or more mRNAs comprise a chemical modification.

In some embodiments, the chemical modification is 1-methylpseudouracil.

In some embodiments, the lipid nanoparticle comprises an ionizable lipid, a neutral lipid, a sterol, and a PEG-modified lipid.

In some embodiments, the lipid nanoparticle comprises 40-60 mol % ionizable lipid, 5-20 mol % neutral lipid, 30-50 mol % sterol, and 0.5-5 mol % PEG-modified lipid.

In some embodiments, the ionizable lipid is a compound of Formula (AIII):

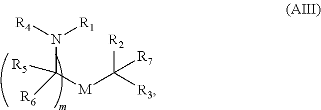

wherein R₁ is R″M′R′ or C₅₋₂₀ alkenyl; R₂ and R₃ are each independently selected from C₁₋₁₄ alkyl and C₂₋₁₄ alkenyl; R₄ is —(CH₂)ₙQ, wherein Q is OH and n is selected from 3, 4, and 5; M and M′ are each independently —OC(O)— or —C(O)O—; R₅, R₆, and R₇ are each H; R′ is a linear C₁₋₁₂ alkyl, or C₁₋₁₂ alkyl substituted with C₆₋₉ alkyl; R″ is C₃₋₄ alkyl; m is selected from 5, 6, 7, 8, 9, 10, 11, 12, and 13.

In some embodiments: R₁ is R″M′R′; R₂ and R₃ are each independently C₁₋₁₄ alkyl; R₄ is —(CH₂)ₙQ, wherein Q is OH and n is 4; M and M′ are each independently —OC(O)—; R₅, R₆, and R₇ are each H; R′ is C₁₋₁₂ alkyl substituted with C₆₋₉ alkyl; R″ is C₃₋₁₄ alkyl; and m is 6.

In some embodiments: R₁ is C₅₋₂₀ alkenyl; R₂ and R₃ are each independently C₁₋₁₄ alkyl; R₄ is —(CH₂)ₙQ, wherein Q is OH and n is 3; M is —C(O)O—; R₅, R₆, and R₇ are each H; and m is 6.

In some embodiments, the ionizable lipid is a compound of Formula (AIII):

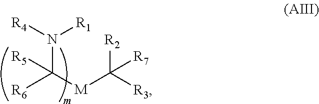

wherein R₁ is selected from the group consisting of C₅₋₃₀ alkyl, C₅₋₂₀ alkenyl, and —R″M′R′; R₂ and R₃ are independently selected from the group consisting of C₁₋₁₄ alkyl and C₂₋₁₄ alkenyl; R₄ is —(CH₂)ₙQ, wherein Q is —OR, and n is selected from 1, 2, 3, 4, and 5; each R₅ is H; each R₆ is H; M and M′ are independently selected from —C(O)O— and —OC(O)—; R₇ is H; R is H; R′ is selected from the group consisting of C₁₋₁₈ alkyl and C₂₋₁₈ alkenyl; R″ is selected from the group consisting of C₃₋₁₄ alkyl and C₃₋₁₄ alkenyl; and m is selected from 5, 6, 7, 8, 9, 10, 11, 12, and 13.

In some embodiments, the compound is:

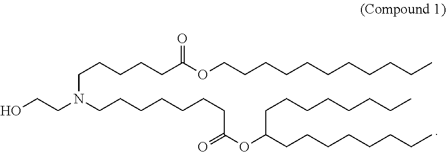 [SMILES: CCCCCCCCCCCOC(=O)CCCCCN(CCO)CCCCCCCC(=O)OC(CCCCCCCC)CCCCCCCC]

In some embodiments, the neutral lipid is 1,2-distearoyl-sn-glycero-3-phosphocholine (DSPC). In some embodiments, the sterol is cholesterol. In some embodiments, the PEG-modified lipid is PEG-DMG.

Other aspects of the present disclosure relate to a method, comprising administering to a subject the EBV mRNA vaccine of any one of the preceding claims in a therapeutically effective amount.

In some embodiments, the therapeutically effective amount is one or more 25-150 μg, 25-100 μg, 25-50 μg, 50-150 μg, or 50-100 μg doses of the EBV mRNA vaccine. In some embodiments, the therapeutically effective amount is one or more 25 μg doses of the EBV mRNA vaccine. In some embodiments, the therapeutically effective amount is one or more 50 μg doses of the EBV mRNA vaccine. In some embodiments, the therapeutically effective amount is one or more 100 μg doses of the EBV mRNA vaccine. In some embodiments, the therapeutically effective amount is one or more 150 μg doses of the EBV mRNA vaccine.

In some embodiments, the subject has been infected with EBV.

In some embodiments, the subject is a patient who has received, or is scheduled to receive, a solid organ transplant or a hematopoietic stem cell transplant.

In some embodiments, the subject has, or is at risk of having, a posttransplant lymphoproliferative disorder.

In some embodiments, the subject has, or is at risk of having, infectious mononucleosis.

In some embodiments, the subject has, or is at risk of having, multiple sclerosis.

In some embodiments, the subject has, or is at risk of having, systemic lupus erythematosus.

In some embodiments, the subject has, or is at risk of having, cancer. In some embodiments, the cancer is selected from Burkitt lymphoma, Hodgkin's lymphoma, nasopharyngeal carcinoma, and gastric cancer.

In some embodiments, the subject is EBV-seropositive. In other embodiments, the subject is EBV-seronegative.

In some embodiments, the subject is 18 to 55 years old.

In some embodiments, the EBV mRNA is administered intramuscularly, intravenously, or intranasally.

In some embodiments, the effective amount induces a neutralizing antibody response.

In some embodiments, the effective amount induces a CD4+ T cell response. In some embodiments, the effective amount induces a CD8+ T cell response.

### BRIEF DESCRIPTION OF THE DRAWINGS

FIGS. 1A-1F shows CD8⁺ T cell responses after stimulation by LMP2 (FIG. 1A), EBNA3A (FIG. 1B), gH (FIG. 1C), gL (FIG. 1D), gp42 (FIG. 1E) or gp220 (FIG. 1F). The y-axis shows the percentage (%) of CD8⁺ T cells that have a marker selected from, CD107a, IFN-γ, TNF-α, or IL-2. The x-axis shows which drug product (DP) were administered and at which dose (μg mRNA).

FIGS. 2A-2F shows CD4⁺ T cell responses after stimulation by LMP2 (FIG. 2A), EBNA3A (FIG. 2B), gH (FIG. 2C), gL (FIG. 2D), gp42 (FIG. 2E) or gp220 (FIG. 2F). The y-axis shows the percentage (%) of CD4⁺ T cells that have a marker selected from, IFN-γ, TNF-α, IL-2, IL-4, IL-5, or IL-13. The x-axis shows which drug product (DP) were administered and at which dose (μg mRNA).

FIGS. 3A-3B show the results of a B cell (FIG. 3A) or epithelial cell (FIG. 3B) neutralization assay. The y-axis shows the levels of 50% neutralizing titer (NT₅₀) (log₁₀). The x-axis shows which drug product (DP) were administered and at which dose (μg mRNA). The dashed line indicates limit of detection (LOD).

FIGS. 4A-4C show results from Luminex® assay for gHgL (FIG. 4A), gp42 (FIG. 4B) or gp220 (FIG. 4C). The y-axis shows the percentage the Luminex Average Net Mean Fluorescence Intensity (MFI) (log₁₀). The x-axis shows which drug product (DP) were administered and at which dose (μg mRNA).

FIG. 5 shows EBNA3A and LMP2b-specific CD8+ T cell responses. CD8⁺ T cell responses measured by ICS in mice immunized with EBNA3A and LMP2b. All groups received 1 μg of the specific antigens on Day 1 and 29. ICS analyses on harvested spleens were performed on Day 36. Y axis indicates the percentage of CD8⁺ T cell population expressing indicated cytokine. Error bars indicate the standard error of the mean (SEM).

FIG. 6 shows EBNA3A and LMP2b-specific CD4+ T cell responses. CD4+ T cell responses measured by ICS in mice immunized with EBNA3A and LMP2b. All groups received 1 μg of the specific antigens on Day 1 and 29. ICS analyses on harvested spleens were performed on Day 36. Y axis indicates the percentage of CD4+ T cell population expressing indicated cytokine. Error bars indicate the standard error of the mean (SEM).

FIGS. 7A-7B are graphs depicting in vitro CD8 T cell cytotoxicity against EBV-transformed lymphoblastoid B cell lines (LCL) EBV latent antigen-specific & non-specific T cells were expanded from healthy donors by coculture with monocyte-derived dendritic cells (DC) transfected with EBNA3A & LMP2b latent mRNA antigens or negative control mRNA for 14 days. Antigen-specific T cells were isolated and cocultured with autologous EBV-transformed lymphoblastoid B cell lines (LCL) for 6 hours at 37 C at increasing T cells:LCL ratios (Effector:Target). Samples were then stained with Caspase 3/7 detection reagent and a Live Dead stain, to detect apoptotic LCLs. (FIGS. 7A-7B) Percentage of apoptotic LCLs after coincubation with autologous antigen specific (EBV or Control) CD8 T cells. Data presented as mean±SEM. Samples run in duplicate. Background apoptosis levels (No T cells) subtracted from CD8 T cell co-incubated samples.

### DETAILED DESCRIPTION

The present disclosure provides therapeutic as well as preventative vaccines that include messenger RNA (mRNA) comprising an open reading frame encoding an Epstein-Barr Virus (EBV) protein. EBV, a γ-herpesvirus, is an enveloped, double-stranded DNA virus in the Herpesviridae family. Epstein-Barr virus is responsible for 90% of infectious mononucleosis cases worldwide. It utilizes several surface glycoproteins to mediate viral entry into B cells and epithelial cells. These include the principal glycoproteins gp350, gH, gL, gp42, and gB. The gH/gL complex is part of the herpesvirus core fusion machinery required for entry into all cell types and serves as receptor binding ligand for epithelial cell entry. Together, gH/gL, gp42, and gp350 are the major targets of neutralizing antibodies preventing EBV infection of B cells and epithelial cells.

In addition to infectious mononucleosis, EBV may be responsible for almost 2% of all cancer deaths globally. Among those cancers with a strong causal relationship with EBV are Burkitt lymphoma, Hodgkin's lymphoma, gastric carcinoma, and nasopharyngeal carcinoma. As these are highly diverse cancers with different pathophysiologic features and clinical presentations, epidemiologic data on risk factors are highly variable.

Autoimmune disorders such as systemic lupus erythematosus and multiple sclerosis have also been linked to EBV infection. Data to support the role of EBV in MS has been accumulating for more than 2 decades and suggest that EBV may be necessary, but not sufficient, to cause multiple sclerosis. For example, multiple sclerosis patients have been found to have increased antibody titers to EBNAs and previous infectious mononucleosis is known to increase the risk of multiple sclerosis. In one recent study of >10 million US active-duty military personnel over 20 years, 97% of individuals who developed multiple sclerosis and were EBV-seronegative at baseline seroconverted during the follow up period while only 57% seroconverted but did not develop multiple sclerosis.

A vaccine that prevents asymptomatic or symptomatic EBV infection could have a significant effect on these EBV-associated diseases. Like all other human herpes viruses, EBV persists in the host for life, following acute infection, cycling between a latent and lytic state. Keys to this process are the so called “latent” viral proteins, which include nuclear antigens and latent membrane proteins. These proteins (antigens) have critical roles in the regulation of gene expression during EBV latency and are potent inducers of T cell-mediated immunity. Inclusion of these antigens in a vaccine that prevents B cell and epithelial cell entry could control reactivation of latent EBV and may also prevent or slow the progression of a number of EBV associated diseases, such as posttransplant lymphoproliferative disease (PTLD), systemic lupus erythematosus, multiple sclerosis, and cancer. Therefore, the present disclosure provides EBV vaccines, which in some embodiments, contain structural glycoproteins (e.g., EBV gp220, gH, gL, and gp42) and latent antigens (e.g., EBNA3A and LMP2B). The EBV mRNA vaccines described herein, containing both lytic and latent antigens, may be useful in people who are both EBV seronegative and EBV seropositive, and are at risk for EBV-associated diseases.

The vaccines disclosed herein are demonstrated to induce humoral and cell mediated immune responses. An advantage of the dual immune response for a viral infection such as that caused by EBV is significant. The latent virus has been difficult to treat. However, the cell mediated immune response induced by the latent antigens found in the vaccines provides an important advantage in reducing latent viral stores.

Messenger RNA is the carrier that transfers the blueprint for protein production from genomic DNA in the nucleus to the cytoplasm, which is the cellular site for protein synthesis. In the normal cell, mRNA is transcribed in the nucleus from genomic DNA and is transported from the nucleus to the cytoplasm, where it is translated into protein. Messenger RNA does not interact with the genome, is nonreplicating, delivers only the genetic elements required for expression of the encoded protein, and its effect is transient and dose dependent. The basic structure of all forms of eukaryotic mRNA is similar: polymers of ribonucleotides of varying lengths with each ribonucleotide joined at its terminal 3′ hydroxyl group to the 5′-phosphate group of the succeeding ribonucleotide. The monomeric ribonucleotides contain the bases guanine, uracil, adenine, or cytosine with the base covalently bound to the ribose-phosphate backbone. mRNA is single stranded and contains ribose as the sugar. In contrast to the deoxyribose sugars of DNA, ribose sugars have an additional hydroxyl group on the second carbon. The cap and 5′ UTR enable binding of the ribosome complex and initiation of translation of the coding region. The coding region starts at an AUG nucleotide sequence, contains a sequence of codons (nucleotide triplets) that encode the individual amino acids of the encoded protein, and is read in the 5′ to 3′ direction before being terminated by a stop codon. The 3′ UTR is at the end of the coding region and is followed by the polyA tail. The polyA tail confers stability to the RNA molecule, plays a role in the termination of transcription, and participates in the export of the mRNA molecule from the nucleus and in initiation of translation of the encoded protein.

### Epstein-Barr Virus Proteins

Some aspects of the present disclosure provide vaccines comprising mRNA having open reading frames that encode multiple EBV antigens, including EBV glycoprotein 220 (gp220), glycoprotein H (gH), glycoprotein L (gL), glycoprotein 42 (gp42), EBV Nuclear Antigen 3A (EBNA3A), and EBV Latent Membrane Protein 2B (LMP2B). In some embodiments, the EBV antigen is a lytic antigen. In some embodiments, the EBV lytic antigen is an EBV glycoprotein.

In some embodiments, the EBV glycoprotein is selected from EBV glycoprotein 350 (gp330), EBV glycoprotein 220 (gp220), EBV glycoprotein H (gH), EBV glycoprotein L (gL), EBV glycoprotein 42 (gp42), and EBV glycoprotein B (gB). In some embodiments, the EBV glycoprotein is selected from EBV gp220, gH, gL, and gp42. In some embodiments, the EBV antigen is an EBV latent antigen. In some embodiments, the EBV latent antigen is selected from EBV nuclear antigen 1 (EBNA1), EBV nuclear antigen 2 (EBNA2), EBV nuclear antigen 3A (EBNA3A), EBV nuclear antigen 3B (EBNA3B), EBV nuclear antigen 3C (EBNA3C), EBV latent membrane protein 1 (LMP1), EBV latent membrane protein 2A (LMP2A), and EBV latent membrane protein 2B (LMP2B). In some embodiments, the EBV latent antigen is selected from EBNA3A and LMP2B.

In some embodiments, a vaccine comprises a first mRNA comprising an open reading frame encoding an EBV gp220, a second mRNA comprising an open reading frame encoding an EBV gH, a third mRNA comprising an open reading frame encoding an EBV gL, a fourth mRNA comprising an open reading frame encoding an EBV gp42, a fifth mRNA comprising an open reading frame encoding an EBV EBNA3A, and a sixth mRNA comprising an open reading frame encoding an EBV LMP2B. Expression of gp220, gH, gL, gp42, EBNA3A and LMP2B by cells containing the mRNA elicits CD8+ and CD4+ T cell responses and neutralizing epithelial and B cell responses. Because EBV latent antigens are expressed in latently infected B cells and are important for efficient EBV-induced transformation of B cells in vitro, such vaccines are useful in generating potent neutralizing antibody responses that prevent or limit EBV infection. Additionally, antigens encoded by RNAs of the vaccines may be modified relative to wild-type EBV antigens to improve the T cell responses elicited by immunization or prevent the deleterious effects (e.g., removing epitopes of autoantibody) of EBV protein expression.

The terms “naturally occurring” and “wild type” are used interchangeably herein. A naturally occurring EBV protein is an unmodified EBV protein of an Epstein-Barr virus (e.g., EBV-1 or EBV-2) that occurs in nature, i.e., which is a naturally occurring isolate. As is known in the art, a naturally occurring protein is not genetically engineered. A naturally occurring protein is not genetically (or otherwise) modified to substitute, remove, or add any amino acids relative to the naturally occurring isolate. In some embodiments, the naturally occurring isolate of EBV is EBV strain B95-8. See, e.g., Bauer R et al. Nature. 1984 July; 310 (5974): 207-11. Amino acid sequences of naturally-occurring gp220, gH, gL, gp42, EBNA3A, and LMP2B of EBV strain B95-8 are provided in UniProt Accession Nos. P03200-1, P03231, P03212, P03205, P12977, and P13285, respectively. Wild-type nucleic acid and/or protein sequences may be obtained, for example, by sequencing the genome or certain genes of one or more viral isolates, and/or proteins expressed by the genome or certain genes of one or more of the viral isolates.

In some embodiments, a wild-type EBV gp220 comprises the amino acid sequence of SEQ ID NO: 1. In some embodiments, a wild-type EBV gH comprises the amino acid sequence of SEQ ID NO: 2. In some embodiments, a wild-type EBV gL comprises the amino acid sequence of SEQ ID NO: 3. In some embodiments, a wild-type EBV gp42 comprises the amino acid sequence of SEQ ID NO: 21. In some embodiments, a wild-type EBV EBNA3A comprises the amino acid sequence of SEQ ID NO: 22. In some embodiments, a wild-type EBV LMP2B comprises the amino acid sequence of SEQ ID NO: 23.

TABLE 1
Protein Sequences From a Naturally-Occurring EBV Isolate

| | | SEQ ID |
|---|---|---|
| Name | Amino Acid Sequence | NO: |
| EBV gp220 | MEAALLVCQY TIQSLIHLTG EDPGFFNVEI PEFPFYPTCN VCTADVNVTI | 1 |
| UniProt/UniRef | NFDVGGKKHQ LDLDFGQLTP HTKAVYQPRG AFGGSENATN LFLLELLGAG | |
| P03200-1 | ELALTMRSKK LPINVTTGEE QQVSLESVDV YFQDVFGTMW CHHAEMQNPV | |
| | YLIPETVPYI KWDNCNSTNI TAVVRAQGLD VTLPLSLPTS AQDSNFSVKT | |
| | EMLGNEIDIE CIMEDGEISQ VLPGDNKFNI TCSGYESHVP SGGILTSTSP | |
| | VATPIPGTGY AYSLRLTPRP VSRFLGNNSI LYVFYSGNGP KASGGDYCIQ | |
| | SNIVFSDEIP ASQDMPINTT DITYVGDNAT YSVPMVTSED ANSPNVTVTA | |
| | FWAWPNNTET DFKCKWTLTS GTPSGCENIS GAFASNRTFD ITVSGLGTAP | |
| | KTLIITRTAT NATTTTHKVI FSKAPESTTT SPTLNTTGFA DPNTTTGLPS | |
| | STHVPTNLTA PASTGPTVST ADVISPTPAG TTSGASPVTP SPSPWDNGTE | |
| | STPPQNATSP QAPSGQKTAV PTVISTGGKA NSTTGGKHTT GHGARTSTEP | |
| | TTDYGGDSTT PRPRYNATTY LPPSTSSKLR PRWTFTSPPV TTAQATVPVP | |
| | PTSQPRFSNL SMLVLQWASL AVLTLLLLLV MADCAFRRNL STSHTYTTPP | |
| | YDDAETYV | |
| EBV gH | MQLLCVFCLV LLWEVGAASL SEVKLHLDIE GHASHYTIPW TELMAKVPGL | 2 |
| UniProt/UniRef | SPEALWREAN VTEDLASMLN RYKLIYKTSG TLGIALAEPV DIPAVSEGSM | |
| P03231 | QVDASKVHPG VISGLNSPAC MLSAPLEKQL FYYIGTMLPN TRPHSYVFYQ | |
| | LRCHLSYVAL SINGDKFQYT GAMTSKFLMG TYKRVTEKGD EHVLSLVFGK | |
| | TKDLPDLRGP FSYPSLISAQ SGDYSLVIVT TFVHYANFHN YFVPNLKDMF | |
| | SRAVTMTAAS YARYVLQKLV LLEMKGGCRE PELDTETLTT MFEVSVAFFK | |
| | VGHAVGETGN GCVDLRWLAK SFFELTVLKD IIGICYGATV KGMQSYGLER | |
| | LAAMLMATVK MEELGHLTTE KQEYALRLAT VGYPKAGVYS GLIGGATSVL | |
| | LSAYNRHPLF QPLHTVMRET LFIGSHVVLR ELRLNVTTQG PNLALYQLLS | |
| | TALCSALEIG EVLRGLALGT ESGLFSPCYL SLRFDLTRDK LLSMAPQEAT | |
| | LDQAAVSNAV DGFLGRLSLE REDRDAWHLP AYKCVDRLDK VLMIIPLINV | |
| | TFIISSDREV RGSALYEAST TYLSSSLFLS PVIMNKCSQG AVAGEPRQIP | |
| | KIQNFTRTQK SCIFCGFALL SYDEKEGLET TTYITSQEVQ NSILSSNYFD | |
| | FDNLHVHYLL LTTNGTVMEI AGLYEERAHV VLAIILYFIA FALGIFLVHK | |
| | IVMFFL | |
| EBV gL | MRAVGVFLAI CLVTIFVLPT WGNWAYPCCH VTQLRAQHLL ALENISDIYL | 3 |
| UniProt/UniRef | VSNQTCDGFS LASLNSPKNG SNQLVISRCA NGLNVVSFFI SILKRSSSAL | |
| P03212 | TGHLRELLTT LETLYGSFSV EDLFGANLNR YAWHRGG | |
| EBV gp42 | MVSFKQVRVP LFTAIALVIV LLLAYFLPPR VRGGGRVAAA AITWVPKPNV | 21 |
| UniProt/UniRef | EVWPVDPPPP VNFNKTAEQE YGDKEVKLPH WTPTLHTFQV PQNYTKANCT | |
| P03205 | YCNTREYTFS YKGCCFYFTK KKHTWNGCFQ ACAELYPCTY FYGPTPDILP | |
| | VVTRNLNAIE SLWVGVYRVG EGNWTSLDGG TFKVYQIFGS HCTYVSKFST | |
| | VPVSHHECSF LKPCLCVSQR SNS | |
| EBV EBNA3 | MDKDRPGPPA LDDNMEEEVP STSVVQEQVS AGDWENVLIE LSDSSSEKEA | 22 |
| UniProt/UniRef | EDAHLEPAQK GTKRKRVDHD AGGSAPARPM LPPQPDLPGR EAILRRFPLD | |
| P12977 | LRTLLQAIGA AATRIDTRAI DQFFGSQISN TEMYIMYAMA IRQAIRDRRR | |
| | NPASRRDQAK WRLQTLAAGW PMGYQAYSSW MYSYTDHQTT PTFVHLQATL | |
| | GCTGGRRCHV TFSAGTFKLP RCTPGDRQWL YVQSSVGNIV QSCNPRYSIF | |
| | FDYMAIHRSL TKIWEEVLTP DQRVSFMEFL GFLQRTDLSY IKSFVSDALG | |
| | TTSIQTPWID DNPSTETAQA WNAGFLRGRA YGIDLLRTEG EHVEGATGET | |
| | REESEDTESD GDDEDLPCIV SRGGPKVKRP PIFIRRLHRL LLMRAGKRTE | |
| | QGKEVLEKAR GSTYGTPRPP VPKPRPEVPQ SDETATSHGS AQVPEPPTIH | |
| | LAAQGMAYPL HEQHGMAPCP VAQAPPTPLP PVSPGDQLPG VFSDGRVACA | |
| | PVPAPAGPIV RPWEPSLTQA AGQAFAPVRP PAPWTPNPPR SPSQMSVRDR | |
| | PKPVRPAPPK IAMQGPGETS GIRRARERWR QHMPVEPVPV PTVALERPVY | |
| | LARLRAEAQV KQASVEVQPP QLTQVSPQQP MEGPLVPEQQ MFPGAPFSQV | |
| | ADVVRAPGVP AMQPQYFDLP LIQPISQGAP VAPLRASMGP VPPVPATQPQ | |
| | YFDIPLTEPI NQGASAAHFL PQQPMEGPLV PEQWMFPGAA LSQSVRPGVA | |
| | QSQYFDLPLT QPINHGAPAA HFLHQPPMEG PWVPEQWMFQ GAPPSQGTDV | |
| | VQHQLDALGY TLHGLNHPGV PVSPAVNQYH LSQAAFGLPI DEDESGEGSD | |
| | TSEPCEALDL SIHGRPCPQA PEWPVQEEGG QDATEVLDLS IHGRPRPRTP | |
| | EWPVQGEGGQ NVTGPETRRV VVSAVVHMCQ DDEFPDLQDP PDEA | |
| EBV LMP2 | MGSLEMVPMG AGPPSPGGDP DGYDGGNNSQ YPSASGSSGN TPTPPNDEER | 23 |
| UniProt/UniRef | ESNEEPPPPY EDPYWGNGDR HSDYQPLGTQ DQSLYLGLQH DGNDGLPPPP | |
| P13285 | YSPRDDSSQH IYEEAGRGSM NPVCLPVIVA PYLFWLAAIA ASCFTASVST | |
| | VVTATGLALS LLLLAAVASS YAAAQRKLLT PVTVLTAVVT FFAICLTWRI | |
| | EDPPFNSLLF ALLAAAGGLQ GIYVLVMLVL LILAYRRRWR RLTVCGGIMF | |
| | LACVLVLIVD AVLQLSPLLG AVTVVSMTLL LLAFVLWLSS PGGLGTLGAA | |
| | LLTLAAALAL LASLILGTLN LTTMFLLMLL WTLVVLLICS SCSSCPLSKI | |
| | LLARLFLYAL ALLLLASALI AGGSILQTNF KSLSSTEFIP NLFCMLLLIV | |
| | AGILFILAIL TEWGSGNRTY GPVFMCLGGL LTMVAGAVWL TVMSNTLLSA | |
| | WILTAGFLIF LIGFALFGVI RCCRYCCYYC LTLESEERPP TPYRNTV | |

In some embodiments, the vaccines provide herein further comprise mRNA having an open reading frame that encodes EBV gp220. In some embodiments, the vaccines provide herein further comprise mRNA having an open reading frame that encodes EBV gH. In some embodiments, the vaccines provide herein further comprise mRNA having an open reading frame that encodes EBV gL. In some embodiments, the vaccines provide herein further comprise mRNA having an open reading frame that encodes EBV gp42. In some embodiments, the vaccines provide herein further comprise mRNA having an open reading frame that encodes EBNA3A. In some embodiments, the vaccines provide herein further comprise mRNA having an open reading frame that encodes EBV LMP2B.

### EBV Glycoprotein 220

EBV Glycoprotein 220 (gp220) is a major outer envelope glycoprotein. EBV gp220 forms a complex with EBV Glycoprotein 350 (gp350). The gp220/gp350 complex initiates B cell infection because it is a ligand for the B-lymphocyte plasma membrane protein, CR2. EBV gp220 binds to CR2 with similar affinity as the gp220/gp350 complex. EBV gp220/gp350 is the predominant EBV ligand for B lymphocytes. An example of a wild-type EBV gp220 is provided in UniProtKB: P03200-1. In some embodiments, an mRNA vaccine comprises an mRNA encoding an EBV gp220 comprising an amino acid sequence having at least 80%, at least 85%, at least 90%, at least 95%, at least 96%, at least 97%, at least 98%, at least 99%, or 100% identity to the amino acid sequence of SEQ ID NO: 1. In some embodiments, an mRNA vaccine comprises an mRNA encoding an EBV gp220 comprising an amino acid sequence having at least 80% identity to the amino acid sequence of SEQ ID NO: 1. In some embodiments, an mRNA vaccine comprises an mRNA encoding an EBV gp220 comprising an amino acid sequence having at least 85% identity to the amino acid sequence of SEQ ID NO: 1. In some embodiments, an mRNA vaccine comprises an mRNA encoding an EBV gp220 comprising an amino acid sequence having at least 90% identity to the amino acid sequence of SEQ ID NO: 1. In some embodiments, an mRNA vaccine comprises an mRNA encoding an EBV gp220 comprising an amino acid sequence having at least 95% identity to the amino acid sequence of SEQ ID NO: 1. In some embodiments, an mRNA vaccine comprises an mRNA encoding an EBV gp220 comprising the amino acid sequence of SEQ ID NO: 1.

In some embodiments, an mRNA comprises an open reading frame comprising a nucleic acid sequence having at least 70% identity to the nucleic acid sequence of SEQ ID NO: 7. In some embodiments, an mRNA comprises an open reading frame comprising a nucleic acid sequence having at least 75% identity to the nucleic acid sequence of SEQ ID NO: 7. In some embodiments, an mRNA comprises an open reading frame comprising a nucleic acid sequence having at least 80% identity to the nucleic acid sequence of SEQ ID NO: 7. In some embodiments, an mRNA comprises an open reading frame comprising a nucleic acid sequence having at least 85% identity to the nucleic acid sequence of SEQ ID NO: 7. In some embodiments, an mRNA comprises an open reading frame comprising a nucleic acid sequence having at least 90% identity to the nucleic acid sequence of SEQ ID NO: 7. In some embodiments, an mRNA comprises an open reading frame comprising a nucleic acid sequence having at least 95% identity to the nucleic acid sequence of SEQ ID NO: 7. In some embodiments, an mRNA comprises an open reading frame comprising the nucleic acid sequence of SEQ ID NO: 7.

### EBV Glycoprotein H

EBV Glycoprotein H (gH) is a glycoprotein that is important for EBV entry into both B cells and epithelial cells. EBV gH forms a complex with gL. The gHgL complex can also form a complex with gp42. The gHgL/gp42 complex noncovalently linked to glycoprotein gB is required for EBV to fuse with the B cell membrane. An example of a wild-type EBV gH is provided in UniProtKB: P03231. In some embodiments, an mRNA vaccine comprises an mRNA encoding an EBV gH comprising an amino acid sequence having at least 80%, at least 85%, at least 90%, at least 95%, at least 96%, at least 97%, at least 98%, at least 99%, or 100% identity to the amino acid sequence of SEQ ID NO: 2. In some embodiments, an mRNA vaccine comprises an mRNA encoding an EBV gH comprising an amino acid sequence having at least 80% identity to the amino acid sequence of SEQ ID NO: 2. In some embodiments, an mRNA vaccine comprises an mRNA encoding an EBV gH comprising an amino acid sequence having at least 85% identity to the amino acid sequence of SEQ ID NO: 2. In some embodiments, an mRNA vaccine comprises an mRNA encoding an EBV gH comprising an amino acid sequence having at least 90% identity to the amino acid sequence of SEQ ID NO: 2. In some embodiments, an mRNA vaccine comprises an mRNA encoding an EBV gH comprising an amino acid sequence having at least 95% identity to the amino acid sequence of SEQ ID NO: 2. In some embodiments, an mRNA vaccine comprises an mRNA encoding an EBV gH comprising the amino acid sequence of SEQ ID NO: 2.

In some embodiments, an mRNA comprises an open reading frame comprising a nucleic acid sequence having at least 70% identity to the nucleic acid sequence of SEQ ID NO: 8. In some embodiments, an mRNA comprises an open reading frame comprising a nucleic acid sequence having at least 75% identity to the nucleic acid sequence of SEQ ID NO: 8. In some embodiments, an mRNA comprises an open reading frame comprising a nucleic acid sequence having at least 80% identity to the nucleic acid sequence of SEQ ID NO: 8. In some embodiments, an mRNA comprises an open reading frame comprising a nucleic acid sequence having at least 85% identity to the nucleic acid sequence of SEQ ID NO: 8. In some embodiments, an mRNA comprises an open reading frame comprising a nucleic acid sequence having at least 90% identity to the nucleic acid sequence of SEQ ID NO: 8. In some embodiments, an mRNA comprises an open reading frame comprising a nucleic acid sequence having at least 95% identity to the nucleic acid sequence of SEQ ID NO: 8. In some embodiments, an mRNA comprises an open reading frame comprising the nucleic acid sequence of SEQ ID NO: 8.

### EBV Glycoprotein L

EBV Glycoprotein L (gL) is a glycoprotein that is important for EBV entry into both B cells and epithelial cells. EBV gL serves as a chaperone for EBV gH. An example of a wild-type EBV gL is provided in UniProtKB: P03212. In some embodiments, an mRNA vaccine comprises an mRNA encoding an EBV gL comprising an amino acid sequence having at least 80%, at least 85%, at least 90%, at least 95%, at least 96%, at least 97%, at least 98%, at least 99%, or 100% identity to the amino acid sequence of SEQ ID NO: 3. In some embodiments, an mRNA vaccine comprises an mRNA encoding an EBV gL comprising an amino acid sequence having at least 80% identity to the amino acid sequence of SEQ ID NO: 3. In some embodiments, an mRNA vaccine comprises an mRNA encoding an EBV gL comprising an amino acid sequence having at least 85% identity to the amino acid sequence of SEQ ID NO: 3. In some embodiments, an mRNA vaccine comprises an mRNA encoding an EBV gL comprising an amino acid sequence having at least 90% identity to the amino acid sequence of SEQ ID NO: 3. In some embodiments, an mRNA vaccine comprises an mRNA encoding an EBV gL comprising an amino acid sequence having at least 95% identity to the amino acid sequence of SEQ ID NO: 3. In some embodiments, an mRNA vaccine comprises an mRNA encoding an EBV gL comprising the amino acid sequence of SEQ ID NO: 3.

In some embodiments, an mRNA comprises an open reading frame comprising a nucleic acid sequence having at least 70% identity to the nucleic acid sequence of SEQ ID NO: 9. In some embodiments, an mRNA comprises an open reading frame comprising a nucleic acid sequence having at least 75% identity to the nucleic acid sequence of SEQ ID NO: 9. In some embodiments, an mRNA comprises an open reading frame comprising a nucleic acid sequence having at least 80% identity to the nucleic acid sequence of SEQ ID NO: 9. In some embodiments, an mRNA comprises an open reading frame comprising a nucleic acid sequence having at least 85% identity to the nucleic acid sequence of SEQ ID NO: 9. In some embodiments, an mRNA comprises an open reading frame comprising a nucleic acid sequence having at least 90% identity to the nucleic acid sequence of SEQ ID NO: 9. In some embodiments, an mRNA comprises an open reading frame comprising a nucleic acid sequence having at least 95% identity to the nucleic acid sequence of SEQ ID NO: 9. In some embodiments, an mRNA comprises an open reading frame comprising the nucleic acid sequence of SEQ ID NO: 9.

### EBV Glycoprotein 42

EBV Glycoprotein 42 (gp42) is a glycoprotein that is important for EBV entry into B cells. The gHgL/gp42 complexes bind HLA class II to activate membrane fusion with B cells, however gp42 inhibits fusion and entry into epithelial cells. EBV gp42 interacts with HLA class II, an essential coreceptor for B-cell infection, because epithelial cells do not express HLA class II, EBV gp42 is dispensable for entry into epithelial cells. An example of a wild-type EBV gp42 protein is provided in UniProtKB: P03205. In some embodiments, the EBV gp42 protein encoded by an mRNA is a soluble (secreted) form of the protein. For example, the EBV gp42 may include an N-terminal truncation relative to a naturally occurring EBV gp42 protein. In some embodiments, an mRNA encodes a soluble EBV gp42 protein that lacks the first 41 amino acid residues, and therefore, lacks the endogenous transmembrane anchor of the protein. In some embodiments, the endogenous transmembrane anchor of the soluble EBV gp42 protein is replaced with a secretion signal, such as the secretion signal from EBV gB, thereby producing the ‘soluble’ or ‘secreted’ form of EBV gp42.

In some embodiments, an mRNA vaccine comprises an mRNA encoding an EBV gp42 comprising an amino acid sequence having at least 80%, at least 85%, at least 90%, at least 95%, at least 96%, at least 97%, at least 98%, at least 99%, or 100% identity to the amino acid sequence of SEQ ID NO: 4. In some embodiments, an mRNA vaccine comprises an mRNA encoding an EBV gp42 comprising an amino acid sequence having at least 80% identity to the amino acid sequence of SEQ ID NO: 4. In some embodiments, an mRNA vaccine comprises an mRNA encoding an EBV gp42 comprising an amino acid sequence having at least 85% identity to the amino acid sequence of SEQ ID NO: 4. In some embodiments, an mRNA vaccine comprises an mRNA encoding an EBV gp42 comprising an amino acid sequence having at least 90% identity to the amino acid sequence of SEQ ID NO: 4. In some embodiments, an mRNA vaccine comprises an mRNA encoding an EBV gp42 comprising an amino acid sequence having at least 95% identity to the amino acid sequence of SEQ ID NO: 4. In some embodiments, an mRNA vaccine comprises an mRNA encoding an EBV gp42 comprising the amino acid sequence of SEQ ID NO: 4.

In some embodiments, an mRNA vaccine comprises an mRNA encoding an EBV gp42 comprising an amino acid sequence having at least 80%, at least 85%, at least 90%, at least 95%, at least 96%, at least 97%, at least 98%, at least 99%, or 100% identity to the amino acid sequence of SEQ ID NO: 21. In some embodiments, an mRNA vaccine comprises an mRNA encoding an EBV gp42 comprising an amino acid sequence having at least 80% identity to the amino acid sequence of SEQ ID NO: 21. In some embodiments, an mRNA vaccine comprises an mRNA encoding an EBV gp42 comprising an amino acid sequence having at least 85% identity to the amino acid sequence of SEQ ID NO: 21. In some embodiments, an mRNA vaccine comprises an mRNA encoding an EBV gp42 comprising an amino acid sequence having at least 90% identity to the amino acid sequence of SEQ ID NO: 21. In some embodiments, an mRNA vaccine comprises an mRNA encoding an EBV gp42 comprising an amino acid sequence having at least 95% identity to the amino acid sequence of SEQ ID NO: 21. In some embodiments, an mRNA vaccine comprises an mRNA encoding an EBV gp42 comprising the amino acid sequence of SEQ ID NO: 21.

In some embodiments, an mRNA comprises an open reading frame comprising a nucleic acid sequence having at least 70% identity to the nucleic acid sequence of SEQ ID NO: 10. In some embodiments, an mRNA comprises an open reading frame comprising a nucleic acid sequence having at least 75% identity to the nucleic acid sequence of SEQ ID NO: 10. In some embodiments, an mRNA comprises an open reading frame comprising a nucleic acid sequence having at least 80% identity to the nucleic acid sequence of SEQ ID NO: 10. In some embodiments, an mRNA comprises an open reading frame comprising a nucleic acid sequence having at least 85% identity to the nucleic acid sequence of SEQ ID NO: 10. In some embodiments, an mRNA comprises an open reading frame comprising a nucleic acid sequence having at least 90% identity to the nucleic acid sequence of SEQ ID NO: 10. In some embodiments, an mRNA comprises an open reading frame comprising a nucleic acid sequence having at least 95% identity to the nucleic acid sequence of SEQ ID NO: 10. In some embodiments, an mRNA comprises an open reading frame comprising the nucleic acid sequence of SEQ ID NO: 10.

### EBV Nuclear Antigen 3A

EBV Nuclear Antigen 3A (EBNA3A) is a latent antigen. EBV EBNA3A is a protein expressed during latency that is produced by B cells. EBNA3A is subcellularly localized in the nucleus and is essential for B cell transformation. EBNA3A is thought to regulate gene transcription of genes involved in cell survival or proliferation. EBNA3A has been shown to regulate a specific subset of gene targets, including Hsp70. Examples of wild-type EBV EBNA3A proteins are provided in UniProtKB: A0A0B6VPX8 and P12977.

In some embodiments, the EBNA3A encoded by an mRNA as provided herein is a variant form of the naturally occurring EBNA3A protein. For example, the EBNA3A variant (e.g., aa 1-523) may span residues 1-523 with point mutations at four nuclear localization signals (NLS) within that region and a deletion of the transcriptional regulator domain located in its C terminus (region spanning residues 524-944). The NLS mutations in EBNA3A (1-523mut) are located at residues 63-66, 146-155, 375-381, and 394-398. As another example, the EBNA3A variant (e.g., 68-523mut) may be similarly truncated at residue 523, with an additional N-terminal deletion that removes 1-56 aa region—a region associated with elevated antibody response in MS patients. The NLS mutations in EBNA3A (68-523mut) are located at residues 146-155, 375-378, and 394-398. In some embodiments, one or more (or all) of the nuclear localization signals in the EBNA3 protein have been mutated. In some embodiments, one or more T cell epitope on the EBNA3A protein is mutated. For example, the second T cell epitope may be mutated. In some embodiments, the RPPIFIRRL (SEQ ID NO: 97) epitope is mutated. In some embodiments, the FLRGRAYGI/L (SEQ ID NO: 98) epitope is mutated.

In some embodiments, the EBNA3A variant is truncated relative to a naturally occurring EBNA3A. In some embodiments, the EBNA3A variant has a deletion of the C-terminus of the EBNA3A relative to the naturally occurring EBNA3A. In some embodiments, the EBNA3A variant lacks the C-terminal transcriptional regulator domain. In some embodiments, the C-terminal transcriptional regulator domain is located at amino acid position 524-944, relative to a wild-type EBNA3A.

In some embodiments, the EBNA3A has a length of about 525 (e.g., 450, 455, 475, 500, 523, 525, 550) amino acids. In some embodiments, the EBNA3A has a length of about 450, about 455, about 475, about 500, about 523, about 525, about 550 amino acids. In some embodiments, the EBNA3A has a length of about 523 amino acids. In some embodiments, the EBNA3A has a length of about 455 amino acids. In some embodiments, the EBNA3A has a length of about 400-550 (e.g., 400-550, 400-525, 400-523, 400-500, 400-475, 400-455, 400-450, 400-425, 425-550, 425-525, 425-523, 425-500, 425-475, 425-455, 425-450, 450-550, 450-525, 450-523, 450-500, 450-475, 450-455, 455-550, 455-525, 455-523, 455-500, 455-475, 475-550, 475-525, 475-523, 475-500, 500-550, 500-525, 500-523, 523-525, 523-550, 525-550) amino acids. In some embodiments, the EBNA3A has a length of about 400-550, 400-525, 400-523, 400-500, 400-475, 400-455, 400-450, 400-425, 425-550, 425-525, 425-523, 425-500, 425-475, 425-455, 425-450, 450-550, 450-525, 450-523, 450-500, 450-475, 450-455, 455-550, 455-525, 455-523, 455-500, 455-475, 475-550, 475-525, 475-523, 475-500, 500-550, 500-525, 500-523, 523-525, 523-550, 525-550 amino acids.

In some embodiments, the EBNA3A comprises one or more nuclear localization signals (NLS) that are mutated relative to the naturally occurring EBNA3A. In some embodiments, about 1-4 (e.g., 1-4, 1-3, 1-2, 2-4, 2-3, 3-4) of the NLS in the EBNA3A are mutated relative to the naturally occurring EBNA3A. In some embodiments, 1-4, 1-3, 1-2, 2-4, 2-3, 3-4 of the NLS in the EBNA3A are mutated relative to the naturally occurring EBNA3A. In some embodiments, 1, 2, 3, or 4 of the NLS in the EBNA3A are mutated relative to the naturally occurring EBNA3A. In some embodiments, 1 of the NLS in the EBNA3A are mutated relative to the naturally occurring EBNA3A. In some embodiments, 2 of the NLS in the EBNA3A are mutated relative to the naturally occurring EBNA3A. In some embodiments, 3 of the NLS in the EBNA3A are mutated relative to the naturally occurring EBNA3A. In some embodiments, 4 of the NLS in the EBNA3A are mutated relative to the naturally occurring EBNA3A.

In some embodiments, an mRNA vaccine comprises an mRNA encoding an EBNA3A comprising an amino acid sequence having at least 80%, at least 85%, at least 90%, at least 95%, at least 96%, at least 97%, at least 98%, at least 99%, or 100% identity to the amino acid sequence of SEQ ID NO: 5. In some embodiments, an mRNA vaccine comprises an mRNA encoding an EBNA3A comprising an amino acid sequence having at least 80% identity to the amino acid sequence of SEQ ID NO: 5. In some embodiments, an mRNA vaccine comprises an mRNA encoding an EBNA3A comprising an amino acid sequence having at least 85% identity to the amino acid sequence of SEQ ID NO: 5. In some embodiments, an mRNA vaccine comprises an mRNA encoding an EBNA3A comprising an amino acid sequence having at least 90% identity to the amino acid sequence of SEQ ID NO: 5. In some embodiments, an mRNA vaccine comprises an mRNA encoding an EBNA3A comprising an amino acid sequence having at least 95% identity to the amino acid sequence of SEQ ID NO: 5. In some embodiments, an mRNA vaccine comprises an mRNA encoding an EBNA3A comprising the amino acid sequence of SEQ ID NO: 5.

In some embodiments, an mRNA vaccine comprises an mRNA encoding an EBNA3A comprising an amino acid sequence having at least 80%, at least 85%, at least 90%, at least 95%, at least 96%, at least 97%, at least 98%, at least 99%, or 100% identity to the amino acid sequence of SEQ ID NO: 22. In some embodiments, an mRNA vaccine comprises an mRNA encoding an EBNA3A comprising an amino acid sequence having at least 80% identity to the amino acid sequence of SEQ ID NO: 22. In some embodiments, an mRNA vaccine comprises an mRNA encoding an EBNA3A comprising an amino acid sequence having at least 85% identity to the amino acid sequence of SEQ ID NO: 22. In some embodiments, an mRNA vaccine comprises an mRNA encoding an EBNA3A comprising an amino acid sequence having at least 90% identity to the amino acid sequence of SEQ ID NO: 22. In some embodiments, an mRNA vaccine comprises an mRNA encoding an EBNA3A comprising an amino acid sequence having at least 95% identity to the amino acid sequence of SEQ ID NO: 22. In some embodiments, an mRNA vaccine comprises an mRNA encoding an EBNA3A comprising the amino acid sequence of SEQ ID NO: 22.

In some embodiments, an mRNA vaccine comprises an mRNA encoding an EBNA3A comprising an amino acid sequence having at least 80%, at least 85%, at least 90%, at least 95%, at least 96%, at least 97%, at least 98%, at least 99%, or 100% identity to the amino acid sequence of SEQ ID NO: 28. In some embodiments, an mRNA vaccine comprises an mRNA encoding an EBNA3A comprising an amino acid sequence having at least 80% identity to the amino acid sequence of SEQ ID NO: 28. In some embodiments, an mRNA vaccine comprises an mRNA encoding an EBNA3A comprising an amino acid sequence having at least 85% identity to the amino acid sequence of SEQ ID NO: 28. In some embodiments, an mRNA vaccine comprises an mRNA encoding an EBNA3A comprising an amino acid sequence having at least 90% identity to the amino acid sequence of SEQ ID NO: 28. In some embodiments, an mRNA vaccine comprises an mRNA encoding an EBNA3A comprising an amino acid sequence having at least 95% identity to the amino acid sequence of SEQ ID NO: 28. In some embodiments, an mRNA vaccine comprises an mRNA encoding an EBNA3A comprising the amino acid sequence of SEQ ID NO: 28.

In some embodiments, an mRNA vaccine comprises an mRNA encoding an EBNA3A comprising an amino acid sequence having at least 80%, at least 85%, at least 90%, at least 95%, at least 96%, at least 97%, at least 98%, at least 99%, or 100% identity to the amino acid sequence of SEQ ID NO: 29. In some embodiments, an mRNA vaccine comprises an mRNA encoding an EBNA3A comprising an amino acid sequence having at least 80% identity to the amino acid sequence of SEQ ID NO: 29. In some embodiments, an mRNA vaccine comprises an mRNA encoding an EBNA3A comprising an amino acid sequence having at least 85% identity to the amino acid sequence of SEQ ID NO: 29. In some embodiments, an mRNA vaccine comprises an mRNA encoding an EBNA3A comprising an amino acid sequence having at least 90% identity to the amino acid sequence of SEQ ID NO: 29. In some embodiments, an mRNA vaccine comprises an mRNA encoding an EBNA3A comprising an amino acid sequence having at least 95% identity to the amino acid sequence of SEQ ID NO: 29. In some embodiments, an mRNA vaccine comprises an mRNA encoding an EBNA3A comprising the amino acid sequence of SEQ ID NO: 29.

In some embodiments, an mRNA vaccine comprises an mRNA encoding an EBNA3A comprising an amino acid sequence having at least 80%, at least 85%, at least 90%, at least 95%, at least 96%, at least 97%, at least 98%, at least 99%, or 100% identity to the amino acid sequence of SEQ ID NO: 30. In some embodiments, an mRNA vaccine comprises an mRNA encoding an EBNA3A comprising an amino acid sequence having at least 80% identity to the amino acid sequence of SEQ ID NO: 30. In some embodiments, an mRNA vaccine comprises an mRNA encoding an EBNA3A comprising an amino acid sequence having at least 85% identity to the amino acid sequence of SEQ ID NO: 30. In some embodiments, an mRNA vaccine comprises an mRNA encoding an EBNA3A comprising an amino acid sequence having at least 90% identity to the amino acid sequence of SEQ ID NO: 30. In some embodiments, an mRNA vaccine comprises an mRNA encoding an EBNA3A comprising an amino acid sequence having at least 95% identity to the amino acid sequence of SEQ ID NO: 30. In some embodiments, an mRNA vaccine comprises an mRNA encoding an EBNA3A comprising the amino acid sequence of SEQ ID NO: 30.

In some embodiments, an mRNA vaccine comprises an mRNA encoding an EBNA3A comprising an amino acid sequence having at least 80%, at least 85%, at least 90%, at least 95%, at least 96%, at least 97%, at least 98%, at least 99%, or 100% identity to the amino acid sequence of SEQ ID NO: 31. In some embodiments, an mRNA vaccine comprises an mRNA encoding an EBNA3A comprising an amino acid sequence having at least 80% identity to the amino acid sequence of SEQ ID NO: 31. In some embodiments, an mRNA vaccine comprises an mRNA encoding an EBNA3A comprising an amino acid sequence having at least 85% identity to the amino acid sequence of SEQ ID NO: 31. In some embodiments, an mRNA vaccine comprises an mRNA encoding an EBNA3A comprising an amino acid sequence having at least 90% identity to the amino acid sequence of SEQ ID NO: 31. In some embodiments, an mRNA vaccine comprises an mRNA encoding an EBNA3A comprising an amino acid sequence having at least 95% identity to the amino acid sequence of SEQ ID NO: 31. In some embodiments, an mRNA vaccine comprises an mRNA encoding an EBNA3A comprising the amino acid sequence of SEQ ID NO: 31.

In some embodiments, an mRNA comprises an open reading frame comprising a nucleic acid sequence having at least 70% identity to the nucleic acid sequence of SEQ ID NO: 11. In some embodiments, an mRNA comprises an open reading frame comprising a nucleic acid sequence having at least 75% identity to the nucleic acid sequence of SEQ ID NO: 11. In some embodiments, an mRNA comprises an open reading frame comprising a nucleic acid sequence having at least 80% identity to the nucleic acid sequence of SEQ ID NO: 11. In some embodiments, an mRNA comprises an open reading frame comprising a nucleic acid sequence having at least 85% identity to the nucleic acid sequence of SEQ ID NO: 11. In some embodiments, an mRNA comprises an open reading frame comprising a nucleic acid sequence having at least 90% identity to the nucleic acid sequence of SEQ ID NO: 11. In some embodiments, an mRNA comprises an open reading frame comprising a nucleic acid sequence having at least 95% identity to the nucleic acid sequence of SEQ ID NO: 11. In some embodiments, an mRNA comprises an open reading frame comprising the nucleic acid sequence of SEQ ID NO: 11.

In some embodiments, an mRNA comprises an open reading frame comprising a nucleic acid sequence having at least 70% identity to the nucleic acid sequence of SEQ ID NO: 24. In some embodiments, an mRNA comprises an open reading frame comprising a nucleic acid sequence having at least 75% identity to the nucleic acid sequence of SEQ ID NO: 24. In some embodiments, an mRNA comprises an open reading frame comprising a nucleic acid sequence having at least 80% identity to the nucleic acid sequence of SEQ ID NO: 24. In some embodiments, an mRNA comprises an open reading frame comprising a nucleic acid sequence having at least 85% identity to the nucleic acid sequence of SEQ ID NO: 24. In some embodiments, an mRNA comprises an open reading frame comprising a nucleic acid sequence having at least 90% identity to the nucleic acid sequence of SEQ ID NO: 24. In some embodiments, an mRNA comprises an open reading frame comprising a nucleic acid sequence having at least 95% identity to the nucleic acid sequence of SEQ ID NO: 24. In some embodiments, an mRNA comprises an open reading frame comprising the nucleic acid sequence of SEQ ID NO: 24.

In some embodiments, an mRNA comprises an open reading frame comprising a nucleic acid sequence having at least 70% identity to the nucleic acid sequence of SEQ ID NO: 25. In some embodiments, an mRNA comprises an open reading frame comprising a nucleic acid sequence having at least 75% identity to the nucleic acid sequence of SEQ ID NO: 25. In some embodiments, an mRNA comprises an open reading frame comprising a nucleic acid sequence having at least 80% identity to the nucleic acid sequence of SEQ ID NO: 25. In some embodiments, an mRNA comprises an open reading frame comprising a nucleic acid sequence having at least 85% identity to the nucleic acid sequence of SEQ ID NO: 25. In some embodiments, an mRNA comprises an open reading frame comprising a nucleic acid sequence having at least 90% identity to the nucleic acid sequence of SEQ ID NO: 25. In some embodiments, an mRNA comprises an open reading frame comprising a nucleic acid sequence having at least 95% identity to the nucleic acid sequence of SEQ ID NO: 25. In some embodiments, an mRNA comprises an open reading frame comprising the nucleic acid sequence of SEQ ID NO: 25.

In some embodiments, an mRNA comprises an open reading frame comprising a nucleic acid sequence having at least 70% identity to the nucleic acid sequence of SEQ ID NO: 26. In some embodiments, an mRNA comprises an open reading frame comprising a nucleic acid sequence having at least 75% identity to the nucleic acid sequence of SEQ ID NO: 26. In some embodiments, an mRNA comprises an open reading frame comprising a nucleic acid sequence having at least 80% identity to the nucleic acid sequence of SEQ ID NO: 26. In some embodiments, an mRNA comprises an open reading frame comprising a nucleic acid sequence having at least 85% identity to the nucleic acid sequence of SEQ ID NO: 26. In some embodiments, an mRNA comprises an open reading frame comprising a nucleic acid sequence having at least 90% identity to the nucleic acid sequence of SEQ ID NO: 26. In some embodiments, an mRNA comprises an open reading frame comprising a nucleic acid sequence having at least 95% identity to the nucleic acid sequence of SEQ ID NO: 26. In some embodiments, an mRNA comprises an open reading frame comprising the nucleic acid sequence of SEQ ID NO: 26.

In some embodiments, an mRNA comprises an open reading frame comprising a nucleic acid sequence having at least 70% identity to the nucleic acid sequence of SEQ ID NO: 27. In some embodiments, an mRNA comprises an open reading frame comprising a nucleic acid sequence having at least 75% identity to the nucleic acid sequence of SEQ ID NO: 27. In some embodiments, an mRNA comprises an open reading frame comprising a nucleic acid sequence having at least 80% identity to the nucleic acid sequence of SEQ ID NO: 27. In some embodiments, an mRNA comprises an open reading frame comprising a nucleic acid sequence having at least 85% identity to the nucleic acid sequence of SEQ ID NO: 27. In some embodiments, an mRNA comprises an open reading frame comprising a nucleic acid sequence having at least 90% identity to the nucleic acid sequence of SEQ ID NO: 27. In some embodiments, an mRNA comprises an open reading frame comprising a nucleic acid sequence having at least 95% identity to the nucleic acid sequence of SEQ ID NO: 27. In some embodiments, an mRNA comprises an open reading frame comprising the nucleic acid sequence of SEQ ID NO: 27.

### EBV Latent Membrane Protein 2B

EBV Latent Membrane Protein 2B (LMP2B), a multi-transmembrane protein, is a naturally occurring isoform of LMP2A, lacking the latter's N-terminal 119 residues. LMP2B, unlike LMP2A, does not modulate B cell receptor signaling or block apoptosis, and instead may serve as a negative regulator of the LMP2A isoform. LMP2B is a latent antigen expressed during latency in EBV-infected B cells both in cell culture and in vivo. LMP2B has been shown to promote motility and cell spread in epithelial cells. Examples of wild-type EBV LMP2B proteins are provided in UniProtKB: A0A0C7U069 and P13285.

In some embodiments, an mRNA vaccine comprises an mRNA encoding an EBV LMP2B comprising an amino acid sequence having at least 80%, at least 85%, at least 90%, at least 95%, at least 96%, at least 97%, at least 98%, at least 99%, or 100% identity to the amino acid sequence of SEQ ID NO: 6. In some embodiments, an mRNA vaccine comprises an mRNA encoding an EBV LMP2B comprising an amino acid sequence having at least 80% identity to the amino acid sequence of SEQ ID NO: 6. In some embodiments, an mRNA vaccine comprises an mRNA encoding an EBV LMP2B comprising an amino acid sequence having at least 85% identity to the amino acid sequence of SEQ ID NO: 6. In some embodiments, an mRNA vaccine comprises an mRNA encoding an EBV LMP2B comprising an amino acid sequence having at least 90% identity to the amino acid sequence of SEQ ID NO: 6. In some embodiments, an mRNA vaccine comprises an mRNA encoding an EBV LMP2B comprising an amino acid sequence having at least 95% identity to the amino acid sequence of SEQ ID NO: 6. In some embodiments, an mRNA vaccine comprises an mRNA encoding an EBV LMP2B comprising the amino acid sequence of SEQ ID NO: 6.

In some embodiments, an mRNA vaccine comprises an mRNA encoding an EBV LMP2B comprising an amino acid sequence having at least 80%, at least 85%, at least 90%, at least 95%, at least 96%, at least 97%, at least 98%, at least 99%, or 100% identity to the amino acid sequence of SEQ ID NO: 23. In some embodiments, an mRNA vaccine comprises an mRNA encoding an EBV LMP2B comprising an amino acid sequence having at least 80% identity to the amino acid sequence of SEQ ID NO: 23. In some embodiments, an mRNA vaccine comprises an mRNA encoding an EBV LMP2B comprising an amino acid sequence having at least 85% identity to the amino acid sequence of SEQ ID NO: 23. In some embodiments, an mRNA vaccine comprises an mRNA encoding an EBV LMP2B comprising an amino acid sequence having at least 90% identity to the amino acid sequence of SEQ ID NO: 23. In some embodiments, an mRNA vaccine comprises an mRNA encoding an EBV LMP2B comprising an amino acid sequence having at least 95% identity to the amino acid sequence of SEQ ID NO: 23. In some embodiments, an mRNA vaccine comprises an mRNA encoding an EBV LMP2B comprising the amino acid sequence of SEQ ID NO: 23.

In some embodiments, an mRNA comprises an open reading frame comprising a nucleic acid sequence having at least 70% identity to the nucleic acid sequence of SEQ ID NO: 12. In some embodiments, an mRNA comprises an open reading frame comprising a nucleic acid sequence having at least 75% identity to the nucleic acid sequence of SEQ ID NO: 12. In some embodiments, an mRNA comprises an open reading frame comprising a nucleic acid sequence having at least 80% identity to the nucleic acid sequence of SEQ ID NO: 12. In some embodiments, an mRNA comprises an open reading frame comprising a nucleic acid sequence having at least 85% identity to the nucleic acid sequence of SEQ ID NO: 12. In some embodiments, an mRNA comprises an open reading frame comprising a nucleic acid sequence having at least 90% identity to the nucleic acid sequence of SEQ ID NO: 12. In some embodiments, an mRNA comprises an open reading frame comprising a nucleic acid sequence having at least 95% identity to the nucleic acid sequence of SEQ ID NO: 12. In some embodiments, an mRNA comprises an open reading frame comprising the nucleic acid sequence of SEQ ID NO: 12.

Non-limiting examples of RNA sequences and corresponding amino acid sequences of EBV proteins of the present disclosure are provided in Table 2.

TABLE 2
Vaccine sequences

| | | |
|---|---|---|
| | | SEQ ID |
| Name | Sequence | NO: |

EBVgp220

| SEQ ID NO: 13 consists of from 5′ end to 3′ end: 5′ UTR SEQ ID NO: 19, mRNA | | 13 |
|---|---|---|
| ORF SEQ ID NO: 7, and 3′ UTR SEQ ID NO: 20. | | |
| Chemical | 1-methylpseudouridine | |
| Modification | | |
| Cap | 7mG(5′)ppp(5′)NlmpNp | |
| 5′ UTR | GAGGAAAUAAGAGAGAAAAGAAGAGUAAGAAGAAAUAUAAGACCCCGGCGCCGCC | 19 |
| | ACC | |
| mRNA ORF | AUGGAGGCAGCACUGCUGGUCUGCCAGUAUACAAUCCAGUCGCUCAUUCAUCUGA | 7 |
| | CAGGCGAGGACCCCGGCUUCUUUAACGUGGAAAUCCCAGAGUUCCCUUUCUAUCC | |
| | AACCUGUAACGUUUGCACUGCUGACGUGAACGUGACAAUCAAUUUCGACGUGGGA | |
| | GGAAAGAAGCAUCAGCUUGAUCUGGAUUUCGGCCAACUGACGCCACACACCAAAG | |
| | CGGUGUACCAACCAAGGGGUGCAUUUGGAGGGAGCGAGAAUGCUACAAACCUGUU | |
| | CUUACUGGAACUGCUUGGUGCAGGAGAGCUGGCACUAACCAUGCGAAGCAAGAAG | |
| | CUCCCCAUCAACGUUACCACCGGGGAAGAACAGCAAGUCAGCCUUGAAUCCGUGG | |
| | AUGUGUACUUUCAGGAUGUUUUCGGCACCAUGUGGUGUCACCACGCUGAGAUGCA | |
| | GAAUCCCGUGUAUCUCAUACCCGAGACUGUACCCUACAUCAAGUGGGAUAAUUGC | |
| | AAUUCAACUAAUAUUACGGCUGUGGUGAGGGCCCAGGGCUUGGACGUGACUCUGC | |
| | CCUUAUCUCUACCUACUUCUGCCCAAGACUCCAAUUUCUCUGUCAAGACCGAGAU | |
| | GCUCGGGAAUGAGAUCGAUAUCGAGUGCAUCAUGGAGGACGGUGAGAUAAGCCAG | |
| | GUUCUGCCCGGCGACAACAAGUUCAAUAUCACUUGUUCUGGCUACGAGUCCCAUG | |
| | UGCCUAGUGGUGGCAUACUCACAAGUACUUCUCCCGUAGCCACGCCCAUUCCCGG | |
| | AACCGGAUACGCCUACAGUCUGCGUCUGACCCCACGGCCUGUGUCCAGAUUCCUG | |
| | GGUAACAAUAGUAUCUUAUACGUGUUUUAUAGCGGAAACGGCCCUAAAGCGUCCG | |
| | GAGGGGACUAUUGUAUUCAGAGUAAUAUCGUUUUCUCUGAUGAGAUUCCUGCCAG | |
| | UCAGGACAUGCCGACAAACACAACUGAUAUUACCUACGUGGGCGACAAUGCCACG | |
| | UAUUCAGUGCCCAUGGUCACGAGCGAGGACGCCAAUUCACCAAAUGUUACUGUAA | |
| | CAGCUUUCUGGGCCUGGCCAAAUAACACUGAGACUGACUUCAAAUGUAAGUGGAC | |
| | UUUGACCUCUGGAACUCCGUCGGGUUGCGAGAAUAUCAGCGGGGCCUUUGCUUCC | |
| | AACAGGACUUUCGACAUCACUGUCUCAGGGCUGGGGACAGCACCGAAGACAUUAA | |
| | UCAUAACACGGACCGCCACCAACGCCACGACUACAACCCAUAAGGUGAUCUUUUC | |
| | CAAGGCACCUGAGUCCACCACUACCUCCCCGACUCUUAACACUACGGGCUUCGCU | |
| | GAUCCCAACACCACUACGGGGUUGCCUAGCUCGACACAUGUGCCGACGAACCUGA | |
| | CUGCCCCUGCAUCGACCGGGCCCACAGUUUCGACCGCCGAUGUGACAUCACCAAC | |
| | GCCCGCAGGUACAACCUCAGGCGCCAGCCCAGUGACCCCUUCCCCAAGCCCCUGG | |
| | GAUAAUGGAACGGAGUCCACGCCUCCCCAGAACGCAACCAGUCCUCAGGCGCCCA | |
| | GCGGGCAGAAGACUGCGGUGCCAACUGUGACCAGCACCGGUGGCAAGGCCAACUC | |
| | AACAACUGGAGGCAAGCAUACGACGGGGCACGGCGCCCGGACCUCCACUGAACCC | |
| | ACGACCGAUUACGGAGGUGACAGCACAACACCGCGGCCACGAUAUAAUGCCACCA | |
| | CUUAUCUGCCACCAUCCACAAGCUCCAAGCUGCGGCCACGGUGGACCUUCACAAG | |
| | CCCACCCGUGACGACUGCCCAAGCGACGGUGCCAGUGCCACCUACAAGCCAGCCA | |
| | CGCUUCUCCAACCUUAGUAUGCUCGUUCUCCAGUGGGCCAGCCUUGCUGUUCUGA | |
| | CCCUCCUCCUGCUGCUCGUGAUGGCCGAUUGCGCCUUUAGGAGAAACCUCAGCAC | |
| | UAGCCACACGUACACCACGCCGCCCUACGAUGACGCCGAGACUUACGUC | |
| 3′ UTR | UGAUAAUAGGCUGGAGCCUCGGUGGCCUAGCUUCUUGCCCCUUGGGCCUCCCCCC | 20 |
| | AGCCCCUCCUCCCCUUCCUGCACCCGUACCCCCGUGGUCUUUGAAUAAAGUCUGA | |
| | GUGGGCGGC | |
| PolyA | 100 nucleotides | |
| Corresponding | MEAALLVCQYTIQSLIHLTGEDPGFFNVEIPEFPFYPTCNVCTADVNVTINFDVG | 1 |
| Protein | GKKHQLDLDFGQLTPHTKAVYQPRGAFGGSENATNLFLLELLGAGELALTMRSKK | |
| | LPINVTTGEEQQVSLESVDVYFQDVFGTMWCHHAEMQNPVYLIPETVPYIKWDNC | |
| | NSTNITAVVRAQGLDVTLPLSLPTSAQDSNFSVKTEMLGNEIDIECIMEDGEISQ | |
| | VLPGDNKFNITCSGYESHVPSGGILTSTSPVATPIPGTGYAYSLRLTPRPVSRFL | |
| | GNNSILYVFYSGNGPKASGGDYCIQSNIVESDEIPASQDMPTNTTDITYVGDNAT | |
| | YSVPMVTSEDANSPNVTVTAFWAWPNNTETDFKCKWTLTSGTPSGCENISGAFAS | |
| | NRTFDITVSGLGTAPKTLIITRTATNATTTTHKVIFSKAPESTTTSPTLNTTGFA | |
| | DPNTTTGLPSSTHVPTNLTAPASTGPTVSTADVTSPTPAGTTSGASPVTPSPSPW | |
| | DNGTESTPPQNATSPQAPSGQKTAVPTVTSTGGKANSTTGGKHTTGHGARTSTEP | |
| | TTDYGGDSTTPRPRYNATTYLPPSTSSKLRPRWTFTSPPVTTAQATVPVPPTSQP | |
| | RFSNLSMLVLQWASLAVLTLLLLLVMADCAFRRNLSTSHTYTTPPYDDAETYV | |

EBV gH

| SEQ ID NO: 14 consists of from 5′ end to 3′ end: 5′ UTR SEQ ID NO: 19, mRNA | | 14 |
|---|---|---|
| ORF SEQ ID NO: 8, and 3′ UTR SEQ ID NO: 20. | | |
| Chemical | 1-methylpseudouridine | |
| Modification | | |
| Cap | 7mG(5′)ppp(5′)NlmpNp | |
| 5′ UTR | GAGGAAAUAAGAGAGAAAAGAAGAGUAAGAAGAAAUAUAAGACCCCGGCGCCGCC | 19 |
| | ACC | |
| mRNA ORF | AUGCAGUUGUUGUGCGUGUUCUGCCUCGUGUUACUCUGGGAGGUGGGCGCCGCCA | 8 |
| | GCCUUAGCGAGGUGAAGCUCCACUUGGACAUCGAGGGCCACGCCAGCCACUACAC | |
| | CAUCCCCUGGACCGAGCUCAUGGCCAAGGUGCCCGGCCUUAGCCCCGAGGCCCUG | |
| | UGGCGGGAGGCCAACGUGACCGAGGACCUGGCCAGCAUGCUGAACCGGUACAAGC | |
| | UGAUCUACAAGACCAGCGGCACCCUGGGCAUCGCCCUGGCCGAGCCCGUGGACAU | |
| | CCCCGCCGUUAGCGAAGGCAGCAUGCAGGUGGACGCCAGCAAGGUGCACCCCGGC | |
| | GUGAUCAGCGGCCUGAACAGCCCCGCCUGUAUGUUGAGCGCCCCACUGGAGAAGC | |
| | AGCUGUUCUACUACAUCGGCACCAUGCUGCCCAACACCCGGCCCCACAGCUACGU | |
| | GUUCUACCAGCUGCGGUGCCACCUGAGCUACGUUGCCCUGAGCAUCAACGGCGAC | |
| | AAGUUCCAGUACACCGGCGCCAUGACCAGCAAGUUCCUGAUGGGCACCUACAAGC | |
| | GGGUCACCGAGAAGGGCGACGAGCACGUGCUGUCACUGGUGUUCGGCAAGACCAA | |
| | GGACCUGCCCGACCUGCGGGGCCCCUUCAGCUACCCUAGUUUGACCAGCGCCCAG | |
| | AGCGGCGACUACAGCUUGGUGAUCGUGACCACCUUCGUGCACUACGCCAACUUCC | |
| | ACAACUACUUCGUGCCCAACCUGAAGGACAUGUUCAGCCGGGCCGUGACCAUGAC | |
| | UGCCGCUUCUUACGCCCGGUACGUGCUGCAGAAGCUGGUCCUGCUGGAGAUGAAG | |
| | GGCGGCUGCCGGGAGCCCGAGCUGGACACCGAAACACUGACCACCAUGUUCGAGG | |
| | UGAGCGUGGCCUUCUUCAAGGUGGGUCACGCGGUGGGCGAAACCGGCAACGGCUG | |
| | CGUGGACUUACGCUGGCUGGCCAAGAGCUUCUUCGAGCUGACCGUGCUGAAGGAU | |
| | AUCAUCGGCAUCUGCUACGGCGCCACCGUGAAGGGCAUGCAGAGCUACGGCCUGG | |
| | AGCGGCUGGCCGCCAUGCUUAUGGCAACAGUGAAGAUGGAGGAGCUGGGACACCU | |
| | GACAACAGAGAAGCAGGAGUACGCCCUGAGACUGGCCACAGUGGGCUACCCAAAG | |
| | GCCGGCGUGUACAGUGGACUGAUCGGCGGCGCAACCAGCGUGCUGCUAUCCGCUU | |
| | ACAACCGGCACCCGCUGUUCCAGCCCCUGCACACCGUGAUGCGGGAAACCCUGUU | |
| | CAUCGGAAGCCACGUCGUGCUGCGGGAGCUGAGGCUGAACGUAACCACCCAGGGC | |
| | CCUAAUCUGGCCCUGUAUCAGCUCCUCAGUACCGCCCUGUGCAGCGCCCUUGAGA | |
| | UCGGCGAGGUGCUCAGAGGCCUGGCCCUCGGUACCGAGAGCGGCCUCUUCAGCCC | |
| | AUGCUACUUAAGCCUGCGGUUCGACCUGACCCGGGACAAGUUGCUGAGCAUGGCC | |
| | CCGCAGGAGGCCACACUGGACCAGGCAGCUGUAUCCAACGCCGUGGACGGCUUCC | |
| | UGGGCAGACUGUCCCUGGAACGGGAGGACCGGGACGCCUGGCACCUGCCUGCCUA | |
| | CAAGUGUGUGGAUCGGCUGGACAAGGUGCUGAUGAUCAUCCCUCUGAUUAAUGUC | |
| | ACCUUCAUCAUCAGCAGCGACCGGGAGGUGCGGGGAUCCGCCCUCUACGAGGCCA | |
| | GCACCACCUAUCUGAGCAGCAGCCUGUUCCUGUCUCCUGUGAUCAUGAACAAGUG | |
| | CAGCCAGGGCGCCGUGGCCGGCGAGCCCCGGCAGAUCCCCAAGAUCCAGAACUUC | |
| | ACCCGGACCCAGAAGUCUUGCAUCUUCUGCGGCUUCGCCCUUUUGUCCUACGACG | |
| | AGAAGGAGGGCUUGGAGACUACAACCUACAUCACCAGCCAGGAGGUGCAGAACAG | |
| | CAUCCUGUCAUCUAAUUACUUCGACUUCGACAACCUGCACGUUCAUUACCUGCUC | |
| | CUCACCACCAACGGUACCGUCAUGGAAAUCGCCGGACUGUACGAGGAGCGGGCCC | |
| | AUGUUGUGCUGGCCAUCAUCCUGUACUUCAUCGCUUUCGCACUUGGCAUCUUCCU | |
| | GGUGCACAAGAUCGUGAUGUUCUUCCUG | |
| 3′ UTR | UGAUAAUAGGCUGGAGCCUCGGUGGCCUAGCUUCUUGCCCCUUGGGCCUCCCCCC | 20 |
| | AGCCCCUCCUCCCCUUCCUGCACCCGUACCCCCGUGGUCUUUGAAUAAAGUCUGA | |
| | GUGGGCGGC | |
| PolyA | 100 nucleotides | |
| Corresponding | MQLLCVFCLVLLWEVGAASLSEVKLHLDIEGHASHYTIPWTELMAKVPGLSPEAL | 2 |
| Protein | WREANVTEDLASMLNRYKLIYKTSGTLGIALAEPVDIPAVSEGSMQVDASKVHPG | |
| | VISGLNSPACMLSAPLEKQLFYYIGTMLPNTRPHSYVFYQLRCHLSYVALSINGD | |
| | KFQYTGAMTSKFLMGTYKRVTEKGDEHVLSLVFGKTKDLPDLRGPFSYPSLTSAQ | |
| | SGDYSLVIVTTFVHYANFHNYFVPNLKDMFSRAVTMTAASYARYVLQKLVLLEMK | |
| | GGCREPELDTETLTTMFEVSVAFFKVGHAVGETGNGCVDLRWLAKSFFELTVLKD | |
| | IIGICYGATVKGMQSYGLERLAAMLMATVKMEELGHLTTEKQEYALRLATVGYPK | |
| | AGVYSGLIGGATSVLLSAYNRHPLFQPLHTVMRETLFIGSHVVLRELRLNVTTQG | |
| | PNLALYQLLSTALCSALEIGEVLRGLALGTESGLFSPCYLSLRFDLTRDKLLSMA | |
| | PQEATLDQAAVSNAVDGFLGRLSLEREDRDAWHLPAYKCVDRLDKVLMIIPLINV | |
| | TFIISSDREVRGSALYEASTTYLSSSLFLSPVIMNKCSQGAVAGEPRQIPKIQNF | |
| | TRTQKSCIFCGFALLSYDEKEGLETTTYITSQEVQNSILSSNYFDFDNLHVHYLL | |
| | LTTNGTVMEIAGLYEERAHVVLAIILYFIAFALGIFLVHKIVMFFL | |

EBV gL

| SEQ ID NO: 15 consists of from 5′ end to 3′ end: 5′ UTR SEQ ID NO: 19, mRNA | | 15 |
|---|---|---|
| ORF SEQ ID NO: 9, and 3′ UTR SEQ ID NO: 20. | | |
| Chemical | 1 -methylpseudouridine | |
| Modification | | |
| Cap | 7mG(5′)ppp(5′)NlmpNp | |
| 5′ UTR | GAGGAAAUAAGAGAGAAAAGAAGAGUAAGAAGAAAUAUAAGACCCCGGCGCCGCC | 19 |
| | ACC | |
| mRNA ORF | AUGAGAGCCGUGGGCGUGUUCCUGGCCAUCUGCCUGGUGACCAUCUUCGUGCUGC | 9 |
| | CCACCUGGGGCAACUGGGCCUACCCCUGCUGCCACGUGACCCAGCUGAGAGCCCA | |
| | GCACCUGCUGGCCCUGGAGAACAUCAGCGACAUCUACCUGGUGAGCAACCAGACC | |
| | UGCGACGGCUUCAGCCUGGCCAGCCUGAACAGCCCCAAGAACGGCAGCAACCAGC | |
| | UGGUGAUCAGCAGAUGCGCCAACGGCCUGAACGUGGUGAGCUUCUUCAUCAGCAU | |
| | CCUGAAGAGAAGCAGCAGCGCCCUGACCGGCCACCUGAGAGAGCUGCUGACCACC | |
| | CUGGAGACCCUGUACGGCAGCUUCAGCGUGGAGGACCUGUUCGGCGCCAACCUGA | |
| | ACAGAUACGCCUGGCACAGAGGCGGC | |
| 3′ UTR | UGAUAAUAGGCUGGAGCCUCGGUGGCCUAGCUUCUUGCCCCUUGGGCCUCCCCCC | 20 |
| | AGCCCCUCCUCCCCUUCCUGCACCCGUACCCCCGUGGUCUUUGAAUAAAGUCUGA | |
| | GUGGGCGGC | |
| PolyA | 100 nucleotides | |
| Corresponding | MRAVGVFLAICLVTIFVLPTWGNWAYPCCHVTQLRAQHLLALENISDIYLVSNQT | 3 |
| Protein | CDGFSLASLNSPKNGSNQLVISRCANGLNVVSFFISILKRSSSALTGHLRELLTT | |
| | LETLYGSFSVEDLFGANLNRYAWHRGG | |

EBVgp42-d41 (soluble/secreted)

| SEQ ID NO: 16 consists of from 5′ end to 3′ end: 5′ UTR SEQ ID NO: 19, mRNA | | 16 |
|---|---|---|
| ORF SEQ ID NO: 10, and 3′ UTR SEQ ID NO: 20. | | |
| Chemical | 1-methylpseudouridine | |
| Modification | | |
| Cap | 7mG(5′)ppp(5′)NlmpNp | |
| 5′ UTR | GAGGAAAUAAGAGAGAAAAGAAGAGUAAGAAGAAAUAUAAGACCCCGGCGCCGCC | 19 |
| | ACC | |
| mRNA ORF | AUGACCCGGCGGCGGGUGUUAAGCGUGGUGGUGCUCUUAGCCGCCUUAGCCUGCC | 10 |
| | GGCUUGGCGCCAUCACCUGGGUGCCCAAGCCCAACGUGGAGGUGUGGCCCGUGGA | |
| | CCCGCCACCUCCCGUGAACUUCAACAAGACCGCCGAGCAGGAGUACGGCGACAAG | |
| | GAGGUGAAGCUGCCCCACUGGACGCCGACCCUGCACACCUUCCAGGUGCCCCAGA | |
| | ACUACACCAAGGCCAACUGCACCUACUGCAACACCAGAGAGUACACCUUCAGCUA | |
| | CAAGGGCUGCUGCUUCUACUUCACCAAGAAGAAGCACACCUGGAACGGCUGCUUC | |
| | CAGGCCUGCGCCGAGCUGUACCCCUGCACCUACUUCUACGGCCCCACCCCAGACA | |
| | UCCUGCCCGUGGUGACCAGAAACCUGAACGCCAUCGAGAGCCUGUGGGUGGGCGU | |
| | GUACAGAGUGGGCGAGGGCAACUGGACCAGCCUGGACGGCGGCACCUUCAAGGUG | |
| | UACCAGAUCUUCGGCAGCCACUGCACCUACGUGAGCAAGUUCAGCACCGUGCCCG | |
| | UGAGCCACCACGAGUGCAGCUUCCUGAAGCCCUGCCUGUGCGUGAGCCAGAGAAG | |
| | CAACAGC | |
| 3′ UTR | UGAUAAUAGGCUGGAGCCUCGGUGGCCUAGCUUCUUGCCCCUUGGGCCUCCCCCC | 20 |
| | AGCCCCUCCUCCCCUUCCUGCACCCGUACCCCCGUGGUCUUUGAAUAAAGUCUGA | |
| | GUGGGCGGC | |
| PolyA | 100 nucleotides | |
| Corresponding | MTRRRVLSVVVLLAALACRLGAITWVPKPNVEVWPVDPPPPVNENKTAEQEYGDK | 4 |
| Protein | EVKLPHWTPTLHTFQVPQNYTKANCTYCNTREYTFSYKGCCFYFTKKKHTWNGCF | |
| | QACAELYPCTYFYGPTPDILPVVTRNLNAIESLWVGVYRVGEGNWTSLDGGTFKV | |
| | YQIFGSHCTYVSKFSTVPVSHHECSFLKPCLCVSQRSNS | |

EBV variant EBNA3A (68-523)-gsgsNLS

| SEQ ID NO: 17 consists of from 5′ end to 3′ end: 5′ UTR SEQ ID NO: 19, mRNA | | 17 |
|---|---|---|
| ORF SEQ ID NO: 11, and 3′ UTR SEQ ID NO: 20. | | |
| Chemical | 1-methylpseudouridine | |
| Modification | | |
| Cap | 7mG(5′)ppp(5′)NlmpNp | |
| 5′ UTR | GAGGAAAUAAGAGAGAAAAGAAGAGUAAGAAGAAAUAUAAGACCCCGGCGCCGCC | 19 |
| | ACC | |
| mRNA ORF | AUGGAUCACGACGCCGGAGGAAGCGCACCCGCCAGACCCAUGCUGCCUCCGCAGC | 11 |
| | CCGACCUUCCCGGACGGGAGGCCAUCCUGCGGCGGUUCCCUCUGGACCUGCGGAC | |
| | CCUGCUGCAGGCUAUCGGCGCUGCCGCUACACGGAUCGACACCCGGGCCAUCGAC | |
| | CAGUUCUUCGGCAGCCAGAUCAGCAACACCGAGAUGUACAUCAUGUACGCCAUGG | |
| | CCAUCCGGCAAGCCAUCGGCAGCGGAAGCGGCAGCGGAAGCGGAAGCAGAGACCA | |
| | GGCCAAGUGGCGGCUGCAAACCUUAGCCGCUGGCUGGCCCAUGGGCUACCAGGCC | |
| | UACAGCAGCUGGAUGUACAGCUACACCGACCACCAGACCACUCCCACCUUCGUGC | |
| | ACCUGCAGGCCACACUCGGCUGCACUGGCGGUAGACGGUGCCACGUGACCUUCAG | |
| | CGCCGGCACCUUCAAGCUGCCUCGGUGUACACCCGGCGACCGUCAGUGGCUGUAC | |
| | GUGCAGAGCAGCGUGGGCAACAUCGUGCAGAGCUGCAAUCCCCGGUACAGCAUCU | |
| | UCUUCGACUACAUGGCCAUCCACCGGAGCCUGACCAAGAUCUGGGAGGAGGUGCU | |
| | GACUCCCGACCAGCGGGUGAGCUUCAUGGAGUUCCUGGGCUUCCUGCAGCGGACC | |
| | GACCUGAGCUACAUCAAGAGCUUCGUGAGCGACGCCCUGGGCACCACCAGCAUCC | |
| | AGACGCCCUGGAUCGACGACAACCCCAGCACCGAGACCGCCCAGGCCUGGAACGC | |
| | CGGCUUUCUACGGGGACGGGCCUACGGAAUCGACCUGCUGCGGACCGAGGGCGAG | |
| | CACGUCGAGGGCGCUACGGGUGAGACCCGGGAGGAGAGCGAGGACACCGAGAGCG | |
| | ACGGCGACGACGAGGACCUGCCCUGCAUCGUGAGCAGAGGCGGCGGCUCCGGCAG | |
| | CAGACCUCCCAUCUUCAUUCGCCGGCUCCACCGUCUGCUGCUGAUGGGCAGCGGC | |
| | AGCGGCACCGAGCAGGGCAAGGAGGUGCUGGAGAAGGCCCGGGGCAGCACCUACG | |
| | GAACCCCACGGCCUCCUGUGCCCAAGCCCAGGCCCGAAGUGCCCCAGAGCGACGA | |
| | GACCGCCACCAGCCACGGCAGUGCCCAGGUGCCCGAGCCUCCAACCAUCCAUCUG | |
| | GCCGCACAGGGCAUGGCCUAUCCCCUGCACGAGCAGCACGGCAUGGCACCCUGUC | |
| | CCGUGGCCCAGGCUCCUCCUACUCCCCUGCCACCCGUGAGCCCUGGCGACCAACU | |
| | CCCCGGGGUGUUCAGCGACGGCAGGGUGGCUUGCGCUCCAGUACCGGCCCCUGCA | |
| | GGACCUAUCGUUCGGCCGUGGGAGCCAAGCCUGACCCAGGCAGCGGGCCAG | |
| 3′ UTR | UGAUAAUAGGCUGGAGCCUCGGUGGCCUAGCUUCUUGCCCCUUGGGCCUCCCCCC | 20 |
| | AGCCCCUCCUCCCCUUCCUGCACCCGUACCCCCGUGGUCUUUGAAUAAAGUCUGA | |
| | GUGGGCGGC | |
| PolyA | 100 nucleotides | |
| Corresponding | MDHDAGGSAPARPMLPPQPDLPGREAILRRFPLDLRTLLQAIGAAATRIDTRAID | 5 |
| Protein | QFFGSQISNTEMYIMYAMAIRQAIGSGSGSGSGSRDQAKWRLQTLAAGWPMGYQA | |
| | YSSWMYSYTDHQTTPTFVHLQATLGCTGGRRCHVTFSAGTFKLPRCTPGDRQWLY | |
| | VQSSVGNIVQSCNPRYSIFFDYMAIHRSLTKIWEEVLTPDQRVSFMEFLGFLQRT | |
| | DLSYIKSFVSDALGTTSIQTPWIDDNPSTETAQAWNAGFLRGRAYGIDLLRTEGE | |
| | HVEGATGETREESEDTESDGDDEDLPCIVSRGGGSGSRPPIFIRRLHRLLLMGSG | |
| | SGTEQGKEVLEKARGSTYGTPRPPVPKPRPEVPQSDETATSHGSAQVPEPPTIHL | |
| | AAQGMAYPLHEQHGMAPCPVAQAPPTPLPPVSPGDQLPGVFSDGRVACAPVPAPA | |
| | GPIVRPWEPSLTQAAGQ | |

EBV variant EBNA3A (68-523)_gsNLS-mFLGGG

| SEQ ID NO: 32 consists of from 5′ end to 3′ end: 5′ UTR SEQ ID NO: 19, mRNA | | 32 |
|---|---|---|
| ORF SEQ ID NO: 24, and 3′ UTR SEQ ID NO: 20. | | |
| Chemical | 1 -methylpseudouridine | |
| Modification | | |
| Cap | 7mG(5′)ppp(5′)NlmpNp | |
| 5′ UTR | GAGGAAAUAAGAGAGAAAAGAAGAGUAAGAAGAAAUAUAAGACCCCGGCGCCGCC | 19 |
| | ACC | |
| mRNA ORF | AUGGAUCACGACGCCGGAGGAAGCGCACCCGCCAGACCCAUGCUGCCUCCGCAGC | 24 |
| | CCGACCUUCCCGGACGGGAGGCCAUCCUGCGGCGGUUCCCUCUGGACCUGCGGAC | |
| | CCUGCUGCAGGCUAUCGGCGCUGCCGCUACACGGAUCGACACCCGGGCCAUCGAC | |
| | CAGUUCUUCGGCAGCCAGAUCAGCAACACCGAGAUGUACAUCAUGUACGCCAUGG | |
| | CCAUCCGGCAAGCCAUCGGCAGCGGAAGCGGCAGCGGAAGCGGAAGCAGAGAUCA | |
| | GGCCAAGUGGCGGCUGCAAACCUUAGCCGCUGGCUGGCCCAUGGGCUACCAGGCC | |
| | UACAGCAGCUGGAUGUACAGCUACACCGACCACCAGACCACUCCCACCUUCGUGC | |
| | ACCUGCAGGCCACACUCGGCUGCACUGGCGGUAGACGGUGCCACGUGACCUUCAG | |
| | CGCCGGCACCUUCAAGCUGCCUCGGUGUACACCCGGCGACCGUCAGUGGCUGUAC | |
| | GUGCAGAGCAGCGUGGGCAACAUCGUGCAGAGCUGCAAUCCCCGGUACAGCAUCU | |
| | UCUUCGACUACAUGGCCAUCCACCGGAGCCUGACCAAGAUCUGGGAGGAGGUGCU | |
| | GACUCCCGACCAGCGGGUGAGCUUCAUGGAGUUCCUGGGCUUCCUGCAGCGGACC | |
| | GACCUGAGCUACAUCAAGAGCUUCGUGAGCGACGCCCUGGGCACCACCAGCAUCC | |
| | AGACGCCCUGGAUCGACGACAACCCCAGCACCGAGACAGCCCAGGCCUGGAACGC | |
| | CGGCUUUCUAGGUGGAGGCGCCUACGGAAUCGACCUGCUGCGGACCGAGGGCGAG | |
| | CACGUCGAGGGCGCUACGGGUGAGACACGGGAGGAGAGCGAGGACACCGAGAGCG | |
| | ACGGCGACGACGAGGACCUGCCCUGCAUCGUGAGCAGAGGCGGCGGCUCCGGCAG | |
| | CGGCAGCGGCAUCUUCAUUCGCCGGCUCCACCGUCUGCUGCUGAUGGGCAGCGGC | |
| | AGCGGCACCGAGCAGGGCAAGGAGGUGCUGGAGAAGGCCCGGGGCAGCACCUACG | |
| | GAACCCCACGGCCUCCUGUGCCCAAGCCCAGGCCCGAAGUGCCCCAGAGCGACGA | |
| | GACAGCCACCAGCCACGGCAGUGCCCAGGUGCCCGAGCCUCCAACCAUCCAUCUG | |
| | GCCGCACAGGGCAUGGCCUAUCCCCUGCACGAGCAGCACGGCAUGGCACCCUGUC | |
| | CCGUGGCCCAGGCUCCUCCUACUCCCCUGCCACCCGUGAGCCCUGGCGACCAACU | |
| | CCCCGGGGUGUUCAGCGACGGCAGGGUGGCUUGCGCUCCAGUACCGGCCCCUGCA | |
| | GGACCUAUCGUUCGGCCGUGGGAGCCAAGCCUGACCCAGGCAGCGGGCCAG | |
| 3′ UTR | UGAUAAUAGGCUGGAGCCUCGGUGGCCUAGCUUCUUGCCCCUUGGGCCUCCCCCC | 20 |
| | AGCCCCUCCUCCCCUUCCUGCACCCGUACCCCCGUGGUCUUUGAAUAAAGUCUGA | |
| | GUGGGCGGC | |
| PolyA | 100 nucleotides | |
| Corresponding | MDHDAGGSAPARPMLPPQPDLPGREAILRRFPLDLRTLLQAIGAAATRIDTRAID | 28 |
| Protein | QFFGSQISNTEMYIMYAMAIRQAIGSGSGSGSGSRDQAKWRLQTLAAGWPMGYQA | |
| | YSSWMYSYTDHQTTPTFVHLQATLGCTGGRRCHVTFSAGTFKLPRCTPGDRQWLY | |
| | VQSSVGNIVQSCNPRYSIFFDYMAIHRSLTKIWEEVLTPDQRVSFMEFLGFLQRT | |
| | DLSYIKSFVSDALGTTSIQTPWIDDNPSTETAQAWNAGELGGGAYGIDLLRTEGE | |
| | HVEGATGETREESEDTESDGDDEDLPCIVSRGGGSGSGSGIFIRRLHRLLLMGSG | |
| | SGTEQGKEVLEKARGSTYGTPRPPVPKPRPEVPQSDETATSHGSAQVPEPPTIHL | |
| | AAQGMAYPLHEQHGMAPCPVAQAPPTPLPPVSPGDQLPGVFSDGRVACAPVPAPA | |
| | GPIVRPWEPSLTQAAGQ | |

EBV variant EBNA3A (68-523)_gsNLS-mIFIGG

| SEQ ID NO: 33 consists of from 5′ end to 3′ end: 5′ UTR SEQ ID NO: 19, mRNA | | 33 |
|---|---|---|
| ORF SEQ ID NO: 25, and 3′ UTR SEQ ID NO: 20. | | |
| Chemical | 1-methylpseudouridine | |
| Modification | | |
| Cap | 7mG(5′)ppp(5′)NlmpNp | |
| 5′ UTR | GAGGAAAUAAGAGAGAAAAGAAGAGUAAGAAGAAAUAUAAGACCCCGGCGCCGCC | 19 |
| | ACC | |
| mRNA ORF | AUGGAUCACGACGCCGGAGGAAGCGCACCCGCCAGACCCAUGCUGCCUCCGCAGC | 25 |
| | CCGACCUUCCCGGACGGGAGGCCAUCCUGCGGCGGUUCCCUCUGGACCUGCGGAC | |
| | CCUGCUGCAGGCUAUCGGCGCUGCCGCUACACGGAUCGACACCCGGGCCAUCGAC | |
| | CAGUUCUUCGGCAGCCAGAUCAGCAACACCGAGAUGUACAUCAUGUACGCCAUGG | |
| | CCAUCCGGCAAGCCAUCGGCAGCGGAAGCGGCAGCGGAAGCGGAAGCAGAGAUCA | |
| | GGCCAAGUGGCGGCUGCAAACCUUAGCCGCUGGCUGGCCCAUGGGCUACCAGGCC | |
| | UACAGCAGCUGGAUGUACAGCUACACCGACCACCAGACCACUCCCACCUUCGUGC | |
| | ACCUGCAGGCCACACUCGGCUGCACUGGCGGUAGACGGUGCCACGUGACCUUCAG | |
| | CGCCGGCACCUUCAAGCUGCCUCGGUGUACACCCGGCGACCGUCAGUGGCUGUAC | |
| | GUGCAGAGCAGCGUGGGCAACAUCGUGCAGAGCUGCAAUCCCCGGUACAGCAUCU | |
| | UCUUCGACUACAUGGCCAUCCACCGGAGCCUGACCAAGAUCUGGGAGGAGGUGCU | |
| | GACUCCCGACCAGCGGGUGAGCUUCAUGGAGUUCCUGGGCUUCCUGCAGCGGACC | |
| | GACCUGAGCUACAUCAAGAGCUUCGUGAGCGACGCCCUGGGCACCACCAGCAUCC | |
| | AGACGCCCUGGAUCGACGACAACCCCAGCACCGAGACAGCCCAGGCCUGGAACGC | |
| | CGGCUUUCUACGGGGACGGGCCUACGGAAUCGACCUGCUGCGGACCGAGGGCGAG | |
| | CACGUCGAGGGCGCUACGGGUGAGACACGGGAGGAGAGCGAGGACACCGAGAGCG | |
| | ACGGCGACGACGAGGACCUGCCCUGCAUCGUGAGCAGAGGCGGCGGCUCCGGCAG | |
| | CGGCAGCGGCAUCUUCAUUGGCGGCCUCCACCGUCUGCUGCUGAUGGGCAGCGGC | |
| | AGCGGCACCGAGCAGGGCAAGGAGGUGCUGGAGAAGGCCCGGGGCAGCACCUACG | |
| | GAACCCCACGGCCUCCUGUGCCCAAGCCCAGGCCCGAAGUGCCCCAGAGCGACGA | |
| | GACAGCCACCAGCCACGGCAGUGCCCAGGUGCCCGAGCCUCCAACCAUCCAUCUG | |
| | GCCGCACAGGGCAUGGCCUAUCCCCUGCACGAGCAGCACGGCAUGGCACCCUGUC | |
| | CCGUGGCCCAGGCUCCUCCUACUCCCCUGCCACCCGUGAGCCCUGGCGACCAACU | |
| | CCCCGGGGUGUUCAGCGACGGCAGGGUGGCUUGCGCUCCAGUACCGGCCCCUGCA | |
| | GGACCUAUCGUUCGGCCGUGGGAGCCAAGCCUGACCCAGGCAGCGGGCCAG | |
| 3′ UTR | UGAUAAUAGGCUGGAGCCUCGGUGGCCUAGCUUCUUGCCCCUUGGGCCUCCCCCC | 20 |
| | AGCCCCUCCUCCCCUUCCUGCACCCGUACCCCCGUGGUCUUUGAAUAAAGUCUGA | |
| | GUGGGCGGC | |
| PolyA | 100 nucleotides | |
| Corresponding | MDHDAGGSAPARPMLPPQPDLPGREAILRRFPLDLRTLLQAIGAAATRIDTRAID | 29 |
| Protein | QFFGSQISNTEMYIMYAMAIRQAIGSGSGSGSGSRDQAKWRLQTLAAGWPMGYQA | |
| | YSSWMYSYTDHQTTPTFVHLQATLGCTGGRRCHVTFSAGTFKLPRCTPGDRQWLY | |
| | VQSSVGNIVQSCNPRYSIFFDYMAIHRSLTKIWEEVLTPDQRVSFMEFLGFLQRT | |
| | DLSYIKSFVSDALGTTSIQTPWIDDNPSTETAQAWNAGFLRGRAYGIDLLRTEGE | |
| | HVEGATGETREESEDTESDGDDEDLPCIVSRGGGSGSGSGIFIGGLHRLLLMGSG | |
| | SGTEQGKEVLEKARGSTYGTPRPPVPKPRPEVPQSDETATSHGSAQVPEPPTIHL | |
| | AAQGMAYPLHEQHGMAPCPVAQAPPTPLPPVSPGDQLPGVFSDGRVACAPVPAPA | |
| | GPIVRPWEPSLTQAAGQ | |

EBV variant EBNA3A (68-523)_gsNLS-mFLGGG-mIFIGG

| SEQ ID NO: 34 consists of from 5′ end to 3′ end: 5′ UTR SEQ ID NO: 19, mRNA | | 34 |
|---|---|---|
| ORF SEQ ID NO: 26, and 3′ UTR SEQ ID NO: 20. | | |
| Chemical | 1-methylpseudouridine | |
| Modification | | |
| Cap | 7mG(5′)ppp(5′)NlmpNp | |
| 5′ UTR | GAGGAAAUAAGAGAGAAAAGAAGAGUAAGAAGAAAUAUAAGACCCCGGCGCCGCC | 19 |
| | ACC | |
| mRNA ORF | AUGGAUCACGACGCCGGAGGAAGCGCACCCGCCAGACCCAUGCUGCCUCCGCAGC | 26 |
| | CCGACCUUCCCGGACGGGAGGCCAUCCUGCGGCGGUUCCCUCUGGACCUGCGGAC | |
| | CCUGCUGCAGGCUAUCGGCGCUGCCGCUACACGGAUCGACACCCGGGCCAUCGAC | |
| | CAGUUCUUCGGCAGCCAGAUCAGCAACACCGAGAUGUACAUCAUGUACGCCAUGG | |
| | CCAUCCGGCAAGCCAUCGGCAGCGGAAGCGGCAGCGGAAGCGGAAGCAGAGAUCA | |
| | GGCCAAGUGGCGGCUGCAAACCUUAGCCGCUGGCUGGCCCAUGGGCUACCAGGCC | |
| | UACAGCAGCUGGAUGUACAGCUACACCGACCACCAGACCACUCCCACCUUCGUGC | |
| | ACCUGCAGGCCACACUCGGCUGCACUGGCGGUAGACGGUGCCACGUGACCUUCAG | |
| | CGCCGGCACCUUCAAGCUGCCUCGGUGUACACCCGGCGACCGUCAGUGGCUGUAC | |
| | GUGCAGAGCAGCGUGGGCAACAUCGUGCAGAGCUGCAAUCCCCGGUACAGCAUCU | |
| | UCUUCGACUACAUGGCCAUCCACCGGAGCCUGACCAAGAUCUGGGAGGAGGUGCU | |
| | GACUCCCGACCAGCGGGUGAGCUUCAUGGAGUUCCUGGGCUUCCUGCAGCGGACC | |
| | GACCUGAGCUACAUCAAGAGCUUCGUGAGCGACGCCCUGGGCACCACCAGCAUCC | |
| | AGACGCCCUGGAUCGACGACAACCCCAGCACCGAGACAGCCCAGGCCUGGAACGC | |
| | CGGCUUUCUAGGUGGAGGCGCCUACGGAAUCGACCUGCUGCGGACCGAGGGCGAG | |
| | CACGUCGAGGGCGCUACGGGUGAGACACGGGAGGAGAGCGAGGACACCGAGAGCG | |
| | ACGGCGACGACGAGGACCUGCCCUGCAUCGUGAGCAGAGGCGGCGGCUCCGGCAG | |
| | CGGCAGCGGCAUCUUCAUUGGCGGCCUCCACCGUCUGCUGCUGAUGGGCAGCGGC | |
| | AGCGGCACCGAGCAGGGCAAGGAGGUGCUGGAGAAGGCCCGGGGCAGCACCUACG | |
| | GAACCCCACGGCCUCCUGUGCCCAAGCCCAGGCCCGAAGUGCCCCAGAGCGACGA | |
| | GACAGCCACCAGCCACGGCAGUGCCCAGGUGCCCGAGCCUCCAACCAUCCAUCUG | |
| | GCCGCACAGGGCAUGGCCUAUCCCCUGCACGAGCAGCACGGCAUGGCACCCUGUC | |
| | CCGUGGCCCAGGCUCCUCCUACUCCCCUGCCACCCGUGAGCCCUGGCGACCAACU | |
| | CCCCGGGGUGUUCAGCGACGGCAGGGUGGCUUGCGCUCCAGUACCGGCCCCUGCA | |
| | GGACCUAUCGUUCGGCCGUGGGAGCCAAGCCUGACCCAGGCAGCGGGCCAG | |
| 3′ UTR | UGAUAAUAGGCUGGAGCCUCGGUGGCCUAGCUUCUUGCCCCUUGGGCCUCCCCCC | 20 |
| | AGCCCCUCCUCCCCUUCCUGCACCCGUACCCCCGUGGUCUUUGAAUAAAGUCUGA | |
| | GUGGGCGGC | |
| PolyA | 100 nucleotides | |
| Corresponding | MDHDAGGSAPARPMLPPQPDLPGREAILRRFPLDLRTLLQAIGAAATRIDTRAID | 30 |
| Protein | QFFGSQISNTEMYIMYAMAIRQAIGSGSGSGSGSRDQAKWRLQTLAAGWPMGYQA | |
| | YSSWMYSYTDHQTTPTFVHLQATLGCTGGRRCHVTFSAGTFKLPRCTPGDRQWLY | |
| | VQSSVGNIVQSCNPRYSIFFDYMAIHRSLTKIWEEVLTPDQRVSFMEFLGFLQRT | |
| | DLSYIKSFVSDALGTTSIQTPWIDDNPSTETAQAWNAGELGGGAYGIDLLRTEGE | |
| | HVEGATGETREESEDTESDGDDEDLPCIVSRGGGSGSGSGIFIGGLHRLLLMGSG | |
| | SGTEQGKEVLEKARGSTYGTPRPPVPKPRPEVPQSDETATSHGSAQVPEPPTIHL | |
| | AAQGMAYPLHEQHGMAPCPVAQAPPTPLPPVSPGDQLPGVFSDGRVACAPVPAPA | |
| | GPIVRPWEPSLTQAAGQ | |

EBV variant EBNA3A (68-523)_gsNLS-RPP-mFLGGG

| SEQ ID NO: 35 consists of from 5′ end to 3′ end: 5′ UTR SEQ ID NO: 19, mRNA | | 35 |
|---|---|---|
| ORF SEQ ID NO: 27, and 3′ UTR SEQ ID NO: 20. | | |
| Chemical | 1-methylpseudouridine | |
| Modification | | |
| Cap | 7mG(5′)ppp(5′)NlmpNp | |
| 5′ UTR | GAGGAAAUAAGAGAGAAAAGAAGAGUAAGAAGAAAUAUAAGACCCCGGCGCCGCC | 19 |
| | ACC | |
| mRNA ORF | AUGGAUCACGACGCCGGAGGAAGCGCACCCGCCAGACCCAUGCUGCCUCCGCAGC | 27 |
| | CCGACCUUCCCGGACGGGAGGCCAUCCUGCGGCGGUUCCCUCUGGACCUGCGGAC | |
| | CCUGCUGCAGGCUAUCGGCGCUGCCGCUACACGGAUCGACACCCGGGCCAUCGAC | |
| | CAGUUCUUCGGCAGCCAGAUCAGCAACACCGAGAUGUACAUCAUGUACGCCAUGG | |
| | CCAUCCGGCAAGCCAUCGGCAGCGGAAGCGGCAGCGGAAGCGGAAGCAGAGAUCA | |
| | GGCCAAGUGGCGGCUGCAAACCUUAGCCGCUGGCUGGCCCAUGGGCUACCAGGCC | |
| | UACAGCAGCUGGAUGUACAGCUACACCGACCACCAGACCACUCCCACCUUCGUGC | |
| | ACCUGCAGGCCACACUCGGCUGCACUGGCGGUAGACGGUGCCACGUGACCUUCAG | |
| | CGCCGGCACCUUCAAGCUGCCUCGGUGUACACCCGGCGACCGUCAGUGGCUGUAC | |
| | GUGCAGAGCAGCGUGGGCAACAUCGUGCAGAGCUGCAAUCCCCGGUACAGCAUCU | |
| | UCUUCGACUACAUGGCCAUCCACCGGAGCCUGACCAAGAUCUGGGAGGAGGUGCU | |
| | GACUCCCGACCAGCGGGUGAGCUUCAUGGAGUUCCUGGGCUUCCUGCAGCGGACC | |
| | GACCUGAGCUACAUCAAGAGCUUCGUGAGCGACGCCCUGGGCACCACCAGCAUCC | |
| | AGACGCCCUGGAUCGACGACAACCCCAGCACCGAGACAGCCCAGGCCUGGAACGC | |
| | CGGCUUUCUAGGUGGAGGCGCCUACGGAAUCGACCUGCUGCGGACCGAGGGCGAG | |
| | CACGUCGAGGGCGCUACGGGUGAGACACGGGAGGAGAGCGAGGACACCGAGAGCG | |
| | ACGGCGACGACGAGGACCUGCCCUGCAUCGUGAGCAGAGGCGGCGGCUCCGGCAG | |
| | CAGACCUCCCAUCUUCAUUCGCCGGCUCCACCGUCUGCUGCUGAUGGGCAGCGGC | |
| | AGCGGCACCGAGCAGGGCAAGGAGGUGCUGGAGAAGGCCCGGGGCAGCACCUACG | |
| | GAACCCCACGGCCUCCUGUGCCCAAGCCCAGGCCCGAAGUGCCCCAGAGCGACGA | |
| | GACAGCCACCAGCCACGGCAGUGCCCAGGUGCCCGAGCCUCCAACCAUCCAUCUG | |
| | GCCGCACAGGGCAUGGCCUAUCCCCUGCACGAGCAGCACGGCAUGGCACCCUGUC | |
| | CCGUGGCCCAGGCUCCUCCUACUCCCCUGCCACCCGUGAGCCCUGGCGACCAACU | |
| | CCCCGGGGUGUUCAGCGACGGCAGGGUGGCUUGCGCUCCAGUACCGGCCCCUGCA | |
| | GGACCUAUCGUUCGGCCGUGGGAGCCAAGCCUGACCCAGGCAGCGGGCCAG | |
| 3′ UTR | UGAUAAUAGGCUGGAGCCUCGGUGGCCUAGCUUCUUGCCCCUUGGGCCUCCCCCC | 20 |
| | AGCCCCUCCUCCCCUUCCUGCACCCGUACCCCCGUGGUCUUUGAAUAAAGUCUGA | |
| | GUGGGCGGC | |
| PolyA | 100 nucleotides | |
| Corresponding | MDHDAGGSAPARPMLPPQPDLPGREAILRRFPLDLRTLLQAIGAAATRIDTRAID | 31 |
| Protein | QFFGSQISNTEMYIMYAMAIRQAIGSGSGSGSGSRDQAKWRLQTLAAGWPMGYQA | |
| | YSSWMYSYTDHQTTPTFVHLQATLGCTGGRRCHVTFSAGTFKLPRCTPGDRQWLY | |
| | VQSSVGNIVQSCNPRYSIFFDYMAIHRSLTKIWEEVLTPDQRVSFMEFLGFLQRT | |
| | DLSYIKSFVSDALGTTSIQTPWIDDNPSTETAQAWNAGFLGGGAYGIDLLRTEGE | |
| | HVEGATGETREESEDTESDGDDEDLPCIVSRGGGSGSRPPIFIRRLHRLLLMGSG | |
| | SGTEQGKEVLEKARGSTYGTPRPPVPKPRPEVPQSDETATSHGSAQVPEPPTIHL | |
| | AAQGMAYPLHEQHGMAPCPVAQAPPTPLPPVSPGDQLPGVFSDGRVACAPVPAPA | |
| | GPIVRPWEPSLTQAAGq | |

EBV LMP2B

| SEQ ID NO: 18 consists of from 5′ end to 3′ end: 5′ UTR SEQ ID NO: 19, mRNA | | 18 |
|---|---|---|
| ORF SEQ ID NO: 12, and 3′ UTR SEQ ID NO: 20. | | |
| Chemical | 1-methylpseudouridine | |
| Modification | | |
| Cap | 7mG(5′)ppp(5′)NlmpNp | |
| 5′ UTR | GAGGAAAUAAGAGAGAAAAGAAGAGUAAGAAGAAAUAUAAGACCCCGGCGCCGCC | 19 |
| | ACC | |
| mRNA ORF | AUGAACCCCGUGUGCCUGCCCGUGAUCGUGGCCCCUUACCUGUUCUGGCUGGCUG | 12 |
| | CCAUCGCCGCCAGCUGCUUCACCGCCAGCGUGAGCACCGUGGUGACCGCCACCGG | |
| | ACUUGCCCUGAGCCUCCUGCUGCUGGCUGCCGUGGCCAGCUCCUACGCCGCCGCU | |
| | CAGCGGAAACUGCUGACUCCCGUGACCGUGCUGACCGCCGUGGUGACCUUCUUCG | |
| | CCAUCUGCCUGACCUGGCGGAUCGAGGAUCCUCCAUUCAACAGCCUGCUGUUUGC | |
| | UCUUCUCGCAGCGGCAGGAGGCCUGCAGGGCAUCUACGUGCUGGUGAUGCUUGUG | |
| | CUGCUGAUACUGGCGUAUCGGAGGCGCUGGCGGAGACUGACCGUGUGCGGCGGCA | |
| | UCAUGUUCCUGGCCUGCGUGCUGGUGCUGAUCGUGGACGCCGUGCUGCAGCUGUC | |
| | ACCCCUGCUGGGUGCCGUGACCGUGGUGAGCAUGACCCUGCUGCUGCUGGCCUUC | |
| | GUGCUGUGGCUGAGCUCUCCUGGUGGCCUGGGAACCCUGGGCGCUGCUCUGCUGA | |
| | CCCUUGCGGCCGCAUUGGCCCUGCUGGCCAGCCUGAUCCUGGGCACCCUGAACCU | |
| | GACCACCAUGUUCCUGCUGAUGCUGCUGUGGACCCUGGUGGUGCUGCUGAUCUGC | |
| | AGCAGCUGUAGCAGCUGCCCUCUGAGCAAGAUCCUGCUGGCCCGGCUGUUCCUGU | |
| | ACGCCCUGGCCCUUCUGCUGCUGGCCAGCGCACUGAUCGCCGGCGGCAGCAUCCU | |
| | GCAGACCAACUUCAAGAGCCUGAGCAGCACCGAGUUCAUCCCCAACCUGUUCUGC | |
| | AUGCUGCUGCUGAUCGUGGCCGGCAUCCUGUUCAUCCUGGCCAUCCUGACCGAGU | |
| | GGGGCAGCGGCAACCGGACCUACGGCCCCGUGUUCAUGUGCCUGGGCGGCCUGCU | |
| | GACCAUGGUUGCCGGGGCAGUGUGGCUGACCGUGAUGAGCAACACCCUGCUGAGC | |
| | GCCUGGAUCCUGACCGCCGGCUUCCUGAUCUUCCUGAUCGGCUUCGCCCUGUUCG | |
| | GCGUGAUCCGGUGCUGCCGGUACUGCUGCUACUACUGCCUGACCCUGGAGAGCGA | |
| | GGAGCGGCCUCCUACGCCCUACCGGAACACCGUG | |
| 3′ UTR | UGAUAAUAGGCUGGAGCCUCGGUGGCCUAGCUUCUUGCCCCUUGGGCCUCCCCCC | 20 |
| | AGCCCCUCCUCCCCUUCCUGCACCCGUACCCCCGUGGUCUUUGAAUAAAGUCUGA | |
| | GUGGGCGGC | |
| PolyA | 100 nucleotides | |
| Corresponding | MNPVCLPVIVAPYLFWLAAIAASCFTASVSTVVTATGLALSLLLLAAVASSYAAA | 6 |
| Protein | QRKLLTPVTVLTAVVTFFAICLTWRIEDPPFNSLLFALLAAAGGLQGIYVLVMLV | |
| | LLILAYRRRWRRLTVCGGIMFLACVLVLIVDAVLQLSPLLGAVTVVSMTLLLLAF | |
| | VLWLSSPGGLGTLGAALLTLAAALALLASLILGTLNLTTMFLLMLLWTLVVLLIC | |
| | SSCSSCPLSKILLARLFLYALALLLLASALIAGGSILQTNFKSLSSTEFIPNLFC | |
| | MLLLIVAGILFILAILTEWGSGNRTYGPVFMCLGGLLTMVAGAVWLTVMSNTLLS | |
| | AWILTAGFLIFLIGFALFGVIRCCRYCCYYCLTLESEERPPTPYRNTV | |

The mRNA of the vaccines described herein encodes an EBV protein of interest, intended to raise an immune response to EBV infection. Thus, the EBV proteins are antigenic, i.e., they are antigens. Antigenicity is the ability to be specifically recognized by antibodies generated as a result of an immune response to a given substance, such as an EBV protein. Thus, an antigens is a protein capable of inducing an immune response (e.g., causing an immune system to produce antibodies against the antigen). In some embodiments, an antigen is an immunogen. Immunogenicity refers to the ability of a substance to induce cellular and humoral immune responses. The mRNA vaccines do not comprise antigens per se, but rather comprise mRNA that have an open reading frame encoding a protein antigen (referred to herein simply as a “EBV protein”) that once delivered to subject is expressed by cells in the subject. Delivery of the mRNA is achieved by formulating the mRNA in appropriate carriers or delivery vehicles (e.g., lipid nanoparticles) such that upon administration to cells, tissues or subjects, the mRNA is taken up by cells which, in turn, express the protein(s) encoded by the mRNA.

It should be understood that the term “protein” encompasses peptides and the term “antigen” encompasses antigenic fragments.

The mRNA vaccines provide a unique advantage over traditional protein-based vaccination approaches in which protein antigens are purified or produced in vitro, e.g., recombinant protein production technologies. The mRNA vaccines comprise mRNA encoding the desired EBV protein antigen(s), which when introduced into the body, i.e., administered to a mammalian subject (for example a human) in vivo, cause the cells of the body to express the desired antigen(s). In order to facilitate delivery of the mRNA to the cells of the body, the mRNA is encapsulated in a lipid nanoparticle (LNP). Upon delivery and uptake by cells of the body, the mRNA is translated in the cytosol and protein antigens are generated by the host cell machinery. The proteins are presented and elicit an adaptive humoral and cellular immune response. Neutralizing antibodies are directed against the expressed proteins, and hence the proteins are considered relevant target antigens for vaccine development.

Many proteins have a quaternary or three-dimensional structure, which includes more than one polypeptide or several polypeptide chains that associate into an oligomeric molecule. As used herein the term “subunit” refers to a single protein molecule, for example, a polypeptide or polypeptide chain resulting from processing of a nascent protein molecule, which subunit assembles (or “coassembles”) with other protein molecules (e.g., subunits or chains) to form a protein complex. Proteins can have a relatively small number of subunits and therefore be described as “oligomeric” or can consist of a large number of subunits and therefore be described as “multimeric”. The subunits of an oligomeric or multimeric protein may be identical, homologous or totally dissimilar and dedicated to disparate tasks.

Proteins or protein subunits can further comprise domains. As used herein, the term “domain” refers to a distinct functional and/or structural unit within a protein. Typically, a “domain” is responsible for a particular function or interaction, contributing to the overall role of a protein. Domains can exist in a variety of biological contexts. Similar domains (i.e., domains sharing structural, functional and/or sequence homology) can exist within a single protein or can exist within distinct proteins having similar or different functions. A protein domain is often a conserved part of a given protein tertiary structure or sequence that can function and exist independently of the rest of the protein or subunit thereof.

As used herein, the term “antigen” is distinct from the term “epitope,” which is a substructure of an antigen. An epitope of a part of an antigen to which an antibody attaches. An epitope may be a peptide, for example, a 7-10 amino acid peptide, or a carbohydrate structure. The art describes protein antigens that are delivered to subjects or immune cells in isolated form, e.g., isolated proteins, however, the design, testing, validation, and production of protein antigens can be costly and time-consuming, especially when producing proteins at large scale. By contrast, mRNA technology is amenable to rapid design and testing of mRNA encoding a variety of antigens. Moreover, rapid production of mRNA coupled with formulation in appropriate delivery vehicles (e.g., lipid nanoparticles), can proceed quickly and can rapidly produce mRNA vaccines at large scale. Potential benefit also arises from the fact that antigens encoded by the mRNAs are expressed by the cells of the subject, e.g., are expressed by the human body, and thus the subject, e.g., the human body, serves as the “factory” to produce the antigens which, in turn, elicits the desired immune response.

The vaccines, as provided herein, may include an mRNA or multiple RNAs encoding two or more antigens of the same or different EBV strains. Also provided herein are combination vaccines that include mRNA encoding one or more EBV antigens and one or more antigen(s) of a different organism. Thus, the mRNA vaccines may be combination vaccines that target one or more antigens of the same strain/species, or one or more antigens of different strains/species, e.g., antigens that induce immunity to organisms that are found in the same geographic areas where the risk of EBV infection is high or organisms to which an individual is likely to be exposed to when exposed to EBV.

The vaccines, as provided herein, may include multiple mRNAs, each encoding a different antigens. The mass percentage (by weight) of each mRNA may vary. In some embodiments, at least 50% of the mRNA in an EBV vaccines may encode one or more EBV glycoprotein (e.g., gp220, gH, gL, and/or soluble gp42). For example, at least 55%, at least 60%, at least 65%, at least 70%, at least 75%, at least 80%, or at least 85% of the mRNA in an EBV vaccines may encode one or more EBV glycoprotein (e.g., gp220, gH, gL, and/or soluble gp42). In some embodiments, about 50% of the mRNAs of the vaccine encodes the EBV lytic antigens. In some embodiments, about 50% to about 80% of the mRNAs of the vaccine encodes the EBV lytic antigens.

In some embodiments, less than 50% of the mRNA in an EBV vaccines encodes one or more EBV latent antigens (e.g., EBNA3A and/or LMP2B). For example, less than 45%, less than 40%, less than 35%, less than 30%, less than 25%, less than 20% of the mRNA in an EBV vaccines encodes one or more EBV latent antigens (e.g., EBNA3A and/or LMP2B). In some embodiments, about 20-50% (e.g., 20-50%, 20-45%, 20-40%, 20-35%, 20-30%, 20-25%, 25-50%, 25-45%, 25-40%, 25-35%, 25-30%, 30-50%, 30-45%, 30-40%, 30-35%, 35-50%, 35-45%, 35-40%, 40-50%, 40-45%, 45-50%) of the mRNAs of the vaccine encodes the EBV latent antigens. In some embodiments, about 20-50%, about 20-45%, about 20-40%, about 20-35%, about 20-30%, about 20-25%, about 25-50%, about 25-45%, about 25-40%, about 25-35%, about 25-30%, about 30-50%, about 30-45%, about 30-40%, about 30-35%, about 35-50%, about 35-45%, about 35-40%, about 40-50%, about 40-45%, about 45-50% of the mRNAs of the vaccine encodes the EBV latent antigens. In some embodiments, about 20% to about 50% of the mRNAs of the vaccine encodes the EBV latent antigens.

In some embodiments, an mRNA vaccine comprises: about 20% to about 35% mRNA encoding EBV gp220; about 5% to about 20% mRNA encoding EBV gH; about 5% to about 20% mRNA encoding EBV gL; about 5% to about 20% mRNA encoding EBV soluble gp42; about 20% to about 35% mRNA encoding EBNA3A; and about 20% to about 35% mRNA encoding EBV LMP2B.

In some embodiments, an mRNA vaccine comprises about 26% to about 27% (e.g., about 26.7%) mRNA encoding EBV gp220; about 9.5% to about 10.5% (e.g., about 10%) mRNA encoding EBV gH; about 6% to about 7% (e.g., about 6.7%) mRNA encoding EBV gL; about 6% to about 7% (e.g., about 6.7%) mRNA encoding EBV soluble gp42; about 24.5% to about 25.5% (e.g., about 25%) mRNA encoding EBNA3A; and about 24.5% to about 25.5% (e.g., about 25%) mRNA encoding EBV LMP2B.

In some embodiments, an mRNA vaccine comprises about 20% to about 35% mRNA encoding EBV gp220. For example, an mRNA may comprise about 20-30%, about 20-25%, about 25-35%, or about 25-30% mRNA encoding EBV gp220. In some embodiments, an mRNA vaccine comprises about 25% mRNA encoding EBV gp220. In some embodiments, an mRNA vaccine comprises about 26% mRNA encoding EBV gp220. In some embodiments, an mRNA vaccine comprises about 27% mRNA encoding EBV gp220. In some embodiments, an mRNA vaccine comprises about 28% mRNA encoding EBV gp220. In some embodiments, an mRNA vaccine comprises about 29% mRNA encoding EBV gp220. In some embodiments, an mRNA vaccine comprises about 30% mRNA encoding EBV gp220. In some embodiments, an mRNA vaccine comprises about 26% to about 27% mRNA encoding EBV gp220. In some embodiments, an mRNA vaccine comprises about 26.7% mRNA encoding EBV gp220.

In some embodiments, an mRNA vaccine comprises about 5% to about 20% mRNA encoding EBV gH. For example, an mRNA may comprise about 5-15%, about 5-10%, about 10-20%, or about 10-15% mRNA encoding EBV gH. In some embodiments, an mRNA vaccine comprises about 8% mRNA encoding EBV gH. In some embodiments, an mRNA vaccine comprises about 9% mRNA encoding EBV gH. In some embodiments, an mRNA vaccine comprises about 10% mRNA encoding EBV gH. In some embodiments, an mRNA vaccine comprises about 11% mRNA encoding EBV gH. In some embodiments, an mRNA vaccine comprises about 12% mRNA encoding EBV gH. In some embodiments, an mRNA vaccine comprises about 13% mRNA encoding EBV gH. In some embodiments, an mRNA vaccine comprises about 9.5% to about 10.5% mRNA encoding EBV gH. In some embodiments, an mRNA vaccine comprises about 10% mRNA encoding EBV gH.

In some embodiments, an mRNA vaccine comprises about 5% to about 20% mRNA encoding EBV gL. For example, an mRNA may comprise about 5-15%, about 5-10%, about 10-20%, or about 10-15% mRNA encoding EBV gL. In some embodiments, an mRNA vaccine comprises about 4% mRNA encoding EBV gL. In some embodiments, an mRNA vaccine comprises about 5% mRNA encoding EBV gL. In some embodiments, an mRNA vaccine comprises about 6% mRNA encoding EBV gL. In some embodiments, an mRNA vaccine comprises about 7% mRNA encoding EBV gL. In some embodiments, an mRNA vaccine comprises about 8% mRNA encoding EBV gL. In some embodiments, an mRNA vaccine comprises about 9% mRNA encoding EBV gL. In some embodiments, an mRNA vaccine comprises about 6% to about 7% mRNA encoding EBV gL. In some embodiments, an mRNA vaccine comprises about 6.7% mRNA encoding EBV gL.

In some embodiments, an mRNA vaccine comprises about 5% to about 20% mRNA encoding EBV soluble gp42. For example, an mRNA may comprise about 5-15%, about 5-10%, about 10-20%, or about 10-15% mRNA encoding EBV soluble gp42. In some embodiments, an mRNA vaccine comprises about 4% mRNA encoding EBV soluble gp42. In some embodiments, an mRNA vaccine comprises about 5% mRNA encoding EBV soluble gp42. In some embodiments, an mRNA vaccine comprises about 6% mRNA encoding EBV soluble gp42. In some embodiments, an mRNA vaccine comprises about 7% mRNA encoding EBV soluble gp42. In some embodiments, an mRNA vaccine comprises about 8% mRNA encoding EBV soluble gp42. In some embodiments, an mRNA vaccine comprises about 9% mRNA encoding EBV soluble gp42. In some embodiments, an mRNA vaccine comprises about 6% to about 7% mRNA encoding EBV soluble gp42. In some embodiments, an mRNA vaccine comprises about 6.7% mRNA encoding EBV soluble gp42.

In some embodiments, an mRNA vaccine comprises about 20% to about 35% mRNA encoding EBNA3A. For example, an mRNA may comprise about 20-30%, about 20-25%, about 25-35%, or about 25-30% mRNA encoding EBNA3A. In some embodiments, an mRNA vaccine comprises about 22% mRNA encoding EBNA3A. In some embodiments, an mRNA vaccine comprises about 23% mRNA encoding EBNA3A. In some embodiments, an mRNA vaccine comprises about 24% mRNA encoding EBNA3A. In some embodiments, an mRNA vaccine comprises about 25% mRNA encoding EBNA3A. In some embodiments, an mRNA vaccine comprises about 26% mRNA encoding EBNA3A. In some embodiments, an mRNA vaccine comprises about 27% mRNA encoding EBNA3A. In some embodiments, an mRNA vaccine comprises about 24.5% to about 25.5% mRNA encoding EBNA3A. In some embodiments, an mRNA vaccine comprises about 25% mRNA encoding EBNA3A.

In some embodiments, an mRNA vaccine comprises about 20% to about 35% mRNA encoding EBV LMP2B. For example, an mRNA may comprise about 20-30%, about 20-25%, about 25-35%, or about 25-30% mRNA encoding EBV LMP2B. In some embodiments, an mRNA vaccine comprises about 22% mRNA encoding EBV LMP2B. In some embodiments, an mRNA vaccine comprises about 23% mRNA encoding EBV LMP2B. In some embodiments, an mRNA vaccine comprises about 24% mRNA encoding EBV LMP2B. In some embodiments, an mRNA vaccine comprises about 25% mRNA encoding EBV LMP2B. In some embodiments, an mRNA vaccine comprises about 26% mRNA encoding EBV LMP2B. In some embodiments, an mRNA vaccine comprises about 27% mRNA encoding EBV LMP2B. In some embodiments, an mRNA vaccine comprises about 24.5% to about 25.5% mRNA encoding EBV LMP2B. In some embodiments, an mRNA vaccine comprises about 25% mRNA encoding EBV LMP2B.

In some embodiments, an mRNA vaccine comprises: about 35% to about 50% mRNA encoding EBV gp220; about 10% to about 25% mRNA encoding EBV gH; about 5% to about 20% mRNA encoding EBV gL; about 5% to about 20% mRNA encoding EBV soluble gp42; about 5% to about 20% mRNA encoding EBNA3A; and about 5% to about 20% mRNA encoding EBV LMP2B.

In some embodiments, an mRNA vaccine comprises about 42% to about 43% (e.g., about 42.1%) mRNA encoding EBV gp220; about 15% to about 16% (e.g., about 15.8%) mRNA encoding EBV gH; about 10% to about 11% (e.g., about 10.5%) mRNA encoding EBV gL; about 10% to about 11% (e.g., about 10.5%) mRNA encoding EBV soluble gp42; about 10% to about 11% (e.g., about 10.5%) mRNA encoding EBNA3A; and about 10% to about 11% (e.g., about 10.5%) mRNA encoding EBV LMP2B.

In some embodiments, an mRNA vaccine comprises about 35% to about 50% mRNA encoding EBV gp220. For example, an mRNA may comprise about 35-45%, about 35-40%, about 40-50%, or about 45-50% mRNA encoding EBV gp220. In some embodiments, an mRNA vaccine comprises about 39% mRNA encoding EBV gp220. In some embodiments, an mRNA vaccine comprises about 40% mRNA encoding EBV gp220. In some embodiments, an mRNA vaccine comprises about 41% mRNA encoding EBV gp220. In some embodiments, an mRNA vaccine comprises about 42% mRNA encoding EBV gp220. In some embodiments, an mRNA vaccine comprises about 43% mRNA encoding EBV gp220. In some embodiments, an mRNA vaccine comprises about 44% mRNA encoding EBV gp220. In some embodiments, an mRNA vaccine comprises about 42% to about 43% mRNA encoding EBV gp220. In some embodiments, an mRNA vaccine comprises about 42.1% mRNA encoding EBV gp220.

In some embodiments, an mRNA vaccine comprises about 10% to about 25% mRNA encoding EBV gH. For example, an mRNA may comprise about 10-20%, about 10-15%, about 15-25%, or about 15-20% mRNA encoding EBV gH. In some embodiments, an mRNA vaccine comprises about 12% mRNA encoding EBV gH. In some embodiments, an mRNA vaccine comprises about 13% mRNA encoding EBV gH. In some embodiments, an mRNA vaccine comprises about 14% mRNA encoding EBV gH. In some embodiments, an mRNA vaccine comprises about 15% mRNA encoding EBV gH. In some embodiments, an mRNA vaccine comprises about 16% mRNA encoding EBV gH. In some embodiments, an mRNA vaccine comprises about 17% mRNA encoding EBV gH. In some embodiments, an mRNA vaccine comprises about 15% to about 16% mRNA encoding EBV gH. In some embodiments, an mRNA vaccine comprises about 15.8% mRNA encoding EBV gH.

In some embodiments, an mRNA vaccine comprises about 5% to about 20% mRNA encoding EBV gL. For example, an mRNA may comprise about 5-15%, about 5-10%, about 10-20%, or about 10-15% mRNA encoding EBV gL. In some embodiments, an mRNA vaccine comprises about 7% mRNA encoding EBV gL. In some embodiments, an mRNA vaccine comprises about 8% mRNA encoding EBV gL. In some embodiments, an mRNA vaccine comprises about 9% mRNA encoding EBV gL. In some embodiments, an mRNA vaccine comprises about 10% mRNA encoding EBV gL. In some embodiments, an mRNA vaccine comprises about 11% mRNA encoding EBV gL. In some embodiments, an mRNA vaccine comprises about 12% mRNA encoding EBV gL. In some embodiments, an mRNA vaccine comprises about 10% to about 11% mRNA encoding EBV gL. In some embodiments, an mRNA vaccine comprises about 10.5% mRNA encoding EBV gL.

In some embodiments, an mRNA vaccine comprises about 5% to about 20% mRNA encoding EBV soluble gp42. For example, an mRNA may comprise about 5-15%, about 5-10%, about 10-20%, or about 10-15% mRNA encoding EBV soluble gp42. In some embodiments, an mRNA vaccine comprises about 7% mRNA encoding EBV soluble gp42. In some embodiments, an mRNA vaccine comprises about 8% mRNA encoding EBV soluble gp42. In some embodiments, an mRNA vaccine comprises about 9% mRNA encoding EBV soluble gp42. In some embodiments, an mRNA vaccine comprises about 10% mRNA encoding EBV soluble gp42. In some embodiments, an mRNA vaccine comprises about 11% mRNA encoding EBV soluble gp42. In some embodiments, an mRNA vaccine comprises about 12% mRNA encoding EBV soluble gp42. In some embodiments, an mRNA vaccine comprises about 10% to about 11% mRNA encoding EBV soluble gp42. In some embodiments, an mRNA vaccine comprises about 10.5% mRNA encoding EBV soluble gp42.

In some embodiments, an mRNA vaccine comprises about 5% to about 20% mRNA encoding EBNA3A. For example, an mRNA may comprise about 5-15%, about 5-10%, about 10-20%, or about 10-15% mRNA encoding EBNA3A. In some embodiments, an mRNA vaccine comprises about 7% mRNA encoding EBNA3A. In some embodiments, an mRNA vaccine comprises about 8% mRNA encoding EBNA3A. In some embodiments, an mRNA vaccine comprises about 9% mRNA encoding EBNA3A. In some embodiments, an mRNA vaccine comprises about 10% mRNA encoding EBNA3A. In some embodiments, an mRNA vaccine comprises about 11% mRNA encoding EBNA3A. In some embodiments, an mRNA vaccine comprises about 12% mRNA encoding EBNA3A. In some embodiments, an mRNA vaccine comprises about 10% to about 11% mRNA encoding EBNA3A. In some embodiments, an mRNA vaccine comprises about 10.5% mRNA encoding EBNA3A.

In some embodiments, an mRNA vaccine comprises about 5% to about 20% mRNA encoding EBV LMP2B. For example, an mRNA may comprise about 5-15%, about 5-10%, about 10-20%, or about 10-15% mRNA encoding EBV LMP2B. In some embodiments, an mRNA vaccine comprises about 7% mRNA encoding EBV LMP2B. In some embodiments, an mRNA vaccine comprises about 8% mRNA encoding EBV LMP2B. In some embodiments, an mRNA vaccine comprises about 9% mRNA encoding EBV LMP2B. In some embodiments, an mRNA vaccine comprises about 10% mRNA encoding EBV LMP2B. In some embodiments, an mRNA vaccine comprises about 11% mRNA encoding EBV LMP2B. In some embodiments, an mRNA vaccine comprises about 12% mRNA encoding EBV LMP2B. In some embodiments, an mRNA vaccine comprises about 10% to about 11% mRNA encoding EBV LMP2B. In some embodiments, an mRNA vaccine comprises about 10.5% mRNA encoding EBV LMP2B.

### Variants of Naturally Occurring Epstein Barr Virus Proteins

In some embodiments, the mRNA vaccines include mRNA that encodes an EBV protein variant. Protein variants are proteins (including full length proteins and peptides) that differ in their amino acid sequence relative to a naturally occurring or reference amino acid sequence. A protein variant may possess one or more substitutions, deletions, and/or insertions at certain positions within its amino acid sequence, as compared to a naturally occurring or reference amino acid sequence. Ordinarily, protein variants have at least 50% identity to a naturally occurring or reference sequence. In some embodiments, a protein variant has at least 60%, at least 70%, at least 75%, at least 80%, at least 85%, at least 90%, or at least 95% identity to a naturally occurring or reference sequence.

A protein variant encoded by an mRNA of the disclosure may contain amino acid changes that confer any of a number of desirable properties, for example, that enhance its immunogenicity, enhance its expression, and/or improve its stability or PK/PD properties in a subject. Protein variants can be made using routine mutagenesis techniques and assayed as appropriate to determine whether they possess the desired property. Assays to determine expression levels and immunogenicity of proteins, including protein variants, are well known in the art. Similarly, PK/PD properties of a protein variant can be measured using art recognized techniques, for example, by determining expression of the protein variant in a vaccinated subject over time and/or by looking at the durability of an induced immune response. The stability of a protein variant encoded by an mRNA may be measured by assaying thermal stability or stability upon urea denaturation or may be measured using in silico prediction, for example. Methods for such experiments and in silico determinations are known in the art. Other methods for determining protein variant expression levels, immunogenicity and/or PK/PD properties of a protein variant may be used.

In some embodiments, an mRNA comprises an open reading frame that comprises a nucleotide sequence of any one of the sequences provided herein, for example, that comprises a nucleotide sequence that has at least 50%, at least 60%, at least 70%, at least 75%, at least 80%, at least 85%, at least 90%, or at least 95% identity to a nucleotide sequence of any one of the sequences provided herein. See, e.g., SEQ ID NOs: 7-12.

In some embodiments, an mRNA comprises an open reading frame that encodes a protein comprising an amino acid sequence of any one of the sequences provided herein, for example, that comprises an amino acid sequence that has at least 80%, at least 85%, at least 90%, or at least 95% identity to the amino acid sequence of any one of the sequences provided herein. See, e.g., SEQ ID NOs: 1-6.

“Identity” refers to a relationship between two or among three or more sequences (e.g., amino acid sequences or nucleotide sequences) as determined by comparing the sequences to each other. Identity also refers to the degree of sequence relatedness between or among sequences as determined by the number of matches between or among strings of amino acids (polypeptides) or strings of nucleotides (polynucleotides). Identity is a measure of the percent of identical matches between the smaller of two or more sequences with gap alignments (if any) addressed by a particular mathematical model or computer program (e.g., “algorithms”). Identity of related polypeptides and polynucleotides can be readily calculated by known methods. “Percent (%) identity” as it applies to polypeptide or polynucleotide sequences is defined as the percentage of residues (amino acid or nucleic acid residues) in the candidate (first) polypeptide or polynucleotide sequence that are identical with the residues in a second polypeptide or polynucleotide sequence after aligning the sequences and introducing gaps, if necessary, to achieve the maximum percent identity.

Methods and computer programs for the alignment are well known in the art. It is understood that identity depends on a calculation of percent identity but may differ in value due to gaps and penalties introduced in the calculation. Generally, variants of a particular polynucleotide or polypeptide have at least 90%, 91%, 92%, 93%, 94%, 95%, 96%, 97%, 98%, 99% but less than 100% sequence identity to that particular naturally occurring or reference sequence as determined by sequence alignment programs and parameters described herein and known to those skilled in the art. Such tools for alignment include but are not limited to those of the BLAST suite (Altschul, S. F., et al. Nucleic Acids Res. 1997; 25:3389-3402); and those based on the Smith-Waterman algorithm (Smith, T. F. & Waterman, M. S. J. Mol. Biol. 1981; 147:195-197). A general global alignment technique based on dynamic programming is the Needleman-Wunsch algorithm (Needleman, S. B. & Wunsch, C. D. J. Mol. Biol. 1920; 48:443-453). A Fast Optimal Global Sequence Alignment Algorithm (FOGSAA) also has been developed that purportedly produces global alignment of nucleotide and protein sequences faster than other optimal global alignment methods, including the Needleman-Wunsch algorithm.

As such, polynucleotides and polypeptides containing substitutions, insertions and/or deletions (e.g., indels), and covalent modifications with respect to naturally occurring or reference sequence, for example, the polypeptide (e.g., protein) sequences disclosed herein, are included within the scope of this disclosure. For example, sequence tags or amino acids, such as one or more lysine(s), can be added to polypeptide sequences (e.g., at the N-terminal and/or C-terminal end). Sequence tags can be used for peptide detection, purification and/or localization. Lysines can be used to increase peptide solubility or to allow for biotinylation. Alternatively, amino acid residues located at the N-terminal and/or C-terminal regions of the amino acid sequence of a protein may optionally be deleted providing for truncated sequences. Certain amino acids (e.g., C-terminal or N-terminal amino acids) may be deleted depending on the use of the sequence, as for example, expression of the sequence as part of a larger sequence that is soluble or linked to a solid support. In some embodiments, sequences for (or encoding) signal sequences, termination sequences, transmembrane domains, linkers, multimerization domains (e.g., foldon regions) and the like are substituted with alternative sequences that achieve the same or a similar function. In some embodiments, cavities in the core of proteins can be filled to improve stability, e.g., by introducing larger amino acids. In other embodiments, buried hydrogen bond networks are replaced with hydrophobic resides to improve stability. In yet other embodiments, glycosylation sites are removed and replaced with appropriate residues. Such sequences are readily identifiable to one of skill in the art. It should also be understood that some of the sequences provided herein contain sequence tags or terminal peptide sequences (e.g., at the N-terminal or C-terminal ends) that may be deleted, for example, prior to use in the preparation of an mRNA vaccine.

As recognized by those skilled in the art, protein fragments, functional protein domains, and homologous proteins are also considered to be within the scope of EBV proteins provided herein. For example, provided herein is any protein fragment of (meaning a polypeptide sequence at least one amino acid residue shorter than but otherwise identical to) a naturally occurring or reference sequence, provided that the fragment is immunogenic and confers a protective immune response to EBV. In addition to protein variants that are identical to the naturally occurring or reference protein but are truncated, in some embodiments, a protein includes 2, 3, 4, 5, 6, 7, 8, 9, 10, or more mutations (e.g., substitutions, insertions and/or deletions), as shown in any of the sequences provided or referenced herein. Protein variants can range in length from about 4, 6, or 8 amino acids to full length proteins.

### Signal Peptides

In some embodiments, an mRNA has an ORF that encodes a signal peptide fused to the EBV protein. Signal peptides, comprising the N-terminal 15-60 amino acids of proteins, are typically needed for the translocation across the membrane on the secretory pathway and, thus, universally control the entry of most proteins both in eukaryotes and prokaryotes to the secretory pathway. In eukaryotes, the signal peptide of a nascent precursor protein (pre-protein) directs the ribosome to the rough endoplasmic reticulum (ER) membrane and initiates the transport of the growing peptide chain across it for processing. ER processing produces mature proteins, wherein the signal peptide is cleaved from precursor proteins, typically by an ER-resident signal peptidase of the host cell, or they remain uncleaved and function as a membrane anchor. A signal peptide may also facilitate the targeting of the protein to the cell membrane.

A signal peptide may have a length of 15-60 amino acids. For example, a signal peptide may have a length of 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, or 60 amino acids. In some embodiments, a signal peptide has a length of 20-60, 25-60, 30-60, 35-60, 40-60, 45-60, 50-60, 55-60, 15-55, 20-55, 25-55, 30-55, 35-55, 40-55, 45-55, 50-55, 15-50, 20-50, 25-50, 30-50, 35-50, 40-50, 45-50, 15-45, 20-45, 25-45, 30-45, 35-45, 40-45, 15-40, 20-40, 25-40, 30-40, 35-40, 15-35, 20-35, 25-35, 30-35, 15-30, 20-30, 25-30, 15-25, 20-25, or 15-20 amino acids.

Signal peptides from heterologous genes (which regulate expression of genes other than EBV proteins in nature) are known in the art and can be tested for desired properties and then incorporated into a nucleic acid of the disclosure.

In some embodiments, an mRNA comprises an open reading frame that encodes an EBV protein fused to a signal peptide comprising an amino acid sequence that has at least 50%, at least 60%, at least 70%, at least 75%, at least 80%, at least 85%, at least 90%, or at least 95% identity to the amino acid sequence of any one of the signal peptide sequences provided herein. See, e.g., SEQ ID NOs: 50-54, which are reproduced below in Table 3. In some embodiments, an mRNA comprises an open reading frame that encodes an EBV protein including an endogenous signal peptide of the wild-type EBV protein (e.g., an mRNA encoding a (wild-type or modified) EBV gB encodes an EBV gB signal peptide).

TABLE 3
Signal Peptides

| Description | Sequence | SEQ ID NO: |
|---|---|---|
| HuIgGₖ signal peptide | METPAQLLFLLLLWLPDTTG | 50 |
| IgE heavy chain epsilon −1 signal | MDWTWILFLVAAATRVHS | 51 |
| peptide | | |
| Japanese encephalitis PRM signal | MLGSNSGQRVVFTILLLLVAPAYS | 52 |
| sequence | | |
| VSVg protein signal sequence | MKCLLYLAFLFIGVNCA | 53 |
| Japanese encephalitis JEV signal | MWLVSLAIVTACAGA | 54 |
| sequence | | |

### Fusion Proteins

In some embodiments, an mRNA encodes a fusion protein. Thus, an encoded protein may include two or more proteins (e.g., protein and/or protein fragment) joined together with or without a linker. Fusion proteins, in some embodiments, retain the functional property of each independent (nonfusion) protein.

In some embodiments, a fusion protein comprises 2, 3, 4, 5, 6, 7, 8, 9, or 10 of the following EBV proteins: gp220, gH, gL, gp42, EBNA3A, and LMP2B.

### Linkers and Cleavable Peptides

In some embodiments, an mRNA that encodes a fusion protein further encodes a linker located between at least one or each domain of the fusion protein. The linker may be, for example, a cleavable linker or protease-sensitive linker. In some embodiments, the linker is selected from the group consisting of F2A linker, P2A linker, T2A linker, E2A linker, and combinations thereof (see, e.g., WO 2017/127750). This family of self-cleaving peptide linkers, referred to as 2A peptides, has been described in the art (see, e.g., Kim, J. H. et al. PLoS ONE 2011; 6:e18556). In some embodiments, the linker is an F2A linker.

In some embodiments, the linker is a GS linker. GS linkers are polypeptide linkers that include glycine and serine amino acids repeats. They comprise flexible and hydrophilic residues and can be used to perform fusion of protein subunits without interfering in the folding and function of the protein domains, and without formation of secondary structures. In some embodiments, an mRNA encodes a fusion protein that comprises a GS linker that is 3 to 20 amino acids long. For example, the GS linker may have a length of (or have a length of at least) 3, 4, 5, 6, 7, 8, 9, 10, 11, 12, 13, 14, 15, 16, 17, 18, 19, or 20 amino acids. In some embodiments, a GS linker is (or is at least) 15 amino acids long (e.g., GGSGGSGGSGGSGGG (SEQ ID NO: 36)). In some embodiments, a GS linker is (or is at least) 8 amino acids long (e.g., GGGSGGGS (SEQ ID NO: 37)). In some embodiments, a GS linker is (or is at least) 7 amino acids long (e.g., GGGSGGG (SEQ ID NO: 38)). In some embodiments, a GS linker comprises the amino acid sequence GGGSGG (SEQ ID NO: 39). In some embodiments, a GS linker is (or is at least) 4 amino acid long (e.g., GGGS (SEQ ID NO: 40)). In some embodiments, the GS linker comprises (GGGS)n (SEQ ID NO: 41), where n is any integer from 1-5. In some embodiments, a GS linker is (or is at least) 4 amino acid long (e.g., GSGG (SEQ ID NO: 42)). In some embodiments, the GS linker comprises (GSGG)n (SEQ ID NO: 43), where n is any integer from 1-5. In some embodiments, a linker is a glycine linker, for example having a length of (or a length of at least) 3 amino acids (e.g., GGG). In some embodiments, a protein encoded by an mRNA includes two or more linkers, which may be the same or different from each other.

The skilled artisan will appreciate that other art-recognized linkers may be suitable for use in the constructs of the disclosure (e.g., encoded by the nucleic acids of the disclosure). The skilled artisan will likewise appreciate that other polycistronic constructs (mRNA encoding more than one protein separately within the same molecule) may be suitable for use as provided herein.

### Nucleic Acids Encoding Epstein-Barr Virus Proteins

Nucleic acids comprise a polymer of nucleotides (nucleotide monomers). Thus, nucleic acids are also referred to as polynucleotides. Nucleic acids may be or may include, for example, deoxyribonucleic acid (DNA), ribonucleic acid (RNA), threose nucleic acid (TNA), glycol nucleic acid (GNA), peptide nucleic acid (PNA), locked nucleic acid (LNA, including LNA having a β-D-ribo configuration, α-LNA having an α-L-ribo configuration (a diastereomer of LNA), 2′-amino-LNA having a 2′-amino functionalization, and 2′-amino-α-LNA having a 2′-amino functionalization), ethylene nucleic acid (ENA), cyclohexenyl nucleic acid (CeNA) and/or chimeras and/or combinations thereof.

mRNA of the mRNA vaccines described herein comprises an open reading frame (ORF) encoding an EBV protein. In some embodiments, the mRNA further comprises a 5′ untranslated region (UTR), 3′ UTR, a poly(A) tail and/or a 5′ cap analog.

### Messenger RNA

Messenger RNA (mRNA) is RNA that encodes a (at least one) protein (a naturally-occurring, non-naturally-occurring, or modified polymer of amino acids) and can be translated to produce the encoded protein in vitro, in vivo, in situ, or ex vivo. It is understood that mRNA is not self-amplifying (i.e., self-replicating) RNA (saRNA) (see, e.g., Bloom K et al. Gene Therapy 2021; 28: 117-129 for a comparison of mRNA and saRNA). saRNAs include alphavirus replicase sequences that encode an RNA-dependent RNA polymerase. mRNA does not include alphavirus replicase sequences.

The skilled artisan will appreciate that, except where otherwise noted, nucleic acid sequences set forth in the instant application may recite “T”s in a representative DNA sequence but where the sequence represents mRNA, the “T”s would be substituted for “U”s. Thus, any of the DNAs disclosed and identified by a particular sequence identification number herein also disclose the corresponding mRNA sequence complementary to the DNA, where each “T” of the DNA sequence is substituted with “U.”

Naturally occurring eukaryotic mRNA molecules can contain stabilizing elements, including, but not limited to, UTRs at their 5′-end (5′ UTR) and/or at their 3′-end (3′ UTR), in addition to other structural features, such as a 5′-cap structure or a 3′-poly(A) tail. Both the 5′ UTR and the 3′ UTR are typically transcribed from the genomic DNA and are elements of the premature mRNA. Characteristic structural features of mature mRNA, such as the 5′-cap and the 3′-poly(A) tail are usually added to the transcribed (premature) mRNA during mRNA processing.

Exemplary sequences of mRNA that encode EBV proteins are provided in the Examples section elsewhere herein. In some embodiments, the mRNA comprises an ORF that is at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or identical to a sequence selected from SEQ ID NOs: 7-12. In some embodiments, the mRNA comprises a nucleotide sequence that is 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or identical to a sequence selected from SEQ ID NOs: 7-12.

### Untranslated Regions (UTRs)

The mRNAs comprise one or more regions or parts that function as an untranslated region. A “5′ untranslated region” (UTR) refers to a region of an mRNA that is directly upstream (i.e., 5′) from the start codon (i.e., the first codon of an mRNA transcript translated by a ribosome) that does not encode a polypeptide. A “3′ untranslated region” (UTR) refers to a region of an mRNA that is directly downstream (i.e., 3′) from the stop codon (i.e., the codon of an mRNA transcript that signals a termination of translation) that does not encode a polypeptide. When RNA transcripts are being generated, the 5′ UTR may comprise a promoter sequence. Such promoter sequences are known in the art. It should be understood that such promoter sequences will not be present in a vaccine of the disclosure.

Where mRNAs are designed to encode a (at least one) EBV protein, the mRNA may comprise a 5′ UTR and/or 3′ UTR. UTRs of an mRNA are transcribed but not translated. In mRNA, the 5′ UTR starts at the transcription start site and continues to the start codon but does not include the start codon; the 3′ UTR starts immediately following the stop codon and continues until the transcriptional termination signal. There is a growing body of evidence about the regulatory roles played by the UTRs in terms of stability of the nucleic acid molecule and translation. The regulatory features of a UTR can be incorporated into the polynucleotides to, among other things, enhance the stability of the molecule. The specific features can also be incorporated to ensure controlled down-regulation of the transcript in case they are misdirected 5 to undesired organs sites. A variety of 5′ UTR and 3′ UTR sequences are known.

It should also be understood that the mRNA may include any 5′ UTR and/or any 3′ UTR. Exemplary UTR sequences include SEQ ID NOs: 76-86; however, other UTR sequences may be used or exchanged for any of the UTR sequences described herein.

In some embodiments, the 5′ UTR comprises a sequence provided in Table 4 or a 10 sequence with at least 80%, 85%, 90%, 95%, 96%, 97%, 98%, 99% or 100% identity to a 5′ UTR sequence provided in Table 4, or a variant or a fragment thereof.

In an embodiment, the 3′ UTR comprises a sequence provided in Table 5 or a sequence with at least 80%, 85%, 90%, 95%, 96%, 97%, 98%, 99% or 100% identity to a 3′ UTR sequence provided in Table 5, or a variant or a fragment thereof.

TABLE 4
5′ UTR sequences

| SEQ ID | Sequence |
|---|---|
| NO: | |
| 55 | GGGAAAUAAGAGAGAAAAGAAGAGUAAGAAGAAAUAUAAGAGCCACC |
| 56 | GGGAAAUAAGAGAGAAAAGAAGAGUAAGAAGAAAUAUAAGACCCCGGCGCCGCCACC |
| 57 | GGAAAUCGCAAAAUUUGCUCUUCGCGUUAGAUUUCUUUUAGUUUUCUCGCAACUAGCAAGCUUUUUG |
| | UUCUCGCC |
| 58 | GGAAAUCCCCACAACCGCCUCAUAUCCAGGCUCAAGAAUAGAGCUCAGUGUUUUGUUGUUUAAUCAU |
| | UCCGACGUGUUUUGCGAUAUUCGCGCAAAGCAGCCAGUCGCGCGCUUGCUUUUAAGUAGAGUUGUUUU |
| | UCCACCCGUUUGCCAGGCAUCUUUAAUUUAACAUAUUUUUAUUUUUCAGGCUAACCUACGCCGCCACC |
| 59 | GGAAAUAAGAGAGAAAAGAAGAGUAAGAAGAAAUAUAAGAUCUCCCUGAGCUUCAGGGAGCCCCGGC |
| | GCCGCCACC |
| 60 | GGAAACCCCCCACCCCCGUAAGAGAGAAAAGAAGAGUAAGAAGAAAUAUAAGAUCUCCCUGAGCUUC |
| | AGGGAGCCCCGGCGCCGCCACC |
| 61 | GGAGAACUUCCGCUUCCGUUGGCGCAAGCGCUUUCAUUUUUUCUGCUACCGUGACUAAG |
| 62 | GGAAAUAAGAGAGAAAAGAAGAGUAAGAAGAAAUAUAAGAGCCACC |
| 63 | GGAAAUAAGAGAGAAAAGAAGAGUAAGAAGAAAUAUAAGACCCCGGCGCCGCCACC |
| 64 | GGAAAUCGCAAAAUUUGCUCUUCGCGUUAGAUUUCUUUUAGUUUUCUCGCAACUAGCAAGCUUUUUG |
| | UUCUCGCCGCCGCC |
| 65 | GGAAAUCGCAAAAUUUUCUUUUCGCGUUAGAUUUCUUUUAGUUUUCUUUCAACUAGCAAGCUUUUUG |
| | UUCUCGCCGCCGCC |
| 66 | G G A A A U C G C A A A A (N₂)ₓ (N₃)ₓ C U (N₄)ₓ (N₅)ₓ C G C G U U A G A |
| | U UU C U U U U A G U U U U C U N₆ N₇ C A A C U A G C A A G C U U U U |
| | U G U U C U C G C C (N₈ C C)x |
| | (N₂)ₓ is a uracil and x is an integer from 0 to 5, e.g., wherein x = 3 |
| | or 4; |
| | (N₃)ₓ is a guanine and x is an integer from 0 to 1; |
| | (N₄)ₓ is a cytosine and x is an integer from 0 to 1; |
| | (N₅)ₓ is a uracil and x is an integer from 0 to 5, e.g., wherein x = 2 |
| | or 3; |
| | N₆ is a uracil or cytosine; |
| | N₇ is a uracil or guanine; |
| | N₈ is adenine or guanine and x is an integer from 0 to 1. |
| 67 | GGAAAAUUUUAGCCUGGAACGUUAGAUAACUGUCCUGUUGUCUUUAUAUACUUGGUCCCCAAGUAGU |
| | UUGUCUUCCAAA |
| 68 | GGAAACUUUAUUUAGUGUUACUUUAUUUUCUGUUUAUUUGUGUUUCUUCAGUGGGUUUGUUCUAAUU |
| | UCCUUGGCCGCC |
| 69 | GGAAAAUCUGUAUUAGGUUGGCGUGUUCUUUGGUCGGUUGUUAGUAUUGUUGUUGAUUCGUUUGUGG |
| | UCGGUUGCCGCC |
| 70 | GGAAAAUUAUUAACAUCUUGGUAUUCUCGAUAACCAUUCGUUGGAUUUUAUUGUAUUCGUAGUUUGG |
| | GUUCCUGCCGCC |
| 71 | GGAAAUUAUUAUUAUUUCUAGCUACAAUUUAUCAUUGUAUUAUUUUAGCUAUUCAUCAUUAUUUACU |
| | UGGUGAUCAACA |
| 72 | GGAAAUAGGUUGUUAACCAAGUUCAAGCCUAAUAAGCUUGGAUUCUGGUGACUUGCUUCACCGUUGG |
| | CGGGCACCGAUC |
| 73 | GGAAAUCGUAGAGAGUCGUACUUAGUACAUAUCGACUAUCGGUGGACACCAUCAAGAUUAUAAACCA |
| | GGCCAGA |
| 74 | GGAAACCCGCCCAAGCGACCCCAACAUAUCAGCAGUUGCCCAAUCCCAACUCCCAACACAAUCCCCA |
| | AGCAACGCCGCC |
| 75 | GGAAAGCGAUUGAAGGCGUCUUUUCAACUACUCGAUUAAGGUUGGGUAUCGUCGUGGGACUUGGAAA |
| | UUUGUUGUUUCC |
| 76 | GGAAACUAAUCGAAAUAAAAGAGCCCCGUACUCUUUUAUUUCUAUUAGGUUAGGAGCCUUAGCAUUU |
| | GUAUCUUAGGUA |
| 77 | GGAAAUGUGAUUUCCAGCAACUUCUUUUGAAUAUAUUGAAUUCCUAAUUCAAAGCGAACAAAUCUAC |
| | AAGCCAUAUACC |
| 78 | GGAAAUCGUAGAGAGUCGUACUUACGUGGUCGCCAUUGCAUAGCGCGCGAAAGCAACAGGAACAAGA |
| | ACGCGCC |
| 79 | GGAAAUCGUAGAGAGUCGUACUUAGAAUAAACAGAGUCGGGUCGACUUGUCUCUGAUACUACGACGU |
| | CACAAUC |
| 80 | GGAAAAUUUGCCUUCGGAGUUGCGUAUCCUGAACUGCCCAGCCUCCUGAUAUACAACUGUUCCGCUU |
| | AUUCGGGCCGCC |
| 81 | GGAAAUCUGAGCAGGAAUCCUUUGUGCAUUGAAGACUUUAGAUUCCUCUCUGCGGUAGACGUGCACU |
| | UAUAAGUAUUUG |
| 82 | GGAAAGCGAUUGAAGGCGUCUUUUCAACUACUCGAUUAAGGUUGGGUAUCGUCGUGGGACUUGGAAA |
| | UUUGUUGCCACC |
| 83 | GGAAAUUUUUUUUUGAUAUUAUAAGAGUUUUUUUUUGAUAUUAAGAAAAUUUUUUUUUGAUAUUAGA |
| | AGAGUAAGAAGAAAUAUAAGACCCCGGCGCCGCCACC |
| 84 | GGAAAUAAGAGAGAAAAGAAGAGUAAGAAGAAAUAUAAGAGCCAAAAAAAAAAAACC |
| 85 | GGAAAUCUCCCUGAGCUUCAGGGAGUAAGAGAGAAAAGAAGAGUAAGAAGAAAUAUAAGACCCCGGC |
| | GCCGCCACC |
| | GCCRCC, wherein R = A or G |
| 86 | GGACUCACUAUUUGUUUUCGCGCCCAGUUGCAAAAA |

TABLE 5
3′ UTR sequences (stop cassette is italicized; miR binding sites are boldened)

| SEQ ID | |
|---|---|
| NO: | Sequence |
| 87 | UGAUAAUAGGCUGGAGCCUCGGUGGCCAUGCUUCUUGCCCCUUGGGCCUCCCCCCAGCC |
| | CCUCCUCCCCUUCCUGCACCCGUACCCCCGUGGUCUUUGAAUAAAGUCUGAGUGGGGGC |
| 20 | UGAUAAUAGGCUGGAGCCUCGGUGGCCUAGCUUCUUGCCCCUUGGGCCUCCCCCCAGCC |
| | CCUCCUCCCCUUCCUGCACCCGUACCCCCGUGGUCUUUGAAUAAAGUCUGAGUGGGCGGC |
| 88 | UAAAGCUCCCCGGGGGCCUCGGUGGCCUAGCUUCUUGCCCCUUGGGCCUCCCCCCAGCCCCUCCUCCCCUUC |
| | CUGCACCCGUACCCCCGUGGUCUUUGAAUAAAGUCUGAGUGGGCGGC |
| 89 | UAAGUCUAAGCUGGAGCCUCCUGAGAGACCUGUGUGAACUAUUGAGAAGAUCGGAACAGCUCCUUACUCUGA |
| | GGAAGUUGGUACCCCCGUGGUCUUUGAAUAAAGUCUGAGUGGGCGGC |
| 87 | UGAUAAUAGGCUGGAGCCUCGGUGGCCAUGCUUCUUGCCCCUUGGGCCUCCCCCCAGCC |
| | CCUCCUCCCCUUCCUGCACCCGUACCCCCGUGGUCUUUGAAUAAAGUCUGAGUGGGCGGC |
| 20 | UGAUAAUAGGCUGGAGCCUCGGUGGCCUAGCUUCUUGCCCCUUGGGCCUCCCCCCAGCC |
| | CCUCCUCCCCUUCCUGCACCCGUACCCCCGUGGUCUUUGAAUAAAGUCUGAGUGGGCGGC |
| 90 | UAAAGCUCCCCGGGGCAAACACCAUUGUCACACUCCAGCCUCGGUGGCCUAGCUUCUUGCCCCUUGGGCCCA |
| | AACACCAUUGUCACACUCCAUCCCCCCAGCCCCUCCUCCCCUUCCUGCACCCGUACCCCCCAAACACCAUUG |
| | UCACACUCCAGUGGUCUUUGAAUAAAGUCUGAGUGGGCGGC |
| | (miR122 binding sites boldened) |
| 91 | UAAAGCUCCCCGGGGUCCAUAAAGUAGGAAACACUACAGCUGGAGCCUCGGUGGCCUAGCUUCUUGCCCCUU |
| | GGGCCCAAACACCAUUGUCACACUCCAUCCCCCCAGCCCCUCCUCCCCUUCCUGCACCCGUACCCCCGUGGU |
| | CUUUGAAUAAAGUCUGAGUGGGCGGC |
| | (miR-142-3p and miR122 binding sites boldened) |
| 92 | UAAAGCUCCCCGGGGGCCUCGGUGGCCUAGCUUCUUGCCCCUUGGGCCUCCCCCCAGCCCCUCCUCCCCUUC |
| | CUGCACCCGUACCCCCCAAACACCAUUGUCACACUCCAGUGGUCUUUGAAUAAAGUCUGAGUGGGCGGC |
| | (miR122 binding site boldened) |
| 93 | UAAGCCCCUCCGGGGCAAACACCAUUGUCACACUCCAGCCUCGGUGGCCUAGCUUCUUGCCCCUUGGGCCCA |
| | AACACCAUUGUCACACUCCAUCCCCCCAGCCCCUCCUCCCCUUCCUGCACCCGUACCCCCCAAACACCAUUG |
| | UCACACUCCAGUGGUCUUUGAAUAAAGUCUGAGUGGGCGGC |
| | (miR122 binding sites boldened) |
| 94 | UAAGCCCCUCCGGGGUCCAUAAAGUAGGAAACACUACAGCCUCGGUGGCCUAGCUUCUUGCCCCUUGGGCCU |
| | CCAUAAAGUAGGAAACACUACAUCCCCCCAGCCCCUCCUCCCCUUCCUGCACCCGUACCCCCCGCAUUAUUA |
| | CUCACGGUACGAGUGGUCUUUGAAUAAAGUCUGAGUGGGGGC |
| | (miR-142-3p and miR-126-3p binding sites boldened) |
| 95 | UAAGCCCCUCCGGGGUCCAUAAAGUAGGAAACACUACAGCUGGAGCCUCGGUGGCCUAGCUUCUUGCCCCUU |
| | GGGCCCAAACACCAUUGUCACACUCCAUCCCCCCAGCCCCUCCUCCCCUUCCUGCACCCGUACCCCCGUGGU |
| | CUUUGAAUAAAGUCUGAGUGGGCGGC |
| | (miR-142-3p and miR122 binding sites boldened) |
| 96 | UAAGCCCCUCCGGGGGCCUCGGUGGCCUAGCUUCUUGCCCCUUGGGCCUCCCCCCAGCCCCUCCUCCCCUUC |
| | CUGCACCCGUACCCCCCAAACACCAUUGUCACACUCCAGUGGUCUUUGAAUAAAGUCUGAGUGGGCGGC |
| | (miR122 binding site boldened) |

A 5′ UTR does not encode a protein (is non-coding). Natural 5′ UTRs have features that play roles in translation initiation. They harbor signatures like Kozak sequences which are commonly known to be involved in the process by which the ribosome initiates translation of many genes. Kozak sequences have the consensus CCR(A/G)CCAUGG, where R is a purine (adenine or guanine) three bases upstream of the start codon (AUG), which is followed by another ‘G’. 5′UTR also have been known to form secondary structures which are involved in elongation factor binding.

In some embodiments of the disclosure, a 5′ UTR is a heterologous UTR, i.e., is a UTR 10 found in nature associated with a different ORF. In other embodiments, a 5′ UTR is a synthetic UTR, i.e., does not occur in nature. Synthetic UTRs include UTRs that have been mutated to improve their properties, e.g., which increase gene expression as well as those which are completely synthetic. Exemplary 5′ UTRs include Xenopus or human derived a-globin or b-globin (U.S. Pat. Nos. 8,278,063; 9,012,219), human cytochrome b-245 a polypeptide, and hydroxysteroid (17b) dehydrogenase, and Tobacco etch virus (U.S. Pat. Nos. 8,278,063, 9,012,219). CMV immediate-early 1 (IE1) gene (US2014/0206753, WO2013/185069), the sequence GGGAUCCUACC (SEQ ID NO: 44) (WO2014/144196) may also be used. In other embodiments, a 5′ UTR is a 5′ UTR of a TOP gene lacking the 5′ TOP motif (the oligopyrimidine tract) (e.g., WO2015/101414, WO2015/101415, WO2015/062738, WO2015/024667, WO2015/024667); 5′ UTR element derived from ribosomal protein Large 32 (L32) gene (WO/2015101414, WO2015101415, WO/2015/062738), 5′ UTR element derived from the 5′ UTR of an hydroxysteroid (17-0) dehydrogenase 4 gene (HSD17B4) (WO2015024667), or a 5′ UTR element derived from the 5′ UTR of ATP5A1 (WO2015/024667) can be used. In some embodiments, an internal ribosome entry site (IRES) is used instead of a 5′ UTR.

A 3′ UTR does not encode a protein (is non-coding). Natural or wild type 3′ UTRs are known to have stretches of adenosines and uridines embedded in them. These AU rich signatures are particularly prevalent in genes with high rates of turnover. Based on their sequence features and functional properties, the AU rich elements (AREs) can be separated into three classes (Chen et al, 1995): Class I AREs contain several dispersed copies of an AUUUA motif within U-rich regions. C-Myc and MyoD contain class I AREs. Class II AREs possess two or more overlapping UUAUUUA(U/A)(U/A) nonamers. Molecules containing this type of AREs include GM-CSF and TNF-α. Class III ARES are less well defined. These U rich regions do not contain an AUUUA motif. c-Jun and Myogenin are two well-studied examples of this class. Most proteins binding to the AREs are known to destabilize the messenger, whereas members of the ELAV family, most notably HuR, have been documented to increase the stability of mRNA. HuR binds to AREs of all the three classes. Engineering the HuR specific binding sites into the 3′ UTR of nucleic acid molecules will lead to HuR binding and thus, stabilization of the message in vivo.

Introduction, removal or modification of 3′ UTR AU rich elements (AREs) can be used to modulate the stability of mRNA of the disclosure. When engineering specific nucleic acids, one or more copies of an ARE can be introduced to make nucleic acids of the disclosure less stable and thereby curtail translation and decrease production of the resultant protein. Likewise, AREs can be identified and removed or mutated to increase the intracellular stability and thus increase translation and production of the resultant protein. Transfection experiments can be conducted in relevant cell lines, using nucleic acids of the disclosure and protein production can be assayed at various time points post-transfection. For example, cells can be transfected with different ARE-engineering molecules and by using an ELISA kit to the relevant protein and assaying protein produced at 6 hours, 12 hours, 1 day, 2 days, and 7 days post-transfection.

Those of ordinary skill in the art will understand that 5′ UTRs that are heterologous or synthetic may be used with any desired 3′ UTR sequence. For example, a heterologous or synthetic 5′ UTR may be used with a synthetic 3′ UTR or with a heterologous 3′ UTR.

Non-UTR sequences may also be used as regions or subregions within a nucleic acid. For example, introns or portions of introns sequences may be incorporated into regions of nucleic acid of the disclosure. Incorporation of intronic sequences may increase protein production as well as nucleic acid levels.

Combinations of features may be included in flanking regions and may be contained within other features. For example, the ORF may be flanked by a 5′ UTR which may contain a strong Kozak translational initiation signal and/or a 3′ UTR which may include an oligo(dT) sequence for templated addition of a poly-A tail. 5′ UTR may comprise a first polynucleotide fragment and a second polynucleotide fragment from the same and/or different genes such as the 5′ UTRs described in US2010/0293625 and WO2015/085318A2, each of which is herein incorporated by reference.

It should be understood that any UTR from any gene may be incorporated into the regions of a nucleic acid. Furthermore, multiple wild-type UTRs of any known gene may be utilized. It is also within the scope of the present disclosure to provide artificial UTRs which are not variants of wild type regions. These UTRs or portions thereof may be placed in the same orientation as in the transcript from which they were selected or may be altered in orientation or location. Hence a 5′ or 3′ UTR may be inverted, shortened, lengthened, made with one or more other 5′ UTRs or 3′ UTRs. As used herein, the term “altered” as it relates to a UTR sequence, means that the UTR has been changed in some way in relation to a reference sequence. For example, a 3′ UTR or 5′ UTR may be altered relative to a wild-type/native UTR by the change in orientation or location as taught above or may be altered by the inclusion of additional nucleotides, deletion of nucleotides, swapping or transposition of nucleotides. Any of these changes producing an “altered” UTR (whether 3′ or 5′) comprise a variant UTR.

In some embodiments, a double, triple or quadruple UTR such as a 5′ UTR or 3′ UTR may be used. As used herein, a “double” UTR is one in which two copies of the same UTR are encoded either in series or substantially in series. For example, a double beta-globin 3′ UTR may be used as described in US2010/0129877, which is incorporated herein by reference.

It is also within the scope of the present disclosure to have patterned UTRs. As used herein “patterned UTRs” are those UTRs which reflect a repeating or alternating pattern, such as ABABAB or AABBAABBAABB or ABCABCABC or variants thereof repeated once, twice, or more than 3 times. In these patterns, each letter, A, B, or C represent a different UTR at the nucleotide level.

In some embodiments, flanking regions are selected from a family of transcripts whose proteins share a common function, structure, feature, or property. For example, polypeptides of interest may belong to a family of proteins which are expressed in a particular cell, tissue or at some time during development. The UTRs from any of these genes may be swapped for any other UTR of the same or different family of proteins to create a new polynucleotide. As used herein, a “family of proteins” is used in the broadest sense to refer to a group of two or more polypeptides of interest which share at least one function, structure, feature, localization, origin, or expression pattern.

The untranslated region may also include translation enhancer elements (TEE). As a non-limiting example, the TEE may include those described in US 2009/0226470, herein incorporated by reference, and those known in the art.

### Open Reading Frames

An open reading frame (ORF) is a continuous stretch of DNA or RNA beginning with a start codon (e.g., methionine (ATG or AUG)) and ending with a stop codon (e.g., TAA, TAG or TGA, or UAA, UAG or UGA). An ORF typically encodes a protein. It will be understood that the sequences disclosed herein may further comprise additional elements, e.g., 5′ and/or 3′ UTRs, but that those elements, unlike the ORF, need not necessarily be present in an mRNA.

### 5′ End Capping

In some embodiments, an mRNA comprises a 5′ terminal cap. 5′-capping of polynucleotides may be completed concomitantly during an in vitro transcription reaction using, for example, the following chemical RNA cap analogs to generate the 5′-guanosine cap structure according to manufacturer protocols: 3′-O-Me-m7G(5′)ppp(5′) G [the ARCA cap]; G(5′)ppp(5′)A; G(5′)ppp(5′)G; m7G(5′)ppp(5′)A; m7G(5′)ppp(5′)G (New England BioLabs, Ipswich, MA). 5′-capping of modified mRNA may be completed post-transcriptionally using, for example, a Vaccinia Virus Capping Enzyme to generate the “Cap 0” structure: m7G(5′)ppp(5′)G (New England BioLabs, Ipswich, MA). Cap 1 structure may be generated using both Vaccinia Virus Capping Enzyme and a 2′-O methyl-transferase to generate: m7G(5′)ppp(5′)G-2′-O-methyl. Cap 2 structure may be generated from the Cap 1 structure followed by the 2′-O-methylation of the 5′-antepenultimate nucleotide using a 2′-O methyl-transferase. Cap 3 structure may be generated from the Cap 2 structure followed by the 2′-O-methylation of the 5′-preantepenultimate nucleotide using a 2′-0 methyl-transferase. Enzymes may be derived from a recombinant source. Other cap analogs may be used.

### Polyadenylation Tailing

A “poly(A) tail” is a region of mRNA that is downstream, e.g., directly downstream (i.e., 3′), from the 3′ UTR that contains multiple, consecutive adenosine monophosphates. A poly(A) tail may contain 10 to 300 adenosine monophosphates. It can, in some instances, comprise up to about 400 adenine nucleotides. For example, a poly(A) tail may contain 10, 20, 30, 40, 50, 60, 70, 80, 90, 100, 110, 120, 130, 140, 150, 160, 170, 180, 190, 200, 210, 220, 230, 240, 250, 260, 270, 280, 290 or 300 adenosine monophosphates. In some embodiments, a poly(A) tail contains 50 to 250 adenosine monophosphates. In a relevant biological setting (e.g., in cells, in vivo) the poly(A) tail functions to protect mRNA from enzymatic degradation, e.g., in the cytoplasm, and aids in transcription termination, and/or export of the mRNA from the nucleus and translation. 5 In some embodiments, the length of the 3′-poly(A) tail may be an essential element with respect to the stability of the individual mRNA. In some embodiments, a poly(A) tail has a length of about 50, about 100, about 150, about 200, about 250, about 300, about 350, or about 400 nucleotides. In some embodiments, a poly(A) tail has a length of 100 nucleotides.

### Additional Stabilizing Elements

mRNA provided herein, in some embodiments, includes an additional stabilizing element. Stabilizing elements may include, for example, a histone stem-loop. A stem-loop binding protein (SLBP), a 32 kDa protein has been identified. It is associated with the histone stem-loop at the 3′-end of the histone messages in both the nucleus and the cytoplasm. Its expression level is regulated by the cell cycle; it peaks during the S-phase, when histone mRNA levels are also elevated. The protein has been shown to be essential for efficient 3′-end processing of histone pre-mRNA by the U7 snRNP. SLBP continues to be associated with the stem-loop after processing, and then stimulates the translation of mature histone mRNAs into histone proteins in the cytoplasm. The RNA binding domain of SLBP is conserved through metazoa and protozoa; its binding to the histone stem-loop depends on the structure of the loop. The minimum binding site includes at least three nucleotides 5′ and two nucleotides 3′ relative to the stem-loop.

In some embodiments, an mRNA includes an open reading frame (coding region), a histone stem-loop, and optionally, a poly(A) sequence or polyadenylation signal. The poly(A) sequence or polyadenylation signal generally should enhance the expression level of the encoded protein. The encoded protein, in some embodiments, is not a histone protein, a reporter protein (e.g., Luciferase, GFP, EGFP, β-Galactosidase, EGFP), or a marker or selection protein (e.g., alpha-Globin, Galactokinase and Xanthine:guanine phosphoribosyl transferase (GPT)).

In some embodiments, an mRNA includes the combination of a poly(A) sequence or polyadenylation signal and at least one histone stem-loop, even though both represent alternative mechanisms in nature, they act synergistically to increase the protein expression beyond the level observed with either of the individual elements. The synergistic effect of the combination of poly(A) and a histone stem-loop does not depend on the order of the elements or the length of the poly(A) sequence.

In some embodiments, an mRNA does not include a histone downstream element (HDE). “Histone downstream element” (HDE) includes a purine-rich polynucleotide stretch of approximately 15 to 20 nucleotides 3′ of naturally-occurring stem-loops, representing the binding site for the U7 snRNA, which is involved in processing of histone pre-mRNA into mature histone mRNA. In some embodiments, the nucleic acid does not include an intron.

An mRNA may or may not contain an enhancer and/or promoter sequence, which may be modified or unmodified or which may be activated or inactivated. In some embodiments, the histone stem-loop is generally derived from histone genes and includes an intramolecular base pairing of two neighbored partially or entirely reverse complementary sequences separated by a spacer, consisting of a short sequence, which forms the loop of the structure. The unpaired loop region is typically unable to base pair with either of the stem loop elements. It occurs more often in RNA, as is a key component of many RNA secondary structures but may be present in single-stranded DNA as well. Stability of the stem-loop structure generally depends on the length, number of mismatches or bulges, and base composition of the paired region. In some embodiments, wobble base pairing (non-Watson-Crick base pairing) may result. In some embodiments, the at least one histone stem-loop sequence comprises a length of 15 to 45 nucleotides.

In some embodiments, an mRNA has one or more AU-rich sequences removed. These sequences, sometimes referred to as AURES are destabilizing sequences found in the 3′ UTR. The AURES may be removed from the mRNA. Alternatively, the AURES may remain in the mRNA.

### Sequence Optimization

In some embodiments, an open reading frame encoding a protein of the disclosure is codon optimized. Codon optimization methods are known in the art. An open reading frame of any one or more of the sequences provided herein may be codon optimized. Codon optimization, in some embodiments, may be used to match codon frequencies in target and host organisms to ensure proper folding; bias GC content to increase mRNA stability or reduce secondary structures; minimize tandem repeat codons or base runs that may impair gene construction or expression; customize transcriptional and translational control regions; insert or remove protein trafficking sequences; remove/add post translation modification sites in encoded protein (e.g., glycosylation sites); add, remove or shuffle protein domains; insert or delete restriction sites; modify ribosome binding sites and mRNA degradation sites; adjust translational rates to allow the various domains of the protein to fold properly; or reduce or eliminate problem secondary structures within the polynucleotide. Codon optimization tools, algorithms and services are known in the art—non-limiting examples include services from GeneArt (Life Technologies), DNA2.0 (Menlo Park CA) and/or proprietary methods. In some embodiments, the open reading frame sequence is optimized using optimization algorithms.

In some embodiments, a codon optimized sequence shares less than 95% sequence identity to a naturally-occurring or wild-type sequence open reading frame (e.g., a naturally-occurring or wild-type mRNA sequence encoding an EBV protein antigen). In some embodiments, a codon optimized sequence shares less than 90% sequence identity to a naturally-occurring or wild-type sequence (e.g., a naturally-occurring or wild-type mRNA sequence encoding an EBV protein). In some embodiments, a codon optimized sequence shares less than 85% sequence identity to a naturally-occurring or wild-type sequence (e.g., a naturally-occurring or wild-type mRNA sequence encoding an EBV protein). In some embodiments, a codon optimized sequence shares less than 80% sequence identity to a naturally-occurring or wild-type sequence (e.g., a naturally-occurring or wild-type mRNA sequence encoding an EBV protein). In some embodiments, a codon optimized sequence shares less than 75% sequence identity to a naturally-occurring or wild-type sequence (e.g., a naturally-occurring or wild-type mRNA sequence encoding an EBV protein).

In some embodiments, a codon optimized sequence shares between 65% and 85% (e.g., between about 67% and about 85% or between about 67% and about 80%) sequence identity to a naturally-occurring or wild-type sequence (e.g., a naturally-occurring or wild-type mRNA sequence encoding an EBV protein). In some embodiments, a codon optimized sequence shares between 65% and 75% or about 80% sequence identity to a naturally-occurring or wild-type sequence (e.g., a naturally-occurring or wild-type mRNA sequence encoding an EBV protein).

In some embodiments, a codon-optimized sequence encodes an antigen that is as immunogenic as, or more immunogenic than (e.g., at least 10%, at least 20%, at least 30%, at least 40%, at least 50%, at least 100%, or at least 200% more), than an EBV protein encoded by a non-codon-optimized sequence.

When transfected into mammalian host cells, the modified mRNAs have a stability of between 12-18 hours, or greater than 18 hours, e.g., 24, 36, 48, 60, 72, or greater than 72 hours and are capable of being expressed by the mammalian host cells.

In some embodiments, a codon optimized mRNA may be one in which the levels of G/C are enhanced. The G/C-content of nucleic acid molecules (e.g., mRNA) may influence the stability of the mRNA. mRNA having an increased amount of guanine (G) and/or cytosine (C) residues may be functionally more stable than RNA containing a large amount of adenine (A) and thymine (T) or uracil (U) nucleotides. As an example, WO02/098443 discloses a pharmaceutical composition containing an mRNA stabilized by sequence modifications in the translated region. Due to the degeneracy of the genetic code, the modifications work by substituting existing codons for those that promote greater RNA stability without changing the resulting amino acid. The approach is limited to coding regions of the mRNA.

### Chemically Unmodified Nucleotide

In some embodiments, an mRNA is not chemically modified and comprises the standard ribonucleotides consisting of adenosine, guanosine, cytosine and uridine. In some embodiments, nucleotides and nucleosides comprise standard nucleoside residues such as those present in transcribed RNA (e.g., A, G, C, or U). In some embodiments, nucleotides and nucleosides comprise standard deoxyribonucleosides such as those present in DNA (e.g., dA, dG, dC, or dT).

### Chemically Modified Nucleotides

The mRNA vaccine comprises, in some embodiments, an RNA having an open reading frame encoding an EBV protein, wherein the nucleic acid comprises nucleotides and/or nucleosides that can be standard (unmodified) or modified as is known in the art. In some embodiments, nucleotides and nucleosides of the mRNA vaccines comprise modified nucleotides or nucleosides. Such modified nucleotides and nucleosides can be naturally-occurring modified nucleotides and nucleosides or non-naturally-occurring modified nucleotides and nucleosides. Such modifications can include those at the sugar, backbone, or nucleobase portion of the nucleotide and/or nucleoside as are recognized in the art.

In some embodiments, a naturally-occurring modified nucleotide or nucleotide of the disclosure is one as is generally known or recognized in the art. Non-limiting examples of such naturally-occurring modified nucleotides and nucleotides can be found, inter alia, in the widely recognized MODOMICS database.

In some embodiments, a non-naturally-occurring modified nucleotide or nucleoside of the disclosure is one as is generally known or recognized in the art. Non-limiting examples of such non-naturally-occurring modified nucleotides and nucleosides can be found, inter alia, in international publication numbers WO2013052523A1; WO2014093924A1; WO2015051173A2; WO2015051169A2; WO2015089511A2; or WO2017153936A1, each of which is herein incorporated by reference.

Hence, nucleic acids of the disclosure (e.g., DNA and RNA, such as mRNA) can comprise standard nucleotides and nucleosides, naturally-occurring nucleotides and nucleosides, non-naturally-occurring nucleotides and nucleosides, or any combination thereof.

Nucleic acids of the disclosure (e.g., DNA and RNA, such as mRNA), in some embodiments, comprise various (more than one) different types of standard and/or modified nucleotides and nucleosides. In some embodiments, a particular region of a nucleic acid contains one, two or more (optionally different) types of standard and/or modified nucleotides and nucleosides.

In some embodiments, a modified mRNA introduced to a cell or organism, exhibits reduced degradation in the cell or organism, respectively, relative to an unmodified nucleic acid comprising standard nucleotides and nucleosides.

In some embodiments, a modified mRNA introduced into a cell or organism, may exhibit reduced immunogenicity in the cell or organism, respectively (e.g., a reduced innate response) relative to an unmodified nucleic acid comprising standard nucleotides and nucleosides.

Nucleic acids (e.g., RNA, such as mRNA), in some embodiments, comprise non-natural modified nucleotides that are introduced during synthesis or post-synthesis of the nucleic acids to achieve desired functions or properties. The modifications may be present on internucleotide linkages, purine or pyrimidine bases, or sugars. The modification may be introduced with chemical synthesis or with a polymerase enzyme at the terminal of a chain or anywhere else in the chain. Any of the regions of a nucleic acid may be chemically modified.

The present disclosure provides for modified nucleosides and nucleotides of a nucleic acid (e.g., RNA, such as mRNA). A “nucleoside” refers to a compound containing a sugar molecule (e.g., a pentose or ribose) or a derivative thereof in combination with an organic base (e.g., a purine or pyrimidine) or a derivative thereof (also referred to herein as “nucleobase”). A “nucleotide” refers to a nucleoside, including a phosphate group. Modified nucleotides may by synthesized by any useful method, such as, for example, chemically, enzymatically, or recombinantly, to include one or more modified or non-natural nucleosides. Nucleic acids can comprise a region or regions of linked nucleosides. Such regions may have variable backbone linkages. The linkages can be standard phosphodiester linkages, in which case the nucleic acids would comprise regions of nucleotides.

Modified nucleotide base pairing encompasses not only the standard adenosine-thymine, adenosine-uracil, or guanosine-cytosine base pairs, but also base pairs formed between nucleotides and/or modified nucleotides comprising non-standard or modified bases, wherein the arrangement of hydrogen bond donors and hydrogen bond acceptors permits hydrogen bonding between a non-standard base and a standard base or between two complementary non-standard base structures, such as, for example, in those nucleic acids having at least one chemical modification. One example of such non-standard base pairing is the base pairing between the modified nucleotide inosine and adenine, cytosine or uracil. Any combination of base/sugar or linker may be incorporated into nucleic acids.

In some embodiments, modified nucleobases in nucleic acids (e.g., RNA, such as mRNA) comprise 1-methyl-pseudouridine (m1ψ), 1-ethyl-pseudouridine (e1ψ), 5-methoxy-uridine (mo5U), 5-methyl-cytidine (m5C), and/or pseudouridine (ψ). In some embodiments, modified nucleobases in nucleic acids (e.g., RNA, such as mRNA) comprise 5-methoxymethyl uridine, 5-methylthio uridine, 1-methoxymethyl pseudouridine, 5-methyl cytidine, and/or 5-methoxy cytidine. In some embodiments, the polyribonucleotide includes a combination of at least two (e.g., 2, 3, 4 or more) of any of the aforementioned modified nucleobases, including but not limited to chemical modifications.

In some embodiments, an mRNA of the disclosure comprises 1-methyl-pseudouridine (m1ψ) substitutions at one or more or all uridine positions of the nucleic acid.

In some embodiments, an mRNA of the disclosure comprises 1-methyl-pseudouridine (m1ψ) substitutions at one or more or all uridine positions of the nucleic acid and 5-methyl cytidine substitutions at one or more or all cytidine positions of the nucleic acid.

In some embodiments, an mRNA of the disclosure comprises pseudouridine (ψ) substitutions at one or more or all uridine positions of the nucleic acid.

In some embodiments, an mRNA of the disclosure comprises pseudouridine (ψ) substitutions at one or more or all uridine positions of the nucleic acid and 5-methyl cytidine substitutions at one or more or all cytidine positions of the nucleic acid.

In some embodiments, an mRNA of the disclosure comprises uridine at one or more or all uridine positions of the nucleic acid.

In some embodiments, mRNAs are uniformly modified (e.g., fully modified, modified throughout the entire sequence) for a particular modification. For example, a nucleic acid can be uniformly modified with 1-methyl-pseudouridine, meaning that all uridine residues in the mRNA sequence are replaced with 1-methyl-pseudouridine. Similarly, a nucleic acid can be uniformly modified for any type of nucleoside residue present in the sequence by replacement with a modified residue such as those set forth above.

The nucleic acids may be partially or fully modified along the entire length of the molecule. For example, one or more or all or a given type of nucleotide (e.g., purine or pyrimidine, or any one or more or all of A, G, U, C) may be uniformly modified in a nucleic acid of the disclosure, or in a predetermined sequence region thereof (e.g., in the mRNA including or excluding the poly(A) tail). In some embodiments, all nucleotides X in a nucleic acid (or in a sequence region thereof) are modified nucleotides, wherein X may be any one of nucleotides A, G, U, C, or any one of the combinations A+G, A+U, A+C, G+U, G+C, U+C, A+G+U, A+G+C, G+U+C or A+G+C.

The nucleic acid may contain from about 1% to about 100% modified nucleotides (either in relation to overall nucleotide content, or in relation to one or more types of nucleotide, i.e., any one or more of A, G, U or C) or any intervening percentage (e.g., from 1% to 20%, from 1% to 25%, from 1% to 50%, from 1% to 60%, from 1% to 70%, from 1% to 80%, from 1% to 90%, from 1% to 95%, from 10% to 20%, from 10% to 25%, from 10% to 50%, from 10% to 60%, from 10% to 70%, from 10% to 80%, from 10% to 90%, from 10% to 95%, from 10% to 100%, from 20% to 25%, from 20% to 50%, from 20% to 60%, from 20% to 70%, from 20% to 80%, from 20% to 90%, from 20% to 95%, from 20% to 100%, from 50% to 60%, from 50% to 70%, from 50% to 80%, from 50% to 90%, from 50% to 95%, from 50% to 100%, from 70% to 80%, from 70% to 90%, from 70% to 95%, from 70% to 100%, from 80% to 90%, from 80% to 95%, from 80% to 100%, from 90% to 95%, from 90% to 100%, and from 95% to 100%). It will be understood that any remaining percentage is accounted for by the presence of unmodified A, G, U, or C.

The mRNA may contain at a minimum 1% and at maximum 100% modified nucleotides, or any intervening percentage, such as at least 5% modified nucleotides, at least 10% modified nucleotides, at least 25% modified nucleotides, at least 50% modified nucleotides, at least 80% modified nucleotides, or at least 90% modified nucleotides. For example, the nucleic acids may contain a modified pyrimidine such as a modified uracil or cytosine. In some embodiments, at least 5%, at least 10%, at least 25%, at least 50%, at least 80%, at least 90% or 100% of the uracil in the nucleic acid is replaced with a modified uracil (e.g., a 5-substituted uracil). The modified uracil can be replaced by a compound having a single unique structure or can be replaced by a plurality of compounds having different structures (e.g., 2, 3, 4 or more unique structures). In some embodiments, at least 5%, at least 10%, at least 25%, at least 50%, at least 80%, at least 90% or 100% of the cytosine in the nucleic acid is replaced with a modified cytosine (e.g., a 5-substituted cytosine). The modified cytosine can be replaced by a compound having a single unique structure or can be replaced by a plurality of compounds having different structures (e.g., 2, 3, 4 or more unique structures).

### Nucleic Acid Production

### Chemical Synthesis

Solid-phase chemical synthesis. Nucleic acids the present disclosure may be manufactured in whole or in part using solid phase techniques. Solid-phase chemical synthesis of nucleic acids is an automated method wherein molecules are immobilized on a solid support and synthesized step by step in a reactant solution. Solid-phase synthesis is useful in site-specific introduction of chemical modifications in the nucleic acid sequences.

The synthesis of nucleic acids by the sequential addition of monomer building blocks may be carried out in a liquid phase.

The synthetic methods discussed above each has its own advantages and limitations. Attempts have been conducted to combine these methods to overcome the limitations. Such combinations of methods are within the scope of the present disclosure. The use of solid-phase or liquid-phase chemical synthesis in combination with enzymatic ligation provides an efficient way to generate long chain nucleic acids that cannot be obtained by chemical synthesis alone.

### Ligation

Assembling nucleic acids by a ligase may also be used. DNA or RNA ligases promote intermolecular ligation of the 5′ and 3′ ends of polynucleotide chains through the formation of a phosphodiester bond. Nucleic acids such as chimeric polynucleotides and/or circular nucleic acids may be prepared by ligation of one or more regions or subregions. DNA fragments can be joined by a ligase catalyzed reaction to create recombinant DNA with different functions. Two oligodeoxynucleotides, one with a 5′ phosphoryl group and another with a free 3′ hydroxyl group, serve as substrates for a DNA ligase.

### Purification

Purification of the nucleic acids described herein may include, but is not limited to, nucleic acid clean-up, quality assurance and quality control. Clean-up may be performed by methods known in the arts such as, but not limited to, AGENCOURT® beads (Beckman Coulter Genomics, Danvers, MA), poly-T beads, LNA™ oligo-T capture probes (EXIQON® Inc, Vedbaek, Denmark) or HPLC based purification methods such as, but not limited to, strong anion exchange HPLC, weak anion exchange HPLC, reverse phase HPLC (RP-HPLC), and hydrophobic interaction HPLC (HIC-HPLC). The term “purified” when used in relation to a nucleic acid such as a “purified nucleic acid” refers to one that is separated from at least one contaminant. A “contaminant” is any substance that makes another unfit, impure or inferior. Thus, a purified nucleic acid (e.g., DNA and RNA) is present in a form or setting different from that in which it is found in nature, or a form or setting different from that which existed prior to subjecting it to a treatment or purification method.

A quality assurance and/or quality control check may be conducted using methods such as, but not limited to, gel electrophoresis, UV absorbance, or analytical HPLC.

In some embodiments, the nucleic acids may be sequenced by methods including, but not limited to reverse-transcriptase-PCR.

### Quantification

In some embodiments, the nucleic acids may be quantified in exosomes or when derived from one or more bodily fluid. Bodily fluids include peripheral blood, serum, plasma, ascites, urine, cerebrospinal fluid (CSF), sputum, saliva, bone marrow, synovial fluid, aqueous humor, amniotic fluid, cerumen, breast milk, broncheoalveolar lavage fluid, semen, prostatic fluid, cowper's fluid or pre-ejaculatory fluid, sweat, fecal matter, hair, tears, cyst fluid, pleural and peritoneal fluid, pericardial fluid, lymph, chyme, chyle, bile, interstitial fluid, menses, pus, sebum, vomit, vaginal secretions, mucosal secretion, stool water, pancreatic juice, lavage fluids from sinus cavities, bronchopulmonary aspirates, blastocyl cavity fluid, and umbilical cord blood. Alternatively, exosomes may be retrieved from an organ selected from the group consisting of lung, heart, pancreas, stomach, intestine, bladder, kidney, ovary, testis, skin, colon, breast, prostate, brain, esophagus, liver, and placenta.

Assays may be performed using construct specific probes, cytometry, qRT-PCR, real-time PCR, PCR, flow cytometry, electrophoresis, mass spectrometry, or combinations thereof while the exosomes may be isolated using immunohistochemical methods such as enzyme linked immunosorbent assay (ELISA) methods. Exosomes may also be isolated by size exclusion chromatography, density gradient centrifugation, differential centrifugation, nanomembrane ultrafiltration, immunoabsorbent capture, affinity purification, microfluidic separation, or combinations thereof.

These methods afford the investigator the ability to monitor, in real time, the level of nucleic acids remaining or delivered. This is possible because the nucleic acids, in some embodiments, differ from the endogenous forms due to the structural or chemical modifications.

In some embodiments, the nucleic acid may be quantified using methods such as, but not limited to, ultraviolet visible spectroscopy (UV/Vis). A non-limiting example of a UV/Vis spectrometer is a NANODROP® spectrometer (ThermoFisher, Waltham, MA). The quantified nucleic acid may be analyzed in order to determine if the nucleic acid may be of proper size, check that no degradation of the nucleic acid has occurred. Degradation of the nucleic acid may be checked by methods such as, but not limited to, agarose gel electrophoresis, HPLC based purification methods such as, but not limited to, strong anion exchange HPLC, weak anion exchange HPLC, reverse phase HPLC (RP-HPLC), and hydrophobic interaction HPLC (HIC-HPLC), liquid chromatography-mass spectrometry (LCMS), capillary electrophoresis (CE) and capillary gel electrophoresis (CGE).

### In Vitro Transcription

cDNA encoding the polynucleotides described herein may be transcribed using an in vitro transcription (IVT) system. In vitro transcription of mRNA is known in the art and is described in International Publication WO 2014/152027, which is incorporated by reference herein in its entirety. In some embodiments, the mRNA is prepared in accordance with any one or more of the methods described in WO 2018/053209 or WO 2019/036682, each of which is incorporated by reference herein.

In some embodiments, the mRNA transcript is generated using a non-amplified, linearized DNA template in an in vitro transcription reaction to generate the mRNA transcript. In some embodiments, the template DNA is isolated DNA. In some embodiments, the template DNA is cDNA. In some embodiments, the cDNA is formed by reverse transcription of an RNA, for example, but not limited to EBV mRNA. In some embodiments, cells, e.g., bacterial cells, e.g., E. coli, e.g., DH-1 cells are transfected with the plasmid DNA template. In some embodiments, the transfected cells are cultured to replicate the plasmid DNA which is then isolated and purified. In some embodiments, the DNA template includes an RNA polymerase promoter, e.g., a T7 promoter located 5′ to and operably linked to the gene of interest.

In some embodiments, an in vitro transcription template encodes a 5′ untranslated (UTR) region, contains an open reading frame, and encodes a 3′ UTR and a poly(A) tail. The particular nucleic acid sequence composition and length of an in vitro transcription template will depend on the mRNA encoded by the template.

In some embodiments, a nucleic acid (e.g., template DNA and/or RNA) includes 200 to 3,000 nucleotides. For example, a nucleic acid may include 200 to 500, 200 to 1000, 200 to 1500, 200 to 3000, 500 to 1000, 500 to 1500, 500 to 2000, 500 to 3000, 1000 to 1500, 1000 to 2000, 1000 to 3000, 1500 to 3000, or 2000 to 3000 nucleotides.

An in vitro transcription system typically comprises a transcription buffer (e.g., with magnesium), nucleotide triphosphates (NTPs), an RNase inhibitor and a polymerase (e.g., T7 RNA polymerase). In some embodiments, one or more of the NTPs is a chemically modified NTP (e.g., with 1-methylpseudouridine or other chemical modifications described herein and/or known in the art).

In some embodiments, the NTPs comprise adenosine triphosphate (ATP), cytidine triphosphate (CTP), uridine triphosphate (UTP), and guanosine triphosphate (GTP), or an analog of each respective NTP. The ratio of NTPs may vary. In some embodiments, the ratio of GTP:ATP:CTP:UTP is 1:1:1:1. In some embodiments, the amount of the GTP or an analogue thereof is greater than an amount of the UTP or an analogue thereof. In some embodiments, the amount of the GTP is greater than the amount of the UTP. In some embodiments, the amount of ATP is greater than the amount of UTP, and the amount of CTP is greater than the amount of UTP. In some embodiments, the amount of the GTP or an analogue thereof is greater than an amount of the UTP or an analogue thereof. In some embodiments, an IVT system comprises an at least 2:1 ratio of GTP concentration to ATP concentration, an at least 2:1 ratio of GTP concentration to CTP concentration, and an at least 4:1 ratio of GTP concentration to UTP concentration. In some embodiments, an IVT system comprises a 2:1 ratio of GTP concentration to ATP concentration, a 2:1 ratio of GTP concentration to CTP concentration, and a 4:1 ratio of GTP concentration to UTP concentration. In some embodiments, an IVT system comprises guanosine diphosphate (GDP). In some embodiments, an IVT system comprises an at least 3:1 ratio of GTP plus GDP concentration to ATP concentration, an at least 6:1 ratio of GTP plus GDP concentration to CTP concentration, and an at least 6:1 ratio of GTP plus GDP concentration to UTP concentration.

The NTPs may be manufactured in house, may be selected from a supplier, or may be synthesized as described herein. The NTPs may be selected from, but are not limited to, those described herein including natural and unnatural (modified) NTPs.

Any number of RNA polymerases or variants may be used in a method of the present disclosure. The polymerase may be selected from, but is not limited to, a phage RNA polymerase, e.g., a T7 RNA polymerase, a T3 RNA polymerase, a SP6 RNA polymerase, and/or mutant polymerases such as, but not limited to, polymerases able to incorporate modified nucleic acids and/or modified nucleotides, including chemically modified nucleic acids and/or nucleotides. Some embodiments exclude the use of DNase.

An IVT system, in some embodiments, comprises magnesium buffer, dithiothreitol (DTT) spermidine, pyrophosphatase, and/or RNase inhibitor. In some embodiments, an IVT system omits an RNase inhibitor. An IVT system may be incubated at 25 degrees Celsius or at 37 degrees Celsius. Other temperatures may be used, depending in part on the polymerase (e.g., use of a variant polymerase).

In some embodiments, the mRNA transcript is capped via enzymatic capping. In some embodiments, the mRNA comprises 5′ terminal cap, for example, 7mG(5′)ppp(5′)NlmpNp.

### Identification and Ratio Determination (IDR) Sequences

In some embodiments, one or more nucleic acids comprises an Identification and Ratio Determination sequence. An Identification and Ratio Determination (IDR) sequence is a sequence of a biological molecule (e.g., nucleic acid or protein) that, when combined with the sequence of a target biological molecule, serves to identify the target biological molecule. Typically, an IDR sequence is a heterologous sequence that is incorporated within or appended to a sequence of a target biological molecule and can be used as a reference to identify the target molecule. Thus, in some embodiments, a nucleic acid (e.g., mRNA) comprises (i) a target sequence of interest (e.g., a coding sequence encoding a therapeutic and/or antigenic peptide or protein); and (ii) a unique IDR sequence.

An RNA species (e.g., RNA having a given coding sequence) may comprise an IDR sequence that differs from the IDR sequence of other RNA species (e.g., RNA(s) having different coding sequence(s)). Each IDR sequence thus identifies a particular RNA species, and so the abundance of IDR sequences may be measured to determine the abundance of each RNA species in an mRNA vaccine. Use of distinct IDR sequences to identify RNA species allows for analysis of multivalent RNA compositions (e.g., containing multiple RNA species) containing RNA species with similar coding sequences and/or lengths, which could otherwise be difficult to distinguish using PCR- or chromatography-based analysis of full-length RNAs.

Each RNA species in a multivalent RNA composition may comprise an IDR sequence that is not a sequence isomer of an IDR sequence of another RNA species in a multivalent RNA composition (e.g., the IDR sequence does not have the same number of adenosine nucleotides, the same number of cytosine nucleotides, the same number of guanine nucleotides, and the same number of uracil nucleotides, as another IDR sequence in the composition, even if those sequences have different sequences). Having identical nucleotide compositions causes sequence isomers to have the same mass, presenting a challenge to distinguishing sequence isomers using mass-based identification methods (e.g., mass spectrometry).

Each RNA species in a multivalent RNA composition may comprise an IDR sequence having a mass that differs from the mass of IDR sequences of each other RNA species in a multivalent RNA composition. For example, the mass of each IDR sequence may differ from the mass of other IDR sequences by at least 9 Da, at least 25 Da, at least 25 Da, or at least 50 Da. Use of IDR sequences with distinct masses allows RNA fragments comprising different IDR sequences to be distinguished using mass-based analysis methods (e.g., mass spectrometry), which do not require reverse transcription, amplification, or sequencing of RNAs.

Each RNA species in an RNA composition may comprises an IDR sequence with a different length. For example, each IDR sequence may have a length independently selected from 0 to 25 nucleotides. The length of a nucleic acid influences the rate at which the nucleic acid traverses a chromatography column, and so the use of IDR sequences of different lengths on different RNA species allows RNA fragments having different IDR sequences to be distinguished using chromatography-based methods (e.g., LC-UV).

IDR sequences may be chosen such that no IDR sequence comprises a start codon, ‘AUG’. Lack of a start codon in an IDR sequence prevents undesired translation of nucleotide sequences within and/or downstream from the IDR sequence.

IDR sequences may be chosen such that no IDR sequence comprises a recognition site for a restriction enzyme. In one example, no IDR sequence comprises a recognition site for XbaI, ‘UCUAG’. Lack of a recognition site for a restriction enzyme (e.g., XbaI recognition site ‘UCUAG’) allows the restriction enzyme to be used in generating and modifying a DNA template for in vitro transcription, without affecting the IDR sequence or sequence of the transcribed RNA.

Non-limiting examples of distinct IDR sequences include: GAGAUUGAGUGUAGUGACUAG (SEQ ID NO: 45), GAGAUUGAGUGUAGUGAC (SEQ ID NO: 46), GAGAUUGAGUGUAGUG (SEQ ID NO: 47), GAUUGAGACUACGGG (SEQ ID NO: 48), and CAUAGACACUACG (SEQ ID NO: 49). In some embodiments of the compositions described herein, each mRNA encoding a distinct protein comprises a 3′ UTR comprising a distinct IDR sequence selected from SEQ ID NOs: 45-49.

### Lipid Compositions

In some embodiments, the nucleic acids are formulated as a lipid composition, such as a composition comprising a lipid nanoparticle, a liposome, and/or a lipoplex. In some embodiments, nucleic acids are formulated as lipid nanoparticle (LNP) compositions. Lipid nanoparticles typically comprise amino lipid, non-cationic lipid, structural lipid, and PEG lipid components along with the nucleic acid cargo of interest. The lipid nanoparticles can be generated using components, compositions, and methods as are generally known in the art, see for example PCT/US2016/052352; PCT/US2016/068300; PCT/US2017/037551; PCT/US2015/027400; PCT/US2016/047406; PCT/US2016/000129; PCT/US2016/014280; PCT/US2017/038426; PCT/US2014/027077; PCT/US2014/055394; PCT/US2016/052117; PCT/US2012/069610; PCT/US2017/027492; PCT/US2016/059575; PCT/US2016/069491; PCT/US2016/069493; and PCT/US2014/066242, all of which are incorporated by reference herein in their entirety.

In some embodiments, the lipid nanoparticle comprises at least one ionizable amino lipid, at least one non-cationic lipid, at least one sterol, and/or at least one polyethylene glycol (PEG)-modified lipid.

In some embodiments, the lipid nanoparticle comprises a molar ratio of 20-60% ionizable amino lipid, 5-25% non-cationic lipid, 25-55% structural lipid, and 0.5-15% PEG-modified lipid.

In some embodiments, the lipid nanoparticle comprises a molar ratio of 20-60% ionizable amino lipid, 5-30% non-cationic lipid, 10-55% structural lipid, and 0.5-15% PEG-modified lipid.

In some embodiments, the lipid nanoparticle comprises 40-50 mol % ionizable lipid, optionally 45-50 mol %, for example, 45-46 mol %, 46-47 mol %, 47-48 mol %, 48-49 mol %, or 49-50 mol % for example about 45 mol %, 45.5 mol %, 46 mol %, 46.5 mol %, 47 mol %, 47.5 mol %, 48 mol %, 48.5 mol %, 49 mol %, or 49.5 mol %.

In some embodiments, the lipid nanoparticle comprises 20-60 mol % ionizable amino lipid. For example, the lipid nanoparticle may comprise 20-50 mol %, 20-40 mol %, 20-30 mol %, 30-60 mol %, 30-50 mol %, 30-40 mol %, 40-60 mol %, 40-50 mol %, or 50-60 mol % ionizable amino lipid. In some embodiments, the lipid nanoparticle comprises 20 mol %, 30 mol %, 40 mol %, 50 mol %, or 60 mol % ionizable amino lipid. In some embodiments, the lipid nanoparticle comprises 35 mol %, 36 mol %, 37 mol %, 38 mol %, 39 mol %, 40 mol %, 41 mol %, 42 mol %, 43 mol %, 44 mol %, 45 mol %, 46 mol %, 47 mol %, 48 mol %, 49 mol %, 50 mol %, 51 mol %, 52 mol %, 53 mol %, 54 mol %, or 55 mol % ionizable amino lipid.

In some embodiments, the lipid nanoparticle comprises 45-55 mole percent (mol %) ionizable amino lipid. For example, lipid nanoparticle may comprise 45, 46, 47, 48, 49, 50, 51, 52, 53, 54, or 55 mol % ionizable amino lipid.

### Ionizable Amino Lipids

### Formula (AI)

In some embodiments, the ionizable amino lipid is a compound of Formula (AI):

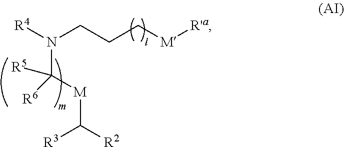

or its N-oxide, or a salt or isomer thereof,
wherein R′^{a} is R′^{branched}; wherein
R′^{branched} is:

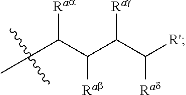

wherein

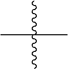

denotes a point of attachment;
wherein R^{aα}, R^{aβ}, R^{aγ}, and R^{aδ} are each independently selected from the group consisting of H, C₂₋₁₂ alkyl, and C₂₋₁₂ alkenyl;
R² and R³ are each independently selected from the group consisting of C₁₋₁₄ alkyl and C₂₋₁₄ alkenyl;
R⁴ is selected from the group consisting of —(CH₂)ₙOH, wherein n is selected from the group consisting of 1, 2, 3, 4, and 5, and

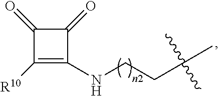

wherein

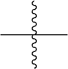

denotes a point of attachment; wherein
R¹⁰ is N(R)₂; each R is independently selected from the group consisting of C₁₋₆ alkyl, C₂₋₃ alkenyl, and H; and n2 is selected from the group consisting of 1, 2, 3, 4, 5, 6, 7, 8, 9, and 10;
each R⁵ is independently selected from the group consisting of C₁₋₃ alkyl, C₂₋₃ alkenyl, and H;
each R⁶ is independently selected from the group consisting of C₁₋₃ alkyl, C₂₋₃ alkenyl, and H;
M and M′ are each independently selected from the group consisting of —C(O)O— and —OC(O)—;
R′ is a C₁₋₁₂ alkyl or C₂₋₁₂ alkenyl;
l is selected from the group consisting of 1, 2, 3, 4, and 5; and
m is selected from the group consisting of 5, 6, 7, 8, 9, 10, 11, 12, and 13.

In some embodiments of the compounds of Formula (AI), R′^{a} is R′^{branched}; R′^{branched} is

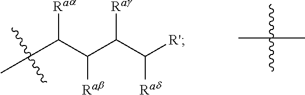

denotes a point of attachment; R^{aα}, R^{aβ}, R^{aγ}, and R^{aδ} are each H; R² and R³ are each C₁₋₁₄ alkyl; R⁴ is —(CH₂)ₙOH; n is 2; each R⁵ is H; each R⁶ is H; M and M′ are each —C(O)O—; R′ is a C₁₋₁₂ alkyl; 1 is 5; and m is 7.

In some embodiments of the compounds of Formula (AI), R′^{a} is R′^{branched}; R′^{branched} is

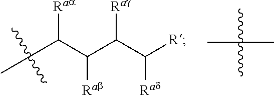

denotes a point of attachment; R^{aα}, R^{aβ}, R^{aγ}, and R^{aδ} are each H; R² and R³ are each C₁₋₁₄ alkyl; R⁴ is —(CH₂)ₙOH; n is 2; each R⁵ is H; each R⁶ is H; M and M′ are each —C(O)O—; R′ is a C₁₋₁₂ alkyl; 1 is 3; and m is 7.

In some embodiments of the compounds of Formula (AI), R′^{a} is R′^{branched}; R′^{branched} is

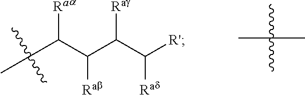

denotes a point of attachment; R^{aα} is C₂₋₁₂ alkyl; R^{aβ}, R^{aγ}, and R^{aδ} are each H; R² and R³ are each C₁₋₁₄ alkyl; R⁴ is

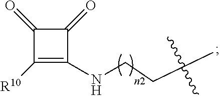

R¹⁰NH(C₁₋₆ alkyl); n2 is 2; R⁵ is H; each R⁶ is H; M and M′ are each —C(O)O—; R′ is a C₁₋₁₂ alkyl; 1 is 5; and m is 7.

In some embodiments of the compounds of Formula (AI), R′^{a} is R′^{branched}; R′^{branched} is

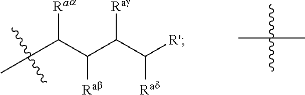

denotes a point of attachment; R^{aα}, R^{aβ}, and R^{aδ} are each H; R^{aγ} is C₂₋₁₂ alkyl; R² and R³ are each C₁₋₁₄ alkyl; R⁴ is —(CH₂)ₙOH; n is 2; each R⁵ is H; each R⁶ is H; M and M′ are each —C(O)O—; R′ is a C₁₋₁₂ alkyl; 1 is 5; and m is 7.

In some embodiments, the compound of Formula (AI) is selected from:

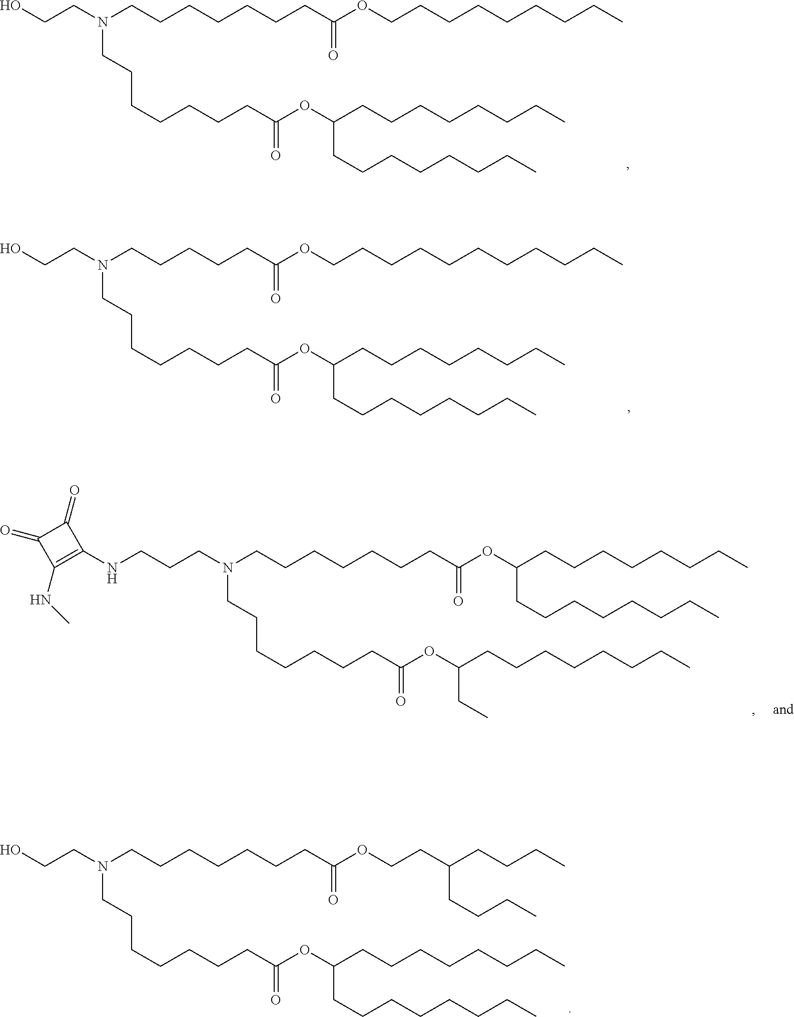 [SMILES: CCCCCCCCCOC(=O)CCCCCCCN(CCO)CCCCCCCC(=O)OC(CCCCCCCC)CCCCCCCC ; CCCCCCCCCCCOC(=O)CCCCCN(CCO)CCCCCCCC(=O)OC(CCCCCCCC)CCCCCCCC ; CCCCCCCCC(CC)OC(=O)CCCCCCCN(CCCCCCCC(=O)OC(CCCCCCCC)CCCCCCCC)CCCNc1c(NC)c(=O)c1=O ; CCCCCCCCC(CCCCCCCC)OC(=O)CCCCCCCN(CCO)CCCCCCCC(=O)OCCC(CCCC)CCCC]

In some embodiments, the ionizable amino lipid of Formula (AI) is a compound of Formula (AIa):

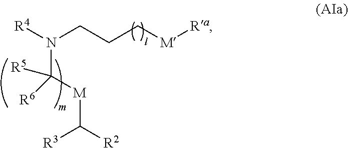

or its N-oxide, or a salt or isomer thereof,
wherein R′^{a} is R′^{branched}; wherein
R′^{branched} is:

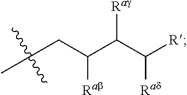

wherein

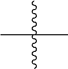

denotes a point of attachment;
wherein Rap, Ray, and Ras are each independently selected from the group consisting of H, C₂₋₁₂ alkyl, and C₂₋₁₂ alkenyl;
R² and R³ are each independently selected from the group consisting of C₁₋₁₄ alkyl and C₂₋₁₄ alkenyl;
R⁴ is selected from the group consisting of —(CH₂)ₙOH wherein n is selected from the group consisting of 1, 2, 3, 4, and 5, and

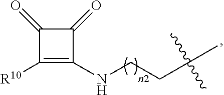

wherein

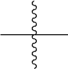

denotes a point of attachment; wherein
R¹⁰ is N(R)₂; each R is independently selected from the group consisting of C₁₋₆ alkyl, C₂₋₃ alkenyl, and H; and n2 is selected from the group consisting of 1, 2, 3, 4, 5, 6, 7, 8, 9, and 10;
each R⁵ is independently selected from the group consisting of C₁₋₃ alkyl, C₂₋₃ alkenyl, and H;
each R⁶ is independently selected from the group consisting of C₁₋₃ alkyl, C₂₋₃ alkenyl, and H;
M and M′ are each independently selected from the group consisting of —C(O)O— and —OC(O)—;
R′ is a C₁₋₁₂ alkyl or C₂₋₁₂ alkenyl;
l is selected from the group consisting of 1, 2, 3, 4, and 5; and
m is selected from the group consisting of 5, 6, 7, 8, 9, 10, 11, 12, and 13.

In some embodiments, the ionizable amino lipid of Formula (AI) is a compound of Formula (AIb):

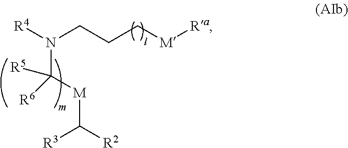

or its N-oxide, or a salt or isomer thereof,
wherein R′^{a} is R′^{branched}; wherein

R′^{branched} is:

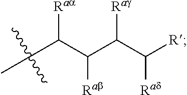

wherein

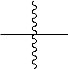

denotes a point of attachment;
wherein R^{aα}, R^{aβ}, R^{aγ}, and R^{aδ} are each independently selected from the group consisting of H, C₂₋₁₂ alkyl, and C₂₋₁₂ alkenyl;
R² and R³ are each independently selected from the group consisting of C₁₋₁₄ alkyl and C₂₋₁₄ alkenyl;
R⁴ is —(CH₂)ₙOH, wherein n is selected from the group consisting of 1, 2, 3, 4, and 5;
each R⁵ is independently selected from the group consisting of C₁₋₃ alkyl, C₂₋₃ alkenyl, and H;
each R⁶ is independently selected from the group consisting of C₁₋₃ alkyl, C₂₋₃ alkenyl, and H;
M and M′ are each independently selected from the group consisting of —C(O)O— and —OC(O)—;
R′ is a C₁₋₁₂ alkyl or C₂₋₁₂ alkenyl;
l is selected from the group consisting of 1, 2, 3, 4, and 5; and m is selected from the group consisting of 5, 6, 7, 8, 9, 10, 11, 12, and 13.

In some embodiments of Formula (AI) or (AIb), R′^{a} is R′^{branched}; R′^{branched} is

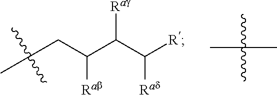

denotes a point of attachment; R^{aβ}, R^{aγ}, and R^{aδ} are each H; R² and R³ are each C₁₋₁₄ alkyl; R⁴ is —(CH₂)ₙOH; n is 2; each R⁵ is H; each R⁶ is H; M and M′ are each —C(O)O—; R′ is a C₁₋₁₂ alkyl; 1 is 5; and m is 7.

In some embodiments of Formula (AI) or (AIb), R′^{a} is R′^{branched}; R′^{branched} is

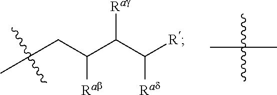

denotes a point of attachment; R^{aβ}, R^{aγ}, and R^{aδ} are each H; R² and R³ are each C₁-14 alkyl; R⁴ is —(CH₂)ₙOH; n is 2; each R⁵ is H; each R⁶ is H; M and M′ are each —C(O)O—; R′ is a C₁₋₁₂ alkyl; 1 is 3; and m is 7.

In some embodiments of Formula (AI) or (Ab), R′^{a} is R′^{branched}; R′^{branched} is

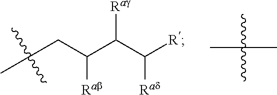

denotes a point of attachment; R^{aβ} and R^{aδ} are each H; R^{aγ} is C₂₋₁₂ alkyl; R² and R³ are each C₁₋₁₄ alkyl; R⁴ is —(CH₂)ₙOH; n is 2; each R⁵ is H; each R⁶ is H; M and M′ are each —C(O)O—; R′ is a C₁₋₁₂ alkyl; 1 is 5; and m is 7.

In some embodiments, the ionizable amino lipid of Formula (AI) is a compound of Formula (AIc):

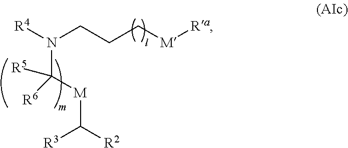

or its N-oxide, or a salt or isomer thereof,
wherein R′^{a} is R′^{branched}; wherein
R′^{branched} is:

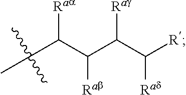

wherein

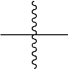

denotes a point of attachment;
wherein R^{aα}, R^{aβ}, R^{aγ}, and R^{aδ} are each independently selected from the group consisting of H, C₂₋₁₂ alkyl, and C₂₋₁₂ alkenyl;
R² and R³ are each independently selected from the group consisting of C₁₋₁₄ alkyl and C₂₋₁₄ alkenyl;

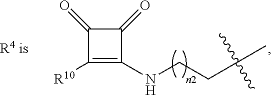

wherein

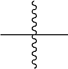

denotes a point of attachment; wherein R¹⁰ is N(R)₂; each R is independently selected from the group consisting of C₁₋₆ alkyl, C₂₋₃ alkenyl, and H; n2 is selected from the group consisting of 1, 2, 3, 4, 5, 6, 7, 8, 9, and 10;
each R⁵ is independently selected from the group consisting of C₁₋₃ alkyl, C₂₋₃ alkenyl, and H;
each R⁶ is independently selected from the group consisting of C₁₋₃ alkyl, C₂₋₃ alkenyl, and H;
M and M′ are each independently selected from the group consisting of —C(O)O— and —OC(O)—;
R′ is a C₁₋₁₂ alkyl or C₂₋₁₂ alkenyl;
l is selected from the group consisting of 1, 2, 3, 4, and 5; and
m is selected from the group consisting of 5, 6, 7, 8, 9, 10, 11, 12, and 13.

In some embodiments, R′^{a} is R′^{branched}; R′^{branched} is

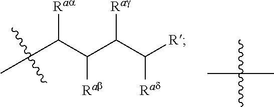

denotes a point of attachment; R^{aβ}, R^{aγ}, and R^{aδ} are each H; R^{aα} is C₂₋₁₂ alkyl; R² and R³ are each C₁₋₁₄ alkyl; R⁴ is

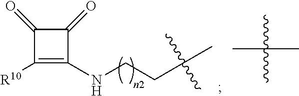

denotes a point of attachment; R¹⁰ is NH(C₁₋₆ alkyl); n2 is 2; each R⁵ is H; each R⁶ is H; M and M′ are each —C(O)O—; R′ is a C₁₋₁₂ alkyl; l is 5; and m is 7.

In some embodiments, the compound of Formula (AIc) is:

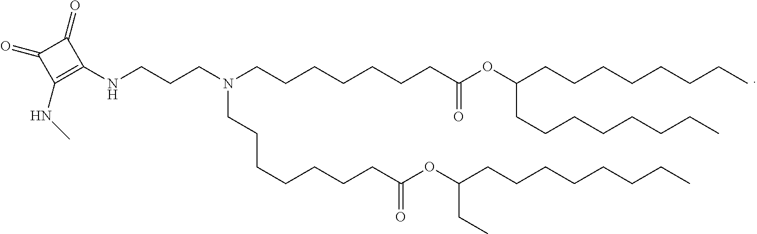 [SMILES: CCCCCCCCC(CC)OC(=O)CCCCCCCN(CCCCCCCC(=O)OC(CCCCCCCC)CCCCCCCC)CCCNc1c(NC)c(=O)c1=O]

### Formula (AII)

In some embodiments, the ionizable amino lipid is a compound of Formula (AII):

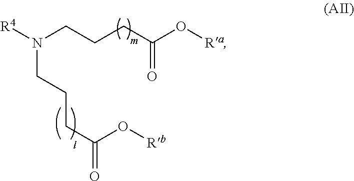

or its N-oxide, or a salt or isomer thereof,
wherein R′^{a} is R′^{branched} or R′^{cyclic}; wherein
R′^{branched} is:

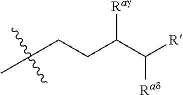

and R′^{cyclic} is:

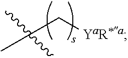

and
R′^{b} is:

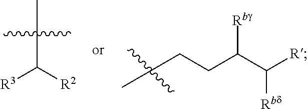

wherein

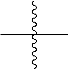

denotes a point of attachment;
R^{aγ} and R^{aδ} are each independently selected from the group consisting of H, C₁₋₁₂ alkyl, and C₂₋₁₂ alkenyl, wherein at least one of R^{aγ} and R^{aδ} is selected from the group consisting of C₁₋₁₂ alkyl and C₂₋₁₂ alkenyl;
R^{bγ} and R^{bδ} are each independently selected from the group consisting of H, C₁₋₁₂ alkyl, and C₂₋₁₂ alkenyl, wherein at least one of R^{bγ} and R^{b}a is selected from the group consisting of C₁₋₁₂ alkyl and C₂₋₁₂ alkenyl;
R² and R³ are each independently selected from the group consisting of C₁₋₁₄ alkyl and C₂₋₁₄ alkenyl;
R⁴ is selected from the group consisting of —(CH₂)ₙOH wherein n is selected from the group consisting of 1, 2, 3, 4, and 5, and

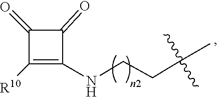

wherein

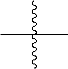

denotes a point of attachment; wherein R¹⁰ is N(R)₂; each R is independently selected from the group consisting of C₁₋₆ alkyl, C₂₋₃ alkenyl, and H; and n2 is selected from the group consisting of 1, 2, 3, 4, 5, 6, 7, 8, 9, and 10;
each R′ independently is a C₁₋₁₂ alkyl or C₂₋₁₂ alkenyl;
Y^{a} is a C₃₋₆ carbocycle;
R*^{″a} is selected from the group consisting of C₁₋₁₅ alkyl and C₂₋₁₅ alkenyl; and
s is 2 or 3;
m is selected from 1, 2, 3, 4, 5, 6, 7, 8, and 9;
l is selected from 1, 2, 3, 4, 5, 6, 7, 8, and 9.

In some embodiments, the ionizable amino lipid of Formula (AII) is a compound of Formula (AII-a):

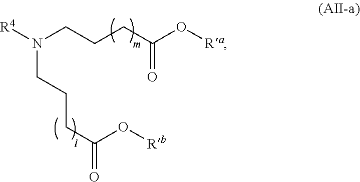

or its N-oxide, or a salt or isomer thereof,
wherein R′^{a} is R′^{branched} or R′^{cyclic}; wherein
R′^{branched} is:

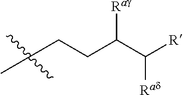

and R′^{b} is:

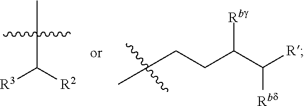

wherein

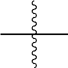

denotes a point of attachment;
R^{aγ} and R^{aδ} are each independently selected from the group consisting of H, C₁₋₁₂ alkyl, and C₂₋₁₂ alkenyl, wherein at least one of R^{aγ} and R^{aδ} is selected from the group consisting of C₁₋₁₂ alkyl and C₂₋₁₂ alkenyl;
R^{bγ} and R^{bδ} are each independently selected from the group consisting of H, C₁₋₁₂ alkyl, and C₂₋₁₂ alkenyl, wherein at least one of R^{bγ} and R^{bδ} is selected from the group consisting of C₁₋₁₂ alkyl and C₂₋₁₂ alkenyl;
R² and R³ are each independently selected from the group consisting of C₁₋₁₄ alkyl and C₂₋₁₄ alkenyl;
R⁴ is selected from the group consisting of —(CH₂)ₙOH wherein n is selected from the group consisting of 1, 2, 3, 4, and 5, and

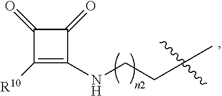

wherein

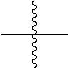

denotes a point of attachment; wherein R¹⁰ is N(R)₂; each R is independently selected from the group consisting of C₁₋₆ alkyl, C₂₋₃ alkenyl, and H; and n2 is selected from the group consisting of 1, 2, 3, 4, 5, 6, 7, 8, 9, and 10;
each R′ independently is a C₁₋₁₂ alkyl or C₂₋₁₂ alkenyl;
m is selected from 1, 2, 3, 4, 5, 6, 7, 8, and 9;
l is selected from 1, 2, 3, 4, 5, 6, 7, 8, and 9.

In some embodiments, the ionizable amino lipid of Formula (AII) is a compound of Formula (AII-b):

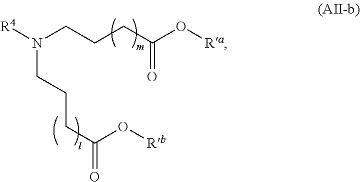

or its N-oxide, or a salt or isomer thereof,
wherein R′^{a} is R′^{branched} or R′^{cyclic}; wherein
R′^{branched} is:

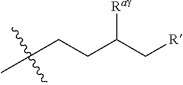

and R′^{b} is:

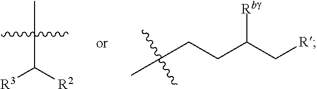

wherein

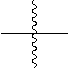

denotes a point of attachment;
R^{aγ} and R^{bγ} are each independently selected from the group consisting of C₁₋₁₂ alkyl and C₂₋₁₂ alkenyl;
R² and R³ are each independently selected from the group consisting of C₁₋₁₄ alkyl and C₂₋₁₄ alkenyl;
R⁴ is selected from the group consisting of —(CH₂)ₙOH wherein n is selected from the group consisting of 1, 2, 3, 4, and 5, and

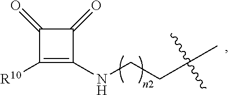

wherein

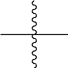

denotes a point of attachment; wherein R¹⁰ is N(R)₂; each R is independently selected from the group consisting of C₁₋₆ alkyl, C₂₋₃ alkenyl, and H; and n2 is selected from the group consisting of 1, 2, 3, 4, 5, 6, 7, 8, 9, and 10;
each R′ independently is a C₁₋₁₂ alkyl or C₂₋₁₂ alkenyl;
m is selected from 1, 2, 3, 4, 5, 6, 7, 8, and 9;
l is selected from 1, 2, 3, 4, 5, 6, 7, 8, and 9.

In some embodiments, the ionizable amino lipid of Formula (AII) is a compound of Formula (AII-c):

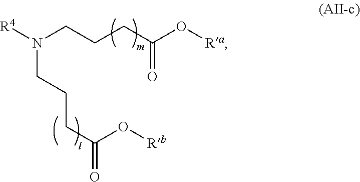

or its N-oxide, or a salt or isomer thereof,
wherein R′^{a} is R′^{branched} or R′^{cyclic}; wherein
R′^{branched} is:

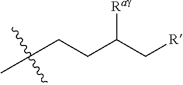

and R′^{b} is:

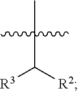

wherein

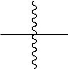

denotes a point of attachment;
wherein R^{aγ} is selected from the group consisting of C₁₋₁₂ alkyl and C₂₋₁₂ alkenyl;
R² and R³ are each independently selected from the group consisting of C₁₋₁₄ alkyl and C₂₋₁₄ alkenyl;
R⁴ is selected from the group consisting of —(CH₂)ₙOH wherein n is selected from the group consisting of 1, 2, 3, 4, and 5, and

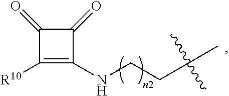

wherein

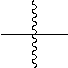

denotes a point of attachment; wherein R¹⁰ is N(R)₂; each R is independently selected from the group consisting of C₁₋₆ alkyl, C₂₋₃ alkenyl, and H; and n2 is selected from the group consisting of 1, 2, 3, 4, 5, 6, 7, 8, 9, and 10;
R′ is a C₁₋₁₂ alkyl or C₂₋₁₂ alkenyl;
m is selected from 1, 2, 3, 4, 5, 6, 7, 8, and 9;
l is selected from 1, 2, 3, 4, 5, 6, 7, 8, and 9.

In some embodiments, the ionizable amino lipid of Formula (AII) is a compound of Formula (AII-d):

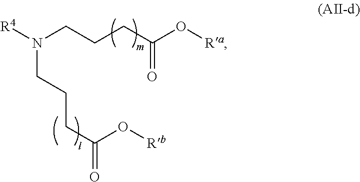

or its N-oxide, or a salt or isomer thereof,
wherein R′^{a} is R′^{branched} or R′^{cyclic}; wherein
R′^{branched} is:

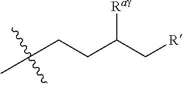

and R′^{b} is:

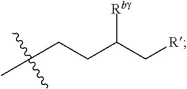

wherein

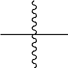

denotes a point of attachment;
wherein R^{aγ} and R^{bγ} are each independently selected from the group consisting of C₁₋₁₂ alkyl and C₂₋₁₂ alkenyl;
R⁴ is selected from the group consisting of —(CH₂)ₙOH wherein n is selected from the group consisting of 1, 2, 3, 4, and 5, and

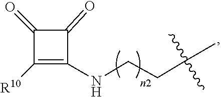

wherein

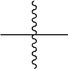

denotes a point of attachment; wherein R¹⁰ is N(R)₂; each R is independently selected from the group consisting of C₁₋₆ alkyl, C₂₋₃ alkenyl, and H; and n2 is selected from the group consisting of 1, 2, 3, 4, 5, 6, 7, 8, 9, and 10;
each R′ independently is a C₁₋₁₂ alkyl or C₂₋₁₂ alkenyl;
m is selected from 1, 2, 3, 4, 5, 6, 7, 8, and 9;
l is selected from 1, 2, 3, 4, 5, 6, 7, 8, and 9.

In some embodiments, the ionizable amino lipid of Formula (AII) is a compound of Formula (AII-e):

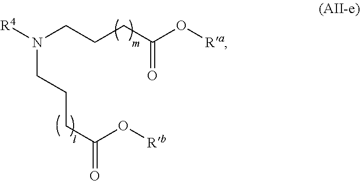

or its N-oxide, or a salt or isomer thereof,
wherein R′^{a} is R′^{branched} or R′^{cyclic}; wherein
R′^{branched} is:

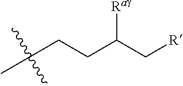

R′^{b} is:

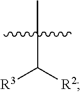

wherein

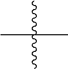

denotes a point of attachment;
wherein R^{aγ} is selected from the group consisting of C₁₋₁₂ alkyl and C₂₋₁₂ alkenyl;
R² and R³ are each independently selected from the group consisting of C₁₋₁₄ alkyl and C₂₋₁₄ alkenyl;
R⁴ is —(CH₂)ₙOH wherein n is selected from the group consisting of 1, 2, 3, 4, and 5;
R′ is a C₁₋₁₂ alkyl or C₂₋₁₂ alkenyl;
m is selected from 1, 2, 3, 4, 5, 6, 7, 8, and 9;
l is selected from 1, 2, 3, 4, 5, 6, 7, 8, and 9.

In some embodiments of the compound of Formula (AII), (AII-a), (AII-b), (AII-c), (AII-d), or (AII-e), m and l are each independently selected from 4, 5, and 6. In some embodiments of the compound of Formula (AII), (AII-a), (AII-b), (AII-c), (AII-d), or (AII-e), m and l are each 5.

In some embodiments of the compound of Formula (AII), (AII-a), (AII-b), (AII-c), (AII-d), or (AII-e), each R′ independently is a C₁₋₁₂ alkyl. In some embodiments of the compound of Formula (AII), (AII-a), (AII-b), (AII-c), (AII-d), or (AII-e), each R′ independently is a C₂₋₅ alkyl.

In some embodiments of the compound of Formula (AII), (AII-a), (AII-b), (AII-c), (AII-d), or (AII-e), R′^{b} is:

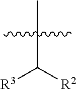

and R² and R³ are each independently a C₁₋₁₄ alkyl. In some embodiments of the compound of Formula (AII), (AII-a), (AII-b), (AII-c), (AII-d), or (AII-e), R′^{b} is:

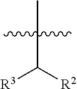

and R² and R³ are each independently a C₆₋₁₀ alkyl. In some embodiments of the compound of Formula (AII), (AII-a), (AII-b), (AII-c), (AII-d), or (AII-e), R′^{b} is:

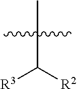

and R² and R³ are each a Cs alkyl.

In some embodiments of the compound of Formula (AII), (AII-a), (AII-b), (AII-c), (AII-d), or (AII-e), R′^{branched} is:

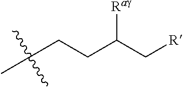

### and R′^{b} is:

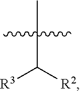

R^{aγ} is a C₁₋₁₂ alkyl and R² and R³ are each independently a C₆₋₁₀ alkyl. In some embodiments of the compound of Formula (AII), (AII-a), (AII-b), (AII-c), (AII-d), or (AII-e), R′^{branched} is:

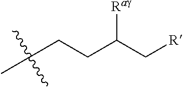

### and R′^{b} is:

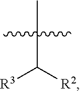

R^{aγ} is a C₂₋₆ alkyl and R² and R³ are each independently a C₆₋₁₀ alkyl. In some embodiments of the compound of Formula (AII), (AII-a), (AII-b), (AII-c), (AII-d), or (AII-e), R′^{branched} is:

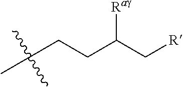

### and R′^{b} is:

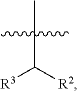

R^{aγ} is a C₂₋₆ alkyl, and R² and R³ are each a C₈ alkyl.

In some embodiments of the compound of Formula (AII), (AII-a), (AII-b), (AII-c), (AII-d), or (AII-e), R′^{branched} is:

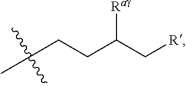

### R′^{b} is:

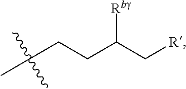

and R^{aγ} and R^{bγ} are each a C₁₋₁₂ alkyl. In some embodiments of the compound of Formula (AII), (AII-a), (AII-b), (AII-c), (AII-d), or (AII-e), R′^{branched} is:

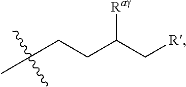

### R′^{b} is:

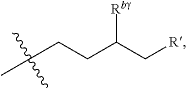

and R^{aγ} and R^{bγ} are each a C₂₋₆ alkyl.

In some embodiments of the compound of Formula (AII), (AII-a), (AII-b), (AII-c), (AII-d), or (AII-e), m and l are each independently selected from 4, 5, and 6 and each R′ independently is a C₁₋₁₂ alkyl. In some embodiments of the compound of Formula (AII), (AII-a), (AII-b), (AII-c), (AII-d), or (AII-e), m and l are each 5 and each R′ independently is a C₂₋₅ alkyl.

In some embodiments of the compound of (AII), (AII-a), (AII-b), (AII-c), (AII-d), or (AII-e), R′^{branched} is:

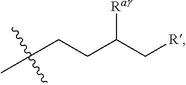

### R′^{b} is:

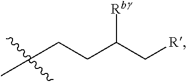

m and l are each independently selected from 4, 5, and 6, each R′ independently is a C₁₋₁₂ alkyl, and R^{aγ} and R^{bγ} are each a C₁₋₁₂ alkyl. In some embodiments of the compound of Formula (AII), (AII-a), (AII-b), (AII-c), (AII-d), or (AII-e), R′^{branched} is:

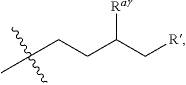

### R′^{b} is:

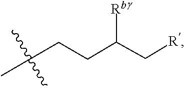

m and l are each 5, each R′ independently is a C₂₋₅ alkyl, and R^{aγ} and R^{bγ} are each a C₂₋₆ alkyl.

In some embodiments of the compound of Formula (AII), (AII-a), (AII-b), (AII-c), (AII-d), or (AII-e), R′^{branched} is:

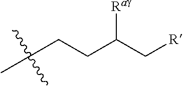

### and R′^{b} is:

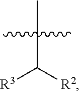

m and l are each independently selected from 4, 5, and 6, R′ is a C₁₋₁₂ alkyl, R^{aγ} is a C₁₋₁₂ alkyl and R² and R³ are each independently a C₆₋₁₀ alkyl.

In some embodiments of the compound of Formula (AII), (AII-a), (AII-b), (AII-c), (AII-d), or (AII-e), R′^{branched} is:

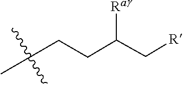

### and R′^{b} is:

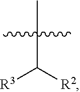

m and l are each 5, R′ is a C₂₋₅ alkyl, R^{aγ} is a C₂₋₆ alkyl, and R² and R³ are each a C₈ alkyl.

In some embodiments of the compound of (AII), (AII-a), (AII-b), (AII-c), (AII-d), or (AII-e), R⁴ is

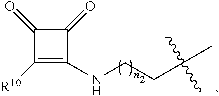

wherein R¹⁰ is NH(C₁₋₆ alkyl) and n2 is 2. In some embodiments of the compound of Formula (AII), (AII-a), (AII-b), (AII-c), (AII-d), or (AII-e), R⁴ is

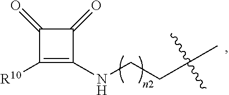

wherein R¹⁰ is NH(CH₃) and n2 is 2.

In some embodiments of the compound of Formula (AII), (AII-a), (AII-b), (AII-c), (AII-d), or (AII-e), R′^{branched} is:

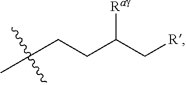

### R′^{b} is:

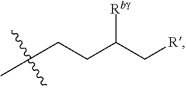

m and l are each independently selected from 4, 5, and 6, each R′ independently is a C₁₋₁₂ alkyl, R^{aγ} and R^{bγ} are each a C₁₋₁₂ alkyl, and R⁴ is

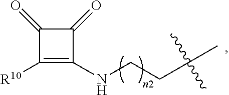

wherein R¹⁰ is NH(C₁₋₆ alkyl), and n2 is 2. In some embodiments of the compound of Formula (AII), (AII-a), (AII-b), (AII-c), (AII-d), or (AI-e), R′^{branched} is:

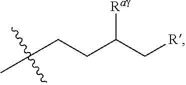

### R′^{b} is:

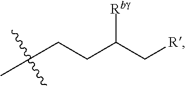

m and l are each 5, each R′ independently is a C₂₋₅ alkyl, R^{aγ} and R^{bγ} are each a C₂₋₆ alkyl, and R⁴ is

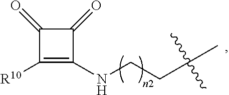

wherein R¹⁰ is NH(CH₃) and n2 is 2.

In some embodiments of the compound of Formula (AII), (AII-a), (AII-b), (AII-c), (AII-d), or (AII-e), R′^{branched} is:

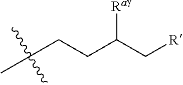

### and R′^{b} is:

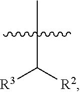

m and l are each independently selected from 4, 5, and 6, R′ is a C₁₋₁₂ alkyl, R² and R³ are each independently a C₆₋₁₀ alkyl, R^{aγ} is a C₁₋₁₂ alkyl, and R⁴ is

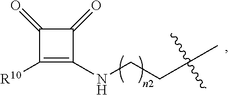

wherein R¹⁰ is NH(C₁₋₆ alkyl) and n2 is 2. In some embodiments of the compound of Formula (AII), (AII-a), (AII-b), (AII-c), (AII-d), or (AII-e), R′^{branched} is:

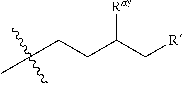

### and R′^{b} is:

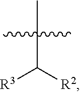

m and l are each 5, R′ is a C₂₋₅ alkyl, R^{aγ} is a C₂₋₆ alkyl, R² and R³ are each a C₈ alkyl, and R⁴ is

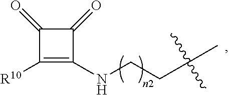

wherein R¹⁰ is NH(CH₃) and n2 is 2.

In some embodiments of the compound of Formula (AII), (AII-a), (AII-b), (AII-c), (AII-d), or (AII-e), R⁴ is —(CH₂)ₙOH and n is 2, 3, or 4. In some embodiments of the compound of Formula (AII), (AII-a), (AII-b), (AII-c), (AII-d), or (AII-e), R⁴ is —(CH₂)ₙOH and n is 2.

In some embodiments of the compound of Formula (AII), (AII-a), (AII-b), (AII-c), (AII-d), or (AII-e), R′^{branched} is:

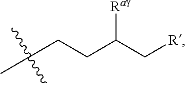

### R′^{b} is:

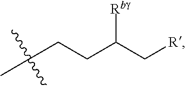

m and l are each independently selected from 4, 5, and 6, each R′ independently is a C₁₋₁₂ alkyl, R^{aγ} and R^{bγ} are each a C₁₋₁₂ alkyl, R⁴ is —(CH₂)ₙOH, and n is 2, 3, or 4. In some embodiments of the compound of Formula (AII), (AII-a), (AII-b), (AII-c), (AII-d), or (AII-e), R′^{branched} is:

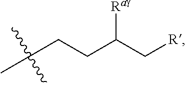

### R′^{b} is:

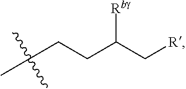

m and l are each 5, each R′ independently is a C₂₋₅ alkyl, R^{aγ} and R^{bγ} are each a C₂₋₆ alkyl, R⁴ is —(CH₂)ₙOH, and n is 2.

In some embodiments, the ionizable amino lipid of Formula (AII) is a compound of Formula (AII-f):

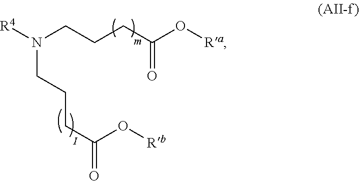

or its N-oxide, or a salt or isomer thereof,
wherein R′^{a} is R′^{branched} or R′^{cyclic}; wherein
R′^{branched} is:

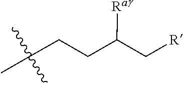

and R′^{b} is:

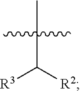

wherein

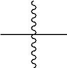

denotes a point of attachment;
R^{aγ} is a C₁₋₁₂ alkyl;
R² and R³ are each independently a C₁₋₁₄ alkyl;
R⁴ is —(CH₂)ₙOH wherein n is selected from the group consisting of 1, 2, 3, 4, and 5;
R′ is a C₁₋₁₂ alkyl;
m is selected from 4, 5, and 6; and
l is selected from 4, 5, and 6.

In some embodiments of the compound of Formula (AII-f), m and l are each 5, and n is 2, 3, or 4.

In some embodiments of the compound of Formula (AII-f) R′ is a C₂₋₅ alkyl, R^{aγ} is a C₂₋₆ alkyl, and R² and R³ are each a C₆₋₁₀ alkyl.

In some embodiments of the compound of Formula (AII-f), m and l are each 5, n is 2, 3, or 4, R′ is a C₂₋₅ alkyl, R^{aγ} is a C₂₋₆ alkyl, and R² and R³ are each a C₆₋₁₀ alkyl.

In some embodiments, the ionizable amino lipid of Formula (AII) is a compound of Formula (AII-g):

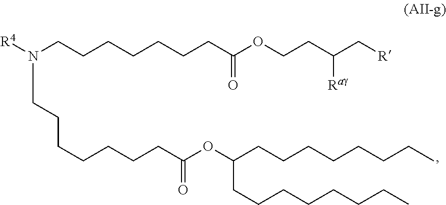

or its N-oxide, or a salt or isomer thereof; wherein
R^{aγ} is a C₂₋₆ alkyl;
R′ is a C₂₋₅ alkyl; and
R⁴ is selected from the group consisting of —(CH₂)ₙOH wherein n is selected from the group consisting of 3, 4, and 5, and

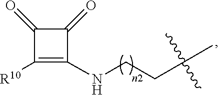

wherein

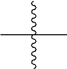

denotes a point of attachment, R¹⁰ is NH(C₁₋₆ alkyl), and n2 is selected from the group consisting of 1, 2, and 3.

In some embodiments, the ionizable amino lipid of Formula (AII) is a compound of Formula (AII-h):

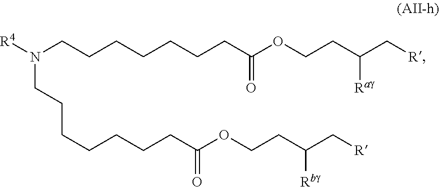

or its N-oxide, or a salt or isomer thereof; wherein
R^{aγ} and R^{bγ} are each independently a C₂₋₆ alkyl;
each R′ independently is a C₂₋₅ alkyl; and
R⁴ is selected from the group consisting of —(CH₂)ₙOH wherein n is selected from the group consisting of 3, 4, and 5, and

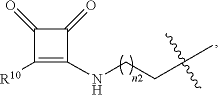

wherein

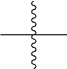

denotes a point of attachment, R¹⁰ is NH(C₁₋₆ alkyl), and n2 is selected from the group consisting of 1, 2, and 3.

In some embodiments of the compound of Formula (AII-g) or (AII-h), R⁴ is

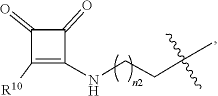

wherein
R¹⁰ is NH(CH₃) and n2 is 2.

In some embodiments of the compound of Formula (AII-g) or (AII-h), R⁴ is —(CH₂)₂OH.

### Formula (AII)

In some embodiments, the ionizable amino lipids may be one or more of compounds of Formula (AIII):

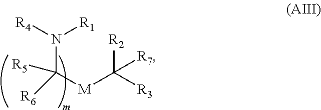

or their N-oxides, or salts or isomers thereof, wherein:
R₁ is selected from the group consisting of C₅₋₃₀ alkyl, C₅₋₂₀ alkenyl, —R*YR″, —YR″, and —R″M′R′;
R₂ and R₃ are independently selected from the group consisting of H, C₁₋₁₄ alkyl, C₂₋₁₄ alkenyl, —R*YR″, —YR″, and —R*OR″, or R₂ and R₃, together with the atom to which they are attached, form a heterocycle or carbocycle;
R₄ is selected from the group consisting of hydrogen, a C₃₋₆ carbocycle, —(CH₂)ₙQ, —(CH₂)ₙCHQR, —CHQR, —CQ(R)₂, and unsubstituted C₁₋₆ alkyl, where Q is selected from a carbocycle, heterocycle, —OR, —O(CH₂)ₙN(R)₂, —C(O)OR, —OC(O)R, —CX₃, —CX₂H, —CXH₂, —CN, —N(R)₂, —C(O)N(R)₂, —N(R)C(O)R, —N(R)S(O)₂R, —N(R)C(O)N(R)₂, —N(R)C(S)N(R)₂, —N(R)R₈, —N(R)S(O)₂R₈, —O(CH₂)ₙOR, —N(R)C(═NR₉)N(R)₂, —N(R)C(═CHR₉)N(R)₂, —OC(O)N(R)₂, —N(R)C(O)OR, —N(OR)C(O)R, —N(OR)S(O)₂R, —N(OR)C(O)OR, —N(OR)C(O)N(R)₂, —N(OR)C(S)N(R)₂, —N(OR)C(═NR₉)N(R)₂, —N(OR)C(═CHR₉)N(R)₂, —C(═NR₉)N(R)₂, —C(═NR₉)R, —C(O)N(R)OR, and —C(R)N(R)₂C(O)OR, and each n is independently selected from 1, 2, 3, 4, and 5;
each R₅ is independently selected from the group consisting of C₁₋₃ alkyl, C₂₋₃ alkenyl, and H;
each R₆ is independently selected from the group consisting of C₁₋₃ alkyl, C₂₋₃ alkenyl, and H;
M and M′ are independently selected from —C(O)O—, —OC(O)—, —OC(O)-M″-C(O)O—, —C(O)N(R′)—, —N(R′)C(O)—, —C(O)—, —C(S)—, —C(S)S—, —SC(S)—, —CH(OH)—, —P(O)(OR′)O—, —S(O)₂—, —S—S—, an aryl group, and a heteroaryl group, in which M″ is a bond, C₁₋₁₃ alkyl or C₂₋₁₃ alkenyl;
R₇ is selected from the group consisting of C₁₋₃ alkyl, C₂₋₃ alkenyl, and H;
R₈ is selected from the group consisting of C₃₋₆ carbocycle and heterocycle;
R₉ is selected from the group consisting of H, CN, NO₂, C₁₋₆ alkyl, —OR, —S(O)₂R, —S(O)₂N(R)₂, C₂₋₆ alkenyl, C₃₋₆ carbocycle and heterocycle;
each R is independently selected from the group consisting of C₁₋₃ alkyl, C₂₋₃ alkenyl, and H;
each R′ is independently selected from the group consisting of C₁₋₁₈ alkyl, C₂₋₁₈ alkenyl, —R*YR″, —YR″, and H;
each R″ is independently selected from the group consisting of C₃₋₁₅ alkyl and C₃₋₁₅ alkenyl;
each R* is independently selected from the group consisting of C₁₋₁₂ alkyl and C₂₋₁₂ alkenyl;
each Y is independently a C₃₋₆ carbocycle;
each X is independently selected from the group consisting of F, Cl, Br, and I; and
m is selected from 5, 6, 7, 8, 9, 10, 11, 12, and 13; and wherein when R₄ is —(CH₂)ₙQ, —(CH₂)ₙCHQR, —CHQR, or —CQ(R)₂, then (i) Q is not —N(R)₂ when n is 1, 2, 3, 4 or 5, or (ii) Q is not 5, 6, or 7-membered heterocycloalkyl when n is 1 or 2.

In some embodiments, another subset of compounds of Formula (AIII) includes those in which:
R₁ is selected from the group consisting of C₅₋₃₀ alkyl, C₅₋₂₀ alkenyl, —R*YR″, —YR″, and —R″M′R′;
R₂ and R₃ are independently selected from the group consisting of H, C₁₋₁₄ alkyl, C₂₋₁₄ alkenyl, —R*YR″, —YR″, and —R*OR″, or R₂ and R₃, together with the atom to which they are attached, form a heterocycle or carbocycle;
R₄ is selected from the group consisting of a C₃₋₆ carbocycle, —(CH₂)ₙQ, —(CH₂)ₙCHQR, —CHQR, —CQ(R)₂, and unsubstituted C₁₋₆ alkyl, where Q is selected from a C₃₋₆ carbocycle, a 5- to 14-membered heteroaryl having one or more heteroatoms selected from N, O, and S, —OR, —O(CH₂)ₙN(R)₂, —C(O)OR, —OC(O)R, —CX₃, —CX₂H, —CXH₂, —CN, —C(O)N(R)₂, —N(R)C(O)R, —N(R)S(O)₂R, —N(R)C(O)N(R)₂, —N(R)C(S)N(R)₂, —CRN(R)₂C(O)OR, —N(R)R₈, —O(CH₂)ₙOR, —N(R)C(═NR₉)N(R)₂, —N(R)C(═CHR₉)N(R)₂, —OC(O)N(R)₂, —N(R)C(O)OR, —N(OR)C(O)R, —N(OR)S(O)₂R, —N(OR)C(O)OR, —N(OR)C(O)N(R)₂, —N(OR)C(S)N(R)₂, —N(OR)C(═NR₉)N(R)₂, —N(OR)C(═CHR₉)N(R)₂, —C(═NR₉)N(R)₂, —C(═NR₉)R, —C(O)N(R)OR, and a 5- to 14-membered heterocycloalkyl having one or more heteroatoms selected from N, O, and S which is substituted with one or more substituents selected from oxo (═O), OH, amino, mono- or di-alkylamino, and C₁₋₃ alkyl, and each n is independently selected from 1, 2, 3, 4, and 5;
each R₅ is independently selected from the group consisting of C₁₋₃ alkyl, C₂₋₃ alkenyl, and H;
each R₆ is independently selected from the group consisting of C₁₋₃ alkyl, C₂₋₃ alkenyl, and H;
M and M′ are independently selected from —C(O)O—, —OC(O)—, —C(O)N(R′)—, —N(R′)C(O)—, —C(O)—, —C(S)—, —C(S)S—, —SC(S)—, —CH(OH)—, —P(O)(OR′)O—, —S(O)₂—, —S—S—, an aryl group, and a heteroaryl group;
R₇ is selected from the group consisting of C₁₋₃ alkyl, C₂₋₃ alkenyl, and H;
R₈ is selected from the group consisting of C₃₋₆ carbocycle and heterocycle;
R₉ is selected from the group consisting of H, CN, NO₂, C₁₋₆ alkyl, —OR, —S(O)₂R, —S(O)₂N(R)₂, C₂₋₆ alkenyl, C₃₋₆ carbocycle and heterocycle;
each R is independently selected from the group consisting of C₁₋₃ alkyl, C₂₋₃ alkenyl, and H;
each R′ is independently selected from the group consisting of C₁₋₁₈ alkyl, C₂₋₁₈ alkenyl, —R*YR″, —YR″, and H;
each R″ is independently selected from the group consisting of C₃₋₁₄ alkyl and C₃₋₁₄ alkenyl;
each R* is independently selected from the group consisting of C₁₋₁₂ alkyl and C₂₋₁₂ alkenyl;
each Y is independently a C₃₋₆ carbocycle;
each X is independently selected from the group consisting of F, Cl, Br, and I; and
m is selected from 5, 6, 7, 8, 9, 10, 11, 12, and 13,
or salts or isomers thereof.

In some embodiments, another subset of compounds of Formula (AIII) includes those in which:
R₁ is selected from the group consisting of C₅₋₃₀ alkyl, C₅₋₂₀ alkenyl, —R*YR″, —YR″, and —R″M′R′;
R₂ and R₃ are independently selected from the group consisting of H, C₁₋₁₄ alkyl, C₂₋₁₄ alkenyl, —R*YR″, —YR″, and —R*OR″, or R₂ and R₃, together with the atom to which they are attached, form a heterocycle or carbocycle;
R₄ is selected from the group consisting of a C₃₋₆ carbocycle, —(CH₂)ₙQ, —(CH₂)ₙCHQR, —CHQR, —CQ(R)₂, and unsubstituted C₁₋₆ alkyl, where Q is selected from a C₃₋₆ carbocycle, a 5- to 14-membered heterocycle having one or more heteroatoms selected from N, O, and S, —OR, —O(CH₂)ₙN(R)₂, —C(O)OR, —OC(O)R, —CX₃, —CX₂H, —CXH₂, —CN, —C(O)N(R)₂, —N(R)C(O)R, —N(R)S(O)₂R, —N(R)C(O)N(R)₂, —N(R)C(S)N(R)₂, —CRN(R)₂C(O)OR, —N(R)R₈, —O(CH₂)ₙOR, —N(R)C(═NR₉)N(R)₂, —N(R)C(═CHR₉)N(R)₂, —OC(O)N(R)₂, —N(R)C(O)OR, —N(OR)C(O)R, —N(OR)S(O)₂R, —N(OR)C(O)OR, —N(OR)C(O)N(R)₂, —N(OR)C(S)N(R)₂, —N(OR)C(═NR₉)N(R)₂, —N(OR)C(═CHR₉)N(R)₂, —C(═NR₉)R, —C(O)N(R)OR, and —C(═NR₉)N(R)₂, and each n is independently selected from 1, 2, 3, 4, and 5; and when Q is a 5- to 14-membered heterocycle and (i) R₄ is —(CH₂)ₙQ in which n is 1 or 2, or (ii) R₄ is —(CH₂)ₙCHQR in which n is 1, or (iii) R₄ is —CHQR, and —CQ(R)₂, then Q is either a 5- to 14-membered heteroaryl or 8- to 14-membered heterocycloalkyl;
each R₅ is independently selected from the group consisting of C₁₋₃ alkyl, C₂₋₃ alkenyl, and H;
each R₆ is independently selected from the group consisting of C₁₋₃ alkyl, C₂₋₃ alkenyl, and H;
M and M′ are independently selected from —C(O)O—, —OC(O)—, —C(O)N(R′)—, —N(R′)C(O)—, —C(O)—, —C(S)—, —C(S)S—, —SC(S)—, —CH(OH)—, —P(O)(OR′)O—, —S(O)₂—, —S—S—, an aryl group, and a heteroaryl group;
R₇ is selected from the group consisting of C₁₋₃ alkyl, C₂₋₃ alkenyl, and H;
R₈ is selected from the group consisting of C₃₋₆ carbocycle and heterocycle;
R₉ is selected from the group consisting of H, CN, NO₂, C₁₋₆ alkyl, —OR, —S(O)₂R, —S(O)₂N(R)₂, C₂₋₆ alkenyl, C₃₋₆ carbocycle and heterocycle;
each R is independently selected from the group consisting of C₁₋₃ alkyl, C₂₋₃ alkenyl, and H;
each R′ is independently selected from the group consisting of C₁₋₁₈ alkyl, C₂₋₁₈ alkenyl, —R*YR″, —YR″, and H;
each R″ is independently selected from the group consisting of C₃₋₁₄ alkyl and C₃₋₁₄ alkenyl;
each R* is independently selected from the group consisting of C₁₋₁₂ alkyl and C₂₋₁₂ alkenyl;
each Y is independently a C₃₋₆ carbocycle;
each X is independently selected from the group consisting of F, Cl, Br, and I; and
m is selected from 5, 6, 7, 8, 9, 10, 11, 12, and 13,
or salts or isomers thereof.

In some embodiments, another subset of compounds of Formula (AIII) includes those in which:
R₁ is selected from the group consisting of C₅₋₃₀ alkyl, C₅₋₂₀ alkenyl, —R*YR″, —YR″, and —R″M′R′;
R₂ and R₃ are independently selected from the group consisting of H, C₁₋₁₄ alkyl, C₂₋₁₄ alkenyl, —R*YR″, —YR″, and —R*OR″, or R₂ and R₃, together with the atom to which they are attached, form a heterocycle or carbocycle;
R₄ is selected from the group consisting of a C₃₋₆ carbocycle, —(CH₂)ₙQ, —(CH₂)ₙCHQR, —CHQR, —CQ(R)₂, and unsubstituted C₁₋₆ alkyl, where Q is selected from a C₃₋₆ carbocycle, a 5- to 14-membered heteroaryl having one or more heteroatoms selected from N, O, and S, —OR, —O(CH₂)ₙN(R)₂, —C(O)OR, —OC(O)R, —CX₃, —CX₂H, —CXH₂, —CN, —C(O)N(R)₂, —N(R)C(O)R, —N(R)S(O)₂R, —N(R)C(O)N(R)₂, —N(R)C(S)N(R)₂, —CRN(R)₂C(O)OR, —N(R)R₈, —O(CH₂)ₙOR, —N(R)C(═NR₉)N(R)₂, —N(R)C(═CHR₉)N(R)₂, —OC(O)N(R)₂, —N(R)C(O)OR, —N(OR)C(O)R, —N(OR)S(O)₂R, —N(OR)C(O)OR, —N(OR)C(O)N(R)₂, —N(OR)C(S)N(R)₂, —N(OR)C(═NR₉)N(R)₂, —N(OR)C(═CHR₉)N(R)₂, —C(═NR₉)R, —C(O)N(R)OR, and —C(═NR₉)N(R)₂, and each n is independently selected from 1, 2, 3, 4, and 5;
each R₅ is independently selected from the group consisting of C₁₋₃ alkyl, C₂₋₃ alkenyl, and H;
each R₆ is independently selected from the group consisting of C₁₋₃ alkyl, C₂₋₃ alkenyl, and H;
M and M′ are independently selected from —C(O)O—, —OC(O)—, —C(O)N(R′)—, —N(R′)C(O)—, —C(O)—, —C(S)—, —C(S)S—, —SC(S)—, —CH(OH)—, —P(O)(OR′)O—, —S(O)₂—, —S—S—, an aryl group, and a heteroaryl group;
R₇ is selected from the group consisting of C₁₋₃ alkyl, C₂₋₃ alkenyl, and H;
R₈ is selected from the group consisting of C₃₋₆ carbocycle and heterocycle;
R₉ is selected from the group consisting of H, CN, NO₂, C₁₋₆ alkyl, —OR, —S(O)₂R, —S(O)₂N(R)₂, C₂₋₆ alkenyl, C₃₋₆ carbocycle and heterocycle;
each R is independently selected from the group consisting of C₁₋₃ alkyl, C₂₋₃ alkenyl, and H;
each R′ is independently selected from the group consisting of C₁₋₁₈ alkyl, C₂₋₁₈ alkenyl, —R*YR″, —YR″, and H;
each R″ is independently selected from the group consisting of C₃₋₁₄ alkyl and C₃₋₁₄ alkenyl;
each R* is independently selected from the group consisting of C₁₋₁₂ alkyl and C₂₋₁₂ alkenyl;
each Y is independently a C₃₋₆ carbocycle;
each X is independently selected from the group consisting of F, Cl, Br, and I; and
m is selected from 5, 6, 7, 8, 9, 10, 11, 12, and 13,
or salts or isomers thereof.

In some embodiments, another subset of compounds of Formula (AIII) includes those in which
R₁ is selected from the group consisting of C₅₋₃₀ alkyl, C₅₋₂₀ alkenyl, —R*YR″, —YR″, and —R″M′R′;
R₂ and R₃ are independently selected from the group consisting of H, C₂₋₁₄ alkyl, C₂₋₁₄ alkenyl, —R*YR″, —YR″, and —R*OR″, or R₂ and R₃, together with the atom to which they are attached, form a heterocycle or carbocycle;
R₄ is —(CH₂)ₙQ or —(CH₂)·CHQR, where Q is —N(R)₂, and n is selected from 3, 4, and 5;
each R₅ is independently selected from the group consisting of C₁₋₃ alkyl, C₂₋₃ alkenyl, and H;
each R₆ is independently selected from the group consisting of C₁₋₃ alkyl, C₂₋₃ alkenyl, and H;
M and M′ are independently selected from —C(O)O—, —OC(O)—, —C(O)N(R′)—, —N(R′)C(O)—, —C(O)—, —C(S)—, —C(S)S—, —SC(S)—, —CH(OH)—, —P(O)(OR′)O—, —S(O)₂—, —S—S—, an aryl group, and a heteroaryl group;
R₇ is selected from the group consisting of C₁₋₃ alkyl, C₂₋₃ alkenyl, and H;
each R is independently selected from the group consisting of C₁₋₃ alkyl, C₂₋₃ alkenyl, and H;
each R′ is independently selected from the group consisting of C₁₋₁₈ alkyl, C₂₋₁₈ alkenyl, —R*YR″, —YR″, and H;
each R″ is independently selected from the group consisting of C₃₋₁₄ alkyl and C₃₋₁₄ alkenyl;
each R* is independently selected from the group consisting of C₁₋₁₂ alkyl and C₁₋₁₂ alkenyl;
each Y is independently a C₃₋₆ carbocycle;
each X is independently selected from the group consisting of F, Cl, Br, and I; and
m is selected from 5, 6, 7, 8, 9, 10, 11, 12, and 13,
or salts or isomers thereof.

In some embodiments, another subset of compounds of Formula (AIII) includes those in which
R₁ is selected from the group consisting of C₅₋₃₀ alkyl, C₅₋₂₀ alkenyl, —R*YR″, —YR″, and —R″M′R′;
R₂ and R₃ are independently selected from the group consisting of C₁₋₁₄ alkyl, C₂₋₁₄ alkenyl, —R*YR″, —YR″, and —R*OR″, or R₂ and R₃, together with the atom to which they are attached, form a heterocycle or carbocycle;
R₄ is selected from the group consisting of —(CH₂)ₙQ, —(CH₂)ₙCHQR, —CHQR, and —CQ(R)₂, where Q is —N(R)₂, and n is selected from 1, 2, 3, 4, and 5;
each R₅ is independently selected from the group consisting of C₁₋₃ alkyl, C₂₋₃ alkenyl, and H;
each R₆ is independently selected from the group consisting of C₁₋₃ alkyl, C₂₋₃ alkenyl, and H;
M and M′ are independently selected from —C(O)O—, —OC(O)—, —C(O)N(R′)—, —N(R′)C(O)—, —C(O)—, —C(S)—, —C(S)S—, —SC(S)—, —CH(OH)—, —P(O)(OR′)O—, —S(O)₂—, —S—S—, an aryl group, and a heteroaryl group;
R₇ is selected from the group consisting of C₁₋₃ alkyl, C₂₋₃ alkenyl, and H;
each R is independently selected from the group consisting of C₁₋₃ alkyl, C₂₋₃ alkenyl, and H;
each R′ is independently selected from the group consisting of C₁₋₁₈ alkyl, C₂₋₁₈ alkenyl, —R*YR″, —YR″, and H;
each R″ is independently selected from the group consisting of C₃₋₁₄ alkyl and C₃₋₁₄ alkenyl;
each R* is independently selected from the group consisting of C₁₋₁₂ alkyl and C₁₋₁₂ alkenyl;
each Y is independently a C₃₋₆ carbocycle;
each X is independently selected from the group consisting of F, Cl, Br, and I; and
m is selected from 5, 6, 7, 8, 9, 10, 11, 12, and 13,
or salts or isomers thereof.

In some embodiments, R₁ is R″M′R′ or C₅₋₂₀ alkenyl; R₂ and R₃ are each independently selected from C₁₋₁₄ alkyl and C₂₋₁₄ alkenyl; R₄ is —(CH₂)ₙQ, wherein Q is OH and n is selected from 3, 4, and 5; M and M′ are each independently —OC(O)— or —C(O)O—; R₅, R₆, and R₇ are each H; R′ is a linear C₁₋₁₂ alkyl, or C₁₋₁₂ alkyl substituted with C₆₋₉ alkyl; R″ is C₃₋₁₄ alkyl; and m is selected from 5, 6, 7, 8, 9, 10, 11, 12, and 13.

In some embodiments, R₁ is R″M′R′; R₂ and R₃ are each independently C₁₋₁₄ alkyl; R₄ is —(CH₂)ₙQ, wherein Q is OH and n is 4; M and M′ are each independently —OC(O)—; R₅, R₆, and R₇ are each H; R′ is C₁₋₁₂ alkyl substituted with C₆₋₉ alkyl; R″ is C₃₋₁₄ alkyl; and m is 6.

In some embodiments, R₁ is C₅₋₂₀ alkenyl; R₂ and R₃ are each independently C₁₋₁₄ alkyl; R₄ is —(CH₂)ₙQ, wherein Q is OH and n is 3; M is —C(O)O—; R₅, R₆, and R₇ are each H; and m is 6.

In certain embodiments, a subset of compounds of Formula (AIII) includes those of Formula (AIII-A):

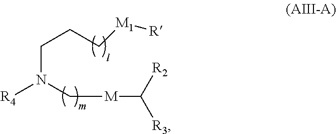

or its N-oxide, or a salt or isomer thereof, wherein 1 is selected from 1, 2, 3, 4, and 5; m is selected from 5, 6, 7, 8, and 9; M₁ is a bond or M′; R₄ is hydrogen, unsubstituted C₁₋₃ alkyl, or —(CH₂)ₙQ, in which Q is —OH, —NHC(S)N(R)₂, —NHC(O)N(R)₂, —N(R)C(O)R, —N(R)S(O)₂R, —N(R)R₈, —NHC(═NR₉)N(R)₂, —NHC(═CHR₉)N(R)₂, —OC(O)N(R)₂, —N(R)C(O)OR, heteroaryl or heterocycloalkyl; M and M′ are independently selected from —C(O)O—, —OC(O)—, —OC(O)-M″-C(O)O—, —C(O)N(R′)—, —P(O)(OR′)O—, —S—S—, an aryl group, and a heteroaryl group, and R₂ and R₃ are independently selected from the group consisting of H, C₁₋₁₄ alkyl, and C₂₋₁₄ alkenyl. For example, m is 5, 7, or 9. For example, Q is OH, —NHC(S)N(R)₂, or —NHC(O)N(R)₂. For example, Q is —N(R)C(O)R, or —N(R)S(O)₂R.

In certain embodiments, a subset of compounds of Formula (AIII) includes those of Formula (AIII-B):

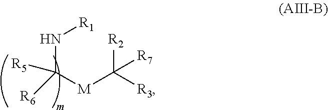

or its N-oxide, or a salt or isomer thereof in which all variables are as defined herein. For example, m is selected from 5, 6, 7, 8, and 9; R₄ is hydrogen, unsubstituted C₁₋₃ alkyl, or —(CH₂)ₙQ, in which Q is H, —NHC(S)N(R)₂, —NHC(O)N(R)₂, —N(R)C(O)R, —N(R)S(O)₂R, —N(R)R₈, —NHC(═NR₉)N(R)₂, —NHC(═CHR₉)N(R)₂, —OC(O)N(R)₂, —N(R)C(O)OR, heteroaryl or heterocycloalkyl; M and M′ are independently selected from —C(O)O—, —OC(O)—, —OC(O)-M″-C(O)O—, —C(O)N(R′)—, —P(O)(OR′)O—, —S—S—, an aryl group, and a heteroaryl group; and R₂ and R₃ are independently selected from the group consisting of H, C₁₋₁₄ alkyl, and C₂₋₁₄ alkenyl. For example, m is 5, 7, or 9. For example, Q is OH, —NHC(S)N(R)₂, or —NHC(O)N(R)₂. For example, Q is —N(R)C(O)R, or —N(R)S(O)₂R.

In certain embodiments, a subset of compounds of Formula (AIII) includes those of Formula (AIII-C):

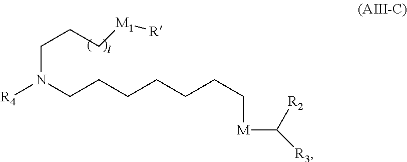

or its N-oxide, or a salt or isomer thereof, wherein 1 is selected from 1, 2, 3, 4, and 5; M₁ is a bond or M′; R₄ is hydrogen, unsubstituted C₁₋₃ alkyl, or —(CH₂)ₙQ, in which n is 2, 3, or 4, and Q is OH, —NHC(S)N(R)₂, —NHC(O)N(R)₂, —N(R)C(O)R, —N(R)S(O)₂R, —N(R)R₈, —NHC(═NR₉)N(R)₂, —NHC(═CHR₉)N(R)₂, —OC(O)N(R)₂, —N(R)C(O)OR, heteroaryl or heterocycloalkyl; M and M′ are independently selected from —C(O)O—, —OC(O)—, —OC(O)-M″-C(O)O—, —C(O)N(R′)—, —P(O)(OR′)O—, —S—S—, an aryl group, and a heteroaryl group; and R₂ and R₃ are independently selected from the group consisting of H, C₁₋₁₄ alkyl, and C₂₋₁₄ alkenyl.

In some embodiments, the compounds of Formula (AIII) are of Formula (AIII-D),

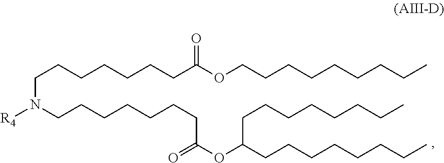

or their N-oxides, or salts or isomers thereof, wherein R₄ is as described herein.

In another embodiment, the compounds of Formula (AIII) are of Formula (AIII-E),

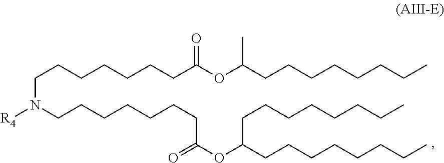

or their N-oxides, or salts or isomers thereof, wherein R₄ is as described herein.

In another embodiment, the compounds of Formula (AIII) are of Formula (AIII-F) or (AIII-G):

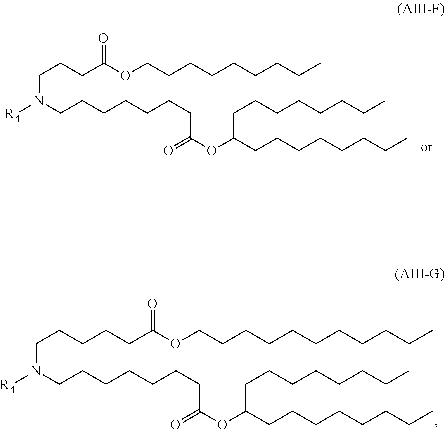

or their N-oxides, or salts or isomers thereof, wherein R₄ is as described herein.

In another embodiment, the compounds of Formula (AIII) are of Formula (AIII-H):

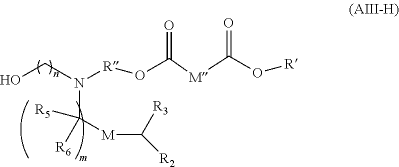

or their N-oxides, or salts or isomers thereof,
wherein M is —C(O)O— or —OC(O)—, M″ is C₁₋₆ alkyl or C₂₋₆ alkenyl, R₂ and R₃ are independently selected from the group consisting of C₅₋₁₄ alkyl and C₅₋₁₄ alkenyl, and n is selected from 2, 3, and 4.

In a further embodiment, the compounds of Formula (AIII) are of Formula (AIII-I):

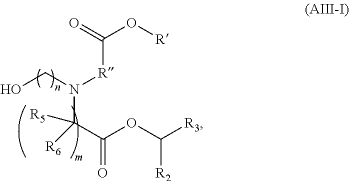

or their N-oxides, or salts or isomers thereof, wherein n is 2, 3, or 4; and m, R′, R″, and R₂ through R₆ are as described herein. For example, each of R₂ and R₃ may be independently selected from the group consisting of C₅₋₁₄ alkyl and C₅₋₁₄ alkenyl.

In some embodiments, an ionizable amino lipid of the disclosure comprises a compound having structure:

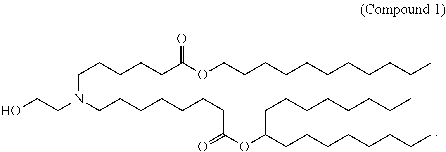 [SMILES: CCCCCCCCCCCOC(=O)CCCCCN(CCO)CCCCCCCC(=O)OC(CCCCCCCC)CCCCCCCC]

In some embodiments, an ionizable amino lipid of the disclosure comprises a compound having structure:

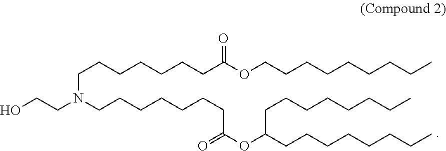 [SMILES: CCCCCCCCCOC(=O)CCCCCCCN(CCO)CCCCCCCC(=O)OC(CCCCCCCC)CCCCCCCC]

In a further embodiment, the compounds of Formula (AIII) are of Formula (AIII-J),

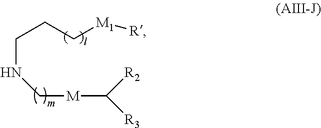

or salts or isomers thereof, wherein 1 is selected from 1, 2, 3, 4, and 5; m is selected from 5, 6, 7, 8, and 9; Mi is a bond or M′; M and M′ are independently selected from —C(O)O—, —OC(O)—, —OC(O)-M″-C(O)O—, —C(O)N(R′)—, —P(O)(OR′)O—, —S—S—, an aryl group, and a heteroaryl group; and R₂ and R₃ are independently selected from the group consisting of H, C₁₋₁₄ alkyl, and C₂₋₁₄ alkenyl. For example, M″ is C₁₋₆ alkyl (e.g., C₁₋₄ alkyl) or C₂₋₆ alkenyl (e.g. C₂₋₄ alkenyl). For example, R₂ and R₃ are independently selected from the group consisting of C₅₋₁₄ alkyl and C₅₋₁₄ alkenyl.

In some embodiments, the ionizable amino lipids are of Formula (AIII), or salts or isomers thereof, wherein:
R₁ is —R″M′R′;
R₂ and R₃ are each independently selected from C₁₋₁₄ alkyl and C₂₋₁₄ alkenyl;
R₄ is —(CH₂)ₙQ, wherein Q is OH and n is selected from 3, 4, and 5;
M and M′ are each independently —OC(O)—;
R₅, R₆, and R₇ are each H;
R′ is a linear C₁₋₁₂ alkyl, or C₁₋₁₂ alkyl substituted with C₆₋₉ alkyl;
R″ is C₃₋₁₄ alkyl;
m is selected from 5, 6, 7, 8, 9, 10, 11, 12, and 13.

In some embodiments, the ionizable amino lipids are of Formula (AIII), or salts or isomers thereof, wherein:
R₁ is R″M′R′;
R₂ and R₃ are each independently C₁₋₁₄ alkyl;
R₄ is —(CH₂)ₙQ, wherein Q is OH and n is 4;
M and M′ are each independently —OC(O)—;
R₅, R₆, and R₇ are each H;
R′ is C₁₋₁₂ alkyl substituted with C₆₋₉ alkyl;
R″ is C₃₋₁₄ alkyl; and
m is 6.

In some embodiments, an ionizable amino lipid of the disclosure comprises a compound having structure:

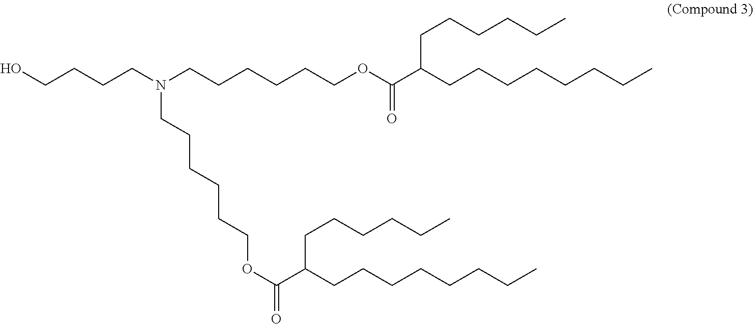 [SMILES: CCCCCCCCC(CCCCCC)C(=O)OCCCCCCN(CCCCO)CCCCCCOC(=O)C(CCCCCC)CCCCCCCC]

In some embodiments, the ionizable amino lipids are of Formula (AIII), or salts or isomers thereof, wherein:
R₁ is C₅₋₂₀ alkenyl;
R₂ and R₃ are each independently selected from C₁₋₁₄ alkyl and C₂₋₁₄ alkenyl;
R₄ is —(CH₂)ₙQ, wherein Q is OH and n is selected from 3, 4, and 5;
M and M′ are each independently C(O)O—;
R₅, R₆, and R₇ are each H;
R′ is a linear C₁₋₁₂ alkyl, or C₁₋₁₂ alkyl substituted with C₆₋₉ alkyl;
R″ is C₃₋₁₄ alkyl;
m is selected from 5, 6, 7, 8, 9, 10, 11, 12, and 13.

In some embodiments, the ionizable amino lipids are of Formula (AIII), or salts or isomers thereof, wherein:
R₁ is C₅₋₂₀ alkenyl;
R₂ and R₃ are each independently C₁₋₁₄ alkyl;
R₄ is —(CH₂)ₙQ, wherein Q is OH and n is 3;
M is —C(O)O—;
R₅, R₆, and R₇ are each H; and
m is 6.

In some embodiments, an ionizable amino lipid of the disclosure comprises a compound having structure:

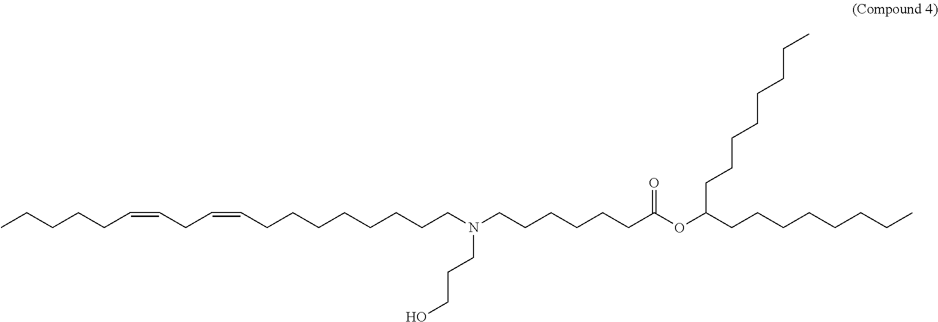 [SMILES: CCCCC/C=C\C/C=C\CCCCCCCCN(CCCO)CCCCCCC(=O)OC(CCCCCCCC)CCCCCCCC]

In some embodiments, the ionizable amino lipids are one or more of the compounds described in U.S. Application Nos. 62/220,091, 62/252,316, 62/253,433, 62/266,460, 62/333,557, 62/382,740, 62/393,940, 62/471,937, 62/471,949, 62/475,140, and 62/475,166, and PCT Application No. PCT/US2016/052352.

The central amine moiety of a lipid according to Formula (AIII), (AIII-A), (AIII-B), (AIII-C), (AIII-D), (AIII-E), (AIII-F), (AIII-G), (AIII-H), (AIII-I), or (AIII-J) may be protonated at a physiological pH. Thus, a lipid may have a positive or partial positive charge at physiological pH. Such amino lipids may be referred to as cationic lipids, ionizable lipids, cationic amino lipids, or ionizable amino lipids. Amino lipids may also be zwitterionic, i.e., neutral molecules having both a positive and a negative charge.

### Formula (AIV)

In some embodiments, the ionizable amino lipids may be one or more of compounds of formula (AIV),

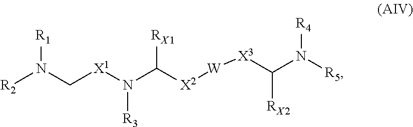

or salts or isomers thereof, wherein
W is

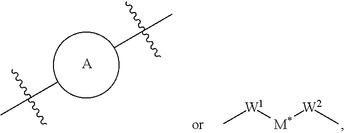

ring A is

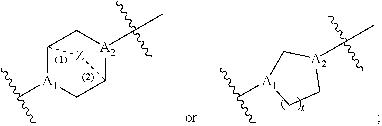

t is 1 or 2;
A₁ and A₂ are each independently selected from CH or N;
Z is CH₂ or absent wherein when Z is CH₂, the dashed lines (1) and (2) each represent a single bond; and when Z is absent, the dashed lines (1) and (2) are both absent;
R₁, R₂, R₃, R₄, and R₅ are independently selected from the group consisting of C₅₋₂₀ alkyl, C₅₋₂₀ alkenyl, —R″MR′, —R*YR″, —YR″, and —R*OR″;
R_{X1} and R_{X2} are each independently H or C₁₋₃ alkyl;
each M is independently selected from the group consisting of —C(O)O—, —OC(O)—, —OC(O)O—, —C(O)N(R′)—, —N(R′)C(O)—, —C(O)—, —C(S)—, —C(S)S—, —SC(S)—, —CH(OH)—, —P(O)(OR′)O—, —S(O)₂—, —C(O)S—, —SC(O)—, an aryl group, and a heteroaryl group;
M* is C₁-C₆ alkyl,
W¹ and W² are each independently selected from the group consisting of —O— and —N(R₆)—;
each R₆ is independently selected from the group consisting of H and C₁₋₅ alkyl;
X¹, X², and X³ are independently selected from the group consisting of a bond, —CH₂—, —(CH₂)₂—, —CHR—, —CHY—, —C(O)—, —C(O)O—, —OC(O)—, —(CH₂)ₙ—C(O)—, —C(O)—(CH₂)ₙ—, —(CH₂)ₙ—C(O)O—, —OC(O)—(CH₂)ₙ—, —(CH₂)ₙ—OC(O)—, —C(O)O—(CH₂)ₙ—, —CH(OH)—, —C(S)—, and —CH(SH)—;
each Y is independently a C₃₋₆ carbocycle;
each R* is independently selected from the group consisting of C₁₋₁₂ alkyl and C₂₋₁₂ alkenyl;
each R is independently selected from the group consisting of C₁₋₃ alkyl and a C₃₋₆ carbocycle;
each R′ is independently selected from the group consisting of C₁₋₁₂ alkyl, C₂₋₁₂ alkenyl, and H;
each R″ is independently selected from the group consisting of C₃₋₁₂ alkyl, C₃₋₁₂ alkenyl and —R*MR′; and
n is an integer from 1-6;
wherein when ring A is

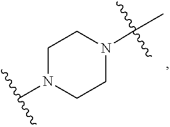 [SMILES: *N1CCN(*)CC1]

then
i) at least one of X¹, X², and X³ is not —CH₂—; and/or
ii) at least one of R₁, R₂, R₃, R₄, and R₅ is —R″MR′.

In some embodiments, the compound is of any of formulae (AIVa)-(AIVh):

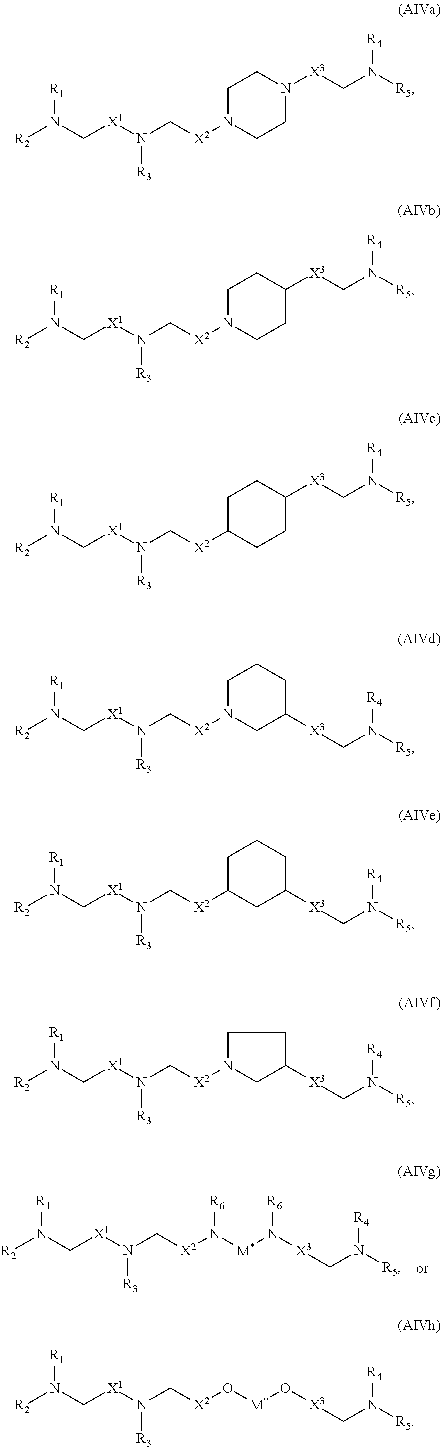

In some embodiments, the ionizable amino lipid is

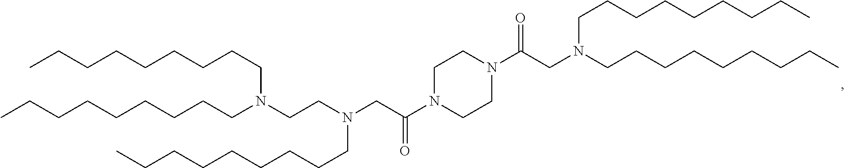 [SMILES: CCCCCCCCCN(CCCCCCCCC)CCN(CCCCCCCCC)CC(=O)N1CCN(C(=O)CN(CCCCCCCCC)CCCCCCCCC)CC1]

or a salt thereof.

The central amine moiety of a lipid according to Formula (AIV), (AIVa), (AIVb), (AIVc), (AIVd), (AIVe), (AIVf), (AIVg), or (AIVh) may be protonated at a physiological pH.

Thus, a lipid may have a positive or partial positive charge at physiological pH.

### Formula (AV)

In some embodiments, the lipid nanoparticle comprises a lipid having the structure:

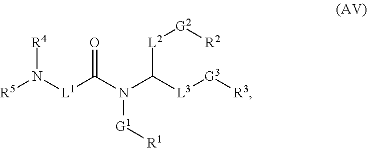

or a pharmaceutically acceptable salt, tautomer, or stereoisomer thereof, wherein
R¹ is optionally substituted C₁-C₂₄ alkyl or optionally substituted C₂-C₂₄ alkenyl;
R² and R³ are each independently optionally substituted C₁-C₃₆ alkyl;
R⁴ and R⁵ are each independently optionally substituted C₁-C₆ alkyl, or R⁴ and R⁵ join, along with the N to which they are attached, to form a heterocyclyl or heteroaryl;
L¹, L², and L³ are each independently optionally substituted C₁-C₁₈ alkylene;
G¹ is a direct bond, —(CH₂)ₙO(C═O)—, —(CH₂)n(C═O)O—, or —(C═O)—;
G² and G³ are each independently —(C═O)O— or —O(C═O)—; and n is an integer greater than 0.

### Formula (AVI)

In some embodiments, the lipid nanoparticle comprises a lipid having the structure:

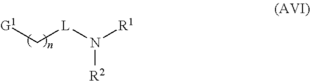

or a pharmaceutically acceptable salt, tautomer, or stereoisomer thereof, wherein:
G¹ is —N(R³)R⁴ or —OR⁵;
R¹ is optionally substituted branched, saturated or unsaturated C₁₂-C₃₆ alkyl;
R² is optionally substituted branched or unbranched, saturated or unsaturated C₁₂-C₃₆ alkyl when L is —C(═O)—; or R² is optionally substituted branched or unbranched, saturated or unsaturated C₄-C₃₆ alkyl when L is C₆-C₁₂ alkylene, C₆-C₁₂ alkenylene, or C₂-C₆ alkynylene;
R³ and R⁴ are each independently H, optionally substituted branched or unbranched, saturated or unsaturated C₁-C₆ alkyl; or R³ and R⁴ are each independently optionally substituted branched or unbranched, saturated or unsaturated C₁-C₆ alkyl when L is C₆-C₁₂ alkylene, C₆-C₁₂ alkenylene, or C₂-C₆ alkynylene; or R³ and R⁴, together with the nitrogen to which they are attached, join to form a heterocyclyl;
R⁵ is H or optionally substituted C₁-C₆ alkyl;
L is —C(═O)—, C₆-C₁₂ alkylene, C₆-C₁₂ alkenylene, or C₂-C₆ alkynylene; and
n is an integer from 1 to 12.

### Formula (AVII)

In some embodiments, the lipid nanoparticle comprises a lipid having the structure:

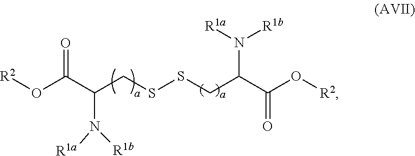

or a pharmaceutically acceptable salt thereof, wherein:
each R^{1a} is independently hydrogen, R^{1c}, or R^{1d};
each R^{1b} is independently R^{1c} or R^{1d};
each R^{1c} is independently —[CH₂]₂C(O)X¹R³;
each R^{1d} is independently —C(O)R⁴;
each R² is independently —[C(R^{2a})₂]_{c}R^{2b};
each R^{2a} is independently hydrogen or C₁-C₆ alkyl; R^{2b} is —N(L₁-B)₂; —(OCH₂CH₂)₆OH; or —(OCH₂CH₂)_{b}OCH₃;
each R³ and R⁴ is independently C₆-C₃₀ aliphatic;
each I.₃ is independently C₁-C₁₀ alkylene;
each B is independently hydrogen or an ionizable nitrogen-containing group;
each X¹ is independently a covalent bond or O;
each a is independently an integer of 1-10;
each b is independently an integer of 1-10; and
each c is independently an integer of 1-10.

### Formula (AVIII)

In some embodiments, the lipid nanoparticle comprises a lipid having the structure:

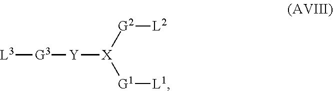

or a pharmaceutically acceptable salt, prodrug or stereoisomer thereof, wherein:
X is N, and Y is absent; or X is CR, and Y is NR;
L¹ is —O(C—O)R¹, —(C═O)ORI, —C(═O)R¹, —OR¹, —S(O)ₓR¹, —S—SR¹, —C(═O)SR¹, —SC(═O)R¹, —NR^{a}C(═O)R¹, —C(═O)NR^{b}R^{c}, —NR^{a}C(═O)NR^{b}R^{c}, —OC(═O)NR^{b}R^{c}, or —NR^{a}C(═O)OR¹;
L² is —O(C═O)R², —(C═O)OR², —C(═O)R², —OR², —S(O)ₓR², —S—SR², —C(═O)SR², —SC(═O)R², —NR^{d}C(═O)R², —C(═O)NR^{e}R^{f}, —NR^{d}C(═O)NR^{e}R^{f}, —OC(═O)NR^{e}R^{f}; —NR^{d}C(═O)OR² or a direct bond to R²;
L³ is —O(C═O)R³ or —(C═O)OR³;
G¹ and G² are each independently C₂-C₁₂ alkylene or C₂-C₁₂ alkenylene;
G³ is C₁-C₂₄ alkylene, C₂-C₂₄ alkenylene, C₁-C₂₄ heteroalkylene or C₂-C₂₄ heteroalkenylene when X is CR, and Y is NR; and G³ is C₁-C₂₄ heteroalkylene or C₂-C₂₄ heteroalkenylene when X is N, and Y is absent;
R^{a}, R^{b}, R^{d} and R^{e} are each independently H or C₁-C₁₂ alkyl or C₁-C₁₂ alkenyl;
R^{c} and R^{f} are each independently C₁-C₁₂ alkyl or C₂-C₁₂ alkenyl;
each R is independently H or C₁-C₁₂ alkyl;
R¹, R² and R³ are each independently C₁-C₂₄ alkyl or C₂-C₂₄ alkenyl; and x is 0, 1 or 2, and
wherein each alkyl, alkenyl, alkylene, alkenylene, heteroalkylene and heteroalkenylene is independently substituted or unsubstituted unless otherwise specified.

### Formula (AIX)

In some embodiments, the lipid nanoparticle comprises a lipid having the structure:

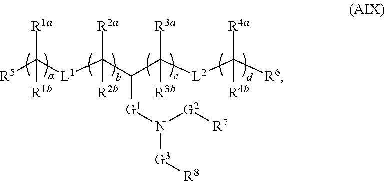

or a pharmaceutically acceptable salt, tautomer, prodrug or stereoisomer thereof, wherein:
L¹ and L² are each independently —O(C═O)—, —(C═O)O—, —C(═O)—, —O—, —S(O)x-, —S—S—, —C(═O)S—, —SC(═O)—, —NR^{a}C(═O)—, —C(═O)NR^{a}—, —NR^{a}C(═O)NR^{a}—, —OC(═O)NR^{a}—, —NR^{a}C(═O)O— or a direct bond;
G¹ is C₁-C₂ alkylene, —(C═O)—, —O(C═O)—, —SC(═O)—, —NR^{a}C(═O)— or a direct bond;
G² is —C(O)—, —(CO)O—, —C(═O)S—, —C(═O)NR^{a}— or a direct bond;
G³ is C₁-C₆ alkylene;
R^{a} is H or C₁-C₁₂ alkyl;
R^{1a} and R^{1b} are, at each occurrence, independently either: (a) H or C₁-C₁₂ alkyl; or (b) R^{1a} is H or C₁-C₁₂ alkyl, and R^{1b} together with the carbon atom to which it is bound is taken together with an adjacent R^{1b} and the carbon atom to which it is bound to form a carbon-carbon double bond;
R^{2a} and R^{2b} are, at each occurrence, independently either: (a) H or C₁-C₁₂ alkyl; or (b) R^{2a} is H or C₁-C₁₂ alkyl, and R^{2b} together with the carbon atom to which it is bound is taken together with an adjacent R^{2b} and the carbon atom to which it is bound to form a carbon-carbon double bond;
R^{3a} and R^{3b} are, at each occurrence, independently either (a): H or C₁-C₁₂ alkyl; or (b) R^{3a} is H or C₁-C₁₂ alkyl, and R^{3b} together with the carbon atom to which it is bound is taken together with an adjacent R and the carbon atom to which it is bound to form a carbon-carbon double bond;
R^{4a} and R^{4b} are, at each occurrence, independently either: (a) H or C₁-C₁₂ alkyl; or (b) R^{4a} is H or C₁-C₁₂ alkyl, and R^{4b} together with the carbon atom to which it is bound is taken together with an adjacent R^{4b} and the carbon atom to which it is bound to form a carbon-carbon double bond;
R⁵ and R⁶ are each independently H or methyl;
R⁷ is H or C₁-C₂₀ alkyl;
R⁸ is OH, —N(R⁹)(C═O)R¹⁰, —(C═O)NR⁹R¹⁰, —NR⁹R¹⁰, —(C═O)OR″¹ or —O(C═O)R″, provided that G³ is C₄-C₆ alkylene when R⁸ is —NR⁹R¹⁰,
R⁹ and R¹⁰ are each independently H or C₁-C₁₂ alkyl;
R″ is aralkyl;
a, b, c and d are each independently an integer from 1 to 24; and x is 0, 1 or 2,
wherein each alkyl, alkylene and aralkyl is optionally substituted.

### Formula (AX)

In some embodiments, the lipid nanoparticle comprises a lipid having the structure:

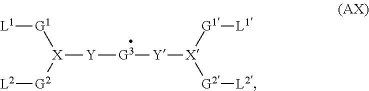

or a pharmaceutically acceptable salt, prodrug or stereoisomer thereof, wherein:
X and X′ are each independently N or CR;
Y and Y are each independently absent, —O(C═O)—, —(C═O)O— or NR, provided that:
   a) Y is absent when X is N;
   b) Y is absent when X′ is N;
   c) Y is —O(C═O)—, —(C═O)O— or NR when X is CR; and
   d) Y is —O(C═O)—, —(C═O)O— or NR when X′ is CR,
L¹ and L^{1′} are each independently —O(C═O)R′, —(C═O)OR′, —C(═O)R′, —OR¹, —S(O)_{z}R′, —S—SR¹, —C(═O)SR′, —SC(═O)R′, —NR^{a}C(═O)R′, —C(═O)NR^{b}R^{c}, —NR^{a}C(═O)NR^{b}R^{c}, —OC(═O)NR^{b}R^{c} or —NR^{a}C(═O)OR′;
L² and L^{2′} are each independently —O(C═O)R², —(C═O)OR², —C(═O)R², —OR², —S(O)_{z}R², —S—SR², —C(═O)SR², —SC(═O)R², —NR^{d}C(═O)R², —C(═O)NR^{e}R^{f}, —NR^{d}C(═O)NR^{e}R^{f}, —OC(═O)NR^{e}R^{f}; —NR^{d}C(═O)OR² or a direct bond to R²;
G¹, G^{1′}, G² and G^{2′} are each independently C₂-C₁₂ alkylene or C₂-C₁₂ alkenylene;
G is C₂-C₂₄ heteroalkylene or C₂-C₂₄ heteroalkenylene;
R^{a}, R^{b}, R^{d} and R^{e} are, at each occurrence, independently H, C₁-C₁₂ alkyl or C₂-C₁₂ alkenyl;
R^{c} and R^{f} are, at each occurrence, independently C₁-C₁₂ alkyl or C₂-C₁₂ alkenyl;
R is, at each occurrence, independently H or C₁-C₁₂ alkyl;
R¹ and R² are, at each occurrence, independently branched C₆-C₂₄ alkyl or branched C₆-C₂₄ alkenyl;
z is 0, 1 or 2, and wherein each alkyl, alkenyl, alkylene, alkenylene, heteroalkylene and heteroalkenylene is independently substituted or unsubstituted unless otherwise specified.

### Formula (AXI)

In some embodiments, the lipid nanoparticle comprises a lipid having the structure:

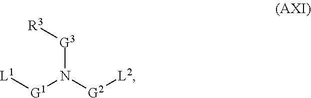

or a pharmaceutically acceptable salt, prodrug or stereoisomer thereof, wherein:
L¹ is —O(C═O)R¹, —(C═O)ORI, —C(═O)R¹, —OR′, —S(O)ₓR¹, —S—SR¹, —C(═O)SR¹, —SC(═O)R¹, —NR^{a}C(═O)R¹, —C(═O)NR^{b}R^{c}, —NR^{a}C(═O)NR^{b}R^{c}, —OC(═O)NR^{b}R^{c} or —NR^{a}C(═O)OR¹;
L² is —O(C═O)R², —(C═O)OR², —C(═O)R², —OR², —S(O)ₓR², —S—SR², —C(═O)SR², —SC(═O)R², —NR^{d}C(═O)R², —C(═O)NR^{e}R^{f}, —NR^{d}C(═O)NR^{e}R^{f}, —OC(═O)NR^{e}R^{f}; —NR^{d}C(═O)OR² or a direct bond to R²;
G¹ and G² are each independently C₂-C₁₂ alkylene or C₂-C₁₂ alkenylene;
G³ is C₁-C₂₄ alkylene, C₂-C₂₄ alkenylene, C₃-C₈ cycloalkylene or C₃-C₈ cycloalkenylene;
R^{a}, R^{b}, R^{d} and R^{e} are each independently H or C₁-C₁₂ alkyl or C₁-C₁₂ alkenyl;
R^{c} and R^{f} are each independently C₁-C₁₂ alkyl or C₂-C₁₂ alkenyl;
R¹ and R² are each independently branched C₆-C₂₄ alkyl or branched C₆-C₂₄ alkenyl;
R³ is —N(R⁴)R⁵;
R⁴ is C₁-C₁₂ alkyl;
R⁵ is substituted C₁-C₁₂ alkyl; and
x is 0, 1 or 2, and
wherein each alkyl, alkenyl, alkylene, alkenylene, cycloalkylene, cycloalkenylene, aryl and aralkyl is independently substituted or unsubstituted unless otherwise specified.

In some embodiments, the lipid nanoparticle comprises a lipid having the structure:

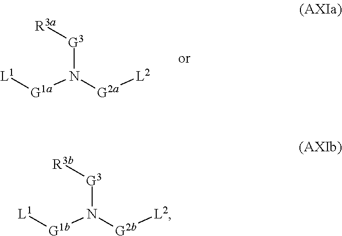

or a pharmaceutically acceptable salt, prodrug or stereoisomer thereof, wherein:
L¹ is —O(C═O)R¹, —(C═O)OR¹, —C(═O)R¹, —OR¹, —S(O)ₓR¹, —S—SR¹, —C(═O)SR¹, —SC(═O)R¹, —NR^{a}C(═O)R¹, —C(═O)NR^{b}R^{c}, —NR^{a}C(═O)NR^{b}R^{c}, —OC(═O)NR^{b}R^{c} or —NR^{a}C(═O)OR¹;
L² is —O(C═O)R², —(C═O)OR², —C(═O)R², —OR², —S(O)ₓR², —S—SR², —C(═O)SR², —SC(═O)R², —NR^{d}C(═O)R², —C(═O)NR^{e}R^{f}, —NR^{d}C(═O)NR^{e}R^{f}, —OC(═O)NR^{e}R^{f}; —NR^{d}C(═O)OR² or a direct bond to R²;
G^{1a} and G^{2b} are each independently C₂-C₁₂ alkylene or C₂-C₁₂ alkenylene;
G^{1b} and G^{2b} are each independently C₁-C₁₂ alkylene or C₂-C₁₂ alkenylene;
G³ is C₁-C₂₄ alkylene, C₂-C₂₄ alkenylene, C₃-C₈ cycloalkylene or C₃-C₈ cycloalkenylene;
R^{a}, R^{b}, R^{d} and R^{e} are each independently H or C₁-C₁₂ alkyl or C₂-C₁₂ alkenyl;
R^{c} and R^{f} are each independently C₁-C₁₂ alkyl or C₂-C₁₂ alkenyl;
R¹ and R² are each independently branched C₆-C₂₄ alkyl or branched C₆-C₂₄ alkenyl;
R^{3a} is —C(═O)N(R^{4a})R^{5a} or —C(═O)OR⁶;
R^{3b} is —NR^{4b}C(═O)R^{5b};
R^{4a} is C₁-C₁₂ alkyl;
R^{4b} is H, C₁-C₁₂ alkyl or C₂-C₁₂ alkenyl;
R^{5a} is H, C₁-C₈ alkyl or C₂-C₈ alkenyl;
R^{5b} is C₂-C₁₂ alkyl or C₂-C₁₂ alkenyl when R^{4b} is H; or R^{5b} is C₁-C₁₂ alkyl or C₂-C₁₂ alkenyl when R^{4b} is C₁-C₁₂ alkyl or C₂-C₁₂ alkenyl;
R⁶ is H, aryl or aralkyl; and
x is 0, 1 or 2, and
wherein each alkyl, alkenyl, alkylene, alkenylene, cycloalkylene, cycloalkenylene, aryl and aralkyl is independently substituted or unsubstituted.

### Formula (AXII)

In some embodiments, the lipid nanoparticle comprises a lipid having the structure:

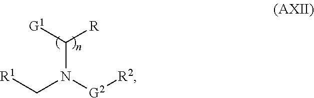

or a pharmaceutically acceptable salt, prodrug or stereoisomer thereof, wherein:
G¹ is —OH, —R³R⁴, —(C═O)R⁵ or —R³(C═O)R⁵;
G² is —CH₂— or —(C═O)—;
R is, at each occurrence, independently H or OH;
R¹ and R² are each independently optionally substituted branched, saturated or unsaturated C₁₂-C₃₆ alkyl;
R³ and R⁴ are each independently H or optionally substituted straight or branched, saturated or unsaturated C₁-C₆ alkyl;
R⁵ is optionally substituted straight or branched, saturated or unsaturated C₁-C₆ alkyl; and
n is an integer from 2 to 6.

### Formula (AXII)

In some embodiments, the lipid nanoparticle comprises a lipid having the structure:

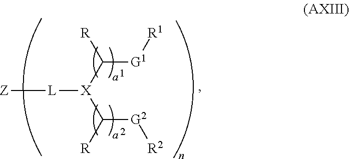

or a pharmaceutically acceptable salt, prodrug or stereoisomer thereof, wherein:
one of G¹ or G² is, at each occurrence, —O(C═O)—, —(C═O)O—, —C(═O)—, —O—, —S(O), —S—S—, —C(═O)S—, SC(═O)—, —N(R^{a})C(═O)—, —C(═O)N(R^{a})—, —N(R^{a})C(═O)N(R^{a})—, —OC(═O)N(R^{a}) or —N(R^{a})C(═O)O—, and the other of G¹ or G² is, at each occurrence, —O(C═O)—, —(C═O)O—, —C(═O)—, —O—, —S(O), —S—S—, —C(═O)S—, —SC(═O)—, —N(R^{a})C(═O)—, —C(═O)N(R^{a})—, —N(R^{a})C(═O)N(R^{a})—, —OC(═O)N(R^{a})— or —N(R^{a})C(═O)O— or a direct bond;
L is, at each occurrence, —O(C═O)—, wherein ~ represents a covalent bond to X;
X is CR^{a};
Z is alkyl, cycloalkyl or a monovalent moiety comprising at least one polar functional group when n is 1; or Z is alkylene, cycloalkylene or a polyvalent moiety comprising at least one polar functional group when n is greater than 1;
R^{a} is, at each occurrence, independently H, C₁-C₁₂ alkyl, C₁-C₁₂ hydroxylalkyl, C₁-C₁₂ aminoalkyl, C₁-C₁₂ alkylaminylalkyl, C₁-C₁₂ alkoxyalkyl, C₁-C₁₂ alkoxycarbonyl, C₁-C₁₂ alkylcarbonyloxy, C₁-C₁₂ alkylcarbonyloxyalkyl or C₁-C₁₂ alkylcarbonyl;
R is, at each occurrence, independently either: (a) H or C₁-C₁₂ alkyl; or (b) R together with the carbon atom to which it is bound is taken together with an adjacent R and the carbon atom to which it is bound to form a carbon-carbon double bond;
R¹ and R² have, at each occurrence, the following structure, respectively:

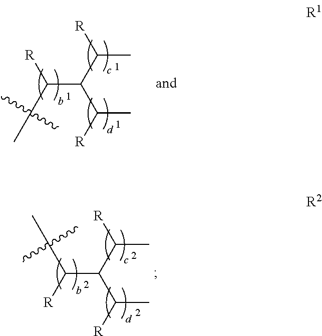

a¹ and a² are, at each occurrence, independently an integer from 3 to 12; b¹ and b² are, at each occurrence, independently 0 or 1;
c¹ and c² are, at each occurrence, independently an integer from 5 to 10; d¹ and d² are, at each occurrence, independently an integer from 5 to 10; y is, at each occurrence, independently an integer from 0 to 2; and n is an integer from 1 to 6,
wherein each alkyl, alkylene, hydroxylalkyl, aminoalkyl, alkylaminylalkyl, alkoxyalkyl, alkoxycarbonyl, alkylcarbonyloxy, alkylcarbonyloxyalkyl and alkylcarbonyl is optionally substituted with one or more substituent.

### Formula (AXIV)

In some embodiments, the lipid nanoparticle comprises a lipid having the structure:

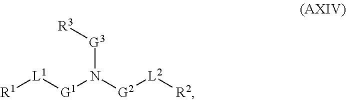

or a pharmaceutically acceptable salt, prodrug or stereoisomer thereof, wherein:
one of L¹ or L² is —O(C═O)—, —(C═O)O—, —C(═O)—, —O—, —S(O)ₓ—, —S—S—, —C(═O)S—, —SC(═O)—, —R^{a}C(═O)—, —C(═O)R^{a}—, —R^{a}C(═O)R^{a}—, —OC(═O)R^{a}— or — R^{a}C(═O)O—, and the other of L¹ or L² is —O(C═O)—, —(C═O)O—, —C(═O)—, —O—, —S(O)ₓ—, —S—S—, —C(═O)S—, —SC(═O)—, —R^{a}C(═O)—, —C(═O)R^{a}—, R^{a}C(═O)R^{a}—, —OC(═O)R^{a}— or —NR^{a}C(═O)O— or a direct bond;
G¹ and G² are each independently unsubstituted C₁-C₁₂ alkylene or C₁-C₁₂ alkenylene;
G³ is C₁-C₂₄ alkylene, C₁-C₂₄ alkenylene, C₃-C₈ cycloalkylene, C₃-C₈ cycloalkenylene;
R^{a} is H or C₁-C₁₂ alkyl;
R¹ and R² are each independently C₆-C₂₄ alkyl or C₆-C₂₄ alkenyl;
R³ is H, OR⁵, CN, —C(═O)OR⁴, —OC(═O)R⁴ or —R^{a}C(═O)R⁴;
R⁴ is C₁-C₁₂ alkyl;
R⁵ is H or C₁-C₆ alkyl; and
x is 0, 1 or 2.

### Formula (AXV)

In some embodiments, the lipid nanoparticle comprises a lipid having the structure:

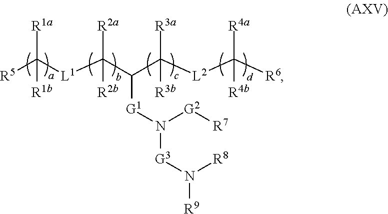

or a pharmaceutically acceptable salt, tautomer, prodrug or stereoisomer thereof, wherein:
L¹ and L² are each independently —O(C═O)—, —(C═O)O—, —C(═O)—, —O—, —S(O)ₓ—, —S—S—, —C(═O)S—, —SC(═O)—, —R^{a}C(═O)—, —C(═O)R^{a}—, —R^{a}C(═O)R^{a}—, —OC(═O)R^{a}—, —R^{a}C(═O)O— or a direct bond;
G¹ is C₁-C₂ alkylene, —(C═O)—, —O(C═O)—, —SC(═O)—, —R^{a}C(═O)— or a direct bond:
G² is —C(═O)—, —(C═O)O—, —C(═O)S—, —C(═O)NR^{a}— or a direct bond;
G³ is C₁-C₆ alkylene;
R^{a} is H or C₁-C₁₂ alkyl;
R^{1a} and R^{1b} are, at each occurrence, independently either: (a) H or C₁-C₁₂ alkyl; or (b) R^{1a} is H or C₁-C₁₂ alkyl, and R^{1b} together with the carbon atom to which it is bound is taken together with an adjacent R^{1b} and the carbon atom to which it is bound to form a carbon-carbon double bond;
R^{2a} and R^{2b} are, at each occurrence, independently either: (a) H or C₁-C₁₂ alkyl; or (b) R^{2a} is H or C₁-C₁₂ alkyl, and R^{2b} together with the carbon atom to which it is bound is taken together with an adjacent R^{2b} and the carbon atom to which it is bound to form a carbon-carbon double bond;
R^{3a} and R^{3b} are, at each occurrence, independently either (a): H or C₁-C₁₂ alkyl; or (b) R^{3a} is H or C₁-C₁₂ alkyl, and R^{3b} together with the carbon atom to which it is bound is taken together with an adjacent R and the carbon atom to which it is bound to form a carbon-carbon double bond;
R^{4a} and R^{4b} are, at each occurrence, independently either: (a) H or C₁-C₁₂ alkyl; or (b) R^{4a} is H or C₁-C₁₂ alkyl, and R^{4b} together with the carbon atom to which it is bound is taken together with an adjacent R^{4b} and the carbon atom to which it is bound to form a carbon-carbon double bond;
R⁵ and R⁶ are each independently H or methyl;
R⁷ is C₄-C₂₀ alkyl;
R⁸ and R⁹ are each independently C₁-C₁₂ alkyl; or R⁸ and R⁹, together with the nitrogen atom to which they are attached, form a 5, 6 or 7-membered heterocyclic ring;
a, b, c and d are each independently an integer from 1 to 24; and x is 0, 1 or 2.

### Formula (AXVI)

In some embodiments, the lipid nanoparticle comprises a lipid having the structure:

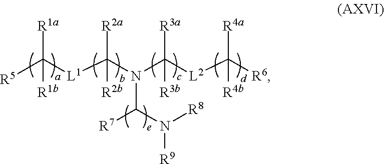

or a pharmaceutically acceptable salt, tautomer, prodrug or stereoisomer thereof, wherein:
L¹ and L² are each independently —O(C═O)—, —(C═O)O— or a carbon-carbon double bond;
R^{1a} and R^{1b} are, at each occurrence, independently either (a) H or C₁-C₁₂ alkyl, or (b) R^{1a} is H or C₁-C₁₂ alkyl, and R^{1b} together with the carbon atom to which it is bound is taken together with an adjacent R^{1b} and the carbon atom to which it is bound to form a carbon-carbon double bond;
R^{2a} and R^{2b} are, at each occurrence, independently either (a) H or C₁-C₁₂ alkyl, or (b) R^{2a} is H or C₁-C₁₂ alkyl, and R^{2b} together with the carbon atom to which it is bound is taken together with an adjacent R^{2b} and the carbon atom to which it is bound to form a carbon-carbon double bond;
R^{3a} and R^{3b} are, at each occurrence, independently either (a) H or C₁-C₁₂ alkyl, or (b) R^{3a} is H or C₁-C₁₂ alkyl, and R^{3b} together with the carbon atom to which it is bound is taken together with an adjacent R^{3b} and the carbon atom to which it is bound to form a carbon-carbon double bond;
R^{4a} and R^{4b} are, at each occurrence, independently either (a) H or C₁-C₁₂ alkyl, or (b) R^{4a} is H or C₁-C₁₂ alkyl, and R^{4b} together with the carbon atom to which it is bound is taken together with an adjacent R^{4b} and the carbon atom to which it is bound to form a carbon-carbon double bond;
R⁵ and R⁶ are each independently methyl or cycloalkyl;
R⁷ is, at each occurrence, independently H or C₁-C₁₂ alkyl; R⁸ and R⁹ are each independently unsubstituted C₁-C₁₂ alkyl; or R⁸ and R⁹, together with the nitrogen atom to which they are attached, form a 5, 6 or 7-membered heterocyclic ring comprising one nitrogen atom;
a and d are each independently an integer from 0 to 24; b and c are each independently an integer from 1 to 24; and e is 1 or 2,
provided that:
at least one of R¹, R^{2a}, R^{3a} or R^{4a} is C₁-C₁₂ alkyl, or at least one of L¹ or L² is —O(C═O)— or —(C═O)O—; and
R^{1a} and R^{1b} are not isopropyl when a is 6 or n-butyl when a is 8.

### Formula (AXVII)

In some embodiments, the lipid nanoparticle comprises a lipid having the structure:

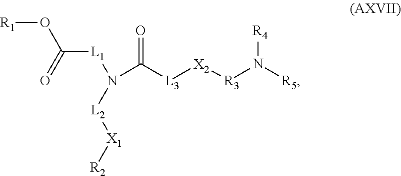

or a pharmaceutically acceptable salt thereof, wherein
R₁ and R₂ are the same or different, each a linear or branched alkyl with 1-9 carbons, or as alkenyl or alkynyl with 2 to 11 carbon atoms,
L₁ and L₂ are the same or different, each a linear alkyl having 5 to 18 carbon atoms, or form a heterocycle with N,
X₁ is a bond, or is —CG-G- whereby L2-CO—O—R₂ is formed,
X₂ is S or O,
L₃ is a bond or a lower alkyl, or form a heterocycle with N,
R₃ is a lower alkyl, and
R₄ and R₅ are the same or different, each a lower alkyl.

### Compounds (A1)-(A11)

In some embodiments, the lipid nanoparticle comprises an ionizable lipid having the structure:

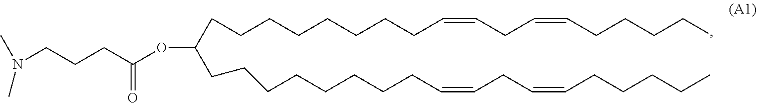

or a pharmaceutically acceptable salt thereof.

In some embodiments, the lipid nanoparticle comprises a lipid having the structure:

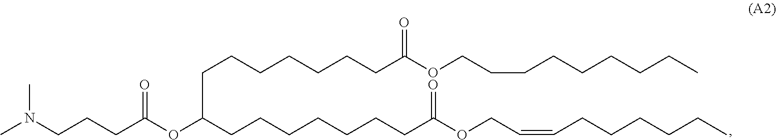 [SMILES: CCCCCC/C=C\COC(=O)CCCCCCCC(CCCCCCCC(=O)OCCCCCCCCC)OC(=O)CCCN(C)C]

or a pharmaceutically acceptable salt thereof.

In some embodiments, the lipid nanoparticle comprises a lipid having the structure:

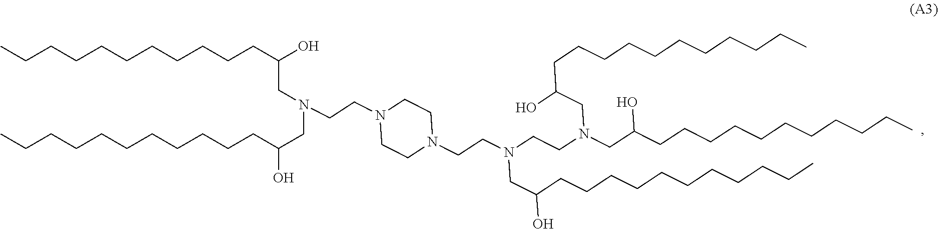 [SMILES: CCCCCCCCCCCC(O)CN(CCN1CCN(CCN(CC(O)CCCCCCCCCCC)CC(O)CCCCCCCCCCC)CC1)CCN(CC(O)CCCCCCCCCCC)CC(O)CCCCCCCCCCC]

pharmaceutically acceptable salt thereof.

In some embodiments, the lipid nanoparticle comprises a lipid having the structure:

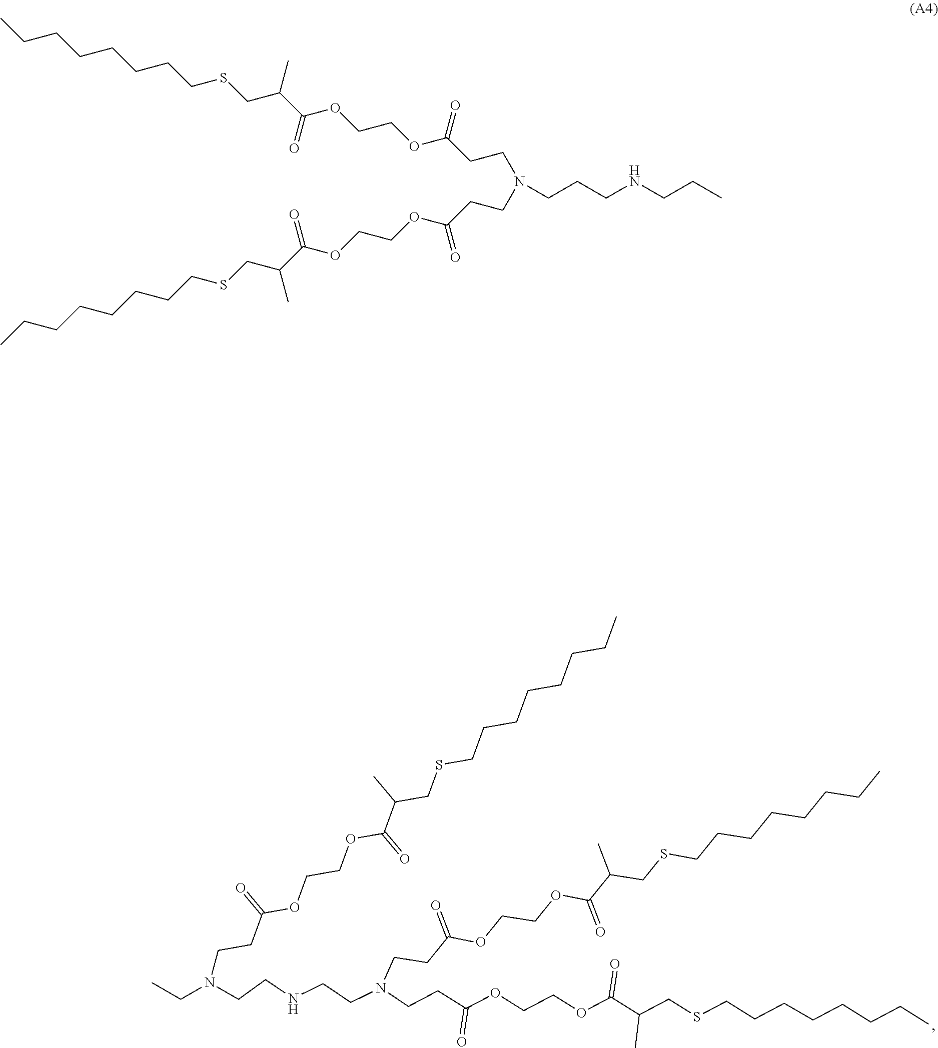

or a pharmaceutically acceptable salt thereof.

In some embodiments, the lipid nanoparticle comprises a lipid having the structure:

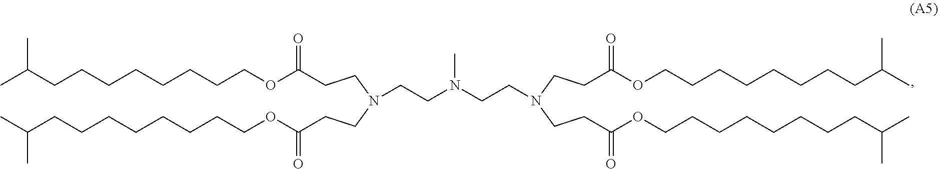 [SMILES: CC(C)CCCCCCCCOC(=O)CCN(CCC(=O)OCCCCCCCCC(C)C)CCN(C)CCN(CCC(=O)OCCCCCCCCC(C)C)CCC(=O)OCCCCCCCCC(C)C]

pharmaceutically acceptable salt thereof.

In some embodiments, the lipid nanoparticle comprises a lipid having the structure:

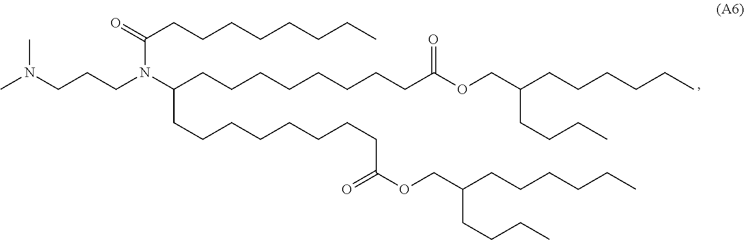 [SMILES: CCCCCCCCC(=O)N(CCCN(C)C)C(CCCCCCCCC(=O)OCC(CCCC)CCCCCC)CCCCCCCCC(=O)OCC(CCCC)CCCCCC]

or a pharmaceutically acceptable salt thereof.

In some embodiments, the lipid nanoparticle comprises a lipid having the structure:

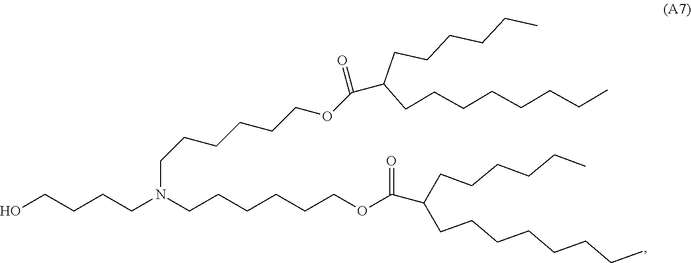 [SMILES: CCCCCCCCC(CCCCCC)C(=O)OCCCCCCN(CCCCO)CCCCCCOC(=O)C(CCCCCC)CCCCCCCC]

or a pharmaceutically acceptable salt thereof.

In some embodiments, the lipid nanoparticle comprises a lipid having the structure:

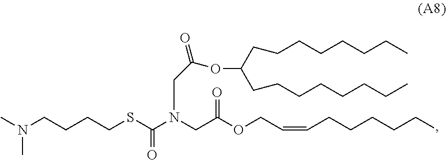

or a pharmaceutically acceptable salt thereof.

In some embodiments the lipid nanoparticle comprises a lipid having the structure:

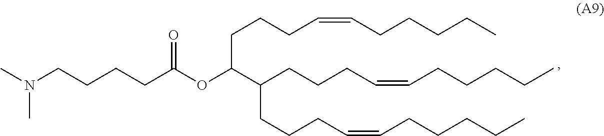 [SMILES: CCCCC/C=C\CCCC(CCC/C=C\CCCCC)C(CCC/C=C\CCCCC)OC(=O)CCCCN(C)C]

or a pharmaceutically acceptable salt thereof.

In some embodiments, the lipid nanoparticle comprises a lipid having the structure:

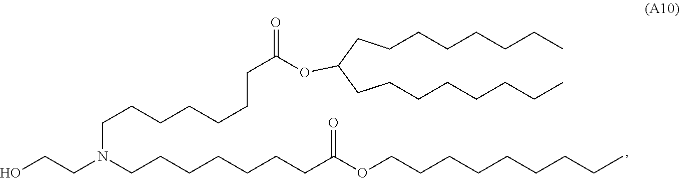 [SMILES: CCCCCCCCCOC(=O)CCCCCCCN(CCO)CCCCCCCC(=O)OC(CCCCCCCC)CCCCCCCC]

or a pharmaceutically acceptable salt thereof.

In some embodiments, the lipid nanoparticle comprises a lipid having the structure:

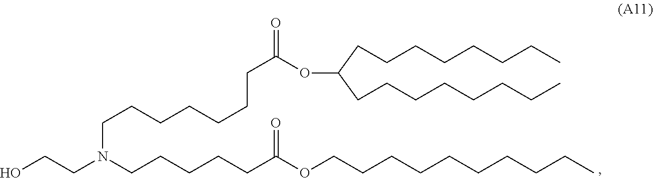 [SMILES: CCCCCCCCCCOC(=O)CCCCCN(CCO)CCCCCCCC(=O)OC(CCCCCCCC)CCCCCCCC]

or a pharmaceutically acceptable salt thereof.

### Non-Cationic Lipids

In certain embodiments, the lipid nanoparticles described herein comprise one or more non-cationic lipids. Non-cationic lipids may be phospholipids.

In some embodiments, the lipid nanoparticle comprises 5-25 mol % non-cationic lipid. For example, the lipid nanoparticle may comprise 5-20 mol %, 5-15 mol %, 5-10 mol %, 10-25 mol %, 10-20 mol %, 10-25 mol %, 15-25 mol %, 15-20 mol %, or 20-25 mol % non-cationic lipid. In some embodiments, the lipid nanoparticle comprises 5 mol %, 10 mol %, 15 mol %, 20 mol %, or 25 mol % non-cationic lipid.

In some embodiments, a non-cationic lipid of the disclosure comprises 1,2-distearoyl-sn-glycero-3-phosphocholine (DSPC), 1,2-dioleoyl-sn-glycero-3-phosphoethanolamine (DOPE), 1,2-dilinoleoyl-sn-glycero-3-phosphocholine (DLPC), 1,2-dimyristoyl-sn-gly cero-phosphocholine (DMPC), 1,2-dioleoyl-sn-glycero-3-phosphocholine (DOPC), 1,2-dipalmitoyl-sn-glycero-3-phosphocholine (DPPC), 1,2-diundecanoyl-sn-glycero-phosphocholine (DUPC), 1-palmitoyl-2-oleoyl-sn-glycero-3-phosphocholine (POPC), 1,2-di-O-octadecenyl-sn-glycero-3-phosphocholine (18:0 Diether PC), 1-oleoyl-2 cholesterylhemisuccinoyl-sn-glycero-3-phosphocholine (OChemsPC), 1-hexadecyl-sn-glycero-3-phosphocholine (C16 Lyso PC), 1,2-dilinolenoyl-sn-glycero-3-phosphocholine, 1,2-diarachidonoyl-sn-glycero-3-phosphocholine, 1,2-didocosahexaenoyl-sn-glycero-3-phosphocholine, 1,2-diphytanoyl-sn-glycero-3-phosphoethanolamine (ME 16.0 PE), 1,2-distearoyl-sn-glycero-3-phosphoethanolamine, 1,2-dilinoleoyl-sn-glycero-3-phosphoethanolamine, 1,2-dilinolenoyl-sn-glycero-3-phosphoethanolamine, 1,2-diarachidonoyl-sn-glycero-3-phosphoethanolamine, 1,2-didocosahexaenoyl-sn-glycero-3-phosphoethanolamine, 1,2-dioleoyl-sn-glycero-3-phospho-rac-(1-glycerol) sodium salt (DOPG), sphingomyelin, or mixtures thereof.

In some embodiments, the lipid nanoparticle comprises 5-15 mol %, 5-10 mol %, or 10-15 mol % DSPC. For example, the lipid nanoparticle may comprise 5, 6, 7, 8, 9, 10, 11, 12, 13, 14, or 15 mol % DSPC.

In certain embodiments, the lipid composition of the lipid nanoparticle composition disclosed herein can comprise one or more phospholipids, for example, one or more saturated or (poly)unsaturated phospholipids or a combination thereof. In general, phospholipids comprise a phospholipid moiety and one or more fatty acid moieties.

A phospholipid moiety can be selected, for example, from the non-limiting group consisting of phosphatidyl choline, phosphatidyl ethanolamine, phosphatidyl glycerol, phosphatidyl serine, phosphatidic acid, 2-lysophosphatidyl choline, and a sphingomyelin.

A fatty acid moiety can be selected, for example, from the non-limiting group consisting of lauric acid, myristic acid, myristoleic acid, palmitic acid, palmitoleic acid, stearic acid, oleic acid, linoleic acid, alpha-linolenic acid, erucic acid, phytanoic acid, arachidic acid, arachidonic acid, eicosapentaenoic acid, behenic acid, docosapentaenoic acid, and docosahexaenoic acid.

Particular phospholipids can facilitate fusion to a membrane. For example, a cationic phospholipid can interact with one or more negatively charged phospholipids of a membrane (e.g., a cellular or intracellular membrane). Fusion of a phospholipid to a membrane can allow one or more elements (e.g., a therapeutic agent) of a lipid-containing composition (e.g., LNPs) to pass through the membrane permitting, e.g., delivery of the one or more elements to a target tissue.

Non-natural phospholipid species including natural species with modifications and substitutions including branching, oxidation, cyclization, and alkynes are also contemplated. For example, a phospholipid can be functionalized with or cross-linked to one or more alkynes (e.g., an alkenyl group in which one or more double bonds is replaced with a triple bond). Under appropriate reaction conditions, an alkyne group can undergo a copper-catalyzed cycloaddition upon exposure to an azide. Such reactions can be useful in functionalizing a lipid bilayer of a nanoparticle composition to facilitate membrane permeation or cellular recognition or in conjugating a nanoparticle composition to a useful component such as a targeting or imaging moiety (e.g., a dye).

Phospholipids include, but are not limited to, glycerophospholipids such as phosphatidylcholines, phosphatidylethanolamines, phosphatidylserines, phosphatidylinositols, phosphatidy glycerols, and phosphatidic acids. Phospholipids also include phosphosphingolipid, such as sphingomyelin.

In some embodiments, a phospholipid comprises 1,2-distearoyl-sn-glycero-3-phosphocholine (DSPC), 1,2-Distearoyl-sn-glycero-3-phosphoethanolamine (DSPE), 1,2-dioleoyl-sn-glycero-3-phosphoethanolamine (DOPE), 1,2-dilinoleoyl-sn-glycero-3-phosphocholine (DLPC), 1,2-dimyristoyl-sn-gly cero-phosphocholine (DMPC), 1,2-dioleoyl-sn-glycero-3-phosphocholine (DOPC), 1,2-dipalmitoyl-sn-glycero-3-phosphocholine (DPPC), 1,2-diundecanoyl-sn-glycero-phosphocholine (DUPC), 1-palmitoyl-2-oleoyl-sn-glycero-3-phosphocholine (POPC), 1,2-di-O-octadecenyl-sn-glycero-3-phosphocholine (18:0 Diether PC), 1-oleoyl-2 cholesterylhemisuccinoyl-sn-glycero-3-phosphocholine (OChemsPC), 1-hexadecyl-sn-glycero-3-phosphocholine (C16 Lyso PC), 1,2-dilinolenoyl-sn-glycero-3-phosphocholine, 1,2-diarachidonoyl-sn-glycero-3-phosphocholine, 1,2-didocosahexaenoyl-sn-glycero-3-phosphocholine, 1,2-diphytanoyl-sn-glycero-3-phosphoethanolamine (ME 16.0 PE), 1,2-distearoyl-sn-glycero-3-phosphoethanolamine, 1,2-dilinoleoyl-sn-glycero-3-phosphoethanolamine, 1,2-dilinolenoyl-sn-glycero-3-phosphoethanolamine, 1,2-diarachidonoyl-sn-glycero-3-phosphoethanolamine, 1,2-didocosahexaenoyl-sn-glycero-3-phosphoethanolamine, 1,2-dioleoyl-sn-glycero-3-phospho-rac-(1-glycerol) sodium salt (DOPG), sphingomyelin, or mixtures thereof.

### Formula (HI)

In certain embodiments, a phospholipid is an analog or variant of DSPC. In certain embodiments, a phospholipid is a compound of Formula (HI):

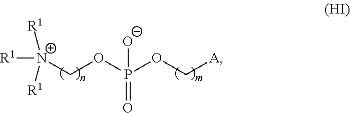

or a salt thereof, wherein:
each R¹ is independently optionally substituted alkyl; or optionally two R¹ are joined together with the intervening atoms to form optionally substituted monocyclic carbocyclyl or optionally substituted monocyclic heterocyclyl; or optionally three R¹ are joined together with the intervening atoms to form optionally substituted bicyclic carbocyclyl or optionally substitute bicyclic heterocyclyl;
n is 1,2,3,4,5,6,7, 8,9,or 10;
m is 0, 1, 2, 3, 4, 5, 6, 7, 8, 9, or 10;
A is of the formula:

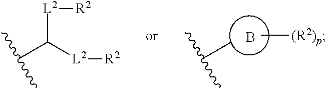

each instance of L² is independently a bond or optionally substituted C₁₋₆ alkylene, wherein one methylene unit of the optionally substituted C₁₋₆ alkylene is optionally replaced with O, N(R^{N}), S, C(O), C(O)N(R^{N}), NR^{N}C(O), C(O)O, OC(O), OC(O)O, OC(O)N(R^{N}), —NR^{N}C(O)O, or NR^{N}C(O)N(RN);
each instance of R² is independently optionally substituted C₁₋₃₀ alkyl, optionally substituted C₁₋₃₀ alkenyl, or optionally substituted C₁₋₃₀ alkynyl; optionally wherein one or more methylene units of R² are independently replaced with optionally substituted carbocyclylene, optionally substituted heterocyclylene, optionally substituted arylene, optionally substituted heteroarylene, N(R^{N}), O, S, C(O), C(O)N(R^{N}), NR^{N}C(O), NR^{N}C(O)N(R^{N}), C(O)O, OC(O), —OC(O)O, OC(O)N(R^{N}), NR^{N}C(O)O, C(O)S, SC(O), C(═NR^{N}), C(═NR^{N})N(R^{N}), NR^{N}C(═NR^{N}), NR^{N}C(═NR^{N})N(R^{N}), C(S), C(S)N(R^{N}), NR^{N}C(S), NR^{N}C(S)N(R^{N}), S(O) OS(O), S(O)O, —OS(O)O, OS(O)₂, S(O)₂O, OS(O)₂O, N(R^{N})S(O), S(O)N(R^{N}), N(R^{N})S(O)N(R^{N}), OS(O)N(R^{N}), N(R^{N})S(O)O, S(O)₂, N(R^{N})S(O)₂, S(O)₂N(R^{N}), N(R^{N})S(O)₂N(R^{N}), OS(O)₂N(R^{N}), or —N(RN)S(O)₂O;
each instance of R^{N} is independently hydrogen, optionally substituted alkyl, or a nitrogen protecting group;
Ring B is optionally substituted carbocyclyl, optionally substituted heterocyclyl, optionally substituted aryl, or optionally substituted heteroaryl; and
p is 1 or 2.

In certain embodiments, the compound is not of the formula:

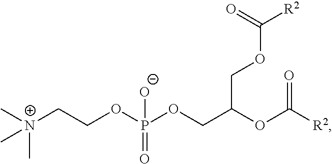

wherein each instance of R² is independently unsubstituted alkyl, unsubstituted alkenyl, or unsubstituted alkynyl.

In some embodiments, the phospholipids may be one or more of the phospholipids described in PCT Application No. PCT/US2018/037922.

In some embodiments, the lipid nanoparticle comprises a molar ratio of 5-25% non-cationic lipid relative to the other lipid components. For example, the lipid nanoparticle may comprise a molar ratio of 5-30%, 5-15%, 5-10%, 10-25%, 10-20%, 10-25%, 15-25%, 15-20%, 20-25%, or 25-30% non-cationic lipid. In some embodiments, the lipid nanoparticle comprises a molar ratio of 5%, 10%, 15%, 20%, 25%, or 30% non-cationic lipid.

In some embodiments, the lipid nanoparticle comprises a molar ratio of 5-25% phospholipid relative to the other lipid components. For example, the lipid nanoparticle may comprise a molar ratio of 5-30%, 5-15%, 5-10%, 10-25%, 10-20%, 10-25%, 15-25%, 15-20%, 20-25%, or 25-30% phospholipid. In some embodiments, the lipid nanoparticle comprises a molar ratio of 5%, 10%, 15%, 20%, 25%, or 30% phospholipid lipid.

### Structural Lipids

The lipid composition of a pharmaceutical composition disclosed herein can comprise one or more structural lipids. As used herein, the term “structural lipid” includes sterols and also to lipids containing sterol moieties.

Incorporation of structural lipids in the lipid nanoparticle may help mitigate aggregation of other lipids in the particle. Structural lipids can be selected from the group including but not limited to, cholesterol, fecosterol, sitosterol, ergosterol, campesterol, stigmasterol, brassicasterol, tomatidine, tomatine, ursolic acid, alpha-tocopherol, hopanoids, phytosterols, steroids, and mixtures thereof. In some embodiments, the structural lipid is a sterol. As defined herein, “sterols” are a subgroup of steroids consisting of steroid alcohols. In certain embodiments, the structural lipid is a steroid. In certain embodiments, the structural lipid is cholesterol. In certain embodiments, the structural lipid is an analog of cholesterol. In certain embodiments, the structural lipid is alpha-tocopherol.

In some embodiments, the structural lipids may be one or more of the structural lipids described in U.S. application Ser. No. 16/493,814.

In some embodiments, the lipid nanoparticle comprises a molar ratio of 25-55% structural lipid relative to the other lipid components. For example, the lipid nanoparticle may comprise a molar ratio of 10-55%, 25-50%, 25-45%, 25-40%, 25-35%, 25-30%, 30-55%, 30-50%, 30-45%, 30-40%, 30-35%, 35-55%, 35-50%, 35-45%, 35-40%, 40-55%, 40-50%, 40-45%, 45-55%, 45-50%, or 50-55% structural lipid. In some embodiments, the lipid nanoparticle comprises a molar ratio of 10%, 15%, 20%, 25%, 30%, 35%, 40%, 45%, 50%, or 55% structural lipid.

In some embodiments, the lipid nanoparticle comprises 30-45 mol % sterol, optionally 35-40 mol %, for example, 30-31 mol %, 31-32 mol %, 32-33 mol %, 33-34 mol %, 34-35 mol %, 35-36 mol %, 36-37 mol %, 37-38 mol %, 38-39 mol %, or 39-40 mol %. In some embodiments, the lipid nanoparticle comprises 25-55 mol % sterol. For example, the lipid nanoparticle may comprise 25-50 mol %, 25-45 mol %, 25-40 mol %, 25-35 mol %, 25-30 mol %, 30-55 mol %, 30-50 mol %, 30-45 mol %, 30-40 mol %, 30-35 mol %, 35-55 mol %, 35-50 mol %, 35-45 mol %, 35-40 mol %, 40-55 mol %, 40-50 mol %, 40-45 mol %, 45-55 mol %, 45-50 mol %, or 50-55 mol % sterol. In some embodiments, the lipid nanoparticle comprises 25 mol %, 30 mol %, 35 mol %, 40 mol %, 45 mol %, 50 mol %, or 55 mol % sterol.

In some embodiments, the lipid nanoparticle comprises 35-40 mol % cholesterol. For example, the lipid nanoparticle may comprise 35, 35.5, 36, 36.5, 37, 37.5, 38, 38.5, 39, 39.5, or 40 mol % cholesterol.

### Polyethylene Glycol (PEG)-Lipids

The lipid composition of a pharmaceutical composition disclosed herein can comprise one or more polyethylene glycol (PEG) lipids.

As used herein, the term “PEG-lipid” or “PEG-modified lipid” refers to polyethylene glycol (PEG)-modified lipids. Non-limiting examples of PEG-lipids include PEG-modified phosphatidylethanolamine and phosphatidic acid, PEG-ceramide conjugates (e.g., PEG-CerC14 or PEG-CerC20), PEG-modified dialkylamines, and PEG-modified 1,2-diacyloxypropan-3-amines. Such lipids are also referred to as PEGylated lipids. For example, a PEG lipid can be PEG-c-DOMG, PEG-DMG, PEG-DLPE, PEG-DMPE, PEG-DPPC, or a PEG-DSPE lipid.

In some embodiments, the PEG-lipid includes, but not limited to 1,2-dimyristoyl-sn-glycerol methoxypolyethylene glycol (PEG-DMG), 1,2-distearoyl-sn-glycero-3-phosphoethanolamine-N-[amino(polyethylene glycol)](PEG-DSPE), PEG-disteryl glycerol (PEG-DSG), PEG-dipalmetoleyl, PEG-dioleyl, PEG-distearyl, PEG-diacylglycamide (PEG-DAG), PEG-dipalmitoyl phosphatidylethanolamine (PEG-DPPE), or PEG-1,2-dimyristyloxlpropyl-3-amine (PEG-c-DMA).

In some embodiments, the PEG-lipid is selected from the group consisting of a PEG-modified phosphatidylethanolamine, a PEG-modified phosphatidic acid, a PEG-modified ceramide, a PEG-modified dialkylamine, a PEG-modified diacylglycerol, a PEG-modified dialkylglycerol, and mixtures thereof. In some embodiments, the PEG-modified lipid is PEG-DMG, PEG-c-DOMG (also referred to as PEG-DOMG), PEG-DSG, and/or PEG-DPG.

In some embodiments, the lipid moiety of the PEG-lipids includes those having lengths of from about C₁₄ to about C₂₂, preferably from about C₁₄ to about C₁₆. In some embodiments, a PEG moiety, for example an mPEG-NH₂, has a size of about 1000, 2000, 5000, 10,000, 15,000 or 20,000 daltons. In some embodiments, the PEG-lipid is PEG₂ₖ-DMG.

In some embodiments, the lipid nanoparticles described herein can comprise a PEG lipid which is a non-diffusible PEG. Non-limiting examples of non-diffusible PEGs include PEG-DSG and PEG-DSPE.

PEG-lipids are known in the art, such as those described in U.S. Pat. No. 8,158,601 and International Publ. No. WO 2015/130584 A2, which are incorporated herein by reference in their entirety.

In general, some of the other lipid components (e.g., PEG lipids) of various formulae described herein may be synthesized as described International Patent Application No. PCT/US2016/000129, filed Dec. 10, 2016, entitled “Compositions and Methods for Delivery of Therapeutic Agents,” which is incorporated by reference in its entirety.

The lipid component of a lipid nanoparticle composition may include one or more molecules comprising polyethylene glycol, such as PEG or PEG-modified lipids. Such species may be alternately referred to as PEGylated lipids. A PEG lipid is a lipid modified with polyethylene glycol. A PEG lipid may be selected from the non-limiting group including PEG-modified phosphatidylethanolamines, PEG-modified phosphatidic acids, PEG-modified ceramides, PEG-modified dialkylamines, PEG-modified diacylglycerols, PEG-modified dialkylglycerols, and mixtures thereof. For example, a PEG lipid may be PEG-c-DOMG, PEG-DMG, PEG-DLPE, PEG-DMPE, PEG-DPPC, or a PEG-DSPE lipid.

In some embodiments the PEG-modified lipids are a modified PEG DMG. PEG-DMG has the following structure:

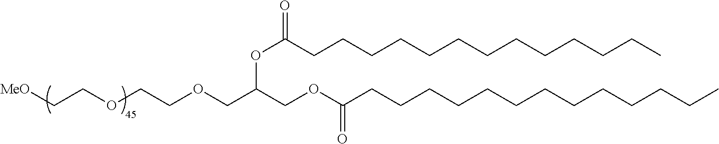

In some embodiments, PEG lipids can be PEGylated lipids described in International Publication No. WO2012099755, the contents of which is herein incorporated by reference in its entirety. Any of these exemplary PEG lipids described herein may be modified to comprise a hydroxyl group on the PEG chain. In certain embodiments, the PEG lipid is a PEG-OH lipid. As 5 generally defined herein, a “PEG-OH lipid” (also referred to herein as “hydroxy-PEGylated lipid”) is a PEGylated lipid having one or more hydroxyl (—OH) groups on the lipid. In certain embodiments, the PEG-OH lipid includes one or more hydroxyl groups on the PEG chain. In certain embodiments, a PEG-OH or hydroxy-PEGylated lipid comprises an —OH group at the terminus of the PEG chain. Each possibility represents a separate embodiment.

### Formula (PI)

In certain embodiments, a PEG lipid is a compound of Formula (PI):

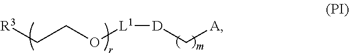

or salts thereof, wherein:
R³ is —OR^{O};
R^{O} is hydrogen, optionally substituted alkyl, or an oxygen protecting group;
r is an integer between 1 and 100, inclusive;
L¹ is optionally substituted C₁₋₁₀ alkylene, wherein at least one methylene of the optionally substituted C₁₋₁₀ alkylene is independently replaced with optionally substituted carbocyclylene, optionally substituted heterocyclylene, optionally substituted arylene, optionally substituted heteroarylene, O, N(R^{N}), S, C(O), C(O)N(R^{N}), NR^{N}C(O), C(O)O, OC(O), OC(O)O, OC(O)N(R^{N}), NR^{N}C(O)O, or NR^{N}C(O)N(R^{N});
D is a moiety obtained by click chemistry or a moiety cleavable under physiological conditions;
m is 0, 1, 2, 3, 4, 5, 6, 7, 8, 9, or 10;
A is of the formula:

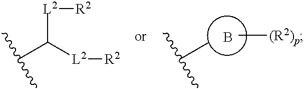

each instance of L² is independently a bond or optionally substituted C₁₋₆ alkylene, wherein one methylene unit of the optionally substituted C₁₋₆ alkylene is optionally replaced with O, N(R^{N}), S, C(O), C(O)N(R^{N}), NR^{N}C(O), C(O)O, OC(O), OC(O)O, OC(O)N(R^{N}), —NR^{N}C(O)O, or NR^{N}C(O)N(RN);
each instance of R² is independently optionally substituted C₁₋₃₀ alkyl, optionally substituted C₁₋₃₀ alkenyl, or optionally substituted C₁₋₃₀ alkynyl; optionally wherein one or more methylene units of R² are independently replaced with optionally substituted carbocyclylene, optionally substituted heterocyclylene, optionally substituted arylene, optionally substituted heteroarylene, N(R^{N}), O, S, C(O), C(O)N(R^{N}), NR^{N}C(O), NR^{N}C(O)N(R^{N}), C(O)O, OC(O), —OC(O)O, OC(O)N(R^{N}), NR^{N}C(O)O, C(O)S, SC(O), C(═NR^{N}), C(═NR^{N})N(R^{N}), NR^{N}C(═NR^{N}), NR^{N}C(═NR^{N})N(R^{N}), C(S), C(S)N(R^{N}), NR^{N}C(S), NR^{N}C(S)N(R^{N}), S(O) OS(O) S(O)O, —OS(O)O, OS(O)₂, S(O)₂O, OS(O)₂O, N(R^{N})S(O), S(O)N(R^{N}), N(R^{N})S(O)N(R^{N}), OS(O)N(R^{N}), N(R^{N})S(O)O, S(O)₂, N(R^{N})S(O)₂, S(O)₂N(R^{N}), N(R^{N})S(O)₂N(R^{N}), OS(O)₂N(R^{N}), or —N(RN)S(O)₂O;
each instance of R^{N} is independently hydrogen, optionally substituted alkyl, or a nitrogen protecting group;
Ring B is optionally substituted carbocyclyl, optionally substituted heterocyclyl, optionally substituted aryl, or optionally substituted heteroaryl; and
p is 1 or 2.

In certain embodiments, the compound of Formula (PI) is a PEG-OH lipid (i.e., R³ is —OR^{O}, and R^{O} is hydrogen). In certain embodiments, the compound of Formula (PI) is of Formula (PI-OH):

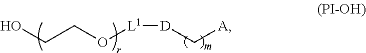

or a salt thereof.

### Formula (PII)

In certain embodiments, a PEG lipid is a PEGylated fatty acid. In certain embodiments, a PEG lipid is a compound of Formula (PII). In some embodiments, compounds of Formula (PII) have the following formula:

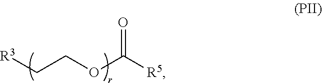

or a salts thereof, wherein:
R³ is-OR^{O};
R^{O} is hydrogen, optionally substituted alkyl or an oxygen protecting group;
r is an integer between 1 and 100, inclusive;
R⁵ is optionally substituted C₁₋₄₀ alkyl, optionally substituted C₁₋₄₀ alkenyl, or optionally substituted C₁₋₄₀ alkynyl; and optionally one or more methylene groups of R⁵ are replaced with optionally substituted carbocyclylene, optionally substituted heterocyclylene, optionally substituted arylene, optionally substituted heteroarylene, N(R^{N}), O, S, C(O), —C(O)N(R^{N}), NR^{N}C(O), NR^{N}C(O)N(R^{N}), C(O)O, OC(O), OC(O)O, OC(O)N(R^{N}), NR^{N}C(O)O, C(O)S, SC(O), C(═NR^{N}), C(═NR^{N})N(R^{N}), NR^{N}C(═NR^{N}), NR^{N}C(═NR^{N})N(R^{N}), C(S), —C(S)N(R^{N}), NR^{N}C(S), NR^{N}C(S)N(R^{N}), S(O), OS(O), S(O)O, OS(O)O, OS(O)₂, S(O)₂O, —OS(O)₂O, N(R^{N})S(O), S(O)N(R^{N}), N(R^{N})S(O)N(R^{N}), OS(O)N(R^{N}), N(R^{N})S(O)O, S(O)₂, —N(R^{N})S(O)₂, S(O)₂N(R^{N}), N(R^{N})S(O)₂N(R^{N}), OS(O)₂N(R^{N}), or N(R^{N})S(O)₂O; and
each instance of R^{N} is independently hydrogen, optionally substituted alkyl, or a nitrogen protecting group.

In certain embodiments, the compound of Formula (PII) is of Formula (PII-OH):

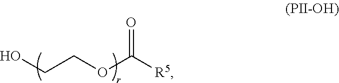

or a salt thereof. In some embodiments, r is 40-50.

In yet other embodiments the compound of Formula (PII) is:

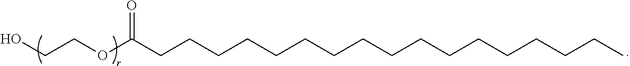

or a salt thereof.

In some embodiments, the compound of Formula (PII) is

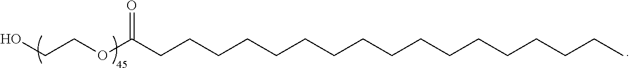

In some embodiments, the lipid composition of the pharmaceutical compositions disclosed herein does not comprise a PEG-lipid.

In some embodiments, the PEG-lipids may be one or more of the PEG lipids described in U.S. Application No. U.S. Ser. No. 15/674,872.

In some embodiments, the lipid nanoparticle comprises a molar ratio of 0.5-15% PEG lipid relative to the other lipid components. For example, the lipid nanoparticle may comprise a molar ratio of 0.5-10%, 0.5-5%, 1-15%, 1-10%, 1-5%, 2-15%, 2-10%, 2-5%, 5-15%, 5-10%, or 10-15% PEG lipid. In some embodiments, the lipid nanoparticle comprises a molar ratio of 0.5%, 1%, 2%, 3%, 4%, 5%, 6%, 7%, 8%, 9%, 10%, 11%, 12%, 13%, 14%, or 15% PEG-lipid.

In some embodiments, the lipid nanoparticle comprises 1-5% PEG-modified lipid, optionally 1-3 mol %, for example 1.5 to 2.5 mol %, 1-2 mol %, 2-3 mol %, 3-4 mol %, or 4-5 mol %. In some embodiments, the lipid nanoparticle comprises 0.5-15 mol % PEG-modified lipid. For example, the lipid nanoparticle may comprise 0.5-10 mol %, 0.5-5 mol %, 1-15 mol %, 1-10 mol %, 1-5 mol %, 2-15 mol %, 2-10 mol %, 2-5 mol %, 5-15 mol %, 5-10 mol %, or 10-15 mol %. In some embodiments, the lipid nanoparticle comprises 0.5 mol %, 1 mol %, 2 mol %, 3 mol %, 4 mol %, 5 mol %, 6 mol %, 7 mol %, 8 mol %, 9 mol %, 10 mol %, 11 mol %, 12 mol %, 13 mol %, 14 mol %, or 15 mol % PEG-modified lipid.

Some embodiments comprise adding PEG to a composition comprising an LNP encapsulating a nucleic acid (e.g., which already includes PEG in the amounts listed above). In embodiments comprise adding about 0.5 mol % or more PEG to an LNP composition, such as about 1 mol %, about 1.5 mol %, about 2 mol %, about 2.5 mol %, about 3 mol %, about 3.5 mol %, about 4 mol %, about 5 mol %, or more after formation of an LNP composition (e.g., which already contains PEG in amount listed elsewhere herein).

In some embodiments, the lipid nanoparticle comprises 20-60 mol % ionizable amino lipid, 5-25 mol % non-cationic lipid, 25-55 mol % sterol, and 0.5-15 mol % PEG-modified lipid.

In some embodiments, a LNP of the disclosure comprises an ionizable amino lipid of Compound 1, wherein the non-cationic lipid is DSPC, the structural lipid that is cholesterol, and the PEG lipid is DMG-PEG.

In some embodiments, a LNP of the disclosure comprises an ionizable amino lipid of Compound 2, wherein the non-cationic lipid is DSPC, the structural lipid that is cholesterol, and the PEG lipid is DMG-PEG.

In some embodiments, a LNP comprises an ionizable amino lipid of any of Formula (AIII), (AIV), or (AV), a phospholipid comprising DSPC, a structural lipid, and a PEG lipid comprising PEG-DMG.

In some embodiments, a LNP comprises an ionizable amino lipid of any of Formula (AIII), (AIV), or (AV), a phospholipid comprising DSPC, a structural lipid, and a PEG lipid comprising a compound having Formula (PII).

In some embodiments, a LNP comprises an ionizable amino lipid of Formula (AIII), (AIV), or (AV), a phospholipid comprising a compound having Formula (HI), a structural lipid, and the PEG lipid comprising a compound having Formula (PI) or (PII).

In some embodiments, a LNP comprises an ionizable amino lipid of Formula (AIII), (AIV), or (AV), a phospholipid comprising a compound having Formula (HI), a structural lipid, and the PEG lipid comprising a compound having Formula (PI) or (PII).

In some embodiments, a LNP comprises an ionizable amino lipid of Formula (AIII), (AIV), or (AV), a phospholipid having Formula (HI), a structural lipid, and a PEG lipid comprising a compound having Formula (PII).

In some embodiments, the lipid nanoparticle comprises 49 mol % ionizable amino lipid, 10 mol % DSPC, 38.5 mol % cholesterol, and 2.5 mol % DMG-PEG.

In some embodiments, the lipid nanoparticle comprises 49 mol % ionizable amino lipid, 11 mol % DSPC, 38.5 mol % cholesterol, and 1.5 mol % DMG-PEG.

In some embodiments, the lipid nanoparticle comprises 48 mol % ionizable amino lipid, 11 mol % DSPC, 38.5 mol % cholesterol, and 2.5 mol % DMG-PEG.

In some embodiments, a LNP comprises an N:P ratio of from about 2:1 to about 30:1.

In some embodiments, a LNP comprises an N:P ratio of about 6:1.

In some embodiments, a LNP comprises an N:P ratio of about 3:1, 4:1, or 5:1.

In some embodiments, a LNP comprises a wt/wt ratio of the ionizable amino lipid component to the mRNA of from about 10:1 to about 100:1.

In some embodiments, a LNP comprises a wt/wt ratio of the ionizable amino lipid component to the mRNA of about 20:1.

In some embodiments, a LNP comprises a wt/wt ratio of the ionizable amino lipid component to the mRNA of about 10:1.

Some embodiments comprise a composition having one or more LNPs having a diameter of about 150 nm or less, such as about 140 nm, 130 nm, 120 nm, 110 nm, 100 nm, 90 nm, 80 nm, 70 nm, 60 nm, 50 nm, 40 nm, 30 nm, or 20 nm or less. Some embodiments comprise a composition having a mean LNP diameter of about 150 nm or less, such as about 140 nm, 130 nm, 120 nm, 110 nm, 100 nm, 90 nm, 80 nm, 70 nm, 60 nm, 50 nm, 40 nm, 30 nm, or 20 nm or less. In some embodiments, the composition has a mean LNP diameter from about 30 nm to about 150 nm, or a mean diameter from about 60 nm to about 120 nm.

A LNP may comprise one or more types of lipids, including but not limited to amino lipids (e.g., ionizable amino lipids), neutral lipids, non-cationic lipids, charged lipids, PEG-modified lipids, phospholipids, structural lipids and sterols. In some embodiments, a LNP may further comprise one or more cargo molecules, including but not limited to nucleic acids, plasmid DNA, DNA or RNA oligonucleotides, siRNA, shRNA, snRNA, snoRNA, lncRNA, etc.), small molecules, proteins and peptides.

In some embodiments, the composition comprises a liposome. A liposome is a lipid particle comprising lipids arranged into one or more concentric lipid bilayers around a central region. The central region of a liposome may comprise an aqueous solution, suspension, or other aqueous composition.

In some embodiments, a lipid nanoparticle may comprise two or more components (e.g., amino lipid and nucleic acid, PEG-lipid, phospholipid, structural lipid). For instance, a lipid nanoparticle may comprise an amino lipid and a nucleic acid. Compositions comprising the lipid nanoparticles, such as those described herein, may be used for a wide variety of applications, including the stealth delivery of therapeutic payloads with minimal adverse innate immune response.

Effective in vivo delivery of nucleic acids represents a continuing medical challenge. Exogenous nucleic acids (i.e., originating from outside of a cell or organism) are readily degraded in the body, e.g., by the immune system. Accordingly, effective delivery of nucleic acids to cells often requires the use of a particulate carrier (e.g., lipid nanoparticles). The particulate carrier should be formulated to have minimal particle aggregation, be relatively stable prior to intracellular delivery, effectively deliver nucleic acids intracellularly, and illicit no or minimal immune response. To achieve minimal particle aggregation and pre-delivery stability, many conventional particulate carriers have relied on the presence and/or concentration of certain components (e.g., PEG-lipid). However, it has been discovered that certain components may decrease the stability of encapsulated nucleic acids (e.g., mRNA). The reduced stability may limit the broad applicability of the particulate carriers. As such, there remains a need for methods by which to improve the stability of nucleic acid (e.g., mRNA) encapsulated within lipid nanoparticles.

In some embodiments, the lipid nanoparticles comprise one or more of ionizable molecules, polynucleotides, and optional components, such as structural lipids, sterols, neutral lipids, phospholipids and a molecule capable of reducing particle aggregation (e.g., polyethylene glycol (PEG), PEG-modified lipid), such as those described above.

In some embodiments, a LNP described herein may include one or more ionizable molecules (e.g., amino lipids or ionizable lipids). The ionizable molecule may comprise a charged group and may have a certain pKa. In certain embodiments, the pKa of the ionizable molecule may be greater than or equal to about 6, greater than or equal to about 6.2, greater than or equal to about 6.5, greater than or equal to about 6.8, greater than or equal to about 7, greater than or equal to about 7.2, greater than or equal to about 7.5, greater than or equal to about 7.8, greater than or equal to about 8. In some embodiments, the pKa of the ionizable molecule may be less than or equal to about 10, less than or equal to about 9.8, less than or equal to about 9.5, less than or equal to about 9.2, less than or equal to about 9.0, less than or equal to about 8.8, or less than or equal to about 8.5. Combinations of the above referenced ranges are also possible (e.g., greater than or equal to 6 and less than or equal to about 8.5). Other ranges are also possible. In embodiments in which more than one type of ionizable molecule are present in a particle, each type of ionizable molecule may independently have a pKa in one or more of the ranges described above.

In general, an ionizable molecule comprises one or more charged groups. In some embodiments, an ionizable molecule may be positively charged or negatively charged. For instance, an ionizable molecule may be positively charged. For example, an ionizable molecule may comprise an amine group. As used herein, the term “ionizable molecule” has its ordinary meaning in the art and may refer to a molecule or matrix comprising one or more charged moiety. As used herein, a “charged moiety” is a chemical moiety that carries a formal electronic charge, e.g., monovalent (+1, or −1), divalent (+2, or −2), trivalent (+3, or −3), etc. The charged moiety may be anionic (i.e., negatively charged) or cationic (i.e., positively charged). Examples of positively-charged moieties include amine groups (e.g., primary, secondary, and/or tertiary amines), ammonium groups, pyridinium group, guanidine groups, and imidazolium groups. In a particular embodiment, the charged moieties comprise amine groups. Examples of negatively-charged groups or precursors thereof, include carboxylate groups, sulfonate groups, sulfate groups, phosphonate groups, phosphate groups, hydroxyl groups, and the like. The charge of the charged moiety may vary, in some cases, with the environmental conditions, for example, changes in pH may alter the charge of the moiety, and/or cause the moiety to become charged or uncharged. In general, the charge density of the molecule and/or matrix may be selected as desired.

In some cases, an ionizable molecule (e.g., an amino lipid or ionizable lipid) may include one or more precursor moieties that can be converted to charged moieties. For instance, the ionizable molecule may include a neutral moiety that can be hydrolyzed to form a charged moiety, such as those described above. As a non-limiting specific example, the molecule or matrix may include an amide, which can be hydrolyzed to form an amine, respectively. Those of ordinary skill in the art will be able to determine whether a given chemical moiety carries a formal electronic charge (for example, by inspection, pH titration, ionic conductivity measurements, etc.), and/or whether a given chemical moiety can be reacted (e.g., hydrolyzed) to form a chemical moiety that carries a formal electronic charge.

The ionizable molecule (e.g., amino lipid or ionizable lipid) may have any suitable molecular weight. In certain embodiments, the molecular weight of an ionizable molecule is less than or equal to about 2,500 g/mol, less than or equal to about 2,000 g/mol, less than or equal to about 1,500 g/mol, less than or equal to about 1,250 g/mol, less than or equal to about 1,000 g/mol, less than or equal to about 900 g/mol, less than or equal to about 800 g/mol, less than or equal to about 700 g/mol, less than or equal to about 600 g/mol, less than or equal to about 500 g/mol, less than or equal to about 400 g/mol, less than or equal to about 300 g/mol, less than or equal to about 200 g/mol, or less than or equal to about 100 g/mol. In some instances, the molecular weight of an ionizable molecule is greater than or equal to about 100 g/mol, greater than or equal to about 200 g/mol, greater than or equal to about 300 g/mol, greater than or equal to about 400 g/mol, greater than or equal to about 500 g/mol, greater than or equal to about 600 g/mol, greater than or equal to about 700 g/mol, greater than or equal to about 1000 g/mol, greater than or equal to about 1,250 g/mol, greater than or equal to about 1,500 g/mol, greater than or equal to about 1,750 g/mol, greater than or equal to about 2,000 g/mol, or greater than or equal to about 2,250 g/mol. Combinations of the above ranges (e.g., at least about 200 g/mol and less than or equal to about 2,500 g/mol) are also possible. In embodiments in which more than one type of ionizable molecules are present in a particle, each type of ionizable molecule may independently have a molecular weight in one or more of the ranges described above.

In some embodiments, the percentage (e.g., by weight, or by mole) of a single type of ionizable molecule (e.g., amino lipid or ionizable lipid) and/or of all the ionizable molecules within a particle may be greater than or equal to about 15%, greater than or equal to about 16%, greater than or equal to about 17%, greater than or equal to about 18%, greater than or equal to about 19%, greater than or equal to about 20%, greater than or equal to about 21%, greater than or equal to about 22%, greater than or equal to about 23%, greater than or equal to about 24%, greater than or equal to about 25%, greater than or equal to about 30%, greater than or equal to about 35%, greater than or equal to about 40%, greater than or equal to about 42%, greater than or equal to about 45%, greater than or equal to about 48%, greater than or equal to about 50%, greater than or equal to about 52%, greater than or equal to about 55%, greater than or equal to about 58%, greater than or equal to about 60%, greater than or equal to about 62%, greater than or equal to about 65%, or greater than or equal to about 68%. In some instances, the percentage (e.g., by weight, or by mole) may be less than or equal to about 70%, less than or equal to about 68%, less than or equal to about 65%, less than or equal to about 62%, less than or equal to about 60%, less than or equal to about 58%, less than or equal to about 55%, less than or equal to about 52%, less than or equal to about 50%, or less than or equal to about 48%. Combinations of the above referenced ranges are also possible (e.g., greater than or equal to 20% and less than or equal to about 60%, greater than or equal to 40% and less than or equal to about 55%, etc.). In embodiments in which more than one type of ionizable molecule is present in a particle, each type of ionizable molecule may independently have a percentage (e.g., by weight, or by mole) in one or more of the ranges described above. The percentage (e.g., by weight, or by mole) may be determined by extracting the ionizable molecule(s) from the dried particles using, e.g., organic solvents, and measuring the quantity of the agent using high pressure liquid chromatography (i.e., HPLC), liquid chromatography-mass spectrometry (LC-MS), nuclear magnetic resonance (NMR), or mass spectrometry (MS). Those of ordinary skill in the art would be knowledgeable of techniques to determine the quantity of a component using the above-referenced techniques. For example, HPLC may be used to quantify the amount of a component, by, e.g., comparing the area under the curve of a HPLC chromatogram to a standard curve.

It should be understood that the terms “charged” or “charged moiety” does not refer to a “partial negative charge” or “partial positive charge” on a molecule. The terms “partial negative charge” and “partial positive charge” are given their ordinary meaning in the art. A “partial negative charge” may result when a functional group comprises a bond that becomes polarized such that electron density is pulled toward one atom of the bond, creating a partial negative charge on the atom. Those of ordinary skill in the art will, in general, recognize bonds that can become polarized in this way.

According to the disclosures herein, a lipid composition may comprise one or more lipids as described herein. Such lipids may include those useful in the preparation of lipid nanoparticle formulations as described above or as known in the art.

### Stabilizing Compounds

Some embodiments of the compositions described herein are stabilized pharmaceutical compositions. Various non-viral delivery systems, including nanoparticle formulations, present attractive opportunities to overcome many challenges associated with mRNA delivery. Lipid nanoparticles (LNPs) have drawn particular attention in recent years as various LNP formulations have shown promise in a variety of pharmaceutical applications. However, lipids have been shown to degrade nucleic acids, including mRNA, and lipid nanoparticle formulations undergo rapid loss of purity when stored as refrigerated liquids. Moreover, the storage stability of mRNA encapsulated within LNPs is lower than that of unencapsulated mRNA.

A class of compounds has been found to stabilize nucleic acids within a lipid carrier such as an LNP, an unexpected and unprecedented discovery which enables applications including extended refrigerated liquid shelf-life, extended in-use periods at room temperature, and extended in-use stability at physiological temperatures up to higher temperatures such as 40° C. Such stabilizing compounds solve a critical problem, as current manufacturing processes and formulations experience a 5-10% purity loss during LNP formation and processing that is typical with current large-scale LNP production.

In some embodiments, the stabilized pharmaceutical composition comprises a nucleic acid formulation comprising a nucleic acid and a stabilizing compound (e.g., a compound of Formula (I), of Formula (II), or a tautomer or solvate thereof). In some embodiments, the stabilized pharmaceutical composition comprises a nucleic acid formulation comprising a nucleic acid and a lipid, and a compound of Formula (I):

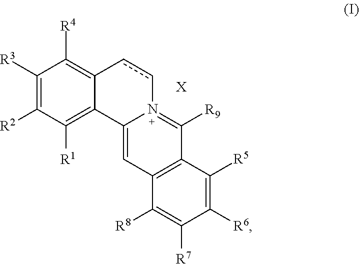

or a tautomer or solvate thereof, wherein:
- - - is a single bond or a double bond;
R¹ is H; R² is OCH₃, or together with R³ is OCH₂O; R³ is OCH₃, or together with R² is OCH₂O; R⁴ is H; R⁵ is H or OCH₃; R⁶ is OCH₃; R⁷ is H or OCH₃; R⁸ is H; R⁹ is H or CH₃; and X is a pharmaceutically acceptable anion, e.g., a halide such as chloride.

In some embodiments, the compound of Formula (I) has the structure of:

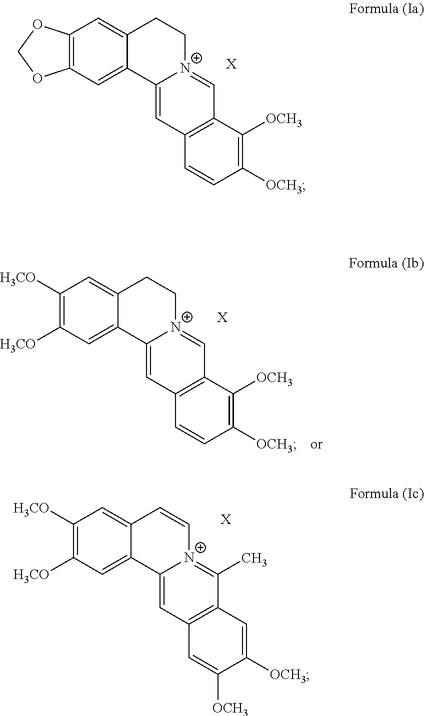

or a tautomer or solvate thereof.

In some embodiments, the stabilized pharmaceutical composition comprises a nucleic acid formulation comprising a nucleic acid and a lipid, and a compound of Formula (II):

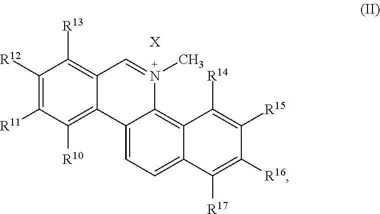

or a tautomer or solvate thereof, wherein:
R¹⁰ is H; R¹¹ is H; R¹² together with R¹³ is OCH₂O; R¹⁴ is H; R¹⁵ together with R¹⁶ is OCH₂O; R¹⁷ is H; and X is a pharmaceutically acceptable anion, e.g., a halide such as chloride.

In some embodiments, the compound of Formula (II) has the structure of:

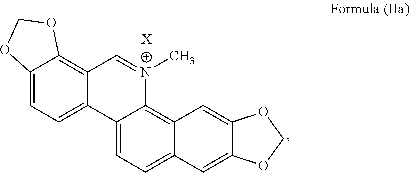

or a tautomer or solvate thereof.

Stabilizing compounds of Formulas (I), (Ia), (Ib), (Ic), (II), and (IIa) are described in International Application No. PCT/US2022/025967, which is incorporated by reference herein in its entirety.

In some embodiments, the nucleic acid formulation comprises lipid nanoparticles. In some embodiments, the nucleic acid is mRNA.

In some embodiments, the stabilizing compound (“the compound”) has a purity of at least 70%, 80%, 90%, 95%, or 99%. In some embodiments, the compound contains fewer than 100 ppm of elemental metals. In some embodiments, the stabilized pharmaceutical composition (“the composition”) comprises a pharmaceutically acceptable metal chelator, e.g., EDTA (ethylenediaminetetraacetic acid) or DTPA (diethylenetriaminepentaacetic acid).

In some embodiments, the composition is an aqueous solution. In some embodiments, the compound is present at a concentration between about 0.1 mM and about 10 mM in the aqueous solution. In some embodiments, the aqueous solution has a pH of or about 5 to 8, including pH of about 5, 5.5, 6, 6.5, 7, 7.5, or 8. In some embodiments, the aqueous solution does not comprise NaCl. In some embodiments, the aqueous solution comprises NaCl in a concentration of or about 150 mM. In some embodiments, the aqueous solution comprises a phosphate buffer, a tris buffer, an acetate buffer, a histidine buffer, or a citrate buffer.

In some embodiments, microbial growth in the composition is inhibited by the compound.

In some embodiments, the composition is characterized as having a mRNA purity level of greater than 60%, greater than 70%, greater than 80%, or greater than 90% main peak mRNA purity after at least thirty days of storage. In some embodiments, the composition comprises a mRNA purity level of greater than 50% main peak mRNA purity after at least six months of storage. In some embodiments, the storage is at room temperature.

In some embodiments, the composition comprises a lipid nanoparticle encapsulating a mRNA, and the composition comprises less than 50%, less than 60%, less than 70%, less than 80%, less than 90%, or less than 95% RNA fragments after at least thirty days of storage. In some embodiments, the storage temperature is greater than room temperature. In some embodiments, the storage temperature is about 4° C.

In some embodiments, the compound interacts with the nucleic acid comprised within a lipid nanostructure (e.g., a lipid nanoparticle, liposome, or lipoplex), e.g., via pi-pi stacking and/or by changing backbone helicity of the nucleic acid. In some embodiments, the compound intercalates with a nucleic acid. In some embodiments, the compound binds with a nucleic acid, e.g., reversible binding, and/or binding to the stranded regions of the nucleic acid. In some embodiments, the compound self-associates, binds to nucleic acid ribose contacts, and/or binds to nucleic acid base contacts. In some embodiments, the compound does not substantially bind to nucleic acid phosphate contacts. In some embodiments, the positive charge of the compound contributes to nucleic acid binding. In some embodiments, the interacts with the nucleic acid with a binding affinity defined by an equilibrium dissociation constant of less than 10⁻³ M (e.g., less than 10⁻⁴ M, less than 10⁻⁵ M, less than 10⁻⁵ M, less than 10⁻⁷ M, less than 10⁻⁸ M, or less than 10⁻⁹ M).

In some embodiments, the compound interacts with a nucleic acid and provides shielding from solvent, e.g., water. In some embodiments, the compound shields ribose from solvent more than the compound shields the phosphate groups of the nucleic acid. In some embodiments, the solvent exposure is measured by the solvent accessible surface area (SASA). In some embodiments, a stabilizing compound decreases the solvent accessible area of ribose to about 5-10 nm². In some embodiments, a stabilizing compound decreases the solvent accessible area of ribose to about 6-8 nm². In some embodiments, a stabilizing compound decreases the solvent accessible area of phosphate to about 9-12 nm². In some embodiments, a stabilizing compound decreases the solvent accessible area of phosphate to about 10-11 nm².

In some embodiments, a nucleic acid that is conformationally stabilized by the compound exhibits thermal unfolding temperatures (measured by circular dichroism or DSC, for example) that are higher than in the absence of the compound. In some embodiments, the compound confers increased stability, e.g., thermal stability, to the nucleic acid in a folded structure, e.g., relative to its unfolded or less folded or more linear form. In some embodiments, the compound causes compaction of the nucleic acid upon interaction with the nucleic acid. In some embodiments, the compound causes a decrease in the hydrodynamic radius of the nucleic acid molecule upon interaction with the nucleic acid. In some embodiments, a stabilizing compound causes compaction or a decrease in the hydrodynamic radius of a nucleic acid molecule by 5%, 10%, 15%, 20%, 25%, 30%, 35%, 40%, 45%, 50%, 55%, 60%, or more. In some embodiments, a stabilizing compound causes compaction or a decrease in the hydrodynamic radius of a nucleic acid molecule when the compound is in a concentration of 1 μM, 2 μM, 3 μM, 4 μM, 5 μM, 6 μM, 7 μM, 8 μM, 9 μM, 10 μM, 15 μM, 20 μM, 25 μM, 30 μM, 35 μM, 40 μM, 45 μM, 50 μM, 60 μM, 70 μM, 80 μM, 90 μM, or 100 μM.

mRNA-Lipid Adducts

It has been determined that certain ionizable lipids are susceptible to the formation of lipid-polynucleotide adducts. In particular, ionizable lipids that comprise a tertiary amine group may decompose into one or both of a secondary amine and a reactive aldehyde species capable of interacting with polynucleotides (such as mRNA) to form an ionizable lipid-polynucleotide adduct impurity that can be detected by reverse phase ion pair chromatography (RP-IP HPLC). For example, oxidation of the tertiary amine may lead to N-oxide formation that can undergo acid/base-catalyzed hydrolysis at the amine to generate aldehydes and secondary amines which may form adducts with mRNA. Thus, in some aspects, the ionizable lipid-polynucleotide adduct impurity is an aldehyde-mRNA adduct impurity.

It also has been determined that such adducts may disrupt mRNA translation and impact the activity of lipid nanoparticle (LNP) formulated mRNA products. Thus, it can be advantageous to prepare and use LNP compositions with a reduced content of ionizable lipid-polynucleotide adduct impurity, such as wherein less than about 20%, less than about 10%, less than about 5%, or less than about 1%, of the mRNA is in the form of ionizable lipid-polynucleotide adduct impurity, as may be measured by RP-IP HPLC. Thus, in accordance with some aspects, an LNP composition is provided wherein less than about 10%, less than about 5%, or less than about 1%, of the mRNA is in the form of ionizable lipid-polynucleotide adduct impurity, including less than 10%, less than 5%, or less than 1%, as may be measured by RP-IP HPLC.

In some aspects, an amount of lipid aldehydes in the composition is less than about 50 ppm, including less than 50 ppm. Additionally or alternatively, in some aspects an amount of N-oxide compounds in the composition is less than about 50 ppm, including less than 50 ppm. Additionally or alternatively, in some aspects an amount of transition metals, such as Fe, in the composition is less than about 50 ppm, including less than 50 ppm. Additionally or alternatively, in some aspects an amount of alkyl halide compounds in the composition is less than about 50 ppm, including less than 50 ppm. Additionally or alternatively, in some aspects an amount of anhydride compounds in the composition is less than about 50 ppm, including less than 50 ppm. Additionally or alternatively, in some aspects an amount of ketone compounds in the composition is less than about 50 ppm, including less than 50 ppm. Additionally or alternatively, in some aspects an amount of conjugated diene compounds in the composition is less than about 50 ppm, including less than 50 ppm.

In some aspects, the composition is stable against the formation of ionizable lipid-polynucleotide adduct impurity. In some aspects, an amount of ionizable lipid-polynucleotide adduct impurity in the composition increases at an average rate of less than about 2% per day when stored at a temperature of about 25° C. or below, including at an average rate of less than 2% per day. In some aspects, an amount of ionizable lipid-polynucleotide adduct impurity in the composition increases at an average rate of less than about 0.5% per day when stored at a temperature of about 5° C. or below, including at an average rate of less than 0.5% per day. In some aspects, an amount of ionizable lipid-polynucleotide adduct impurity in the composition increases at an average rate of less than about 0.5% per day when stored at a refrigerated temperature, optionally wherein the refrigerated temperature is about 5° C.

Lipid vehicle (e.g., LNP) compositions with a reduced content of ionizable lipid-polynucleotide adduct impurity can be prepared by methods that inhibit formation of one or both of N-oxides and aldehydes. Such methods may comprise treating a composition comprising an ionizable lipid comprising a tertiary amine group to inhibit formation of one or both of N-oxides and aldehydes, such as by treating the composition with a reducing agent; treating the composition with a chelating agent; adjusting the pH of the composition; adjusting the temperature of the composition; and adjusting the buffer in the composition. Such methods may comprise, prior to combining the ionizable lipid with a polynucleotide, one or more of treating the ionizable lipid with a scavenging agent; treating the ionizable lipid with a reductive treatment agent; treating the ionizable lipid with a reducing agent; treating the ionizable lipid with a chelating agent; treating the polynucleotide with a reducing agent; and treating the polynucleotide with a chelating agent.

In accordance with any of the foregoing, the scavenging agent, reductive treatment agent, and/or reducing agent may be an agent that reacts with aldehyde, ketone, anhydride and/or diene compounds. A scavenging agent may comprise one or more selected from (O-(2,3,4,5,6-Pentafluorobenzyl)hydroxylamine hydrochloride) (PFBHA), methoxyamine (e.g., methoxyamine hydrochloride), benzyloxyamine (e.g., benzyloxyamine hydrochloride), ethoxyamine (e.g., ethoxyamine hydrochloride), 4-[2-(aminooxy)ethyl]morpholine dihydrochloride, butoxyamine (e.g., tert-butoxyamine hydrochloride), 4-Dimethylaminopyridine (DMAP), 1,4-diazabicyclo[2.2.2]octane (DABCO), Triethylamine (TEA), Piperidine 4-carboxylate (BPPC), and combinations thereof. A reductive treatment agent may comprise a boron compound (e.g., sodium borohydride and/or bis(pinacolato)diboron). A reductive treatment agent may comprise a boron compound, such as one or both of sodium borohydride and bis(pinacolato)diboron). A chelating agent may comprise immobilized iminodiacetic acid. A reducing agent may comprise an immobilized reducing agent, such as immobilized diphenylphosphine on silica (Si-DPP), immobilized thiol on agarose (Ag-Thiol), immobilized cysteine on silica (Si-Cysteine), immobilized thiol on silica (Si-Thiol), or a combination thereof. A reducing agent may comprise a free reducing agent, such as potassium metabisulfite, sodium thioglycolate, tris(2-carboxyethyl)phosphine (TCEP), sodium thiosulfate, N-acetyl cysteine, glutathione, dithiothreitol (DTT), cystamine, dithioerythritol (DTE), dichlorodiphenyltrichloroethane (DDT), homocysteine, lipoic acid, or a combination thereof.

In accordance with any of the foregoing, the pH may be, or adjusted to be, a pH of from about 7 to about 9.

In accordance with any of the foregoing, a buffer may be selected from sodium phosphate, sodium citrate, sodium succinate, histidine, histidine-HCl, sodium malate, sodium carbonate, and TRIS (tris(hydroxymethyl)aminomethane). In accordance with any of the foregoing, a buffer may be TRIS and may be, or adjusted to be, from about 20 mM to about 150 mM TRIS.

In accordance with any of the foregoing, the temperature of the composition may be, or adjusted to be, 25° C. or less.

The composition may also comprise a free reducing agent or antioxidant.

### Pharmaceutical Formulations

Provided herein are compositions (e.g., pharmaceutical compositions, such as vaccines), methods, kits and reagents for prevention or treatment of EBV in humans and other mammals, for example. In some embodiments, an EBV mRNA vaccine is administered to a subject to prevent EBV infection or a disease associated with EBV infection (e.g., infectious mononucleosis, nasopharyngeal carcinoma, Burkitt lymphoma, Hodgkin's lymphoma, gastric cancer, posttransplant lymphoproliferative disorders (PTLDs), systemic lupus erythematosus, and/or multiple sclerosis). In some embodiments, an EBV mRNA vaccine is administered to a subject to treat EBV infection or a disease associated with EBV infection (e.g., infectious mononucleosis, nasopharyngeal carcinoma, Burkitt lymphoma, Hodgkin's lymphoma, gastric cancer, posttransplant lymphoproliferative disorders (PTLDs), systemic lupus erythematosus, and/or multiple sclerosis). The vaccine compositions provided herein can be used as therapeutic or prophylactic agents. They may be used in medicine to prevent and/or treat an EBV infection or a disease associated with EBV infection.

In some embodiments, the vaccine compositions containing mRNA as described herein can be administered to a subject (e.g., a mammalian subject, such as a human subject), and the mRNA are translated in vivo to produce an antigenic polypeptide (antigen).

An “effective amount” of a composition, such as an mRNA vaccine, is based, at least in part, on the target tissue, target cell type, means of administration, physical characteristics of the mRNA (e.g., length, nucleotide composition, and/or extent of modified nucleosides), other components of the vaccine, and other determinants, such as age, body weight, height, sex and general health of the subject. Typically, an effective amount of a composition provides an induced or boosted immune response as a function of antigen production in the cells of the subject. In some embodiments, an effective amount of the composition containing mRNA having at least one chemical modification are more efficient than a composition containing a corresponding unmodified polynucleotide encoding the same antigen or a peptide antigen. Increased antigen production may be demonstrated by increased cell transfection (the percentage of cells transfected with the mRNA vaccine), increased protein translation and/or expression from the polynucleotide, decreased nucleic acid degradation (as demonstrated, for example, by increased duration of protein translation from a modified polynucleotide), or altered antigen specific immune response of the host cell.

The term “pharmaceutical composition” refers to the combination of an active agent (e.g., mRNA) with a carrier (e.g., lipid composition, e.g., LNP)), inert or active, making the composition especially suitable for diagnostic or therapeutic use in vivo or ex vivo. A “pharmaceutically acceptable carrier,” after administered to or upon a subject, does not cause undesirable physiological effects. The carrier in the pharmaceutical composition must be “acceptable” also in the sense that it is compatible with the active ingredient and can be capable of stabilizing it. One or more solubilizing agents can be utilized as pharmaceutical carriers for delivery of an active agent. Examples of a pharmaceutically acceptable carrier include, but are not limited to, biocompatible vehicles, adjuvants, additives, and diluents to achieve a composition usable as a dosage form. Examples of other carriers include colloidal silicon oxide, magnesium stearate, cellulose, and sodium lauryl sulfate. Additional suitable pharmaceutical carriers and diluents, as well as pharmaceutical necessities for their use, are described in Remington's Pharmaceutical Sciences.

In some embodiments, the compositions (comprising polynucleotides and their encoded polypeptides) in accordance with the present disclosure may be used for treatment or prevention of an EBV infection. A composition may be administered prophylactically or therapeutically as part of an active immunization scheme to healthy individuals or early in infection during the incubation phase or during active infection after onset of symptoms. In some embodiments, the amount of mRNA provided to a cell, a tissue or a subject may be an amount effective for immune prophylaxis.

A composition may be administered with other prophylactic or therapeutic compounds. As a non-limiting example, a prophylactic or therapeutic compound may be an adjuvant or a booster. As used herein, when referring to a prophylactic composition, such as a vaccine, the term “booster” refers to an extra administration of the prophylactic (vaccine) composition. A booster (or booster vaccine) may be given after an earlier administration of the prophylactic composition. The time of administration between the initial administration of the prophylactic composition and the booster may be, but is not limited to, 12 hours, 1 day, 36 hours, 2 days, 3 days, 4 days, 5 days, 6 days, 1 week, 10 days, 2 weeks, 3 weeks, 1 month, 2 months, 3 months, 4 months, 5 months, 6 months, 7 months, 8 months, 9 months, 10 months, 11 months, 1 year, 18 months, 2 years, 3 years, 4 years, 5 years, 6 years, 7 years, 8 years, 9 years, 10 years, or more. In exemplary embodiments, the time of administration between the initial administration of the prophylactic composition and the booster may be, but is not limited to, 1 week, 2 weeks, 3 weeks, 1 month, 2 months, 3 months, or 6 months. As is described herein, the booster may comprise the same or different mRNA as compared to the earlier administration of the prophylactic composition. The booster, in some embodiments is monovalent (e.g., the mRNA encodes a single antigen). In some embodiments, the booster is multivalent (e.g., the mRNA encodes more than one antigen).

In some embodiments, “administering” or “administration” means providing a material to a subject in a manner that is pharmacologically useful. In some embodiments, an mRNA vaccine disclosed herein is administered to a subject enterally. In some embodiments, an enteral administration of the composition is oral. In some embodiments, an mRNA vaccine disclosed herein is administered to the subject parenterally. In some embodiments, an mRNA vaccine disclosed herein is administered to a subject subcutaneously, intraocularly, intravitreally, subretinally, intravenously (IV), intracerebro-ventricularly, intramuscularly, intranasally, intrathecally (IT), intracisternally, intraperitoneally, via inhalation, topically, or by direct injection to one or more cells, tissues, or organs.

A composition may be utilized in various settings depending on the prevalence of the infection or the degree or level of unmet medical need. As a non-limiting example, the mRNA vaccines may be utilized to treat and/or prevent EBV. mRNA vaccines have superior properties in that they produce much larger antibody titers, better neutralizing immunity, produce more durable immune responses, and/or produce responses earlier than commercially available vaccines.

Provided herein are pharmaceutical compositions including mRNA and/or complexes optionally in combination with one or more pharmaceutically acceptable excipients.

The mRNA may be formulated or administered alone or in conjunction with one or more other components. For example, a vaccine may comprise other components including, but not limited to, adjuvants.

In some embodiments, a vaccine does not include an adjuvant (they are adjuvant free).

An mRNA may be formulated or administered in combination with one or more pharmaceutically-acceptable excipients. In some embodiments, vaccine compositions comprise at least one additional active substance, such as, for example, a therapeutically-active substance, a prophylactically-active substance, or a combination of both. Vaccine compositions may be sterile, pyrogen-free or both sterile and pyrogen-free. General considerations in the formulation and/or manufacture of pharmaceutical agents, such as vaccine compositions, may be found, for example, in Remington: The Science and Practice of Pharmacy 21st ed., Lippincott Williams & Wilkins, 2005 (incorporated herein by reference in its entirety).

In some embodiments, a vaccine is administered to humans, human patients or subjects. For the purposes of the present disclosure, the phrase “active ingredient” generally refers to the mRNA contained therein, for example, mRNA encoding EBV protein antigens.

Formulations of the vaccine compositions described herein may be prepared by any method known or hereafter developed in the art of pharmacology. In general, such preparatory methods include the step of bringing the active ingredient (e.g., mRNA) into association with an excipient and/or one or more other accessory ingredients, and then, if necessary and/or desirable, dividing, shaping and/or packaging the product into a desired single- or multi-dose unit.

Relative amounts of the active ingredient, the pharmaceutically acceptable excipient, and/or any additional ingredients in a pharmaceutical composition in accordance with the disclosure will vary, depending upon the identity, size, and/or condition of the subject treated and further depending upon the route by which the composition is to be administered. By way of example, the composition may comprise between 0.1% and 100%, e.g., between 0.5 and 50%, between 1-30%, between 5-80%, at least 80% (w/w) active ingredient.

In some embodiments, an mRNA is formulated using one or more excipients to: (1) increase stability; (2) increase cell transfection; (3) permit the sustained or delayed release (e.g., from a depot formulation); (4) alter the biodistribution (e.g., target to specific tissues or cell types); (5) increase the translation of encoded protein in vivo; and/or (6) alter the release profile of encoded protein (antigen) in vivo. In addition to traditional excipients such as any and all solvents, dispersion media, diluents, or other liquid vehicles, dispersion or suspension aids, surface active agents, isotonic agents, thickening or emulsifying agents, preservatives, excipients can include, without limitation, lipidoids, liposomes, lipid nanoparticles, polymers, lipoplexes, core-shell nanoparticles, peptides, proteins, cells transfected with the mRNA (e.g., for transplantation into a subject), hyaluronidase, nanoparticle mimics and combinations thereof.

### Methods of Prophylaxis/Treatment

Epstein-Barr Virus (EBV) is a complex, large, double-stranded DNA gamma-herpesvirus with high rates of seroprevalence (>90%) in adults worldwide. It is responsible for 90% of Infectious mononucleosis (IM) cases worldwide, most commonly in adolescents and young adults contracting a primary infection. IM is a clinical syndrome that presents with fever, fatigue, sore throat, and lymphadenopathy and can result in prolonged symptoms as well as hospitalization and splenic rupture. EBV has also been associated with other serious diseases including infectious mononucleosis, nasopharyngeal carcinoma, Burkitt lymphoma, Hodgkin's lymphoma, gastric cancer, posttransplant lymphoproliferative disorders (PTLDs) and, more recently, systemic lupus erythematosus and multiple sclerosis. The mRNA vaccines provided here may be used to treat and/or prevent such diseases associated with EBV infection.

Provided herein are mRNA vaccines, methods, kits and reagents for prevention and/or treatment of an EBV infection in humans and other mammals. The mRNA vaccines can be used as therapeutic or prophylactic agents. In some embodiments, the mRNA vaccines are used to provide prophylactic protection from an EBV infection. In some embodiments, the mRNA vaccines are used to treat an EBV infection. In some embodiments, the mRNA vaccines are used in the priming of immune effector cells, for example, to activate peripheral blood mononuclear cells (PBMCs) ex vivo, which are then infused (re-infused) into a subject.

A subject may be any mammal, including non-human primate and human subjects. Typically, a subject is a human subject.

In some embodiments, an mRNA vaccine is administered to a subject (e.g., a mammalian subject, such as a human subject) in an effective amount to induce an antigen-specific immune response. The RNA encoding the EBV protein is expressed and translated in vivo to produce the antigen, which then stimulates an immune response in the subject.

In some embodiments, an mRNA vaccine is administered to a subject that has been infected with EBV. In some embodiments, the subject has received, or is scheduled to receive, a solid organ transplant or a hematopoietic stem cell transplant. In some embodiments, the subject has or is at risk of having, a posttransplant lymphoproliferative disorder. In some embodiments, the subject has, or is at risk of having, infectious mononucleosis. In some embodiments, the subject has or is at risk of having, multiple sclerosis. In some embodiments, the subject has, or is at risk of having, systemic lupus erythematosus. In some embodiments, the subject has, or is at risk of having, cancer. In some embodiments, the cancer is selected from Burkitt lymphoma, Hodgkin's lymphoma, nasopharyngeal carcinoma, and gastric cancer. In some embodiments, the subject is EBV-seropositive. In some embodiments, the subject the subject is EBV-seronegative. In some embodiments, the subject is 18 to 55 years old.

In some embodiments, the subject is about 18-55 (e.g., 18-100, 18-90, 18-80, 18-70, 18-60, 18-55, 18-50, 18-25, 25-100, 25-90, 25-80, 25-70, 25-60, 25-55, 25-50, 50-100, 50-90, 50-80, 50-70, 50-60, 50-55, 55-100, 55-90, 55-80, 55-70, 55-60, 60-100, 60-90, 60-80, 60-70, 70-100, 70-90, 70-80, 80-100, 80-90, 90-100) years old. In some embodiments, the subject is about 18-100, 18-90, 18-80, 18-70, 18-60, 18-55, 18-50, 18-25, 25-100, 25-90, 25-80, 25-70, 25-60, 25-55, 25-50, 50-100, 50-90, 50-80, 50-70, 50-60, 50-55, 55-100, 55-90, 55-80, 55-70, 55-60, 60-100, 60-90, 60-80, 60-70, 70-100, 70-90, 70-80, 80-100, 80-90, 90-100 years old. In some embodiments, the subject is about 18 to 55 years old.

Prophylactic protection from an EBV can be achieved following administration of an mRNA vaccine. The compositions can be administered once, twice, three times, four times or more but it is likely sufficient to administer the composition once (optionally followed by a single booster). It is possible, although less desirable, to administer an mRNA vaccine to an infected individual to achieve a therapeutic response. Dosing may need to be adjusted accordingly.

A method of eliciting an immune response in a subject against an EBV protein (or multiple antigens) is provided in aspects of the present disclosure. In some embodiments, a method involves administering to the subject a vaccine comprising a mRNA having an open reading frame encoding an EBV protein (or multiple antigens), thereby inducing in the subject an immune response specific to the EBV protein (or multiple antigens), wherein anti-antigen antibody titer in the subject is increased following vaccination relative to anti-antigen antibody titer in a subject vaccinated with a prophylactically effective dose of a traditional vaccine against the antigen. An “anti-antigen antibody” is a serum antibody the binds specifically to the antigen.

A prophylactically effective dose is an effective dose that prevents infection with the virus at a clinically acceptable level. In some embodiments, the effective dose is a dose listed in a package insert for the vaccine. A traditional vaccine, as used herein, refers to a vaccine other than the mRNA vaccines of the present disclosure. For instance, a traditional vaccine includes, but is not limited, to live microorganism vaccines, killed microorganism vaccines, subunit vaccines, protein antigen vaccines, DNA vaccines, virus like particle (VLP) vaccines, etc. In exemplary embodiments, a traditional vaccine is a vaccine that has achieved regulatory approval and/or is registered by a national drug regulatory body, for example the Food and Drug Administration (FDA) in the United States or the European Medicines Agency (EMA).

In some embodiments, the anti-antigen antibody titer in the subject is increased 1 log to 10 log following vaccination relative to anti-antigen antibody titer in a subject vaccinated with a prophylactically effective dose of a traditional vaccine against EBV or an unvaccinated subject. In some embodiments, the anti-antigen antibody titer in the subject is increased 1 log, 2 log, 3 log, 4 log, 5 log, or 10 log following vaccination relative to anti-antigen antibody titer in a subject vaccinated with a prophylactically effective dose of a traditional vaccine against EBV or an unvaccinated subject.

A method of eliciting an immune response in a subject against EBV is provided in other aspects of the disclosure. The method involves administering to the subject an mRNA vaccine comprising an mRNA comprising an open reading frame encoding an EBV protein, thereby inducing in the subject an immune response specific to EBV, wherein the immune response in the subject is equivalent to an immune response in a subject vaccinated with a traditional vaccine against EBV at 2 times to 100 times the dosage level relative to the composition.

In some embodiments, the immune response in the subject is equivalent to an immune response in a subject vaccinated with a traditional vaccine at twice the dosage level relative to an mRNA vaccine of the present disclosure. In some embodiments, the immune response in the subject is equivalent to an immune response in a subject vaccinated with a traditional vaccine at three times the dosage level relative to an mRNA vaccine of the present disclosure. In some embodiments, the immune response in the subject is equivalent to an immune response in a subject vaccinated with a traditional vaccine at 4 times, 5 times, 10 times, 50 times, or 100 times the dosage level relative to an mRNA vaccine of the present disclosure. In some embodiments, the immune response in the subject is equivalent to an immune response in a subject vaccinated with a traditional vaccine at 10 times to 1000 times the dosage level relative to an mRNA vaccine of the present disclosure. In some embodiments, the immune response in the subject is equivalent to an immune response in a subject vaccinated with a traditional vaccine at 100 times to 1000 times the dosage level relative to an mRNA vaccine of the present disclosure.

In other embodiments, the immune response is assessed by determining [protein]antibody titer in the subject. In other embodiments, the ability of serum or antibody from an immunized subject is tested for its ability to neutralize viral uptake or reduce EBV transformation of human B lymphocytes. In other embodiments, the ability to promote a robust T cell response(s) is measured using art recognized techniques.

Other aspects the disclosure provide methods of eliciting an immune response in a subject against EBV by administering to the subject composition comprising an mRNA having an open reading frame encoding an EBV protein, thereby inducing in the subject an immune response specific to the EBV protein, wherein the immune response in the subject is induced 2 days to 10 weeks earlier relative to an immune response induced in a subject vaccinated with a prophylactically effective dose of a traditional vaccine against EBV. In some embodiments, the immune response in the subject is induced in a subject vaccinated with a prophylactically effective dose of a traditional vaccine at 2 times to 100 times the dosage level relative to an mRNA vaccine of the present disclosure.

In some embodiments, the immune response in the subject is induced 2 days, 3 days, 1 week, 2 weeks, 3 weeks, 5 weeks, or 10 weeks earlier relative to an immune response induced in a subject vaccinated with a prophylactically effective dose of a traditional vaccine.

Also provided herein are methods of eliciting an immune response in a subject against EBV by administering to the subject an mRNA having an open reading frame encoding at least one EBV protein, wherein the mRNA does not include a stabilization element, and wherein an adjuvant is not co-formulated or co-administered with the vaccine.

A composition may be administered by any route that results in a therapeutically effective outcome. These include, but are not limited, to intradermal, intramuscular, intranasal, and/or subcutaneous administration. The present disclosure provides methods comprising administering mRNA vaccines to a subject in need thereof. The exact amount required will vary from subject to subject, depending on the species, age, and general condition of the subject, the severity of the disease, the particular composition, its mode of administration, its mode of activity, and the like. The mRNA is typically formulated in dosage unit form for ease of administration and uniformity of dosage. It will be understood, however, that the total daily usage of the mRNA may be decided by the attending physician within the scope of sound medical judgment. The specific therapeutically effective, prophylactically effective, or appropriate imaging dose level for any particular patient will depend upon a variety of factors including the disorder being treated and the severity of the disorder; the activity of the specific compound employed; the specific composition employed; the age, body weight, general health, sex and diet of the patient; the time of administration, route of administration, and rate of excretion of the specific compound employed; the duration of the treatment; drugs used in combination or coincidental with the specific compound employed; and like factors well known in the medical arts.

The effective amount of the mRNA (e.g., an effective dose), as provided herein, may be, for example, about 25 μg to about 150 μg (e.g., administered as a single dose), about 50 μg to about 300 μg (e.g., administered as two separate doses), or about 75 μg to about 450 μg (e.g., administered as three separate doses). In some embodiments, a single dose of the mRNA vaccine is 25 μg (which may be administered once, twice or three or more times). In some embodiments, a single dose of the mRNA vaccine is 50 μg (which may be administered once, twice or three or more times). In some embodiments, a single dose of the mRNA vaccine is 75 μg (which may be administered once, twice or three or more times). In some embodiments, a single dose of the mRNA vaccine is 100 μg (which may be administered once, twice or three or more times). In some embodiments, a single dose of the mRNA vaccine is 125 μg (which may be administered once, twice or three or more times). In some embodiments, a single dose of the mRNA vaccine is 150 μg (which may be administered once, twice or three or more times). In some embodiments, two doses of the mRNA vaccine are administered. In some embodiments, three doses of the mRNA vaccine are administered. In some embodiments, the first dose and the second dose are the same amount. In some embodiments, the first dose, the second dose, and the third dose are the same amount. In some embodiments, the first dose and the second dose are different amounts. In some embodiments, the first dose and the second dose are different amounts. In some embodiments, escalating doses of the mRNA vaccine are used.

When multiple (booster) doses are administered, the multiple doses may be administered several weeks or several months apart. For example, two different doses may be administered about 2, about 3, about 4, about 5, or about 6 weeks apart. In some embodiments, two different doses are administered about 1 month, about 2 months, about 3 months, about 4 months, about 5 months, or about 6 months apart.

The mRNA described herein can be formulated into a dosage form described herein, such as an intranasal, intratracheal, or injectable (e.g., intravenous, intraocular, intravitreal, intramuscular, intradermal, intracardiac, intraperitoneal, and subcutaneous).

### Vaccine Efficacy

Some aspects provide formulations of the compositions (e.g., RNA vaccines), wherein the mRNA is formulated in an effective amount to produce an antigen specific immune response in a subject (e.g., production of antibodies specific to an EBV antigen). “An effective amount” is a dose of the mRNA effective to produce an antigen-specific immune response. Also provided herein are methods of inducing an antigen-specific immune response in a subject.

As used herein, an immune response to an mRNA vaccine is the development in a subject of a humoral and/or a cellular immune response to a (one or more) EBV protein(s) encoded by the mRNA present in the vaccine. For purposes of the present disclosure, a “humoral” immune response refers to an immune response mediated by antibody molecules, including, e.g., secretory (IgA) or IgG molecules, while a “cellular” immune response is one mediated by T-lymphocytes (e.g., CD4+ helper and/or CD8+ T cells (e.g., CTLs) and/or other white blood cells. One important aspect of cellular immunity involves an antigen-specific response by cytolytic T-cells (CTLs). CTLs have specificity for peptide antigens that are presented in association with proteins encoded by the major histocompatibility complex (MHC) and expressed on the surfaces of cells. CTLs help induce and promote the destruction of intracellular microbes or the lysis of cells infected with such microbes. Another aspect of cellular immunity involves and antigen-specific response by helper T-cells. Helper T-cells act to help stimulate the function and focus the activity nonspecific effector cells against cells displaying peptide antigens in association with MHC molecules on their surface. A cellular immune response also leads to the production of cytokines, chemokines, and other such molecules produced by activated T-cells and/or other white blood cells including those derived from CD4+ and CD8+ T-cells. Cellular immune responses may be further divided into Th1 and Th2 responses, resulting the production of Th1-type cytokines and Th2-type cytokines, respectively. Th1-type cytokines tend to produce the proinflammatory responses responsible for killing intracellular parasites (e.g., viruses) and for perpetuating autoimmune responses. The main Th1 cytokine is interferon gamma (IFN-γ). Proinflammatory responses (e.g., Th1-based responses), in some embodiments, are counteracted by the Th2-type cytokines. The Th2-type cytokines include interleukins 4, 5, and 13, which are associated with the promotion of IgE and eosinophilic responses in atopy, and also interleukin-10, which is anti-inflammatory. In excess, Th2 responses will counteract the Th1-mediated microbicidal action. In some embodiments, Th2 responses balance excess Th1 responses to mitigate tissue damage due to inflammation. However, Th2 responses also hinder the antiviral activity of Th1 cells, which may be deleterious in EBV infection. In some embodiments, administration of the vaccines provided herein may result in a Th17 response. T helper 17 cells (Th17) are a subset of pro-inflammatory T helper cells defined by their production of interleukin 17. Th17 cells maintain mucosal barriers and contribute to pathogen clearance at the mucosal surfaces. The Th17-type cytokines target innate immune cells and epithelial cells to produce G-CSF and Il-8, leading to neutrophil production and recruitment. In some embodiments, the mRNA vaccines produce a Th1 response. In some embodiments, the mRNA vaccines produce a Th2 response. In some embodiments, the mRNA vaccines produce a Th17 response. In some embodiments, the mRNA vaccines produce Th1 and Th2 responses, Th1 and Th17 responses, Th2 and Th17 responses, or Th1, Th2, and Th17 responses.

Some embodiments of the vaccines described herein elicit T cell responses that are polarized towards a Th1 phenotype (i.e., include more EBV-specific Th1 cells than EBV-specific Th2 cells). Polarization towards a Th1 phenotype is beneficial for preventing or treating EBV infection, at least in part because Th1 cells secrete pro-inflammatory cytokines including IFN-γ and TNF-α, which promote phagocytosis of virions and clearance of infected cells. By contrast Th2 cells and the cytokines they secrete (e.g., IL-4, IL-5, IL-9, IL-10, or IL-13) are pathogenic in EBV-2 infection, increasing morbidity and mortality rather than contributing to viral control or clearance. See, e.g., Nakajima et al., J Neuroimmunol. 2000. 110(1-2):106-113. Thus, polarizing the CD4+ T cell response towards a Th1 phenotype increases the prophylactic and/or therapeutic efficacy of the EBV vaccines described herein. In some embodiments, polarization towards a Th1 phenotype is characterized by at least 50% of CD4+ T cells specific to an EBV antigen encoded by an mRNA of the vaccine (EBV-specific CD4+ T cells) producing a Th1 cytokine (e.g., IFN-γ, TNF-α, and/or IL-2). In some embodiments, at least 50% of CD4+ T cells specific to EBV gB produce a Th1 cytokine. In some embodiments, at least 50% of CD4+ T cells specific to EBV gC produce a Th1 cytokine. In some embodiments, at least 50% of CD4+ T cells specific to EBV gD produce a Th1 cytokine. In some embodiments, at least 50%, at least 60%, at least 70%, at least 75%, at least 80%, at least 85%, at least 90%, at least 95%, or up to 100% of EBV-specific CD4+ T cells produce IFN-γ. In some embodiments, at least 50%, at least 60%, at least 70%, at least 75%, at least 80%, at least 85%, at least 90%, at least 95%, or up to 100% of EBV-specific CD4+ T cells produce TNF-α. In some embodiments, at least 50%, at least 60%, at least 70%, at least 75%, at least 80%, at least 85%, at least 90%, at least 95%, or up to 100% of EBV-specific CD4+ T cells produce IL-2. In some embodiments, polarization towards a Th1 phenotype is characterized by fewer than 50% of EBV-specific CD4+ T cells producing a Th2 cytokine (e.g., IL-4, IL-5, and/or IL-13). In some embodiments, fewer than 50%, fewer than 40%, fewer than 30%, fewer than 25%, fewer than 20%, fewer than 15%, fewer than 10%, fewer than 5%, or as few as 0% of EBV-specific CD4+ T cells produce IL-4. In some embodiments, fewer than 50%, fewer than 40%, fewer than 30%, fewer than 25%, fewer than 20%, fewer than 15%, fewer than 10%, fewer than 5%, or as few as 0% of EBV-specific CD4+ T cells produce IL-5. In some embodiments, fewer than 50%, fewer than 40%, fewer than 30%, fewer than 25%, fewer than 20%, fewer than 15%, fewer than 10%, fewer than 5%, or as few as 0% of EBV-specific CD4+ T cells produce IL-13.

In some embodiments, the antigen-specific immune response is characterized by measuring an anti-herpesvirus (e.g., anti-EBV) antigen antibody titer produced in a subject administered an mRNA vaccine as provided herein. An antibody titer is a measurement of the amount of antibodies within a subject, for example, antibodies that are specific to a particular antigen or epitope of an antigen. Antibody titer is typically expressed as the inverse of the greatest dilution that provides a positive result. Enzyme-linked immunosorbent assay (ELISA) is a common assay for determining antibody titers, for example.

A variety of serological tests can be used to measure antibody against encoded antigen of interest, for example, an EBV antigen. These tests include the hemagglutination-inhibition test, complement fixation test, fluorescent antibody test, enzyme-linked immunosorbent assay (ELISA), and plaque reduction neutralization test (PRNT). Each of these tests measures different antibody activities. In exemplary embodiments, A plaque reduction neutralization test, or PRNT (e.g., PRNT50 or PRNT90) is used as a serological correlate of protection. PRNT measures the biological parameter of in vitro virus neutralization and is the most serologically virus-specific test among certain classes of viruses, correlating well to serum levels of protection from virus infection. The basic design of the PRNT allows for virus-antibody interaction to occur in a test tube or microtiter plate, and then measuring antibody effects on viral infectivity by plating the mixture on virus-susceptible cells, preferably cells of mammalian origin. The cells are overlaid with a semi-solid media that restricts spread of progeny virus. Each virus that initiates a productive infection produces a localized area of infection (a plaque), that can be detected in a variety of ways. Plaques are counted and compared back to the starting concentration of virus to determine the percent reduction in total virus infectivity. In PRNT, the serum sample being tested is usually subjected to serial dilutions prior to mixing with a standardized amount of virus. The concentration of virus is held constant such that, when added to susceptible cells and overlaid with semi-solid media, individual plaques can be discerned and counted. In this way, PRNT end-point titers can be calculated for each serum sample at any selected percent reduction of virus activity.

In functional assays intended to assess vaccinal immunogenicity, the serum sample dilution series for antibody titration should ideally start below the “seroprotective” threshold 10 titer. Regarding anti-herpesvirus (e.g., anti-EBV) neutralizing antibodies, a seropositivity threshold of 1:10 can be considered a seroprotection threshold in certain embodiments.

PRNT end-point titers are expressed as the reciprocal of the last serum dilution showing the desired percent reduction in plaque counts. The PRNT titer can be calculated based on a 50% or greater reduction in plaque counts (PRNT₅₀). A PRNT₅₀ titer is preferred over titers using higher cut-offs (e.g., PRNT₉₀, reflecting 90% or greater reduction) for vaccine sera, providing more accurate results from the linear portion of the titration curve.

There are several ways to calculate PRNT titers. The simplest and most widely used way to calculate titers is to count plaques and report the titer as the reciprocal of the last serum dilution to show >50% reduction of the input plaque count as based on the back-titration of input plaques. Use of curve fitting methods from several serum dilutions may permit calculation of a more precise result. There are a variety of computer analysis programs available for this (e.g., SPSS or GraphPad Prism).

In some embodiments, an antibody titer is used to assess whether a subject has had an infection or to determine whether immunizations are required. In some embodiments, an antibody titer is used to determine the strength of an autoimmune response, to determine whether a booster immunization is needed, to determine whether a previous vaccine was effective, and to identify any recent or prior infections. In accordance with the present disclosure, an antibody titer may be used to determine the strength of an immune response induced in a subject by an RNA vaccine.

In some embodiments, the anti-herpesvirus (e.g., anti-EBV) antigen antibody titer produced in a subject is increased by at least 1 log relative to a control. For example, the anti-herpesvirus (e.g., anti-EBV) antigen antibody titer produced in a subject may be increased by at least 1.5, at least 2, at least 2.5, or at least 3 log relative to a control. In some embodiments, the anti-herpesvirus (e.g., anti-EBV) antigen antibody titer produced in the subject is increased by 1, 1.5, 2, 2.5 or 3 log relative to a control. In some embodiments, the anti-herpesvirus (e.g., anti-EBV) antigen antibody titer produced in the subject is increased by 1-3 log relative to a control. For example, the anti-herpesvirus (e.g., anti-EBV) antigen antibody titer produced in a subject may be increased by 1-1.5, 1-2, 1-2.5, 1-3, 1.5-2, 1.5-2.5, 1.5-3, 2-2.5, 2-3, or 2.5-3 log relative to a control.

In some embodiments, the anti-herpesvirus (e.g., anti-EBV) antigen antibody titer produced in a subject is increased at least 2 times relative to a control. For example, the anti-herpesvirus (e.g., anti-EBV) antigen antibody titer produced in a subject may be increased at least 3 times, at least 4 times, at least 5 times, at least 6 times, at least 7 times, at least 8 times, at least 9 times, or at least 10 times relative to a control. In some embodiments, the anti-herpesvirus (e.g., anti-EBV) antigen antibody titer produced in the subject is increased 2, 3, 4, 5, 6, 7, 8, 9, or 10 times relative to a control. In some embodiments, the anti-herpesvirus (e.g., anti-EBV) antigen antibody titer produced in a subject is increased 2-10 times relative to a control. For example, the anti-herpesvirus (e.g., anti-EBV) antigen antibody titer produced in a subject may be increased 2-10, 2-9, 2-8, 2-7, 2-6, 2-5, 2-4, 2-3, 3-10, 3-9, 3-8, 3-7, 3-6, 3-5, 3-4, 4-10, 4-9, 4-8, 4-7, 4-6, 4-5, 5-10, 5-9, 5-8, 5-7, 5-6, 6-10, 6-9, 6-8, 6-7, 7-10, 7-9, 7-8, 8-10, 8-9, or 9-10 times relative to a control.

In some embodiments, an antigen-specific immune response is measured as a ratio of geometric mean titer (GMT), referred to as a geometric mean ratio (GMR), of serum neutralizing antibody titers to a herpesvirus (e.g., EBV). A geometric mean titer (GMT) is the average antibody titer for a group of subjects calculated by multiplying all values and taking the nth root of the number, where n is the number of subjects with available data.

A control, in some embodiments, is an anti-herpesvirus (e.g., anti-EBV) antigen antibody titer produced in a subject who has not been administered an mRNA vaccine. In some embodiments, a control is an anti-herpesvirus (e.g., anti-EBV) antigen antibody titer produced in a subject administered a recombinant or purified protein vaccine. Recombinant protein vaccines typically include protein antigens that either have been produced in a heterologous expression system (e.g., bacteria or yeast) or purified from large amounts of the pathogenic organism.

In some embodiments, the ability of an mRNA vaccine to be effective is measured in a murine model. For example, an mRNA vaccine may be administered to a murine model and the murine model assayed for induction of neutralizing antibody titers. Bacterial challenge studies may also be used to assess the efficacy of an mRNA vaccine. For example, an mRNA vaccine may be administered to a murine model, the murine model challenged with virus, and the murine model assayed for survival and/or immune response (e.g., neutralizing antibody response, T cell response (e.g., cytokine response)).

In some embodiments, an effective amount of an mRNA vaccine is a dose that is reduced compared to the standard of care dose of a recombinant protein vaccine. A “standard of care,” as provided herein, refers to a medical or psychological treatment guideline and can be general or specific. “Standard of care” specifies appropriate treatment based on scientific evidence and collaboration between medical professionals involved in the treatment of a given condition. It is the diagnostic and treatment process that a physician/clinician should follow for a certain type of patient, illness or clinical circumstance. A “standard of care dose,” as provided herein, refers to the dose of a recombinant or purified protein vaccine, or a live attenuated or inactivated vaccine, or a VLP vaccine, that a physician/clinician or other medical professional would administer to a subject to treat or prevent EBV infections or a related condition, while following the standard of care guideline for treating or preventing EBV infection or a related condition.

In some embodiments, the anti-herpesvirus (e.g., anti-EBV) antigen antibody titer produced in a subject administered an effective amount of an mRNA vaccine is equivalent to the anti-herpesvirus (e.g., anti-EBV) antigen antibody titer produced in a control subject administered a standard of care dose of a recombinant or purified protein vaccine, or a live attenuated or inactivated vaccine, or a VLP vaccine.

Vaccine efficacy may be assessed using standard analyses (see, e.g., Weinberg et al., J Infect Dis. 2010 Jun. 1; 201(11):1607-10). For example, vaccine efficacy may be measured by double-blind, randomized, clinical controlled trials. Vaccine efficacy may be expressed as a proportionate reduction in disease attack rate (AR) between the unvaccinated (ARU) and vaccinated (ARV) study cohorts and can be calculated from the relative risk (RR) of disease among the vaccinated group with use of the following formulas:

$$Efficacy = ( ARU - ARV ) / ARU \times 100 ; and$$

$$Efficacy = ( 1 - RR ) \times 100.$$

Likewise, vaccine effectiveness may be assessed using standard analyses (see, e.g., Weinberg et al., J Infect Dis. 2010 Jun. 1; 201(11):1607-10). Vaccine effectiveness is an assessment of how a vaccine (which may have already proven to have high vaccine efficacy) reduces disease in a population. This measure can assess the net balance of benefits and adverse effects of a vaccination program, not just the vaccine itself, under natural field conditions rather than in a controlled clinical trial. Vaccine effectiveness is proportional to vaccine efficacy (potency) but is also affected by how well target groups in the population are immunized, as well as by other non-vaccine-related factors that influence the ‘real-world’ outcomes of hospitalizations, ambulatory visits, or costs. For example, a retrospective case control analysis may be used, in which the rates of vaccination among a set of infected cases and appropriate controls are compared. Vaccine effectiveness may be expressed as a rate difference, with use of the odds ratio (OR) for developing infection despite vaccination:

$$Effectiveness = ( 1 - OR ) \times 100.$$

In some embodiments, efficacy of an mRNA vaccine is at least 60% relative to unvaccinated control subjects. For example, efficacy of the composition may be at least 65%, at least 70%, at least 75%, at least 80%, at least 85%, at least 95%, at least 98%, or 100% relative to unvaccinated control subjects.

Sterilizing Immunity. Sterilizing immunity refers to a unique immune status that prevents effective pathogen infection into the host. In some embodiments, the effective amount of an mRNA vaccine is sufficient to provide sterilizing immunity in the subject for at least 1 year. For example, the effective amount of an mRNA vaccine is sufficient to provide sterilizing immunity in the subject for at least 2 years, at least 3 years, at least 4 years, at least 5 years, at least 6 years, at least 7 years, at least 8 years, at least 9 years, at least 10 years, or more. In some embodiments, the effective amount of an mRNA vaccine is sufficient to provide sterilizing immunity in the subject at an at least 5-fold lower dose relative to control. For example, the effective amount may be sufficient to provide sterilizing immunity in the subject at an at least 10-fold lower, 15-fold, or 20-fold lower dose relative to a control.

Detectable Antigen. In some embodiments, the effective amount of an mRNA vaccine is sufficient to produce detectable levels of EBV antigen as measured in serum of the subject at 1-72 hours post administration.

Titer. An antibody titer is a measurement of the number of antibodies within a subject, for example, antibodies that are specific to a particular antigen (e.g., an anti-EBV antigen). Antibody titer is typically expressed as the inverse of the greatest dilution that provides a positive result. Enzyme-linked immunosorbent assay (ELISA) is a common assay for determining antibody titers, for example.

In some embodiments, the effective amount of an mRNA vaccine is sufficient to produce a 1,000-10,000 neutralizing antibody titer produced by neutralizing antibody against the specific antigen as measured in serum of the subject at 1-72 hours post administration. In some embodiments, the effective amount is sufficient to produce a 1,000-5,000 neutralizing antibody titer produced by neutralizing antibody against the specific antigen as measured in serum of the subject at 1-72 hours post administration. In some embodiments, the effective amount is sufficient to produce a 5,000-10,000 neutralizing antibody titer produced by neutralizing antibody against the specific antigen as measured in serum of the subject at 1-72 hours post administration.

In some embodiments, the neutralizing antibody titer is at least 100 NT₅₀. For example, the neutralizing antibody titer may be at least 200, 300, 400, 500, 600, 700, 800, 900 or 1000 NT₅₀. In some embodiments, the neutralizing antibody titer is at least 10,000 NT₅₀.

In some embodiments, the neutralizing antibody titer is at least 100 neutralizing units per milliliter (NU/mL). For example, the neutralizing antibody titer may be at least 200, 300, 400, 500, 600, 700, 800, 900 or 1000 NU/mL. In some embodiments, the neutralizing antibody titer is at least 10,000 NU/mL.

In some embodiments, an anti-herpesvirus (e.g., anti-EBV) antigen antibody titer produced in the subject is increased by at least 1 log relative to a control. For example, an anti-herpesvirus (e.g., anti-EBV) antigen antibody titer produced in the subject may be increased by at least 2, 3, 4, 5, 6, 7, 8, 9 or 10 log relative to a control.

In some embodiments, an anti-herpesvirus (e.g., anti-EBV) antigen antibody titer produced in the subject is increased at least 2 times relative to a control. For example, an anti-herpesvirus (e.g., anti-EBV) antigen antibody titer produced in the subject is increased by at least 3, 4, 5, 6, 7, 8, 9 or 10 times relative to a control.

In some embodiments, a geometric mean, which is the nth root of the product of n numbers, is generally used to describe proportional growth. Geometric mean, in some embodiments, is used to characterize antibody titer produced in a subject.

A control may be, for example, an unvaccinated subject, or a subject administered a recombinant protein vaccine or an attenuated virus vaccine.

### EXAMPLES

With a genome approximately 175 kb in size, EBV utilizes several surface glycoproteins to mediate viral entry into B cells and epithelial cells. These include the principal glycoproteins gp350, gH/gL, gp42, and gB. The gH/gL complex is part of the herpesvirus core fusion machinery required for entry into all cell types. In addition, it serves as receptor binding moiety for epithelial cell entry, contributing to the generation of approximately 75% neutralizing antibodies (nAbs) against epithelial cell entry and 22% nAbs against B cell entry in natural infection. gp350 (and its naturally occurring isoform gp220) facilitates virus attachment to B cells by engaging with complement receptor 2 on the B cell surface and generates approximately 57% of nAb response against B cell entry following natural infection. gp42 forms part of a ternary complex with gH/gL to bind to human leukocyte antigen class II surface antigen on B cells, serving as a tropism switch for EBV from epithelial to B cell entry, and accounting for approximately 14% of neutralization activity in human sera.

Like all other human herpesviruses EBV persists in the host for life following acute infection, cycling between a latent and lytic state. Key to this process is the so called “latent” viral proteins, which include Epstein-Barr Nuclear Antigens (EBNAs) and latent membrane proteins (LMPs). These antigens have critical roles in the regulation of gene expression during EBV latency and are potent inducers of T cell-mediated immunity.

To this end, the vaccine candidate under investigation in these Examples contains the same four mRNA sequences encoding structural glycoproteins (gp220, gp42, and gH/gL) with the addition of two mRNA sequences encoding latent antigens EBNA3A and LMP2B. Such a vaccine, containing both lytic and latent antigens, may be useful in both EBV-seronegative and EBV-seropositive populations at risk for EBV-associated diseases such as multiple sclerosis and PTLD. To better understand potential interference between the glycoprotein and latent antigen components and determine their final relative mRNA ratios, two different drug products with differing ratios of the mRNAs (see Example 1 below) are tested. Drug product 1 (DP1, 100 μg) contains 78.9 μg of mRNA encoding glycoprotein antigens (42.1 μg gp220, 15.8 μg gH, 10.5 μg gL, and 10.5 μg gp42) plus 21.0 μg of mRNA encoding latent antigens (10.5 m μg g EBNA3A and 10.5 μg LMP2b). Drug product 2 (DP2, 100 μg) contains 50.0 μg of mRNA encoding glycoprotein antigens (26.7 μg gp220, 10.0 μg gH, 6.7 μg gL, and 6.7 μg gp42) plus 50.0 μg of mRNA encoding latent antigens (25 μg EBNA3A and 25 μg LMP2b).

According to the present disclosure, the manufacture of polynucleotides and/or parts or regions thereof may be accomplished utilizing the methods taught in International Publication WO 2014/152027, entitled “Manufacturing Methods for Production of RNA Transcripts,” the content of which is incorporated herein by reference in its entirety.

Purification methods may include those taught in International Publication WO 2014/152030 and International Publication WO 2014/152031, each of which is incorporated herein by reference in its entirety.

Detection and characterization methods of the polynucleotides may be performed as taught in International Publication WO 2014/144039, which is incorporated herein by reference in its entirety.

Characterization of the polynucleotides of the disclosure may be accomplished using polynucleotide mapping, reverse transcriptase sequencing, charge distribution analysis, detection of RNA impurities, or any combination of two or more of the foregoing. “Characterizing” comprises determining the mRNA transcript sequence, determining the purity of the mRNA transcript, or determining the charge heterogeneity of the mRNA transcript, for example. Such methods are taught in, for example, International Publication WO 2014/144711 and International Publication WO 2014/144767, the contents of each of which are incorporated herein by reference in their entirety.

Production, purification, and evaluation of mRNA vaccines encoding EBV antigens may be accomplished using methods described in International Publication WO 2017/070623 and International Publication WO 2018/170256, the contents of each of which are incorporated herein by reference in their entirety.

In experiments where a lipid nanoparticle (LNP) formulation was used, the formulation included 48 mol % ionizable lipid of Compound 1, 11 mol % 1,2 distearoyl-sn-glycero-3-phosphocholine (DSPC), 38.5 mol % cholesterol, and 2.5 mol % PEG-modified 1,2 dimyristoyl-sn-glycerol, methoxypolyethyleneglycol (PEG2500 DMG).

### Example 1: Immunogenicity of Drug Products at Different Component Ratios

The following studies were performed to evaluate the immunogenicity of vaccines comprising mRNA encoding EBV latent antigens (EBNA3A (SEQ ID NO: 5) and LMP2B (SEQ ID NO: 6)) and glycoprotein antigens (gp220 (SEQ ID NO: 1), gH (SEQ ID NO: 2), gL (SEQ ID NO: 3), and soluble gp42 (SEQ ID NO: 4)) formulated in a lipid nanoparticle. The mRNA vaccines, referred to in this Example as drug products DP1, DP2, and DP3, contained different component ratios of EBV latent antigens and glycoproteins, as shown in Table 6. The low latent antigen drug product, DP1, was produced at a component ratio of 21% latent antigen to 78.9% glycoprotein. The high latent antigen drug product, DP2, was produced at a component ratio of 50% latent antigen to 50% glycoprotein. Lastly, DP3 contained only glycoproteins and no latent antigens.

TABLE 6
Amount of individual mRNA and component ratios of drug products

| | Antigens | DP1 | | | | DP2 | | | |
|---|---|---|---|---|---|---|---|---|---|
| Components | Encoded | (21% latent antigens) | | | | (50% latent antigens) | | | |
| Total | by mRNA | 25 μg | 50 μg | 100 μg | % | 25 μg | 50 μg | 100 μg | % |
| Glycoproteins | gp220 | 10.6 | 21.1 | 42.1 | 78.9 | 6.7 | 13.3 | 26.7 | 50 |
| | gH | 4.0 | 7.9 | 15.8 | | 2.5 | 5.0 | 10.0 | |
| | gL | 2.7 | 5.3 | 10.5 | | 1.7 | 3.3 | 6.7 | |
| | gp42 (soluble) | 2.7 | 5.3 | 10.5 | | 1.7 | 3.3 | 6.7 | |
| Latent | EBNA3A | 2.7 | 5.3 | 10.5 | 21 | 6.3 | 12.5 | 25.0 | 50 |
| antigens | LMP2B | 2.7 | 5.3 | 10.5 | | 6.3 | 12.5 | 25.0 | |

To test immunogenicity 8-10-week-old female CB6F1 mice with Haplotype H-2^{b/d} were immunized according to Table 7 Three doses were tested. Doses were administered intramuscularly in either a full dose (10 μg), a ⅓ dose (3.33 μg), or a 1/10 dose (1 μg). Mice were immunized with either DP1 or DP2 at three different doses (10 μg, 3.33 μg, or 1 μg) or DP3 at two different doses (10 μg, or 3.33 μg) or PBS was administered as a control. Immunizations were administered on day 1 (d1), next a booster was administered on day 29 (d29), followed 7-days later by spleen and blood collection on day 36 (d36). A total of 12 mice were tested per group.

The readout assays performed were as follows: intracellular cytokine staining (ICS), B cell neutralization assays and Luminex® assays were performed on full and ⅓ doses of DP1, DP2, DP3 and PBS (control). For 1/10 doses of DP1 and DP2, only B cell neutralization assays and Luminex® assays were performed.

TABLE 7
Study design for immunogenicity study

| | | Total Dose Level | | | |
|---|---|---|---|---|---|
| | Composition | (μg/mouse) | | N | Readouts |
| 1 | DP1 (low latent antigen) | 10 | μg | 12 | ICS (n = 8), Neut, Luminex ® |
| 2 | DP1 (low latent antigen) (⅓ dose) | 3.33 | μg | 12 | ICS (n = 8), Neut, Luminex ® |
| 3 | DP1 (low latent antigen) ( 1/10 dose) | 1 | μg | 12 | Neut, Luminex ® (no ICS) |
| 4 | DP2 (high latent antigen) | 10 | μg | 12 | ICS (n = 8), Neut, Luminex ® |
| 5 | DP2 (high latent antigen) (⅓ dose) | 3.33 | μg | 12 | ICS (n = 8), Neut, Luminex ® |
| 6 | DP2 (high latent antigen) ( 1/10 dose) | 1 | μg | 12 | Neut, Luminex ® (no ICS) |
| 7 | DP3 | 10 | μg | 12 | ICS (n = 8), Neut, Luminex ® |
| 8 | DP3 (⅓ dose) | 3.33 | μg | 12 | ICS (n = 8), Neut, Luminex ® |

| | | | | |
|---|---|---|---|---|
| 9 | PBS Control | — | 12 | ICS (n = 8), Neut, Luminex ® |

ICS assays were performed biological samples collected 7-days post booster administration on d36. Briefly, samples were stimulated with peptides of either LMP2B (1 μg/ml), EBNA3A (1 μg/ml), gH (1 μg/ml), gL (1 μg/ml), gp42 (1 μg/ml), or gp220 (1 μg/ml). Cells were stained with CD107a, IFN-γ, TNF-α, IL-2, IL-4, IL-5 or IL-13 and quantified using flow cytometry.

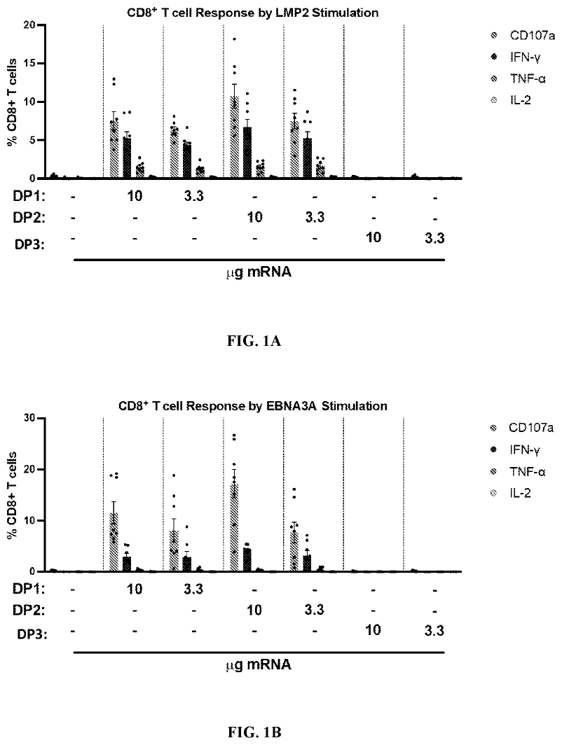

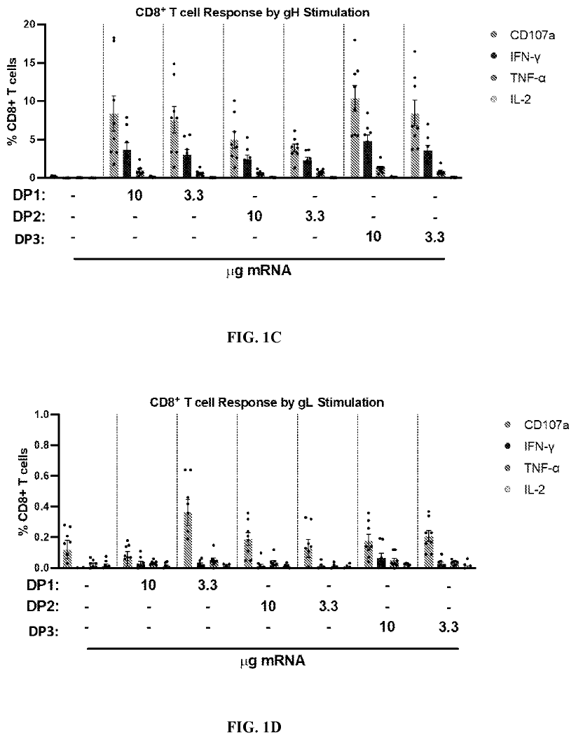

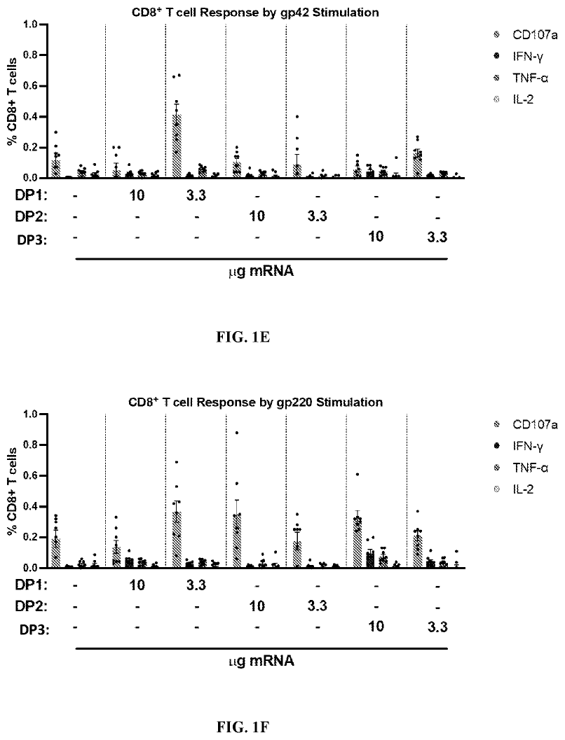

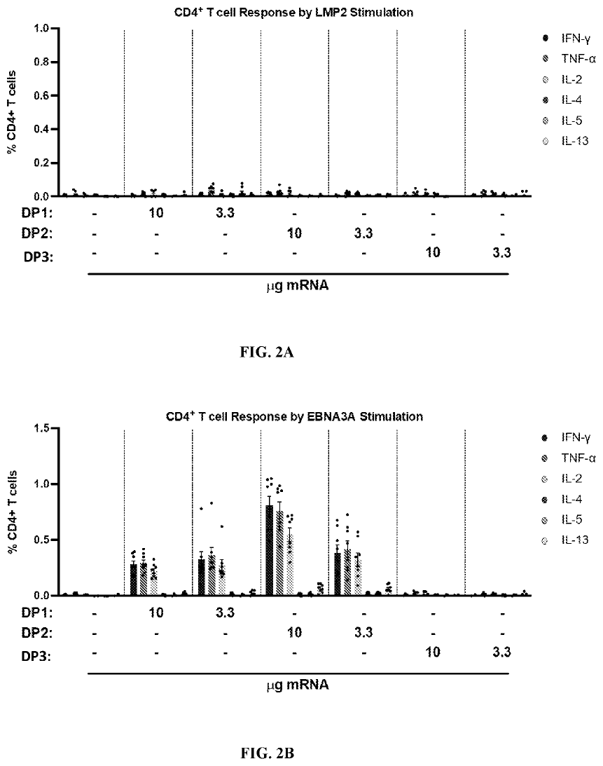

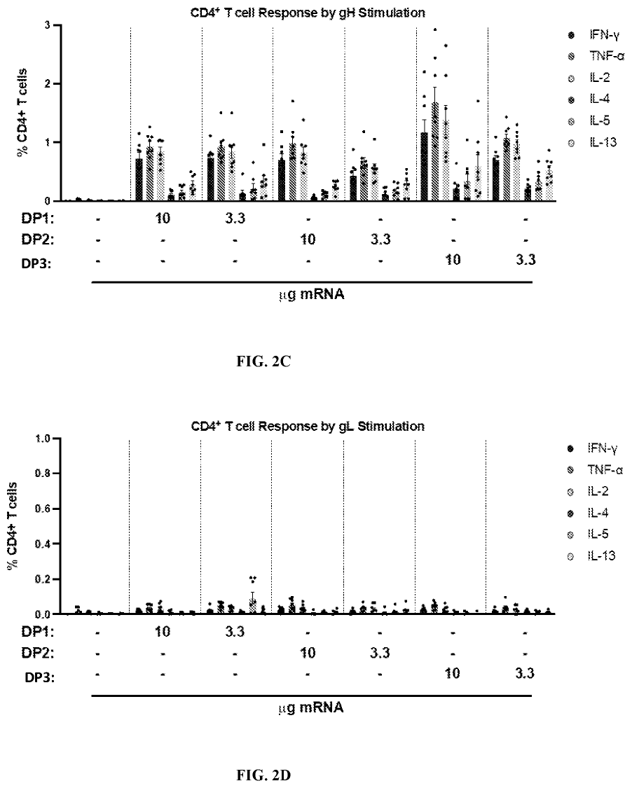

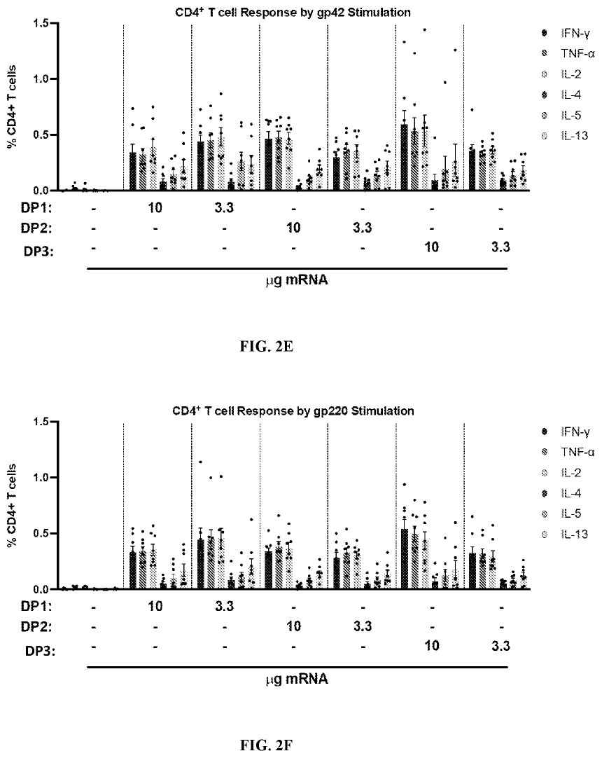

Results from the ICS assay stimulation with LMP2B indicate that LMP2B does not elicit strong CD4⁺ T cell responses and higher doses of LMP2B in DP2 results in stronger CD8⁺ T cell CD107a and IFN-γ responses. Also, the ⅓ dose (3.33 μg) DP2 has comparable CD8⁺ T cell responses to DP1 administered at a full dose (10 μg), and a ⅓ dose (3.33 μg). DP3 and PBS (control) did not elicit either a strong CD4⁺ T cell or CD8⁺ T cell responses (FIGS. 1A, 2A).

Results from the ICS assay stimulation with EBNA3A indicate that CD4⁺ T cell and CD8⁺ T cell responses show dose-dependence in DP2, but not DP1. Also, a higher dose of EBNA3A in DP2 results in significantly stronger CD4⁺ T cell and CD8⁺ T cell CD107a responses. Additionally, the ⅓ dose (3.33 μg) of DP2 has comparable CD4⁺ T cell and CD8⁺ T cell responses to DP1 administered at a full dose (10 μg), and a ⅓ dose (3.33 μg). DP3 and PBS (control) did not elicit either a strong CD4⁺ T cell or CD8⁺ T cell responses (FIGS. 1B, 2B).

Results from the ICS assay stimulation with gH indicate that administration of DP1 or DP2 decreases the CD4⁺ T cell responses to gH to a level comparable to a ⅓ dose (3.33 μg) of DP3 (FIG. 2C). Additionally, CD8⁺ T cell responses for administrations of DP1 are comparable to DP3 and CD8⁺ T cell responses measured by CD107a and IFN-7 are decreased in groups that were administered DP2 (FIG. 1C).

Results from the ICS assay stimulation with gL indicate that administration of DP1, DP2 and DP3 do not elicit either a strong CD4⁺ T cell or CD8⁺ T cell responses and are like PBS (control) (FIGS. 1D and 2D).

Results from the ICS assay stimulation with gp42 indicate that CD4⁺ T cell responses in DP1, DP2, and DP3 are comparable (FIG. 2E). Comparatively, gp42 does not elicit a strong CD8⁺ T cell responses in either DP1, DP2 or DP3 (FIG. 1E). PBS (control) does not elicit either a CD4⁺ T cell or CD8⁺ T cell responses.

Results from the ICS assay stimulation with gp220 indicate that CD4⁺ T cell responses in DP1, DP2, and DP3 are comparable (FIG. 2F). Comparatively, gp220 does not elicit a strong CD8⁺ T cell responses in either DP1, DP2 or DP3 (FIG. 1F). PBS (control) does not elicit either a CD4⁺ T cell or CD8⁺ T cell responses.

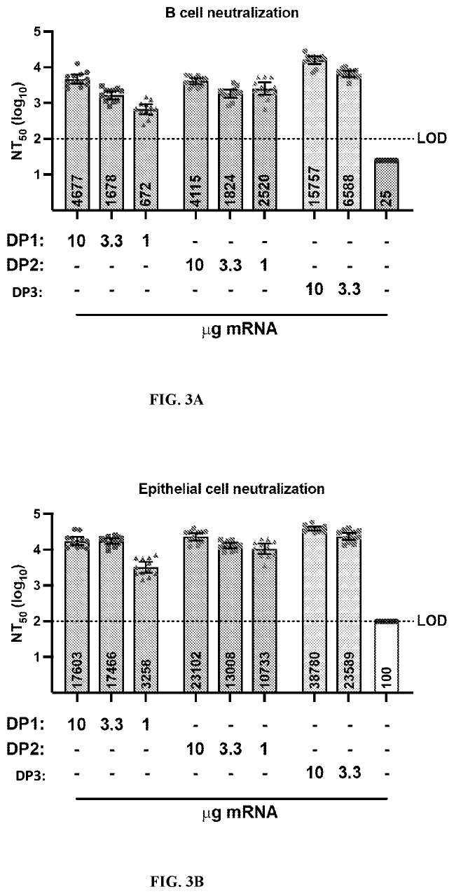

Results from the B cell neutralization assay indicate that administration of DP1 and DP2 result in a neutralizing antibody (nAb) response. The nAb response of DP1 and DP2 are approximately 3-fold lower compared to DP3. The nAb response of DP1 exhibited dose dependence. The administration of PBS (control) did not result in a nAb response (FIG. 3A). Similar results were observed in an epithelial cell neutralization study where administration of PBS (control) did not result in nAb response, however DP1, and DP2 administration resulted in a nAb response that was 3-fold lower than the response to DP3. Additionally, DP1 resulted in a nAb response that was dose dependent (FIG. 3B).

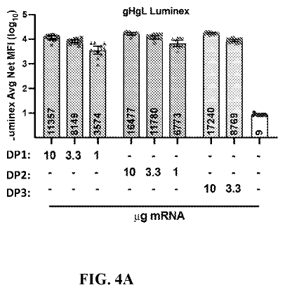

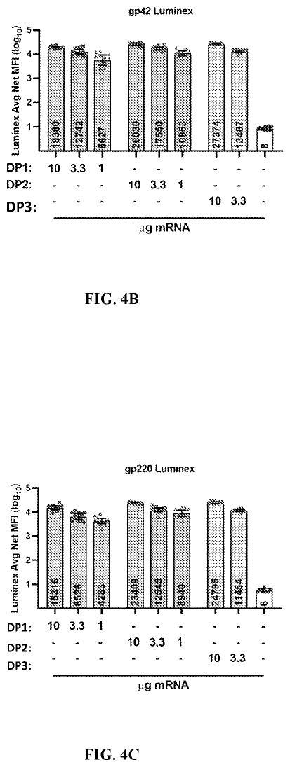

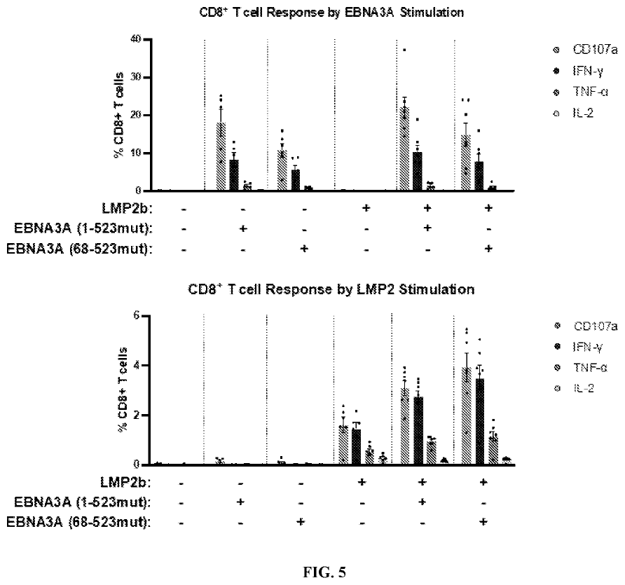

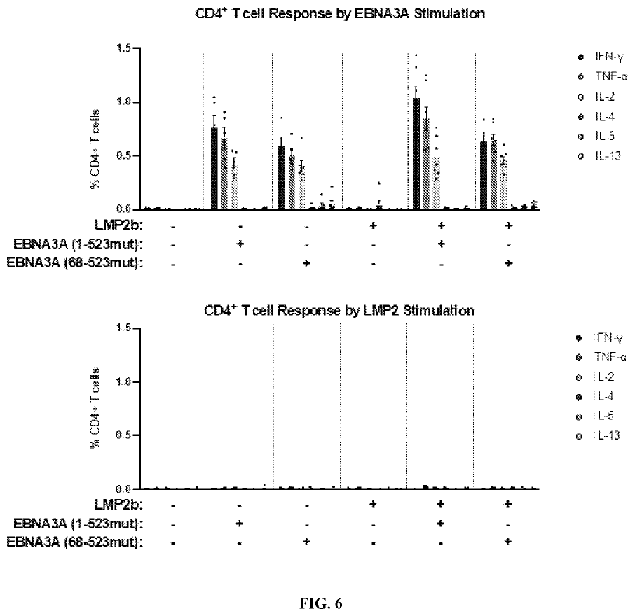

Binding titers to the glycoprotein antigens (gHgL, gp42, and gp220) were also measured using a Luminex® assay. Binding titers measured using Luminex® showed less dose dependence than those observed in the ELISA assay. PBS controls did not result in measurable levels of binding titers. Administration of DP1, DP2 and DP3 resulted in binding antibody titers for all glycoprotein antigens tested that were like the ELISA assay, although the difference between DP1/DP2 and DP3 was decreased (FIGS. 4A-4C).

Collectively, the results of the ICS, B cell neutralization, ELISA and Luminex® assays demonstrated CD8+ T cell and CD4+ T cell responses to EBNA3A latent antigen were detected in both DP1 and DP2. Additionally, CD8+ T cell responses to LMP2B latent antigen were detected in both DP1 and DP2. The glycoprotein antigen gH elicits both CD8+ and CD4+ T cell responses, whereas gp42 and gp220 elicit CD4+ T cell responses. Lastly, binding, and neutralizing antibody responses were detected in both DP1 and DP2.

### Example 2

The aim of the Example was to evaluate the cell-mediated immunogenicity of EBV latent antigens EBNA3A and LMP2B for potential inclusion in an EBV therapeutic vaccine. The antigens were evaluated in CB6F1 mice, a cross between female BALB/c and male C57BL/6 mice. The results of the two-dose immunization series of mRNA constructs encoding EBV wild-type latent antigen LMP2B and two truncated forms of latent antigen EBNA3A (aa 1-523 or aa 68-523 with mutations at four NLSs10) are provided. The EBNA3A (1-523mut) construct included in the study spans residues 1-523 with point mutations at four nuclear localization signals within that region and a deletion of the transcriptional regulator domain located in its C terminus (region spanning residues 524-944). The EBNA3A (68-523mut) is similarly truncated at residue 523, with an additional N-terminal deletion that removes 1-56 aa region recently reported to be associated with elevated antibody response in MS patients. LMP2B, a multi-transmembrane protein, is a naturally occurring isoform of LMP2A, lacking the latter's N-terminal 119 residues. LMP2B, unlike LMP2A, does not modulate B cell receptor signaling or block apoptosis, and instead may serve as a negative regulator of the LMP2A isoform.

Animals received a two-dose immunization schedule on Day 1 and 29. One-week post-boost on Day 36, T cell responses were assessed by intracellular cytokine staining (ICS).

The study groups, immunogens, doses, and dosing schedule are shown in Table 8. Study groups contained five to seven 8-week-old female CB6F1 mice (Charles River Laboratories), as specified. On Day 1, all animals received a priming dose of 1 μg of the specified formulated mRNA/LNP mixture via intramuscular injection in a volume of 50 μl. Control animals were injected with PBS. A repeat dose was administered on Day 29. Spleens were collected from all animals per group on Day 36, and immediately processed. Study endpoints are listed in Table 9.

TABLE 8
Study Design

| Group | N | Name | Dose | | Schedule |
|---|---|---|---|---|---|
| 1 | 6 | EBNA3A (1-523mut) | 1 | μg | Days 1 & 29 |
| 2 | 6 | EBNA3A (68-523mut) | 1 | μg | |
| 3 | 6 | LMP2b | 1 | μg | |
| 4 | 7 | LMP2b + EBNA3A (1-523mut) | 1 | μg each | |
| 5 | 7 | LMP2b + EBNA3A (68-523mut) | 1 | μg each | |

| | | | |
|---|---|---|---|
| 6 | 5 | PBS | — |

TABLE 9
Endpoints

| Endpoint | Method | Day |
|---|---|---|
| CD4+ and CD8+ T cell responses | Intracellular cytokine staining (ICS) | 36 |

Three separate mRNA Drug Substances were used in the Example. The sequences encoded by each individual mRNA expressed i) EBNA3A (1-523mut) that is a truncated form of EBNA3A (1-523 amino acids out of 944) with mutations in four NLS sites or ii) EBNA3A (68-523mut) that is a truncated form of EBNA3A (68-523 amino acids our of 944) with mutations in three remaining NLS sites and/or iii) wild-type LMP2b.

Wild type EBNA3A is a transcriptional regulator that localizes to the nucleus and interacts with several cellular factors involved in chromatin regulation11. The 524-944 truncation in EBNA3A (1-523mut) antigen was designed to remove C-terminal portion containing a proline-rich region and binding site for transcriptional regulator Carboxyl-terminal-binding Protein (CtBP)12. Four NLSs10 present in the first 523 amino acids of the protein were mutated in the EBNA3A (1-523mut) antigen ORF to modulate its subcellular trafficking. The mutations are described in Table 10.

EBNA3A (68-523mut) antigen has an N-terminal truncation of amino acids 1-68 designed to remove amino acids 1-56 of EBNA3A as this region may be associated with elevated antibody response in MS patients3. The truncation was extended until residue 68 to remove sequences containing the first NLS. In addition, a sequence motif (RPP) was reverted in the NLS3 (Table 10) to restore a known T-cell epitope13 (amino acid sequence 379RPPIFIRRL387 (SEQ ID NO: 97)).

LMP2b is encoded by the LMP2 gene that also encodes for another isoform called LMP2a, both multi-transmembrane proteins. LMP2a modulates B-cell receptor signaling and blocks apoptosis through its cytoplasmic tail. LMP2b, which lacks LMP2a's cytoplasmic tail (residues 1-119), does not affect B-cell receptor signaling but rather may be a negative regulator of its isoform LMP2a14.

TABLE 10
EBNA3A NLS mutations

| | | | | NLS | | |
|---|---|---|---|---|---|---|
| | | Original | | start-end | Mutated | |
| | | NLS | SEQ | amino acid | NLS | SEQ |
| NLS | Antigen Name | sequence | ID NO: | coordinates | sequence | ID NO: |
| NLS1 | EBNA3A (1-523mut) | KRKR | 99 | 63-66 | GSGS | 104 |
| | only | | | | | |
| NLS2 | EBNA3A (1-523mut) | RDRRRNPASR | 100 | 146-155 | GSGSGSGSGS | 105 |
| | and EBNA3A (68-523mut) | | | | | |
| NLS3 | EBNA3A (1-523mut) | PKVKRPP | 101 | 375-381 | GSGSGSG | 106 |
| | only | | | | | |
| NLS3 | EBNA3A (68-523mut) | PKVK | 102 | 375-378 | GSGS | 104 |
| | only | | | | | |
| NLS4 | EBNA3A (1-523mut) | RAGKR | 103 | 394-398 | GSGSG | 107 |
| | and EBNA3A (68-523mut) | | | | | |

The mRNA Drug Substance is formulated in a mixture of 4 lipids to form a drug lipid complex (lipid nanoparticle [LNP]) to form the respective drug products. The four lipids are heptadecan-9-yl 8-((2-hydroxyethyl)(6-oxo-6(undecyloxy)hexyl) amino) octanoate (SM-102); 1,2-dimyristoyl-rac-glycero-3-methoxypolyethylene glycol-2000 (PEG2000-DMG); 1,2-distearoyl-sn-glycero-3-phosphocholine (DSPC); and cholesterol.

The Drug Product lots evaluated in this study are shown in Table. All drug products were stored at either −20° C. or −80° C. as per manufacturer's instructions. On the day of dosing, the drug products were thawed at room temperature and adjusted as necessary with PBS to reach the target mRNA dose level within the 50 l/animal injection volume. The compositions were stored at 4C until time of dosing (<2 hours).

Immunization was performed by i.m. injection.

TABLE 11
Test Articles Details

| | | | Drug | Drug | |
|---|---|---|---|---|---|
| | | | Substance | Product | Stock |
| Group | Name | Composition | Lot # | Lot # | Concentration |
| 1 | EBNA3A (1- | mRNA/LNP | 18502.1 | 84609 | 0.519 mg/ml |
| | 523mut) | | | | |
| 2 | EBNA3A (68- | mRNA/LNP | 24332.1 | 108215 | 0.482 mg/ml |
| | 523mut) | | | | |
| 3 | LMP2b | mRNA/LNP | 17907.1 | 70862 | 0.43 mg/ml |
| 4 | LMP2b + | mRNA/LNP | 17907.1 and | 70862 and | 0.43 mg/ml and |
| | EBNA3A (1- | | 18502.1 | 84609 | 0.519 mg/ml |
| | 523mut) | | | | |
| 5 | LMP2b + | mRNA/LNP | 17907.1 and | 70862 and | 0.43 mg/ml and |
| | EBNA3A (68- | | 24332.1 | 108215 | 0.482 mg/ml |
| | 523mut) | | | | |
| 6 | PBS | — | — | — | — |

Following euthanization, mouse spleens were collected and stored in at room temperature. A gentleMACS tissue dissociator was used to generate mononuclear single-cell suspensions from CB6F1 mouse whole spleens. Following tissue dissociation, the cells were sieved through a filter. Cells from each mouse were counted and resuspended in sterile-filtered media and incubated at 37° C. with 5% CO₂ with Protein Transport Inhibitor, EBNA3A or LMP2b peptide pools, and CD107a PE. Control cells were incubated with no peptide stimulation or with PMA/Ionomycin. Cells were then washed with PBS prior to staining with LIVE/DEAD Fixable UV Blue Dead Cell Stain at room temperature. Cells were subsequently washed with sterile-filtered FC stain buffer. Staining was then performed at room temperature for 15 minutes with a surface stain cocktail containing the following antibodies in Brilliant Stain Buffer: FC Block, CD4 BV480, CD62L APC-R700, CD44 BUV395, I-A/I-E AF488, and CD8 BUV805. Cells were subsequently washed with buffer then fixed and permeabilized. Cells were then washed. Intracellular staining was then performed at 4° C. for 30 minutes using a cocktail of antibodies in Brilliant Stain Buffer: CD3 BV605, IFN-γ BV421, TNF-α BV711, IL-5 APC, IL-4 PE-CF594, IL-13 PE-Cy7, and IL-2 BV785. Finally, cells were washed, filtered and resuspended in stain buffer prior to running on an Aurora Spectral Flow Cytometer. Analysis was done using FlowJo software (version 10.7.1) using the following gating strategy: Lymphocytes were gated from the total population on FSC-A/SSC-A plot, followed by doublet removal via the Singlet gate on an FSC-A/FSC-H plot, dead cell exclusion was obtained via the Live gate on a Live Dead Blue/SSC-A plot. CD3+MHCII-cells are gated from the live, single cell population in a MHCII(IA-IE)/CD3 bivariate to exclude antigen presenting cells. The CD4⁺ and CD8⁺ T cell populations were then separated with a CD8/CD4 bivariate plot. Cytokine (IFN-γ, TNF-α, IL-2, IL-4, IL-5, and/or IL-13) and degranulation marker CD107a (CD8⁺ T cells only) expression from the CD4 and CD8 populations was assessed via co-expression with the T cell activation marker CD44 on a CD44/Cytokine bivariate plot. Background cytokine expression in the no-peptide condition was subtracted from that measured in the peptide pools for each individual mouse.

LMP2b and EBNA3A-Specific CD4⁺ and CD8⁺ T Cell Responses

To assess the induction of LMP2b and EBNA3A-specific T cell responses after immunization, spleens were collected from mice one-week post-dose 2 (Day 36). Splenocytes were stimulated with a LMP2b and EBNA3A peptide libraries and intracellular cytokine staining (ICS) was performed to quantify antigen-specific T cell response for Th1 and Th2 cytokines.

CD8⁺ T cell responses were detected for both EBNA3A and LMP2b in all groups (Error! Reference source not found. 5). All three mRNA antigens induced antigen-specific CD8⁺ T cells that expressed IFN-g and TNF-α as well as CD107a (a surrogate marker of CD8⁺ T cell degranulation).

Induction of a similar proportion of CD8⁺ T cells expressing CD107a and IFN-g cytokines was observed in animals immunized with EBNA3A (1-523mut) and EBNA3A (68-523mut), with higher response for EBNA3A (1-523mut). IL-2 cytokine responses to EBNA3A were minimal yet detected. The responses to EBNA3A were slightly higher in the groups containing LMP2b.

All four CD8⁺ cytokine responses (CD107a, IFN-g, TNF-α, IL-2) were also observed for LMP2b. The level of responses to LMP2b were increased in the presence of EBNA3A, and were higher when LMP2b was administered in combination with EBNA3A (68-523mut) compared to EBNA3A (1-523mut).

The analysis of the CD4⁺ T cell population response revealed that only EBNA3A, and not LMP2b, was able to induce a robust CD4⁺ T response (FIG. 6). CD4⁺ T cells responses were detectable in all groups containing EBNA3A (1-523mut) or EBNA3A (68-523mut). These were predominantly Th1 responses (IFN-g, TNF-a, and IL-2). Expression of Th2 cytokines (IL-4, IL-5, and IL-13) was minimal for all groups. Inclusion of LMP2b increased Th1 responses to EBNA3A (1-523mut) but this effect was not seen with EBNA3A (68-523mut).

LNP-formulated mRNA encoding EBV LMP2b, EBNA3A (1-523mut), and EBNA3A (68-523mut) generated antigen-specific T cell responses.

EBNA3A antigens induced a CD4+ T cell response with Th1-like signature while both LMP2b and EBNA3A antigens induced a CD8+ T cell response characterized by upregulation of cytotoxicity markers.

EBNA3A (68-523mut) induces marginally lower CD4+ Th1 and CD8+ responses compared to EBNA3A (1-523mut).

There was no significant interference in EBNA3A-specific responses when LMP2b and EBNA3A antigens were co-immunized. LMP2b CD8+ T cell response increases in the presence of EBNA3A.

### Example 3

To evaluate the ability of latent antigens which can be incorporated in an mRNA vaccine to induce an immune response against EBV+ cells, specifically a cytotoxic CD8 T cell response that can kill EBV-transformed lymphoblastoid B cell lines (LCL) a human autologous T cell cytotoxicity assay was performed.

### Methods:

### LCL Generation Method:

To generate EBV-immortalized cell lines, PBMCs (Table 12) were thawed and resuspended at 2×10⁶ per mL in a total volume of 5-10 mL of RPMI+10% FBS. FK506 (Tacrolimus) was added to cells at a concentration of 20 nM and cells were incubated for 1 hour at 37° C. Following incubation, EBV (Akata or 293-2089Luc) was added at a 1:15 dilution. Flasks were incubated upright in a CO₂ incubator. Media was doubled after 14 days. Afterwards, cells were split every 3-5 days when media turned yellow. Cells were spun down at 350 g for 5 minutes and resuspended to dissociate cell rosettes. Cells were brought to a concentration of 2×10⁵ per mL in 40 mL of media.

LCL immortalization was confirmed by morphology and by flow cytometry. For flow cytometry, 1 mL aliquots of cell culture were spun down and resuspended in 50 μl of PBS containing 1 μl of Live/Dead Aqua in a 96-well round bottom plate and incubated for 30 minutes in the dark. Cells were washed twice with 200 μl of PBS and then resuspended in a surface stain of 1 μl each of CD23-PE, CD19-APC and CD58-BV421 and incubated for 30 minutes in the dark. Cells were washed twice and then fixed in 100 μl of 4% PFA. Cells were washed and resuspended in 100 μl of QSol buffer. Cells were analyzed on an iQue and gated on live, CD19+ cells. Cells were considered immortalized when the population was CD23+CD58+ with most of the population CD23+CD58 hi.

TABLE 12
PBMC source information and HLA-types (Class A)

| Human | PBMC | Supplier | | | | | | |
|---|---|---|---|---|---|---|---|---|
| donor | source | donor ID | HLA Class A | | | | | |
| O | StemCell | RG2030 | A*02:01:01 | A*24:02:01 | B*07:02:01 | B*40:01:02 | C*03:04:01 | C*07:02:01 |
| F | StemCell | 110048259 | A*02:01 | A*03:01 | B*07:02 | B*40:01 | C*03:04 | C*07:02 |

### CD8 T Cell Cytotoxicity Assay Method:

Two HLA-typed healthy PBMC donors were used to isolate monocytes using Miltenyi Pan-monocyte isolation kit. Briefly, an antibody cocktail of lineage antibodies was applied to the PBMC samples, labelling all non-monocyte immune cells. Magnetic microbeads were used to separate unlabeled monocytes from other immune cells.

Monocytes undergo differentiation into monocyte-derived dendritic cells (Mo-DC) using CellXVivo Human Mo-DC differentiation kit (R&D Systems). Monocytes were cultured in serum free DC base media with recombinant IL-4 and GM-CSF for 7 days. Media was replenished with fresh cytokines every 2-3 days. At day 7, recombinant TNFa was added to the media to induce maturation. At day 10 flow cytometry was performed to assess the expression of DC and activation markers (CD208, CD83, CD40, CD86).

Mo-DC were incubated with the mRNAs encoding EBV antigens LMP2b, and EBNA3 or negative control mRNA (2.5ug/ml) for 24 hours at 37° C.

CD8 T cells were isolated from the same donors PBMCs using human CD8 T cell isolation kit. As above, an antibody cocktail of lineage antibodies was applied to the PBMC samples, labelling all non-CD8 T cells. Magnetic microbeads were used to separate unlabeled CD8 T cells from other immune cells.

CD8 T cells were labelled with Cell Trace Violet (CTV) and rested overnight at 37° C. CD8 T cells were then co-incubated with the EBV mRNA encoding antigens or negative control mRNA treated Mo-DC for 7 days. At day 7 the media was replaced, and recombinant IL-2 was added (50 ng/ml) to increase antigen specific T cell proliferation. T cells were incubated for a further 7 days.

At day 14 antigen specific T cells were identified by flow cytometry as CTVlo (the dye was diluted when cells proliferate). These CTVlo cells were sorted and rested overnight at 37° C.

The following day, antigen specific CD8 T cells were co-incubated with autologous LCLs for 6 hours. LCLs were pre-labelled with CTV (CTVhi). After 6 hours, samples were stained with CellEvent caspase 3/7 detection reagent (Invitrogen) and a fixable Live Dead Blue dye (Invitrogen). Apoptotic LCLs were identified by flow cytometry (Cytek Aurora cytometer) as caspase 3/7+ LiveDead Blue+/−. These two populations were combined to calculate total cytotoxicity.

### Results:

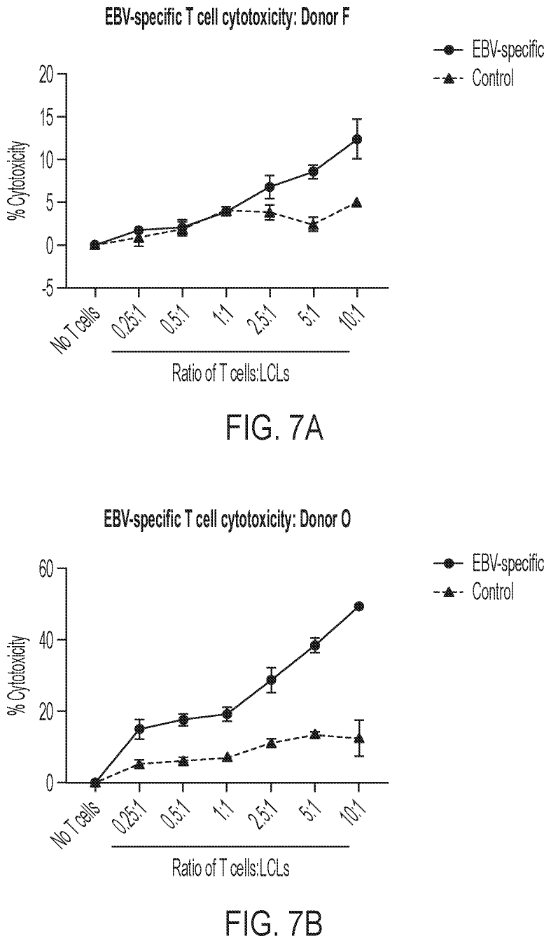

After co-incubation with autologous LCLs, EBV specific CD8 T cells induced Effector:Target dependent cytotoxicity of LCLs (FIGS. 7A and 7B, EBV-specific). In contrast, non-specific T cells did not show any dose dependent cytotoxicity against autologous LCLs (FIGS. 7A and 7B, Control). Mo-DC transfected with mRNA encoding EBV antigens LMP2b, and EBNA3, can effectively expand EBV specific CD8 T cells. These cells were capable of antigen recognition of autologous LCLs and induce cytotoxicity in an Effector:Target ratio dependent manner. Different maximum levels of cytotoxicity were observed, Donor O max cytotoxicity at 10:1 was 49%, while Donor F max cytotoxicity at 10:1 was 12%.

The data support the utility of mRNA vaccines based on the results achieved with EBNA3A & LMP2b latent mRNA antigens f for use in EBV sero-positive and sero-negative populations at risk of EBV associated disease, such as PTLD and MS, by demonstrating induction of cytotoxic CD8 T cells which recognize and kill EBV latently infected B cells.

### ADDITIONAL EMBODIMENTS

All of the features disclosed in this specification may be combined in any combination. Each feature disclosed in this specification may be replaced by an alternative feature serving the same, equivalent, or similar purpose. Thus, unless expressly stated otherwise, each feature disclosed is only an example of a generic series of equivalent or similar features.

From the above description, one skilled in the art can easily ascertain the essential characteristics of the present disclosure, and without departing from the spirit and scope thereof, can make various changes and modifications of the disclosure to adapt it to various usages and conditions. Thus, other embodiments are also within the claims. For example, the Additional Embodiments below are expressly contemplated:

### Additional Embodiments

1. An Epstein-Barr virus (EBV) messenger ribonucleic acid (mRNA) vaccine comprising:
(a) one or more mRNAs, each encoding an EBV lytic antigen;
(b) one or more mRNAs, each encoding EBV latent antigens; and
(c) a lipid nanoparticle,
wherein at least 50% of the mRNAs of the vaccine encodes the EBV lytic antigens.
   2. The EBV mRNA vaccine of embodiment 1, wherein 50% of the mRNAs of the vaccine encodes the EBV lytic antigens.
   3. The EBV mRNA vaccine of embodiment 1, wherein about 50% to about 80% of the mRNAs of the vaccine encodes the EBV lytic antigens.
   4. The EBV mRNA vaccine of any one of the preceding embodiments, wherein about 20% to about 50% of the mRNAs of the vaccine encodes the EBV latent antigens.
   5. The EBV mRNA vaccine of any one of the preceding embodiments, wherein the EBV lytic antigens are EBV glycoproteins.
   6. The EBV mRNA vaccine of embodiment 5, wherein the EBV glycoproteins are selected from EBV glycoprotein 350 (gp330), EBV glycoprotein 220 (gp220), EBV glycoprotein H (gH), EBV glycoprotein L (gL), EBV glycoprotein 42 (gp42), and EBV glycoprotein B (gB).
   7. The EBV mRNA vaccine of embodiment 6, wherein the EBV glycoproteins comprise EBV gp220, EBV gH, EBV gL, and EBV gp42.
   8. The EBV mRNA vaccine of any one of the preceding embodiments, wherein the EBV latent antigens are selected from EBV nuclear antigen 1 (EBNA1), EBV nuclear antigen 2 (EBNA2), EBV nuclear antigen 3A (EBNA3A), EBV nuclear antigen 3B (EBNA3B), EBV nuclear antigen 3C (EBNA3C), EBV latent membrane protein 1 (LMP1), EBV latent membrane protein 2A (LMP2A), and EBV latent membrane protein 2B (LMP2B).
   9. The EBV mRNA vaccine of embodiment 8, wherein the EBV latent antigens comprise EBNA3A and LMP2B.
   10. The EBV mRNA vaccine of any one of the preceding embodiments, wherein:
about 25% to about 45% of the mRNAs encode EBV gp220;
about 10% to about 20% of the mRNAs encode EBV gH;
about 5% to about 15% of the mRNAs encode EBV gL;
about 5% to about 15% of the mRNAs encode EBV gp42;
about 10% to about 25% of the mRNAs encode EBBA3A; and
about 10% to about 25% of the mRNAs encode EBV LMP2B.
   11. The EBV mRNA vaccine of embodiment 10, wherein:
about 40% to about 45% of the mRNAs encode EBV gp220;
about 15% to about 20% of the mRNAs encode EBV gH;
about 10% to about 15% of the mRNAs encode EBV gL;
about 10% to about 15% of the mRNAs encode EBV gp42;
about 10% to about 15% of the mRNAs encode EBBA3A; and
about 10% to about 15% of the mRNAs encode EBV LMP2B.
   12. The EBV mRNA vaccine of embodiment 11, wherein:
about 42% of the mRNAs encode EBV gp220;
about 16% of the mRNAs encode EBV gH;
about 10.5% of the mRNAs encode EBV gL;
about 10.5% of the mRNAs encode EBV gp42;
about 10.5% of the mRNAs encode EBBA3A; and
about 10.5% of the mRNAs encode EBV LMP2B.
   13. The EBV mRNA vaccine of embodiment 10, wherein:
about 25% to about 30% of the mRNAs encode EBV gp220;
about 10% to about 15% of the mRNAs encode EBV gH;
about 5% to about 10% of the mRNAs encode EBV gL;
about 5% to about 10% of the mRNAs encode EBV gp42;
about 20% to about 25% of the mRNAs encode EBBA3A; and
about 20% to about 25% of the mRNAs encode EBV LMP2B.
   14. The EBV mRNA vaccine of embodiment 13, wherein:
about 27% of the mRNAs encode EBV gp220;
about 10% of the mRNAs encode EBV gH;
about 6.5% of the mRNAs encode EBV gL;
about 6.5% of the mRNAs encode EBV gp42;
about 25% of the mRNAs encode EBBA3A; and
about 25% of the mRNAs encode EBV LMP2B.
   15. The EBV mRNA vaccine of any one of the preceding embodiments, wherein the C-terminus of the EBNA3A is truncated relative to the naturally occurring EBNA3A.
   16. The EBV mRNA vaccine of any one of the preceding embodiments, wherein the EBNA3A lacks the C-terminal transcriptional regulator domain.
   17. The EBV mRNA vaccine of embodiment 15 or 16, wherein the EBNA3A has a length of about 523 amino acids.
   18. The EBV mRNA vaccine of any one of embodiments 15-16, wherein the EBNA3A comprises amino acid residues corresponding to amino acid residues 1-523 of the naturally occurring EBNA3A.
   19. The EBV mRNA vaccine of any one of embodiments 15-18, wherein the EBNA3A does not include amino acid residues corresponding to amino acid residues 524-944 of the naturally occurring EBNA3A.
   20. The EBV mRNA vaccine of embodiment 15, wherein the N-terminus of the EBNA3A is truncated relative to the naturally occurring EBNA3A.
   21. The EBV mRNA vaccine of embodiment 20, wherein the EBNA3A has a length of about 455 amino acids.
   22. The EBV mRNA vaccine of embodiment 20 or 21, wherein the EBNA3A comprises amino acid residues corresponding to amino acid residues 68-523 of the naturally occurring EBNA3A.
   23. The EBV mRNA vaccine of any one of embodiments 20-22, wherein the EBNA3A does not include amino acid residues corresponding to amino acid residues 1-67 and 524-944 of the naturally occurring EBNA3A.
   24. The EBV mRNA vaccine of any one of the preceding embodiments, wherein one or more nuclear localization signals (NLS) in the EBNA3A are mutated relative to the naturally occurring EBNA3A.
   25. The EBV mRNA vaccine of embodiment 24, wherein 1-4, 2-4, 3-4, or 4 of the NLS in the EBNA3A are mutated relative to the naturally occurring EBNA3A.
   26. The EBV mRNA vaccine of embodiment 25, wherein the mutated NLS are at amino acid positions selected from: 63-66, 146-155, 375-381, and 394-398, relative to the naturally occurring EBNA3A.
   27. The EBV mRNA vaccine of embodiment 15, wherein the C-terminus of the EBNA3A is truncated at amino acid residue 68, the N-terminus of the EBNA3A is truncated at amino acid residue 523, and the EBNA3 comprises NLS mutations at amino acid residues 146-155, 375-378, and 394-398, relative to the naturally occurring EBNA3A.
   28. The EBV mRNA vaccine of any one of the preceding embodiments, wherein the EBV gp220 comprises an amino acid sequence having at least 80%, at least 85%, at least 90%, at least 95%, or 100% identity to the amino acid sequence of SEQ ID NO: 1.
   29. The EBV mRNA vaccine of embodiment 28, wherein the mRNA encoding EBV gp220 comprises an open reading frame comprising a nucleic acid sequence having at least 80%, at least 85%, at least 90%, at least 95%, or 100% identity to the nucleic acid sequence of SEQ ID NO: 7.
   30. The EBV mRNA vaccine of embodiment 29, wherein the mRNA encoding EBV gp220 comprises a nucleic acid sequence having at least 80%, at least 85%, at least 90%, at least 95%, or 100% identity to the nucleic acid sequence of SEQ ID NO: 13.
   31. The EBV mRNA vaccine of any one of the preceding embodiments, wherein the EBV gH comprises an amino acid sequence having at least 80%, at least 85%, at least 90%, at least 95%, or 100% identity to the amino acid sequence of SEQ ID NO: 2.
   32. The EBV mRNA vaccine of embodiment 31, wherein the mRNA encoding EBV gH comprises an open reading frame comprising a nucleic acid sequence having at least 80%, at least 85%, at least 90%, at least 95%, or 100% identity to the nucleic acid sequence of SEQ ID NO: 8.
   33. The EBV mRNA vaccine of embodiment 32, wherein the mRNA encoding EBV gH comprises a nucleic acid sequence having at least 80%, at least 85%, at least 90%, at least 95%, or 100% identity to the nucleic acid sequence of SEQ ID NO: 14.
   34. The EBV mRNA vaccine of any one of the preceding embodiments, wherein the EBV gL comprises an amino acid sequence having at least 80%, at least 85%, at least 90%, at least 95%, or 100% identity to the amino acid sequence of SEQ ID NO: 2.
   35. The EBV mRNA vaccine of embodiment 34, wherein the mRNA encoding EBV gL comprises an open reading frame comprising a nucleic acid sequence having at least 80%, at least 85%, at least 90%, at least 95%, or 100% identity to the nucleic acid sequence of SEQ ID NO: 9.
   36. The EBV mRNA vaccine of embodiment 35, wherein the mRNA encoding EBV gL comprises a nucleic acid sequence having at least 80%, at least 85%, at least 90%, at least 95%, or 100% identity to the nucleic acid sequence of SEQ ID NO: 15.
   37. The EBV mRNA vaccine of any one of the preceding embodiments, wherein the EBV gp42 is soluble.
   38. The EBV mRNA vaccine of any one of the preceding embodiments, wherein the EBV gp42 comprises an amino acid sequence having at least 80%, at least 85%, at least 90%, at least 95%, or 100% identity to the amino acid sequence of SEQ ID NO: 4.
   38b. The EBV mRNA vaccine of any one of the preceding embodiments, wherein the EBV gp42 comprises an amino acid sequence having at least 80%, at least 85%, at least 90%, at least 95%, or 100% identity to the amino acid sequence of SEQ ID NO: 21.
   39. The EBV mRNA vaccine of embodiment 38, wherein the mRNA encoding EBV gp42 comprises an open reading frame comprising a nucleic acid sequence having at least 80%, at least 85%, at least 90%, at least 95%, or 100% identity to the nucleic acid sequence of SEQ ID NO: 10.
   40. The EBV mRNA vaccine of embodiment 39, wherein the mRNA encoding EBV gp42 comprises a nucleic acid sequence having at least 80%, at least 85%, at least 90%, at least 95%, or 100% identity to the nucleic acid sequence of SEQ ID NO: 16.
   41. The EBV mRNA vaccine of any one of the preceding embodiments, wherein the EBNA3A comprises an amino acid sequence having at least 80%, at least 85%, at least 90%, at least 95%, or 100% identity to the amino acid sequence of SEQ ID NO: 5; or having at least 80%, at least 85%, at least 90%, at least 95%, or 100% identity to the amino acid sequence of any one of SEQ ID NOs: 28-31.
   41b. The EBV mRNA vaccine of any one of the preceding embodiments, wherein the EBNA3A comprises an amino acid sequence having at least 80%, at least 85%, at least 90%, at least 95%, or 100% identity to the amino acid sequence of SEQ ID NO: 22.
   42. The EBV mRNA vaccine of embodiment 41, wherein the mRNA encoding EBNA3A comprises an open reading frame comprising a nucleic acid sequence having at least 80%, at least 85%, at least 90%, at least 95%, or 100% identity to the nucleic acid sequence of SEQ ID NO: 11; or having at least 80%, at least 85%, at least 90%, at least 95%, or 100% identity to the nucleic acid sequence of any one of SEQ ID NOs: 24-27.
   43. The EBV mRNA vaccine of embodiment 42, wherein the mRNA encoding EBNA3A comprises a nucleic acid sequence having at least 80%, at least 85%, at least 90%, at least 95%, or 100% identity to the nucleic acid sequence of SEQ ID NO: 17; or having at least 80%, at least 85%, at least 90%, at least 95%, or 100% identity to the nucleic acid sequence of any one of SEQ ID NOs: 32-35.
   44. The EBV mRNA vaccine of any one of the preceding embodiments, wherein the EBV LMP2B comprises an amino acid sequence having at least 80%, at least 85%, at least 90%, at least 95%, or 100% identity to the amino acid sequence of SEQ ID NO: 6.
   44b. The EBV mRNA vaccine of any one of the preceding embodiments, wherein the EBV LMP2B comprises an amino acid sequence having at least 80%, at least 85%, at least 90%, at least 95%, or 100% identity to the amino acid sequence of SEQ ID NO: 23.
   45. The EBV mRNA vaccine of embodiment 44, wherein the mRNA encoding EBV LMP2B comprises an open reading frame comprising a nucleic acid sequence having at least 80%, at least 85%, at least 90%, at least 95%, or 100% identity to the nucleic acid sequence of SEQ ID NO: 12.
   46. The EBV mRNA vaccine of embodiment 46, wherein the mRNA encoding EBV LMP2B comprises a nucleic acid sequence having at least 80%, at least 85%, at least 90%, at least 95%, or 100% identity to the nucleic acid sequence of SEQ ID NO: 18.
   47. The EBV mRNA vaccine of any one of the preceding embodiments, wherein one or more of the mRNAs comprise a chemical modification.
   48. The EBV mRNA vaccine of any one of the preceding embodiments, wherein 100% of the uracil nucleotides of the one or more mRNAs comprise a chemical modification.
   49. The EBV mRNA vaccine of embodiment 47 or 48, wherein the chemical modification is 1-methylpseudouracil.
   50. The EBV mRNA vaccine of any one of the preceding embodiments, wherein the lipid nanoparticle comprises an ionizable lipid, a neutral lipid, a sterol, and a PEG-modified lipid.
   51. The EBV mRNA vaccine of embodiment 50, wherein the lipid nanoparticle comprises 40-60 mol % ionizable lipid, 5-20 mol % neutral lipid, 30-50 mol % sterol, and 0.5-5 mol % PEG-modified lipid.
   52. The EBV mRNA vaccine of embodiment 50 or 51, wherein the ionizable lipid is a compound of Formula (AIII):

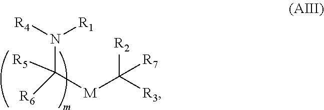

wherein
R₁ is R″M′R′ or C₅₋₂₀ alkenyl;
R₂ and R₃ are each independently selected from C₁₋₁₄ alkyl and C₂₋₁₄ alkenyl;
R₄ is —(CH₂)ₙQ, wherein Q is OH and n is selected from 3, 4, and 5;
M and M′ are each independently —OC(O)— or —C(O)O—;
R₅, R₆, and R₇ are each H;
R′ is a linear C₁₋₁₂ alkyl, or C₁₋₁₂ alkyl substituted with C₆₋₉ alkyl;
R″ is C₃₋₁₄ alkyl;
m is selected from 5, 6, 7, 8, 9, 10, 11, 12, and 13.
   53. The EBV mRNA vaccine of embodiment 33, wherein:
R₁ is R″M′R′;
R₂ and R₃ are each independently C₁₋₁₄ alkyl;
R₄ is —(CH₂)ₙQ, wherein Q is OH and n is 4;
M and M′ are each independently —OC(O)—;
R₅, R₆, and R₇ are each H;
R′ is C₁₋₁₂ alkyl substituted with C₆₋₉ alkyl;
R″ is C₃₋₁₄ alkyl; and
m is 6.
   54. The EBV mRNA vaccine of embodiment 33, wherein:
R₁ is C₅₋₂₀ alkenyl;
R₂ and R₃ are each independently C₁₋₁₄ alkyl;
R₄ is —(CH₂)ₙQ, wherein Q is OH and n is 3;
M is —C(O)O—;
R₅, R₆, and R₇ are each H; and
m is 6.
   55. The EBV mRNA vaccine of embodiment 33, wherein:
the compound is:

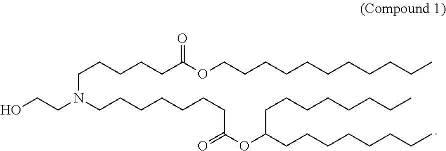 [SMILES: CCCCCCCCCCCOC(=O)CCCCCN(CCO)CCCCCCCC(=O)OC(CCCCCCCC)CCCCCCCC]

56. The EBV mRNA vaccine of any one of embodiments 50-55, wherein the neutral lipid is 1,2-distearoyl-sn-glycero-3-phosphocholine (DSPC).
57. The EBV mRNA vaccine of any one of embodiments 50-56, wherein the sterol is cholesterol.
58. The EBV mRNA vaccine of any one of embodiments 50-57, wherein the PEG-modified lipid is PEG-DMG.
59. A method, comprising administering to a subject the EBV mRNA vaccine of any one of the preceding embodiments in a therapeutically effective amount.
60. The method of embodiment 59, wherein the therapeutically effective amount is one or more 25-150 μg, 25-100 μg, 25-50 μg, 50-150 μg, or 50-100 μg of the EBV mRNA vaccine.
61. The method of embodiment 60, wherein the therapeutically effective amount is one or more 25 μg doses of the EBV mRNA vaccine.
62. The method of embodiment 60, wherein the therapeutically effective amount is one or more 50 μg doses of the EBV mRNA vaccine.
63. The method of embodiment 60, wherein the therapeutically effective amount is one or more 100 μg doses of the EBV mRNA vaccine.
64. The method of embodiment 60, wherein the therapeutically effective amount is one or more 150 μg doses of the EBV mRNA vaccine.
65. The method of any one of the preceding embodiments, wherein the subject has been infected with EBV.
66. The method of any one of the preceding embodiments, wherein the subject is a patient who has received, or is scheduled to receive, a solid organ transplant or a hematopoietic stem cell transplant.
67. The method of any one of the preceding embodiments, wherein the subject has, or is at risk of having, a posttransplant lymphoproliferative disorder.
68. The method of any one of the preceding embodiments, wherein the subject has, or is at risk of having, infectious mononucleosis.
69. The method of any one of the preceding embodiments, wherein the subject has, or is at risk of having, multiple sclerosis.
70. The method of any one of the preceding embodiments, wherein the subject has, or is at risk of having, systemic lupus erythematosus.
71. The method of any one of the preceding embodiments, wherein the subject has, or is at risk of having, cancer.
72. The method of embodiment 71, wherein the cancer is selected from Burkitt lymphoma, Hodgkin's lymphoma, nasopharyngeal carcinoma, and gastric cancer.
73. The method of any one of embodiments 59-72, wherein the subject is EBV-seropositive.
74. The method of any one of embodiments 59-72, wherein the subject is EBV-seronegative.
75. The method of any one of the preceding embodiments, wherein the subject is 18 to 55 years old.
76. The method of any one of the preceding embodiments, wherein the EBV mRNA is administered intramuscularly, intravenously, or intranasally.
77. The method of any one of the preceding embodiments, wherein the effective amount induces a neutralizing antibody response.
78. The method of any one of the preceding embodiments, wherein the effective amount induces a CD4+ T cell response.
79. The method of any one of the preceding embodiments, wherein the effective amount induces a CD8+ T cell response.

### EQUIVALENTS AND SCOPE

While several inventive embodiments have been described and illustrated herein, those of ordinary skill in the art will readily envision a variety of other means and/or structures for performing the function and/or obtaining the results and/or one or more of the advantages described herein, and each of such variations and/or modifications is deemed to be within the scope of the inventive embodiments described herein. More generally, those skilled in the art will readily appreciate that all parameters, dimensions, materials, and configurations described herein are meant to be exemplary and that the actual parameters, dimensions, materials, and/or configurations will depend upon the specific application or applications for which the inventive teachings is/are used. Those skilled in the art will recognize, or be able to ascertain using no more than routine experimentation, many equivalents to the specific inventive embodiments described herein. It is, therefore, to be understood that the foregoing embodiments are presented by way of example only and that, within the scope of the appended claims and equivalents thereto, inventive embodiments may be practiced otherwise than as specifically described and claimed. Inventive embodiments are directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the inventive scope of the present disclosure.

All definitions, as defined and used herein, should be understood to control over dictionary definitions, definitions in documents incorporated by reference, and/or ordinary meanings of the defined terms.

All references, patents and patent applications disclosed herein are incorporated by reference with respect to the subject matter for which each is cited, which in some cases may encompass the entirety of the document.

The indefinite articles “a” and “an,” as used herein in the specification and in the claims, unless clearly indicated to the contrary, should be understood to mean “at least one.” The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in some embodiments, to A only (optionally including elements other than B); in another embodiment, to B only (optionally including elements other than A); in yet another embodiment, to both A and B (optionally including other elements); etc. As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e., “one or the other but not both”) when preceded by terms of exclusivity, such as “either,” “one of,” “only one of,” or “exactly one of.” “Consisting essentially of,” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in some embodiments, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc. Each possibility represents a separate embodiment of the present invention.

It should be understood that, unless clearly indicated to the contrary, the disclosure of numerical values and ranges of numerical values in the specification includes both i) the exact value(s) or range specified, and ii) values that are “about” the value(s) or ranges specified (e.g., values or ranges falling within a reasonable range (e.g., about 10% similar)) as would be understood by a person of ordinary skill in the art.

It should also be understood that, unless clearly indicated to the contrary, in any methods disclosed herein that include more than one step or act, the order of the steps or acts of the method is not necessarily limited to the order in which the steps or acts of the method are disclosed.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03.

What is claimed is:

1. An Epstein-Barr virus (EBV) messenger ribonucleic acid (mRNA) vaccine comprising:
(a) one or more mRNAs, each comprising an open reading frame (ORF) encoding an EBV lytic antigen;
(b) one or more mRNAs, each comprising an ORF encoding an EBV latent antigen; and
(c) a lipid nanoparticle,
wherein at least 50% of the mass of the mRNAs in the vaccine are mRNAs encoding the EBV lytic antigen.

2. The EBV mRNA vaccine of claim 1, wherein about 50% to about 80% of the mass of the mRNAs in the vaccine are mRNAs encoding the EBV lytic antigens and wherein about 20% to about 50% of the mass of the mRNAs in the vaccine are mRNAs encoding the EBV latent antigens.

3. The EBV mRNA vaccine of any one of claims 1-2, wherein the EBV lytic antigen is an EBV glycoprotein, optionally selected from EBV glycoprotein 350 (gp330), EBV glycoprotein 220 (gp220), EBV glycoprotein H (gH), EBV glycoprotein L (gL), EBV glycoprotein 42 (gp42), and EBV glycoprotein B (gB).

4. The EBV mRNA vaccine of any one of claims 1-3, wherein the EBV latent antigen is selected from EBV nuclear antigen 1 (EBNA1), EBV nuclear antigen 2 (EBNA2), EBV nuclear antigen 3A (EBNA3A), EBV nuclear antigen 3B (EBNA3B), EBV nuclear antigen 3C (EBNA3C), EBV latent membrane protein 1 (LMP1), EBV latent membrane protein 2A (LMP2A), and EBV latent membrane protein 2B (LMP2B).

5. The EBV mRNA vaccine of any one of claims 1-4, wherein:
about 25% to about 45%, or optionally about 40% to about 45%, or about 42% of the mass of the mRNAs in the vaccine are mRNAs encoding EBV gp220;
about 10% to about 20%, or optionally about 15% to about 20%, or about 16% of the mass of the mRNAs in the vaccine are mRNAs encoding EBV gH;
about 5% to about 15%, or optionally about 10% to about 15%, or about 10.5% of the mass of the mRNAs in the vaccine are mRNAs encoding EBV gL;
about 5% to about 15%, or optionally about 10% to about 15%, or about 10.5% of the mass of the mRNAs in the vaccine are mRNAs encoding EBV gp42;
about 10% to about 25%, or optionally about 10% to about 15%, or about 10.5% of the mass of the mRNAs in the vaccine are mRNAs encoding EBBA3A; and
about 10% to about 25%, or optionally about 10% to about 15%, or about 10.5% of the mass of the mRNAs in the vaccine are mRNAs encoding EBV LMP2B.

6. The EBV mRNA vaccine of any one of claims 1-4, wherein:
about 25% to about 30%, or optionally about 27% of the mass of the mRNAs in the vaccine are mRNAs encoding EBV gp220;
about 10% to about 15%, or optionally about 10% of the mass of the mRNAs in the vaccine are mRNAs encoding EBV gH;
about 5% to about 10%, or optionally about 6.5% of the mass of the mRNAs in the vaccine are mRNAs encoding EBV gL;
about 5% to about 10%, or optionally about 6.5% of the mass of the mRNAs in the vaccine are mRNAs encoding EBV gp42;
about 20% to about 25%, or optionally about 25% of the mass of the mRNAs in the vaccine are mRNAs encoding EBBA3A; and
about 20% to about 25%, or optionally about 25% of the mass of the mRNAs in the vaccine are mRNAs encoding EBV LMP2B.

7. The EBV mRNA vaccine of any one of the preceding claims, wherein the EBNA3A has one or more of the following features:
a. lacks a C-terminal transcriptional regulator domain,
b. has a length of about 523 amino acids,
c. comprises amino acid residues corresponding to amino acid residues 1-523 of the naturally occurring EBNA3A,
d. does not include amino acid residues corresponding to amino acid residues 524-944 of the naturally occurring EBNA3A,
e. the N-terminus of the EBNA3A is truncated relative to the naturally occurring EBNA3A, wherein the C-terminus of the EBNA3A is truncated relative to the naturally occurring EBNA3A,
f. has a length of about 455 amino acids,
g. comprises amino acid residues corresponding to amino acid residues 68-523 of the naturally occurring EBNA3A,
h. does not include amino acid residues corresponding to amino acid residues 1-67 and 524-944 of the naturally occurring EBNA3A,
i. one or more nuclear localization signals (NLS) in the EBNA3A are mutated relative to the naturally occurring EBNA3A,
j. 1-4, 2-4, 3-4, or 4 of the NLS in the EBNA3A are mutated relative to the naturally occurring EBNA3A, optionally wherein the mutated NLS are at amino acid positions selected from: 63-66, 146-155, 375-381, and 394-398, relative to the naturally occurring EBNA3A, and/or,
k. has a C-terminus that is truncated at amino acid residue 68, the N-terminus of the EBNA3A is truncated at amino acid residue 523, and the EBNA3 comprises NLS mutations at amino acid residues 146-155, 375-378, and 394-398, relative to the naturally occurring EBNA3A.

8. The EBV mRNA vaccine of any one of claims 1-7, wherein the mRNA vaccine comprises mRNAs encoding antigens selected from gp220, gH, gL, gp42, EBBA3A and LMP2B and having the following features:
a. wherein EBV gp220 comprises an amino acid sequence having at least 80%, at least 85%, at least 90%, at least 95%, or 100% identity to the amino acid sequence of SEQ ID NO: 1, and/or the mRNA encoding EBV gp220 comprises an open reading frame comprising a nucleic acid sequence having at least 80%, at least 85%, at least 90%, at least 95%, or 100% identity to the nucleic acid sequence of SEQ ID NO: 7 and/or the mRNA encoding EBV gp220 comprises a nucleic acid sequence having at least 80%, at least 85%, at least 90%, at least 95%, or 100% identity to the nucleic acid sequence of SEQ ID NO: 13;
b. wherein the EBV gH comprises an amino acid sequence having at least 80%, at least 85%, at least 90%, at least 95%, or 100% identity to the amino acid sequence of SEQ ID NO: 2, and/or the mRNA encoding EBV gH comprises an open reading frame comprising a nucleic acid sequence having at least 80%, at least 85%, at least 90%, at least 95%, or 100% identity to the nucleic acid sequence of SEQ ID NO: 8, and/or the mRNA encoding EBV gH comprises a nucleic acid sequence having at least 80%, at least 85%, at least 90%, at least 95%, or 100% identity to the nucleic acid sequence of SEQ ID NO: 14;
c. wherein the EBV gL comprises an amino acid sequence having at least 80%, at least 85%, at least 90%, at least 95%, or 100% identity to the amino acid sequence of SEQ ID NO: 2, and/or the mRNA encoding EBV gL comprises an open reading frame comprising a nucleic acid sequence having at least 80%, at least 85%, at least 90%, at least 95%, or 100% identity to the nucleic acid sequence of SEQ ID NO: 9, and/or the mRNA encoding EBV gL comprises a nucleic acid sequence having at least 80%, at least 85%, at least 90%, at least 95%, or 100% identity to the nucleic acid sequence of SEQ ID NO: 15;
d. wherein the EBV gp42 is soluble, and/or the EBV gp42 comprises an amino acid sequence having at least 80%, at least 85%, at least 90%, at least 95%, or 100% identity to the amino acid sequence of SEQ ID NO: 4, and/or the mRNA encoding EBV gp42 comprises an open reading frame comprising a nucleic acid sequence having at least 80%, at least 85%, at least 90%, at least 95%, or 100% identity to the nucleic acid sequence of SEQ ID NO: 10, and/or the mRNA encoding EBV gp42 comprises a nucleic acid sequence having at least 80%, at least 85%, at least 90%, at least 95%, or 100% identity to the nucleic acid sequence of SEQ ID NO: 16;
e. wherein the EBNA3A comprises an amino acid sequence having at least 80%, at least 85%, at least 90%, at least 95%, or 100% identity to the amino acid sequence of SEQ ID NO: 5; or having at least 80%, at least 85%, at least 90%, at least 95%, or 100% identity to the amino acid sequence of any one of SEQ ID NOs: 28-31, and/or the mRNA encoding EBNA3A comprises an open reading frame comprising a nucleic acid sequence having at least 80%, at least 85%, at least 90%, at least 95%, or 100% identity to the nucleic acid sequence of SEQ ID NO: 11; or having at least 80%, at least 85%, at least 90%, at least 95%, or 100% identity to the nucleic acid sequence of any one of SEQ ID NOs: 24-27, and/or the mRNA encoding EBNA3A comprises a nucleic acid sequence having at least 80%, at least 85%, at least 90%, at least 95%, or 100% identity to the nucleic acid sequence of SEQ ID NO: 17; or having at least 80%, at least 85%, at least 90%, at least 95%, or 100% identity to the nucleic acid sequence of any one of SEQ ID NOs: 32-35; and/or,
f. wherein the EBV LMP2B comprises an amino acid sequence having at least 80%, at least 85%, at least 90%, at least 95%, or 100% identity to the amino acid sequence of SEQ ID NO: 6, and/or the mRNA encoding EBV LMP2B comprises an open reading frame comprising a nucleic acid sequence having at least 80%, at least 85%, at least 90%, at least 95%, or 100% identity to the nucleic acid sequence of SEQ ID NO: 12 and/or wherein the mRNA encoding EBV LMP2B comprises a nucleic acid sequence having at least 80%, at least 85%, at least 90%, at least 95%, or 100% identity to the nucleic acid sequence of SEQ ID NO: 18.

9. The EBV mRNA vaccine of any one of claims 1-8, wherein one or more of the mRNAs comprise a chemical modification.

10. A therapeutic Epstein-Barr virus (EBV) messenger ribonucleic acid (mRNA) vaccine 10 comprising:
one or more mRNAs encoding at least two EBV latent antigens; and a lipid nanoparticle, wherein the EBV latent antigens are selected from EBV nuclear antigen 1 (EBNA1), EBV nuclear antigen 2 (EBNA2), EBV nuclear antigen 3A (EBNA3A), EBV nuclear antigen 3B (EBNA3B), EBV nuclear antigen 3C (EBNA3C), EBV latent membrane protein 1 (LMP1), 15 EBV latent membrane protein 2A (LMP2A), and EBV latent membrane protein 2B (LMP2B).

11. The therapeutic EBV mRNA vaccine of claim 10, wherein 50% or less of the mass of the mRNAs in the vaccine are mRNAs encoding the EBV latent antigens.

12. The therapeutic EBV mRNA vaccine of claim 10 or 11, further comprising one or more mRNAs encoding at least one EBV lytic antigen wherein about 50% to about 80% of the mass of the mRNAs in the vaccine are mRNAs encoding the EBV lytic antigens and wherein about 20% to about 50% of the mass of the mRNAs in the vaccine are mRNAs encoding the EBV latent antigens.

13. The therapeutic EBV mRNA vaccine of claim 12, wherein the EBV lytic antigen is an EBV glycoprotein, optionally selected from EBV glycoprotein 350 (gp330), EBV glycoprotein 220 (gp220), EBV glycoprotein H (gH), EBV glycoprotein L (gL), EBV glycoprotein 42 (gp42), and EBV glycoprotein B (gB).

14. The therapeutic EBV mRNA vaccine of any one of claims 12-13, wherein:
about 25% to about 45%, or optionally about 40% to about 45%, or about 42% of the mass of the mRNAs in the vaccine are mRNAs encoding EBV gp220;
about 10% to about 20%, or optionally about 15% to about 20%, or about 16% of the mass of the mRNAs in the vaccine are mRNAs encoding EBV gH;
about 5% to about 15%, or optionally about 10% to about 15%, or about 10.5% of the mass of the mRNAs in the vaccine are mRNAs encoding EBV gL;
about 5% to about 15%, or optionally about 10% to about 15%, or about 10.5% of the mass of the mRNAs in the vaccine are mRNAs encoding EBV gp42;
about 10% to about 25%, or optionally about 10% to about 15%, or about 10.5% of the mass of the mRNAs in the vaccine are mRNAs encoding EBBA3A; and
about 10% to about 25%, or optionally about 10% to about 15%, or about 10.5% of the mass of the mRNAs in the vaccine are mRNAs encoding EBV LMP2B.

15. The therapeutic EBV mRNA vaccine of any one of claims 10-14, wherein the EBNA3A has one or more of the following features:
a. lacks a C-terminal transcriptional regulator domain,
b. has a length of about 523 amino acids,
c. comprises amino acid residues corresponding to amino acid residues 1-523 of the naturally occurring EBNA3A,
d. does not include amino acid residues corresponding to amino acid residues 524-944 of the naturally occurring EBNA3A,
e. the N-terminus of the EBNA3A is truncated relative to the naturally occurring EBNA3A, wherein the C-terminus of the EBNA3A is truncated relative to the naturally occurring EBNA3A,
f. has a length of about 455 amino acids,
g. comprises amino acid residues corresponding to amino acid residues 68-523 of the naturally occurring EBNA3A,
h. does not include amino acid residues corresponding to amino acid residues 1-67 and 524-944 of the naturally occurring EBNA3A,
i. one or more nuclear localization signals (NLS) in the EBNA3A are mutated relative to the naturally occurring EBNA3A,
j. 1-4, 2-4, 3-4, or 4 of the NLS in the EBNA3A are mutated relative to the naturally occurring EBNA3A, optionally wherein the mutated NLS are at amino acid positions selected from: 63-66, 146-155, 375-381, and 394-398, relative to the naturally occurring EBNA3A, and/or,
k. has a C-terminus that is truncated at amino acid residue 68, the N-terminus of the EBNA3A is truncated at amino acid residue 523, and the EBNA3 comprises NLS mutations at amino acid residues 146-155, 375-378, and 394-398, relative to the naturally occurring EBNA3A.

16. A therapeutic Epstein-Barr virus (EBV) messenger ribonucleic acid (mRNA) vaccine comprising:
one or more mRNAs encoding a EBNA3A antigen; and a lipid nanoparticle, wherein the EBNA3A antigen has one or more of the following features:
a. lacks a C-terminal transcriptional regulator domain,
b. has a length of about 523 amino acids,
c. comprises amino acid residues corresponding to amino acid residues 1-523 of the naturally occurring EBNA3A,
d. does not include amino acid residues corresponding to amino acid residues 524-944 of the naturally occurring EBNA3A,
e. the N-terminus of the EBNA3A is truncated relative to the naturally occurring EBNA3A, wherein the C-terminus of the EBNA3A is truncated relative to the naturally occurring EBNA3A,
f. has a length of about 455 amino acids,
g. comprises amino acid residues corresponding to amino acid residues 68-523 of the 25 naturally occurring EBNA3A,
h. does not include amino acid residues corresponding to amino acid residues 1-67 and 524-944 of the naturally occurring EBNA3A,
i. one or more nuclear localization signals (NLS) in the EBNA3A are mutated relative to the naturally occurring EBNA3A,
j. 1-4, 2-4, 3-4, or 4 of the NLS in the EBNA3A are mutated relative to the naturally occurring EBNA3A, optionally wherein the mutated NLS are at amino acid positions selected from: 63-66, 146-155, 375-381, and 394-398, relative to the naturally occurring EBNA3A, and/or,
k. has a C-terminus that is truncated at amino acid residue 68, the N-terminus of the EBNA3A is truncated at amino acid residue 523, and the EBNA3 comprises NLS mutations at amino acid residues 146-155, 375-378, and 394-398, relative to the naturally occurring EBNA3A.

17. A method, comprising administering to a subject the EBV mRNA vaccine of any one of the preceding claims in a therapeutically effective amount.

18. A method for treating an EBV infection, comprising administering to a subject an EBV mRNA vaccine comprising one or more mRNAs encoding at least two latent EBV antigens to induce a therapeutically effective cytotoxic CD8 T cell response against EBV infected cells.

19. The method of claim 17 or 18, wherein the therapeutically effective amount is one or more doses of 25-150 μg, 25-100 μg, 25-50 μg, 50-150 μg, 50-100 μg, 5-25 μg, 25 μg, 30 μg, 40 μg, 50 μg, 75 μg, or 100 μg of the EBV mRNA vaccine.

20. The method of any one of claims 17-19, wherein the subject has been infected with EBV.

21. The method of any one of claims 17-19, wherein the subject is a patient who has received, or is scheduled to receive, a solid organ transplant or a hematopoietic stem cell transplant.

22. The method of any one of claims 17-19, wherein the subject has, or is at risk of having, a posttransplant lymphoproliferative disorder.

23. The method of any one of claims 17-19, wherein the subject has, or is at risk of having, infectious mononucleosis.

24. The method of any one of claims 17-19, wherein the subject has, or is at risk of having, multiple sclerosis.

25. The method of any one of claims 17-19, wherein the subject has, or is at risk of having, systemic lupus erythematosus.

26. The method of any one of claims 17-19, wherein the subject has, or is at risk of having, cancer.

27. The method of claim 26, wherein the cancer is selected from Burkitt lymphoma, Hodgkin's lymphoma, nasopharyngeal carcinoma, and gastric cancer.

28. The method of any one of claims 17-27, wherein the subject is EBV-seropositive.

29. The method of any one of claims 17-27, wherein the subject is EBV-seronegative.

30. The method of any one of claims 17-19, wherein the effective amount induces a CD8+ T cell response.


SEQUENCE LISTING (107 sequences)

SEQ ID NO: 1 - protein, 658 aa, Human gammaherpesvirus 4
MEAALLVCQYTIQSLIHLTGEDPGFFNVEIPEFPFYPTCNVCTADVNVTINFDVGGKKHQLDLDFGQLTPHTKAVYQPRGAFGGSENATNLFLLELLGAGELALTMRSKKLPINVTTGEEQQVSLESVDVYFQDVFGTMWCHHAEMQNPVYLIPETVPYIKWDNCNSTNITAVVRAQGLDVTLPLSLPTSAQDSNFSVKTEMLGNEIDIECIMEDGEISQVLPGDNKFNITCSGYESHVPSGGILTSTSPVATPIPGTGYAYSLRLTPRPVSRFLGNNSILYVFYSGNGPKASGGDYCIQSNIVFSDEIPASQDMPTNTTDITYVGDNATYSVPMVTSEDANSPNVTVTAFWAWPNNTETDFKCKWTLTSGTPSGCENISGAFASNRTFDITVSGLGTAPKTLIITRTATNATTTTHKVIFSKAPESTTTSPTLNTTGFADPNTTTGLPSSTHVPTNLTAPASTGPTVSTADVTSPTPAGTTSGASPVTPSPSPWDNGTESTPPQNATSPQAPSGQKTAVPTVTSTGGKANSTTGGKHTTGHGARTSTEPTTDYGGDSTTPRPRYNATTYLPPSTSSKLRPRWTFTSPPVTTAQATVPVPPTSQPRFSNLSMLVLQWASLAVLTLLLLLVMADCAFRRNLSTSHTYTTPPYDDAETYV

SEQ ID NO: 2 - protein, 706 aa, Human gammaherpesvirus 4
MQLLCVFCLVLLWEVGAASLSEVKLHLDIEGHASHYTIPWTELMAKVPGLSPEALWREANVTEDLASMLNRYKLIYKTSGTLGIALAEPVDIPAVSEGSMQVDASKVHPGVISGLNSPACMLSAPLEKQLFYYIGTMLPNTRPHSYVFYQLRCHLSYVALSINGDKFQYTGAMTSKFLMGTYKRVTEKGDEHVLSLVFGKTKDLPDLRGPFSYPSLTSAQSGDYSLVIVTTFVHYANFHNYFVPNLKDMFSRAVTMTAASYARYVLQKLVLLEMKGGCREPELDTETLTTMFEVSVAFFKVGHAVGETGNGCVDLRWLAKSFFELTVLKDIIGICYGATVKGMQSYGLERLAAMLMATVKMEELGHLTTEKQEYALRLATVGYPKAGVYSGLIGGATSVLLSAYNRHPLFQPLHTVMRETLFIGSHVVLRELRLNVTTQGPNLALYQLLSTALCSALEIGEVLRGLALGTESGLFSPCYLSLRFDLTRDKLLSMAPQEATLDQAAVSNAVDGFLGRLSLEREDRDAWHLPAYKCVDRLDKVLMIIPLINVTFIISSDREVRGSALYEASTTYLSSSLFLSPVIMNKCSQGAVAGEPRQIPKIQNFTRTQKSCIFCGFALLSYDEKEGLETTTYITSQEVQNSILSSNYFDFDNLHVHYLLLTTNGTVMEIAGLYEERAHVVLAIILYFIAFALGIFLVHKIVMFFL

SEQ ID NO: 3 - protein, 137 aa, Human gammaherpesvirus 4
MRAVGVFLAICLVTIFVLPTWGNWAYPCCHVTQLRAQHLLALENISDIYLVSNQTCDGFSLASLNSPKNGSNQLVISRCANGLNVVSFFISILKRSSSALTGHLRELLTTLETLYGSFSVEDLFGANLNRYAWHRGG

SEQ ID NO: 4 - protein, 204 aa, synthetic construct
MTRRRVLSVVVLLAALACRLGAITWVPKPNVEVWPVDPPPPVNFNKTAEQEYGDKEVKLPHWTPTLHTFQVPQNYTKANCTYCNTREYTFSYKGCCFYFTKKKHTWNGCFQACAELYPCTYFYGPTPDILPVVTRNLNAIESLWVGVYRVGEGNWTSLDGGTFKVYQIFGSHCTYVSKFSTVPVSHHECSFLKPCLCVSQRSNS

SEQ ID NO: 5 - protein, 457 aa, synthetic construct
MDHDAGGSAPARPMLPPQPDLPGREAILRRFPLDLRTLLQAIGAAATRIDTRAIDQFFGSQISNTEMYIMYAMAIRQAIGSGSGSGSGSRDQAKWRLQTLAAGWPMGYQAYSSWMYSYTDHQTTPTFVHLQATLGCTGGRRCHVTFSAGTFKLPRCTPGDRQWLYVQSSVGNIVQSCNPRYSIFFDYMAIHRSLTKIWEEVLTPDQRVSFMEFLGFLQRTDLSYIKSFVSDALGTTSIQTPWIDDNPSTETAQAWNAGFLRGRAYGIDLLRTEGEHVEGATGETREESEDTESDGDDEDLPCIVSRGGGSGSRPPIFIRRLHRLLLMGSGSGTEQGKEVLEKARGSTYGTPRPPVPKPRPEVPQSDETATSHGSAQVPEPPTIHLAAQGMAYPLHEQHGMAPCPVAQAPPTPLPPVSPGDQLPGVFSDGRVACAPVPAPAGPIVRPWEPSLTQAAGQ

SEQ ID NO: 6 - protein, 378 aa, synthetic construct
MNPVCLPVIVAPYLFWLAAIAASCFTASVSTVVTATGLALSLLLLAAVASSYAAAQRKLLTPVTVLTAVVTFFAICLTWRIEDPPFNSLLFALLAAAGGLQGIYVLVMLVLLILAYRRRWRRLTVCGGIMFLACVLVLIVDAVLQLSPLLGAVTVVSMTLLLLAFVLWLSSPGGLGTLGAALLTLAAALALLASLILGTLNLTTMFLLMLLWTLVVLLICSSCSSCPLSKILLARLFLYALALLLLASALIAGGSILQTNFKSLSSTEFIPNLFCMLLLIVAGILFILAILTEWGSGNRTYGPVFMCLGGLLTMVAGAVWLTVMSNTLLSAWILTAGFLIFLIGFALFGVIRCCRYCCYYCLTLESEERPPTPYRNTV

SEQ ID NO: 7 - RNA, 1974 nt, synthetic construct
  source 1..1974: mol_type = other RNA
atggaggcagcactgctggtctgccagtatacaatccagtcgctcattcatctgacaggcgaggaccccggcttctttaacgtggaaatcccagagttccctttctatccaacctgtaacgtttgcactgctgacgtgaacgtgacaatcaatttcgacgtgggaggaaagaagcatcagcttgatctggatttcggccaactgacgccacacaccaaagcggtgtaccaaccaaggggtgcatttggagggagcgagaatgctacaaacctgttcttactggaactgcttggtgcaggagagctggcactaaccatgcgaagcaagaagctccccatcaacgttaccaccggggaagaacagcaagtcagccttgaatccgtggatgtgtactttcaggatgttttcggcaccatgtggtgtcaccacgctgagatgcagaatcccgtgtatctcatacccgagactgtaccctacatcaagtgggataattgcaattcaactaatattacggctgtggtgagggcccagggcttggacgtgactctgcccttatctctacctacttctgcccaagactccaatttctctgtcaagaccgagatgctcgggaatgagatcgatatcgagtgcatcatggaggacggtgagataagccaggttctgcccggcgacaacaagttcaatatcacttgttctggctacgagtcccatgtgcctagtggtggcatactcacaagtacttctcccgtagccacgcccattcccggaaccggatacgcctacagtctgcgtctgaccccacggcctgtgtccagattcctgggtaacaatagtatcttatacgtgttttatagcggaaacggccctaaagcgtccggaggggactattgtattcagagtaatatcgttttctctgatgagattcctgccagtcaggacatgccgacaaacacaactgatattacctacgtgggcgacaatgccacgtattcagtgcccatggtcacgagcgaggacgccaattcaccaaatgttactgtaacagctttctgggcctggccaaataacactgagactgacttcaaatgtaagtggactttgacctctggaactccgtcgggttgcgagaatatcagcggggcctttgcttccaacaggactttcgacatcactgtctcagggctggggacagcaccgaagacattaatcataacacggaccgccaccaacgccacgactacaacccataaggtgatcttttccaaggcacctgagtccaccactacctccccgactcttaacactacgggcttcgctgatcccaacaccactacggggttgcctagctcgacacatgtgccgacgaacctgactgcccctgcatcgaccgggcccacagtttcgaccgccgatgtgacatcaccaacgcccgcaggtacaacctcaggcgccagcccagtgaccccttccccaagcccctgggataatggaacggagtccacgcctccccagaacgcaaccagtcctcaggcgcccagcgggcagaagactgcggtgccaactgtgaccagcaccggtggcaaggccaactcaacaactggaggcaagcatacgacggggcacggcgcccggacctccactgaacccacgaccgattacggaggtgacagcacaacaccgcggccacgatataatgccaccacttatctgccaccatccacaagctccaagctgcggccacggtggaccttcacaagcccacccgtgacgactgcccaagcgacggtgccagtgccacctacaagccagccacgcttctccaaccttagtatgctcgttctccagtgggccagccttgctgttctgaccctcctcctgctgctcgtgatggccgattgcgcctttaggagaaacctcagcactagccacacgtacaccacgccgccctacgatgacgccgagacttacgtc

SEQ ID NO: 8 - RNA, 2118 nt, synthetic construct
  source 1..2118: mol_type = other RNA
atgcagttgttgtgcgtgttctgcctcgtgttactctgggaggtgggcgccgccagccttagcgaggtgaagctccacttggacatcgagggccacgccagccactacaccatcccctggaccgagctcatggccaaggtgcccggccttagccccgaggccctgtggcgggaggccaacgtgaccgaggacctggccagcatgctgaaccggtacaagctgatctacaagaccagcggcaccctgggcatcgccctggccgagcccgtggacatccccgccgttagcgaaggcagcatgcaggtggacgccagcaaggtgcaccccggcgtgatcagcggcctgaacagccccgcctgtatgttgagcgccccactggagaagcagctgttctactacatcggcaccatgctgcccaacacccggccccacagctacgtgttctaccagctgcggtgccacctgagctacgttgccctgagcatcaacggcgacaagttccagtacaccggcgccatgaccagcaagttcctgatgggcacctacaagcgggtcaccgagaagggcgacgagcacgtgctgtcactggtgttcggcaagaccaaggacctgcccgacctgcggggccccttcagctaccctagtttgaccagcgcccagagcggcgactacagcttggtgatcgtgaccaccttcgtgcactacgccaacttccacaactacttcgtgcccaacctgaaggacatgttcagccgggccgtgaccatgactgccgcttcttacgcccggtacgtgctgcagaagctggtcctgctggagatgaagggcggctgccgggagcccgagctggacaccgaaacactgaccaccatgttcgaggtgagcgtggccttcttcaaggtgggtcacgcggtgggcgaaaccggcaacggctgcgtggacttacgctggctggccaagagcttcttcgagctgaccgtgctgaaggatatcatcggcatctgctacggcgccaccgtgaagggcatgcagagctacggcctggagcggctggccgccatgcttatggcaacagtgaagatggaggagctgggacacctgacaacagagaagcaggagtacgccctgagactggccacagtgggctacccaaaggccggcgtgtacagtggactgatcggcggcgcaaccagcgtgctgctatccgcttacaaccggcacccgctgttccagcccctgcacaccgtgatgcgggaaaccctgttcatcggaagccacgtcgtgctgcgggagctgaggctgaacgtaaccacccagggccctaatctggccctgtatcagctcctcagtaccgccctgtgcagcgcccttgagatcggcgaggtgctcagaggcctggccctcggtaccgagagcggcctcttcagcccatgctacttaagcctgcggttcgacctgacccgggacaagttgctgagcatggccccgcaggaggccacactggaccaggcagctgtatccaacgccgtggacggcttcctgggcagactgtccctggaacgggaggaccgggacgcctggcacctgcctgcctacaagtgtgtggatcggctggacaaggtgctgatgatcatccctctgattaatgtcaccttcatcatcagcagcgaccgggaggtgcggggatccgccctctacgaggccagcaccacctatctgagcagcagcctgttcctgtctcctgtgatcatgaacaagtgcagccagggcgccgtggccggcgagccccggcagatccccaagatccagaacttcacccggacccagaagtcttgcatcttctgcggcttcgcccttttgtcctacgacgagaaggagggcttggagactacaacctacatcaccagccaggaggtgcagaacagcatcctgtcatctaattacttcgacttcgacaacctgcacgttcattacctgctcctcaccaccaacggtaccgtcatggaaatcgccggactgtacgaggagcgggcccatgttgtgctggccatcatcctgtacttcatcgctttcgcacttggcatcttcctggtgcacaagatcgtgatgttcttcctg

SEQ ID NO: 9 - RNA, 411 nt, synthetic construct
  source 1..411: mol_type = other RNA
atgagagccgtgggcgtgttcctggccatctgcctggtgaccatcttcgtgctgcccacctggggcaactgggcctacccctgctgccacgtgacccagctgagagcccagcacctgctggccctggagaacatcagcgacatctacctggtgagcaaccagacctgcgacggcttcagcctggccagcctgaacagccccaagaacggcagcaaccagctggtgatcagcagatgcgccaacggcctgaacgtggtgagcttcttcatcagcatcctgaagagaagcagcagcgccctgaccggccacctgagagagctgctgaccaccctggagaccctgtacggcagcttcagcgtggaggacctgttcggcgccaacctgaacagatacgcctggcacagaggcggc

SEQ ID NO: 10 - RNA, 612 nt, synthetic construct
  source 1..612: mol_type = other RNA
atgacccggcggcgggtgttaagcgtggtggtgctcttagccgccttagcctgccggcttggcgccatcacctgggtgcccaagcccaacgtggaggtgtggcccgtggacccgccacctcccgtgaacttcaacaagaccgccgagcaggagtacggcgacaaggaggtgaagctgccccactggacgccgaccctgcacaccttccaggtgccccagaactacaccaaggccaactgcacctactgcaacaccagagagtacaccttcagctacaagggctgctgcttctacttcaccaagaagaagcacacctggaacggctgcttccaggcctgcgccgagctgtacccctgcacctacttctacggccccaccccagacatcctgcccgtggtgaccagaaacctgaacgccatcgagagcctgtgggtgggcgtgtacagagtgggcgagggcaactggaccagcctggacggcggcaccttcaaggtgtaccagatcttcggcagccactgcacctacgtgagcaagttcagcaccgtgcccgtgagccaccacgagtgcagcttcctgaagccctgcctgtgcgtgagccagagaagcaacagc

SEQ ID NO: 11 - RNA, 1371 nt, synthetic construct
  source 1..1371: mol_type = other RNA
atggatcacgacgccggaggaagcgcacccgccagacccatgctgcctccgcagcccgaccttcccggacgggaggccatcctgcggcggttccctctggacctgcggaccctgctgcaggctatcggcgctgccgctacacggatcgacacccgggccatcgaccagttcttcggcagccagatcagcaacaccgagatgtacatcatgtacgccatggccatccggcaagccatcggcagcggaagcggcagcggaagcggaagcagagaccaggccaagtggcggctgcaaaccttagccgctggctggcccatgggctaccaggcctacagcagctggatgtacagctacaccgaccaccagaccactcccaccttcgtgcacctgcaggccacactcggctgcactggcggtagacggtgccacgtgaccttcagcgccggcaccttcaagctgcctcggtgtacacccggcgaccgtcagtggctgtacgtgcagagcagcgtgggcaacatcgtgcagagctgcaatccccggtacagcatcttcttcgactacatggccatccaccggagcctgaccaagatctgggaggaggtgctgactcccgaccagcgggtgagcttcatggagttcctgggcttcctgcagcggaccgacctgagctacatcaagagcttcgtgagcgacgccctgggcaccaccagcatccagacgccctggatcgacgacaaccccagcaccgagaccgcccaggcctggaacgccggctttctacggggacgggcctacggaatcgacctgctgcggaccgagggcgagcacgtcgagggcgctacgggtgagacccgggaggagagcgaggacaccgagagcgacggcgacgacgaggacctgccctgcatcgtgagcagaggcggcggctccggcagcagacctcccatcttcattcgccggctccaccgtctgctgctgatgggcagcggcagcggcaccgagcagggcaaggaggtgctggagaaggcccggggcagcacctacggaaccccacggcctcctgtgcccaagcccaggcccgaagtgccccagagcgacgagaccgccaccagccacggcagtgcccaggtgcccgagcctccaaccatccatctggccgcacagggcatggcctatcccctgcacgagcagcacggcatggcaccctgtcccgtggcccaggctcctcctactcccctgccacccgtgagccctggcgaccaactccccggggtgttcagcgacggcagggtggcttgcgctccagtaccggcccctgcaggacctatcgttcggccgtgggagccaagcctgacccaggcagcgggccag

SEQ ID NO: 12 - RNA, 1134 nt, synthetic construct
  source 1..1134: mol_type = other RNA
atgaaccccgtgtgcctgcccgtgatcgtggccccttacctgttctggctggctgccatcgccgccagctgcttcaccgccagcgtgagcaccgtggtgaccgccaccggacttgccctgagcctcctgctgctggctgccgtggccagctcctacgccgccgctcagcggaaactgctgactcccgtgaccgtgctgaccgccgtggtgaccttcttcgccatctgcctgacctggcggatcgaggatcctccattcaacagcctgctgtttgctcttctcgcagcggcaggaggcctgcagggcatctacgtgctggtgatgcttgtgctgctgatactggcgtatcggaggcgctggcggagactgaccgtgtgcggcggcatcatgttcctggcctgcgtgctggtgctgatcgtggacgccgtgctgcagctgtcacccctgctgggtgccgtgaccgtggtgagcatgaccctgctgctgctggccttcgtgctgtggctgagctctcctggtggcctgggaaccctgggcgctgctctgctgacccttgcggccgcattggccctgctggccagcctgatcctgggcaccctgaacctgaccaccatgttcctgctgatgctgctgtggaccctggtggtgctgctgatctgcagcagctgtagcagctgccctctgagcaagatcctgctggcccggctgttcctgtacgccctggcccttctgctgctggccagcgcactgatcgccggcggcagcatcctgcagaccaacttcaagagcctgagcagcaccgagttcatccccaacctgttctgcatgctgctgctgatcgtggccggcatcctgttcatcctggccatcctgaccgagtggggcagcggcaaccggacctacggccccgtgttcatgtgcctgggcggcctgctgaccatggttgccggggcagtgtggctgaccgtgatgagcaacaccctgctgagcgcctggatcctgaccgccggcttcctgatcttcctgatcggcttcgccctgttcggcgtgatccggtgctgccggtactgctgctactactgcctgaccctggagagcgaggagcggcctcctacgccctaccggaacaccgtg

SEQ ID NO: 13 - RNA, 2151 nt, synthetic construct
  source 1..2151: mol_type = other RNA
gaggaaataagagagaaaagaagagtaagaagaaatataagaccccggcgccgccaccatggaggcagcactgctggtctgccagtatacaatccagtcgctcattcatctgacaggcgaggaccccggcttctttaacgtggaaatcccagagttccctttctatccaacctgtaacgtttgcactgctgacgtgaacgtgacaatcaatttcgacgtgggaggaaagaagcatcagcttgatctggatttcggccaactgacgccacacaccaaagcggtgtaccaaccaaggggtgcatttggagggagcgagaatgctacaaacctgttcttactggaactgcttggtgcaggagagctggcactaaccatgcgaagcaagaagctccccatcaacgttaccaccggggaagaacagcaagtcagccttgaatccgtggatgtgtactttcaggatgttttcggcaccatgtggtgtcaccacgctgagatgcagaatcccgtgtatctcatacccgagactgtaccctacatcaagtgggataattgcaattcaactaatattacggctgtggtgagggcccagggcttggacgtgactctgcccttatctctacctacttctgcccaagactccaatttctctgtcaagaccgagatgctcgggaatgagatcgatatcgagtgcatcatggaggacggtgagataagccaggttctgcccggcgacaacaagttcaatatcacttgttctggctacgagtcccatgtgcctagtggtggcatactcacaagtacttctcccgtagccacgcccattcccggaaccggatacgcctacagtctgcgtctgaccccacggcctgtgtccagattcctgggtaacaatagtatcttatacgtgttttatagcggaaacggccctaaagcgtccggaggggactattgtattcagagtaatatcgttttctctgatgagattcctgccagtcaggacatgccgacaaacacaactgatattacctacgtgggcgacaatgccacgtattcagtgcccatggtcacgagcgaggacgccaattcaccaaatgttactgtaacagctttctgggcctggccaaataacactgagactgacttcaaatgtaagtggactttgacctctggaactccgtcgggttgcgagaatatcagcggggcctttgcttccaacaggactttcgacatcactgtctcagggctggggacagcaccgaagacattaatcataacacggaccgccaccaacgccacgactacaacccataaggtgatcttttccaaggcacctgagtccaccactacctccccgactcttaacactacgggcttcgctgatcccaacaccactacggggttgcctagctcgacacatgtgccgacgaacctgactgcccctgcatcgaccgggcccacagtttcgaccgccgatgtgacatcaccaacgcccgcaggtacaacctcaggcgccagcccagtgaccccttccccaagcccctgggataatggaacggagtccacgcctccccagaacgcaaccagtcctcaggcgcccagcgggcagaagactgcggtgccaactgtgaccagcaccggtggcaaggccaactcaacaactggaggcaagcatacgacggggcacggcgcccggacctccactgaacccacgaccgattacggaggtgacagcacaacaccgcggccacgatataatgccaccacttatctgccaccatccacaagctccaagctgcggccacggtggaccttcacaagcccacccgtgacgactgcccaagcgacggtgccagtgccacctacaagccagccacgcttctccaaccttagtatgctcgttctccagtgggccagccttgctgttctgaccctcctcctgctgctcgtgatggccgattgcgcctttaggagaaacctcagcactagccacacgtacaccacgccgccctacgatgacgccgagacttacgtctgataataggctggagcctcggtggcctagcttcttgccccttgggcctccccccagcccctcctccccttcctgcacccgtacccccgtggtctttgaataaagtctgagtgggcggc

SEQ ID NO: 14 - RNA, 2295 nt, synthetic construct
  source 1..2295: mol_type = other RNA
gaggaaataagagagaaaagaagagtaagaagaaatataagaccccggcgccgccaccatgcagttgttgtgcgtgttctgcctcgtgttactctgggaggtgggcgccgccagccttagcgaggtgaagctccacttggacatcgagggccacgccagccactacaccatcccctggaccgagctcatggccaaggtgcccggccttagccccgaggccctgtggcgggaggccaacgtgaccgaggacctggccagcatgctgaaccggtacaagctgatctacaagaccagcggcaccctgggcatcgccctggccgagcccgtggacatccccgccgttagcgaaggcagcatgcaggtggacgccagcaaggtgcaccccggcgtgatcagcggcctgaacagccccgcctgtatgttgagcgccccactggagaagcagctgttctactacatcggcaccatgctgcccaacacccggccccacagctacgtgttctaccagctgcggtgccacctgagctacgttgccctgagcatcaacggcgacaagttccagtacaccggcgccatgaccagcaagttcctgatgggcacctacaagcgggtcaccgagaagggcgacgagcacgtgctgtcactggtgttcggcaagaccaaggacctgcccgacctgcggggccccttcagctaccctagtttgaccagcgcccagagcggcgactacagcttggtgatcgtgaccaccttcgtgcactacgccaacttccacaactacttcgtgcccaacctgaaggacatgttcagccgggccgtgaccatgactgccgcttcttacgcccggtacgtgctgcagaagctggtcctgctggagatgaagggcggctgccgggagcccgagctggacaccgaaacactgaccaccatgttcgaggtgagcgtggccttcttcaaggtgggtcacgcggtgggcgaaaccggcaacggctgcgtggacttacgctggctggccaagagcttcttcgagctgaccgtgctgaaggatatcatcggcatctgctacggcgccaccgtgaagggcatgcagagctacggcctggagcggctggccgccatgcttatggcaacagtgaagatggaggagctgggacacctgacaacagagaagcaggagtacgccctgagactggccacagtgggctacccaaaggccggcgtgtacagtggactgatcggcggcgcaaccagcgtgctgctatccgcttacaaccggcacccgctgttccagcccctgcacaccgtgatgcgggaaaccctgttcatcggaagccacgtcgtgctgcgggagctgaggctgaacgtaaccacccagggccctaatctggccctgtatcagctcctcagtaccgccctgtgcagcgcccttgagatcggcgaggtgctcagaggcctggccctcggtaccgagagcggcctcttcagcccatgctacttaagcctgcggttcgacctgacccgggacaagttgctgagcatggccccgcaggaggccacactggaccaggcagctgtatccaacgccgtggacggcttcctgggcagactgtccctggaacgggaggaccgggacgcctggcacctgcctgcctacaagtgtgtggatcggctggacaaggtgctgatgatcatccctctgattaatgtcaccttcatcatcagcagcgaccgggaggtgcggggatccgccctctacgaggccagcaccacctatctgagcagcagcctgttcctgtctcctgtgatcatgaacaagtgcagccagggcgccgtggccggcgagccccggcagatccccaagatccagaacttcacccggacccagaagtcttgcatcttctgcggcttcgcccttttgtcctacgacgagaaggagggcttggagactacaacctacatcaccagccaggaggtgcagaacagcatcctgtcatctaattacttcgacttcgacaacctgcacgttcattacctgctcctcaccaccaacggtaccgtcatggaaatcgccggactgtacgaggagcgggcccatgttgtgctggccatcatcctgtacttcatcgctttcgcacttggcatcttcctggtgcacaagatcgtgatgttcttcctgtgataataggctggagcctcggtggcctagcttcttgccccttgggcctccccccagcccctcctccccttcctgcacccgtacccccgtggtctttgaataaagtctgagtgggcggc

SEQ ID NO: 15 - RNA, 588 nt, synthetic construct
  source 1..588: mol_type = other RNA
gaggaaataagagagaaaagaagagtaagaagaaatataagaccccggcgccgccaccatgagagccgtgggcgtgttcctggccatctgcctggtgaccatcttcgtgctgcccacctggggcaactgggcctacccctgctgccacgtgacccagctgagagcccagcacctgctggccctggagaacatcagcgacatctacctggtgagcaaccagacctgcgacggcttcagcctggccagcctgaacagccccaagaacggcagcaaccagctggtgatcagcagatgcgccaacggcctgaacgtggtgagcttcttcatcagcatcctgaagagaagcagcagcgccctgaccggccacctgagagagctgctgaccaccctggagaccctgtacggcagcttcagcgtggaggacctgttcggcgccaacctgaacagatacgcctggcacagaggcggctgataataggctggagcctcggtggcctagcttcttgccccttgggcctccccccagcccctcctccccttcctgcacccgtacccccgtggtctttgaataaagtctgagtgggcggc

SEQ ID NO: 16 - RNA, 789 nt, synthetic construct
  source 1..789: mol_type = other RNA
gaggaaataagagagaaaagaagagtaagaagaaatataagaccccggcgccgccaccatgacccggcggcgggtgttaagcgtggtggtgctcttagccgccttagcctgccggcttggcgccatcacctgggtgcccaagcccaacgtggaggtgtggcccgtggacccgccacctcccgtgaacttcaacaagaccgccgagcaggagtacggcgacaaggaggtgaagctgccccactggacgccgaccctgcacaccttccaggtgccccagaactacaccaaggccaactgcacctactgcaacaccagagagtacaccttcagctacaagggctgctgcttctacttcaccaagaagaagcacacctggaacggctgcttccaggcctgcgccgagctgtacccctgcacctacttctacggccccaccccagacatcctgcccgtggtgaccagaaacctgaacgccatcgagagcctgtgggtgggcgtgtacagagtgggcgagggcaactggaccagcctggacggcggcaccttcaaggtgtaccagatcttcggcagccactgcacctacgtgagcaagttcagcaccgtgcccgtgagccaccacgagtgcagcttcctgaagccctgcctgtgcgtgagccagagaagcaacagctgataataggctggagcctcggtggcctagcttcttgccccttgggcctccccccagcccctcctccccttcctgcacccgtacccccgtggtctttgaataaagtctgagtgggcggc

SEQ ID NO: 17 - RNA, 1548 nt, synthetic construct
  source 1..1548: mol_type = other RNA
gaggaaataagagagaaaagaagagtaagaagaaatataagaccccggcgccgccaccatggatcacgacgccggaggaagcgcacccgccagacccatgctgcctccgcagcccgaccttcccggacgggaggccatcctgcggcggttccctctggacctgcggaccctgctgcaggctatcggcgctgccgctacacggatcgacacccgggccatcgaccagttcttcggcagccagatcagcaacaccgagatgtacatcatgtacgccatggccatccggcaagccatcggcagcggaagcggcagcggaagcggaagcagagaccaggccaagtggcggctgcaaaccttagccgctggctggcccatgggctaccaggcctacagcagctggatgtacagctacaccgaccaccagaccactcccaccttcgtgcacctgcaggccacactcggctgcactggcggtagacggtgccacgtgaccttcagcgccggcaccttcaagctgcctcggtgtacacccggcgaccgtcagtggctgtacgtgcagagcagcgtgggcaacatcgtgcagagctgcaatccccggtacagcatcttcttcgactacatggccatccaccggagcctgaccaagatctgggaggaggtgctgactcccgaccagcgggtgagcttcatggagttcctgggcttcctgcagcggaccgacctgagctacatcaagagcttcgtgagcgacgccctgggcaccaccagcatccagacgccctggatcgacgacaaccccagcaccgagaccgcccaggcctggaacgccggctttctacggggacgggcctacggaatcgacctgctgcggaccgagggcgagcacgtcgagggcgctacgggtgagacccgggaggagagcgaggacaccgagagcgacggcgacgacgaggacctgccctgcatcgtgagcagaggcggcggctccggcagcagacctcccatcttcattcgccggctccaccgtctgctgctgatgggcagcggcagcggcaccgagcagggcaaggaggtgctggagaaggcccggggcagcacctacggaaccccacggcctcctgtgcccaagcccaggcccgaagtgccccagagcgacgagaccgccaccagccacggcagtgcccaggtgcccgagcctccaaccatccatctggccgcacagggcatggcctatcccctgcacgagcagcacggcatggcaccctgtcccgtggcccaggctcctcctactcccctgccacccgtgagccctggcgaccaactccccggggtgttcagcgacggcagggtggcttgcgctccagtaccggcccctgcaggacctatcgttcggccgtgggagccaagcctgacccaggcagcgggccagtgataataggctggagcctcggtggcctagcttcttgccccttgggcctccccccagcccctcctccccttcctgcacccgtacccccgtggtctttgaataaagtctgagtgggcggc

SEQ ID NO: 18 - RNA, 1311 nt, synthetic construct
  source 1..1311: mol_type = other RNA
gaggaaataagagagaaaagaagagtaagaagaaatataagaccccggcgccgccaccatgaaccccgtgtgcctgcccgtgatcgtggccccttacctgttctggctggctgccatcgccgccagctgcttcaccgccagcgtgagcaccgtggtgaccgccaccggacttgccctgagcctcctgctgctggctgccgtggccagctcctacgccgccgctcagcggaaactgctgactcccgtgaccgtgctgaccgccgtggtgaccttcttcgccatctgcctgacctggcggatcgaggatcctccattcaacagcctgctgtttgctcttctcgcagcggcaggaggcctgcagggcatctacgtgctggtgatgcttgtgctgctgatactggcgtatcggaggcgctggcggagactgaccgtgtgcggcggcatcatgttcctggcctgcgtgctggtgctgatcgtggacgccgtgctgcagctgtcacccctgctgggtgccgtgaccgtggtgagcatgaccctgctgctgctggccttcgtgctgtggctgagctctcctggtggcctgggaaccctgggcgctgctctgctgacccttgcggccgcattggccctgctggccagcctgatcctgggcaccctgaacctgaccaccatgttcctgctgatgctgctgtggaccctggtggtgctgctgatctgcagcagctgtagcagctgccctctgagcaagatcctgctggcccggctgttcctgtacgccctggcccttctgctgctggccagcgcactgatcgccggcggcagcatcctgcagaccaacttcaagagcctgagcagcaccgagttcatccccaacctgttctgcatgctgctgctgatcgtggccggcatcctgttcatcctggccatcctgaccgagtggggcagcggcaaccggacctacggccccgtgttcatgtgcctgggcggcctgctgaccatggttgccggggcagtgtggctgaccgtgatgagcaacaccctgctgagcgcctggatcctgaccgccggcttcctgatcttcctgatcggcttcgccctgttcggcgtgatccggtgctgccggtactgctgctactactgcctgaccctggagagcgaggagcggcctcctacgccctaccggaacaccgtgtgataataggctggagcctcggtggcctagcttcttgccccttgggcctccccccagcccctcctccccttcctgcacccgtacccccgtggtctttgaataaagtctgagtgggcggc

SEQ ID NO: 19 - RNA, 58 nt, synthetic construct
  source 1..58: mol_type = other RNA
gaggaaataagagagaaaagaagagtaagaagaaatataagaccccggcgccgccacc

SEQ ID NO: 20 - RNA, 119 nt, synthetic construct
  source 1..119: mol_type = other RNA
tgataataggctggagcctcggtggcctagcttcttgccccttgggcctccccccagcccctcctccccttcctgcacccgtacccccgtggtctttgaataaagtctgagtgggcggc

SEQ ID NO: 21 - protein, 223 aa, Human gammaherpesvirus 4
MVSFKQVRVPLFTAIALVIVLLLAYFLPPRVRGGGRVAAAAITWVPKPNVEVWPVDPPPPVNFNKTAEQEYGDKEVKLPHWTPTLHTFQVPQNYTKANCTYCNTREYTFSYKGCCFYFTKKKHTWNGCFQACAELYPCTYFYGPTPDILPVVTRNLNAIESLWVGVYRVGEGNWTSLDGGTFKVYQIFGSHCTYVSKFSTVPVSHHECSFLKPCLCVSQRSNS

SEQ ID NO: 22 - protein, 944 aa, Human gammaherpesvirus 4
MDKDRPGPPALDDNMEEEVPSTSVVQEQVSAGDWENVLIELSDSSSEKEAEDAHLEPAQKGTKRKRVDHDAGGSAPARPMLPPQPDLPGREAILRRFPLDLRTLLQAIGAAATRIDTRAIDQFFGSQISNTEMYIMYAMAIRQAIRDRRRNPASRRDQAKWRLQTLAAGWPMGYQAYSSWMYSYTDHQTTPTFVHLQATLGCTGGRRCHVTFSAGTFKLPRCTPGDRQWLYVQSSVGNIVQSCNPRYSIFFDYMAIHRSLTKIWEEVLTPDQRVSFMEFLGFLQRTDLSYIKSFVSDALGTTSIQTPWIDDNPSTETAQAWNAGFLRGRAYGIDLLRTEGEHVEGATGETREESEDTESDGDDEDLPCIVSRGGPKVKRPPIFIRRLHRLLLMRAGKRTEQGKEVLEKARGSTYGTPRPPVPKPRPEVPQSDETATSHGSAQVPEPPTIHLAAQGMAYPLHEQHGMAPCPVAQAPPTPLPPVSPGDQLPGVFSDGRVACAPVPAPAGPIVRPWEPSLTQAAGQAFAPVRPQHMPVEPVPVPTVALERPVYPKPVRPAPPKIAMQGPGETSGIRRARERWRPAPWTPNPPRSPSQMSVRDRLARLRAEAQVKQASVEVQPPQLTQVSPQQPMEGPLVPEQQMFPGAPFSQVADVVRAPGVPAMQPQYFDLPLIQPISQGAPVAPLRASMGPVPPVPATQPQYFDIPLTEPINQGASAAHFLPQQPMEGPLVPEQWMFPGAALSQSVRPGVAQSQYFDLPLTQPINHGAPAAHFLHQPPMEGPWVPEQWMFQGAPPSQGTDVVQHQLDALGYTLHGLNHPGVPVSPAVNQYHLSQAAFGLPIDEDESGEGSDTSEPCEALDLSIHGRPCPQAPEWPVQEEGGQDATEVLDLSIHGRPRPRTPEWPVQGEGGQNVTGPETRRVVVSAVVHMCQDDEFPDLQDPPDEA

SEQ ID NO: 23 - protein, 497 aa, Human gammaherpesvirus 4
MGSLEMVPMGAGPPSPGGDPDGYDGGNNSQYPSASGSSGNTPTPPNDEERESNEEPPPPYEDPYWGNGDRHSDYQPLGTQDQSLYLGLQHDGNDGLPPPPYSPRDDSSQHIYEEAGRGSMNPVCLPVIVAPYLFWLAAIAASCFTASVSTVVTATGLALSLLLLAAVASSYAAAQRKLLTPVTVLTAVVTFFAICLTWRIEDPPFNSLLFALLAAAGGLQGIYVLVMLVLLILAYRRRWRRLTVCGGIMFLACVLVLIVDAVLQLSPLLGAVTVVSMTLLLLAFVLWLSSPGGLGTLGAALLTLAAALALLASLILGTLNLTTMFLLMLLWTLVVLLICSSCSSCPLSKILLARLFLYALALLLLASALIAGGSILQTNFKSLSSTEFIPNLFCMLLLIVAGILFILAILTEWGSGNRTYGPVFMCLGGLLTMVAGAVWLTVMSNTLLSAWILTAGFLIFLIGFALFGVIRCCRYCCYYCLTLESEERPPTPYRNTV

SEQ ID NO: 24 - RNA, 1371 nt, synthetic construct
  source 1..1371: mol_type = other RNA
atggatcacgacgccggaggaagcgcacccgccagacccatgctgcctccgcagcccgaccttcccggacgggaggccatcctgcggcggttccctctggacctgcggaccctgctgcaggctatcggcgctgccgctacacggatcgacacccgggccatcgaccagttcttcggcagccagatcagcaacaccgagatgtacatcatgtacgccatggccatccggcaagccatcggcagcggaagcggcagcggaagcggaagcagagatcaggccaagtggcggctgcaaaccttagccgctggctggcccatgggctaccaggcctacagcagctggatgtacagctacaccgaccaccagaccactcccaccttcgtgcacctgcaggccacactcggctgcactggcggtagacggtgccacgtgaccttcagcgccggcaccttcaagctgcctcggtgtacacccggcgaccgtcagtggctgtacgtgcagagcagcgtgggcaacatcgtgcagagctgcaatccccggtacagcatcttcttcgactacatggccatccaccggagcctgaccaagatctgggaggaggtgctgactcccgaccagcgggtgagcttcatggagttcctgggcttcctgcagcggaccgacctgagctacatcaagagcttcgtgagcgacgccctgggcaccaccagcatccagacgccctggatcgacgacaaccccagcaccgagacagcccaggcctggaacgccggctttctaggtggaggcgcctacggaatcgacctgctgcggaccgagggcgagcacgtcgagggcgctacgggtgagacacgggaggagagcgaggacaccgagagcgacggcgacgacgaggacctgccctgcatcgtgagcagaggcggcggctccggcagcggcagcggcatcttcattcgccggctccaccgtctgctgctgatgggcagcggcagcggcaccgagcagggcaaggaggtgctggagaaggcccggggcagcacctacggaaccccacggcctcctgtgcccaagcccaggcccgaagtgccccagagcgacgagacagccaccagccacggcagtgcccaggtgcccgagcctccaaccatccatctggccgcacagggcatggcctatcccctgcacgagcagcacggcatggcaccctgtcccgtggcccaggctcctcctactcccctgccacccgtgagccctggcgaccaactccccggggtgttcagcgacggcagggtggcttgcgctccagtaccggcccctgcaggacctatcgttcggccgtgggagccaagcctgacccaggcagcgggccag

SEQ ID NO: 25 - RNA, 1371 nt, synthetic construct
  source 1..1371: mol_type = other RNA
atggatcacgacgccggaggaagcgcacccgccagacccatgctgcctccgcagcccgaccttcccggacgggaggccatcctgcggcggttccctctggacctgcggaccctgctgcaggctatcggcgctgccgctacacggatcgacacccgggccatcgaccagttcttcggcagccagatcagcaacaccgagatgtacatcatgtacgccatggccatccggcaagccatcggcagcggaagcggcagcggaagcggaagcagagatcaggccaagtggcggctgcaaaccttagccgctggctggcccatgggctaccaggcctacagcagctggatgtacagctacaccgaccaccagaccactcccaccttcgtgcacctgcaggccacactcggctgcactggcggtagacggtgccacgtgaccttcagcgccggcaccttcaagctgcctcggtgtacacccggcgaccgtcagtggctgtacgtgcagagcagcgtgggcaacatcgtgcagagctgcaatccccggtacagcatcttcttcgactacatggccatccaccggagcctgaccaagatctgggaggaggtgctgactcccgaccagcgggtgagcttcatggagttcctgggcttcctgcagcggaccgacctgagctacatcaagagcttcgtgagcgacgccctgggcaccaccagcatccagacgccctggatcgacgacaaccccagcaccgagacagcccaggcctggaacgccggctttctacggggacgggcctacggaatcgacctgctgcggaccgagggcgagcacgtcgagggcgctacgggtgagacacgggaggagagcgaggacaccgagagcgacggcgacgacgaggacctgccctgcatcgtgagcagaggcggcggctccggcagcggcagcggcatcttcattggcggcctccaccgtctgctgctgatgggcagcggcagcggcaccgagcagggcaaggaggtgctggagaaggcccggggcagcacctacggaaccccacggcctcctgtgcccaagcccaggcccgaagtgccccagagcgacgagacagccaccagccacggcagtgcccaggtgcccgagcctccaaccatccatctggccgcacagggcatggcctatcccctgcacgagcagcacggcatggcaccctgtcccgtggcccaggctcctcctactcccctgccacccgtgagccctggcgaccaactccccggggtgttcagcgacggcagggtggcttgcgctccagtaccggcccctgcaggacctatcgttcggccgtgggagccaagcctgacccaggcagcgggccag

SEQ ID NO: 26 - RNA, 1371 nt, synthetic construct
  source 1..1371: mol_type = other RNA
atggatcacgacgccggaggaagcgcacccgccagacccatgctgcctccgcagcccgaccttcccggacgggaggccatcctgcggcggttccctctggacctgcggaccctgctgcaggctatcggcgctgccgctacacggatcgacacccgggccatcgaccagttcttcggcagccagatcagcaacaccgagatgtacatcatgtacgccatggccatccggcaagccatcggcagcggaagcggcagcggaagcggaagcagagatcaggccaagtggcggctgcaaaccttagccgctggctggcccatgggctaccaggcctacagcagctggatgtacagctacaccgaccaccagaccactcccaccttcgtgcacctgcaggccacactcggctgcactggcggtagacggtgccacgtgaccttcagcgccggcaccttcaagctgcctcggtgtacacccggcgaccgtcagtggctgtacgtgcagagcagcgtgggcaacatcgtgcagagctgcaatccccggtacagcatcttcttcgactacatggccatccaccggagcctgaccaagatctgggaggaggtgctgactcccgaccagcgggtgagcttcatggagttcctgggcttcctgcagcggaccgacctgagctacatcaagagcttcgtgagcgacgccctgggcaccaccagcatccagacgccctggatcgacgacaaccccagcaccgagacagcccaggcctggaacgccggctttctaggtggaggcgcctacggaatcgacctgctgcggaccgagggcgagcacgtcgagggcgctacgggtgagacacgggaggagagcgaggacaccgagagcgacggcgacgacgaggacctgccctgcatcgtgagcagaggcggcggctccggcagcggcagcggcatcttcattggcggcctccaccgtctgctgctgatgggcagcggcagcggcaccgagcagggcaaggaggtgctggagaaggcccggggcagcacctacggaaccccacggcctcctgtgcccaagcccaggcccgaagtgccccagagcgacgagacagccaccagccacggcagtgcccaggtgcccgagcctccaaccatccatctggccgcacagggcatggcctatcccctgcacgagcagcacggcatggcaccctgtcccgtggcccaggctcctcctactcccctgccacccgtgagccctggcgaccaactccccggggtgttcagcgacggcagggtggcttgcgctccagtaccggcccctgcaggacctatcgttcggccgtgggagccaagcctgacccaggcagcgggccag

SEQ ID NO: 27 - RNA, 1371 nt, synthetic construct
  source 1..1371: mol_type = other RNA
atggatcacgacgccggaggaagcgcacccgccagacccatgctgcctccgcagcccgaccttcccggacgggaggccatcctgcggcggttccctctggacctgcggaccctgctgcaggctatcggcgctgccgctacacggatcgacacccgggccatcgaccagttcttcggcagccagatcagcaacaccgagatgtacatcatgtacgccatggccatccggcaagccatcggcagcggaagcggcagcggaagcggaagcagagatcaggccaagtggcggctgcaaaccttagccgctggctggcccatgggctaccaggcctacagcagctggatgtacagctacaccgaccaccagaccactcccaccttcgtgcacctgcaggccacactcggctgcactggcggtagacggtgccacgtgaccttcagcgccggcaccttcaagctgcctcggtgtacacccggcgaccgtcagtggctgtacgtgcagagcagcgtgggcaacatcgtgcagagctgcaatccccggtacagcatcttcttcgactacatggccatccaccggagcctgaccaagatctgggaggaggtgctgactcccgaccagcgggtgagcttcatggagttcctgggcttcctgcagcggaccgacctgagctacatcaagagcttcgtgagcgacgccctgggcaccaccagcatccagacgccctggatcgacgacaaccccagcaccgagacagcccaggcctggaacgccggctttctaggtggaggcgcctacggaatcgacctgctgcggaccgagggcgagcacgtcgagggcgctacgggtgagacacgggaggagagcgaggacaccgagagcgacggcgacgacgaggacctgccctgcatcgtgagcagaggcggcggctccggcagcagacctcccatcttcattcgccggctccaccgtctgctgctgatgggcagcggcagcggcaccgagcagggcaaggaggtgctggagaaggcccggggcagcacctacggaaccccacggcctcctgtgcccaagcccaggcccgaagtgccccagagcgacgagacagccaccagccacggcagtgcccaggtgcccgagcctccaaccatccatctggccgcacagggcatggcctatcccctgcacgagcagcacggcatggcaccctgtcccgtggcccaggctcctcctactcccctgccacccgtgagccctggcgaccaactccccggggtgttcagcgacggcagggtggcttgcgctccagtaccggcccctgcaggacctatcgttcggccgtgggagccaagcctgacccaggcagcgggccag

SEQ ID NO: 28 - protein, 457 aa, synthetic construct
MDHDAGGSAPARPMLPPQPDLPGREAILRRFPLDLRTLLQAIGAAATRIDTRAIDQFFGSQISNTEMYIMYAMAIRQAIGSGSGSGSGSRDQAKWRLQTLAAGWPMGYQAYSSWMYSYTDHQTTPTFVHLQATLGCTGGRRCHVTFSAGTFKLPRCTPGDRQWLYVQSSVGNIVQSCNPRYSIFFDYMAIHRSLTKIWEEVLTPDQRVSFMEFLGFLQRTDLSYIKSFVSDALGTTSIQTPWIDDNPSTETAQAWNAGFLGGGAYGIDLLRTEGEHVEGATGETREESEDTESDGDDEDLPCIVSRGGGSGSGSGIFIRRLHRLLLMGSGSGTEQGKEVLEKARGSTYGTPRPPVPKPRPEVPQSDETATSHGSAQVPEPPTIHLAAQGMAYPLHEQHGMAPCPVAQAPPTPLPPVSPGDQLPGVFSDGRVACAPVPAPAGPIVRPWEPSLTQAAGQ

SEQ ID NO: 29 - protein, 457 aa, synthetic construct
MDHDAGGSAPARPMLPPQPDLPGREAILRRFPLDLRTLLQAIGAAATRIDTRAIDQFFGSQISNTEMYIMYAMAIRQAIGSGSGSGSGSRDQAKWRLQTLAAGWPMGYQAYSSWMYSYTDHQTTPTFVHLQATLGCTGGRRCHVTFSAGTFKLPRCTPGDRQWLYVQSSVGNIVQSCNPRYSIFFDYMAIHRSLTKIWEEVLTPDQRVSFMEFLGFLQRTDLSYIKSFVSDALGTTSIQTPWIDDNPSTETAQAWNAGFLRGRAYGIDLLRTEGEHVEGATGETREESEDTESDGDDEDLPCIVSRGGGSGSGSGIFIGGLHRLLLMGSGSGTEQGKEVLEKARGSTYGTPRPPVPKPRPEVPQSDETATSHGSAQVPEPPTIHLAAQGMAYPLHEQHGMAPCPVAQAPPTPLPPVSPGDQLPGVFSDGRVACAPVPAPAGPIVRPWEPSLTQAAGQ

SEQ ID NO: 30 - protein, 457 aa, synthetic construct
MDHDAGGSAPARPMLPPQPDLPGREAILRRFPLDLRTLLQAIGAAATRIDTRAIDQFFGSQISNTEMYIMYAMAIRQAIGSGSGSGSGSRDQAKWRLQTLAAGWPMGYQAYSSWMYSYTDHQTTPTFVHLQATLGCTGGRRCHVTFSAGTFKLPRCTPGDRQWLYVQSSVGNIVQSCNPRYSIFFDYMAIHRSLTKIWEEVLTPDQRVSFMEFLGFLQRTDLSYIKSFVSDALGTTSIQTPWIDDNPSTETAQAWNAGFLGGGAYGIDLLRTEGEHVEGATGETREESEDTESDGDDEDLPCIVSRGGGSGSGSGIFIGGLHRLLLMGSGSGTEQGKEVLEKARGSTYGTPRPPVPKPRPEVPQSDETATSHGSAQVPEPPTIHLAAQGMAYPLHEQHGMAPCPVAQAPPTPLPPVSPGDQLPGVFSDGRVACAPVPAPAGPIVRPWEPSLTQAAGQ

SEQ ID NO: 31 - protein, 457 aa, synthetic construct
MDHDAGGSAPARPMLPPQPDLPGREAILRRFPLDLRTLLQAIGAAATRIDTRAIDQFFGSQISNTEMYIMYAMAIRQAIGSGSGSGSGSRDQAKWRLQTLAAGWPMGYQAYSSWMYSYTDHQTTPTFVHLQATLGCTGGRRCHVTFSAGTFKLPRCTPGDRQWLYVQSSVGNIVQSCNPRYSIFFDYMAIHRSLTKIWEEVLTPDQRVSFMEFLGFLQRTDLSYIKSFVSDALGTTSIQTPWIDDNPSTETAQAWNAGFLGGGAYGIDLLRTEGEHVEGATGETREESEDTESDGDDEDLPCIVSRGGGSGSRPPIFIRRLHRLLLMGSGSGTEQGKEVLEKARGSTYGTPRPPVPKPRPEVPQSDETATSHGSAQVPEPPTIHLAAQGMAYPLHEQHGMAPCPVAQAPPTPLPPVSPGDQLPGVFSDGRVACAPVPAPAGPIVRPWEPSLTQAAGQ

SEQ ID NO: 32 - RNA, 1548 nt, synthetic construct
  source 1..1548: mol_type = other RNA
gaggaaataagagagaaaagaagagtaagaagaaatataagaccccggcgccgccaccatggatcacgacgccggaggaagcgcacccgccagacccatgctgcctccgcagcccgaccttcccggacgggaggccatcctgcggcggttccctctggacctgcggaccctgctgcaggctatcggcgctgccgctacacggatcgacacccgggccatcgaccagttcttcggcagccagatcagcaacaccgagatgtacatcatgtacgccatggccatccggcaagccatcggcagcggaagcggcagcggaagcggaagcagagatcaggccaagtggcggctgcaaaccttagccgctggctggcccatgggctaccaggcctacagcagctggatgtacagctacaccgaccaccagaccactcccaccttcgtgcacctgcaggccacactcggctgcactggcggtagacggtgccacgtgaccttcagcgccggcaccttcaagctgcctcggtgtacacccggcgaccgtcagtggctgtacgtgcagagcagcgtgggcaacatcgtgcagagctgcaatccccggtacagcatcttcttcgactacatggccatccaccggagcctgaccaagatctgggaggaggtgctgactcccgaccagcgggtgagcttcatggagttcctgggcttcctgcagcggaccgacctgagctacatcaagagcttcgtgagcgacgccctgggcaccaccagcatccagacgccctggatcgacgacaaccccagcaccgagacagcccaggcctggaacgccggctttctaggtggaggcgcctacggaatcgacctgctgcggaccgagggcgagcacgtcgagggcgctacgggtgagacacgggaggagagcgaggacaccgagagcgacggcgacgacgaggacctgccctgcatcgtgagcagaggcggcggctccggcagcggcagcggcatcttcattcgccggctccaccgtctgctgctgatgggcagcggcagcggcaccgagcagggcaaggaggtgctggagaaggcccggggcagcacctacggaaccccacggcctcctgtgcccaagcccaggcccgaagtgccccagagcgacgagacagccaccagccacggcagtgcccaggtgcccgagcctccaaccatccatctggccgcacagggcatggcctatcccctgcacgagcagcacggcatggcaccctgtcccgtggcccaggctcctcctactcccctgccacccgtgagccctggcgaccaactccccggggtgttcagcgacggcagggtggcttgcgctccagtaccggcccctgcaggacctatcgttcggccgtgggagccaagcctgacccaggcagcgggccagtgataataggctggagcctcggtggcctagcttcttgccccttgggcctccccccagcccctcctccccttcctgcacccgtacccccgtggtctttgaataaagtctgagtgggcggc

SEQ ID NO: 33 - RNA, 1548 nt, synthetic construct
  source 1..1548: mol_type = other RNA
gaggaaataagagagaaaagaagagtaagaagaaatataagaccccggcgccgccaccatggatcacgacgccggaggaagcgcacccgccagacccatgctgcctccgcagcccgaccttcccggacgggaggccatcctgcggcggttccctctggacctgcggaccctgctgcaggctatcggcgctgccgctacacggatcgacacccgggccatcgaccagttcttcggcagccagatcagcaacaccgagatgtacatcatgtacgccatggccatccggcaagccatcggcagcggaagcggcagcggaagcggaagcagagatcaggccaagtggcggctgcaaaccttagccgctggctggcccatgggctaccaggcctacagcagctggatgtacagctacaccgaccaccagaccactcccaccttcgtgcacctgcaggccacactcggctgcactggcggtagacggtgccacgtgaccttcagcgccggcaccttcaagctgcctcggtgtacacccggcgaccgtcagtggctgtacgtgcagagcagcgtgggcaacatcgtgcagagctgcaatccccggtacagcatcttcttcgactacatggccatccaccggagcctgaccaagatctgggaggaggtgctgactcccgaccagcgggtgagcttcatggagttcctgggcttcctgcagcggaccgacctgagctacatcaagagcttcgtgagcgacgccctgggcaccaccagcatccagacgccctggatcgacgacaaccccagcaccgagacagcccaggcctggaacgccggctttctacggggacgggcctacggaatcgacctgctgcggaccgagggcgagcacgtcgagggcgctacgggtgagacacgggaggagagcgaggacaccgagagcgacggcgacgacgaggacctgccctgcatcgtgagcagaggcggcggctccggcagcggcagcggcatcttcattggcggcctccaccgtctgctgctgatgggcagcggcagcggcaccgagcagggcaaggaggtgctggagaaggcccggggcagcacctacggaaccccacggcctcctgtgcccaagcccaggcccgaagtgccccagagcgacgagacagccaccagccacggcagtgcccaggtgcccgagcctccaaccatccatctggccgcacagggcatggcctatcccctgcacgagcagcacggcatggcaccctgtcccgtggcccaggctcctcctactcccctgccacccgtgagccctggcgaccaactccccggggtgttcagcgacggcagggtggcttgcgctccagtaccggcccctgcaggacctatcgttcggccgtgggagccaagcctgacccaggcagcgggccagtgataataggctggagcctcggtggcctagcttcttgccccttgggcctccccccagcccctcctccccttcctgcacccgtacccccgtggtctttgaataaagtctgagtgggcggc

SEQ ID NO: 34 - RNA, 1548 nt, synthetic construct
  source 1..1548: mol_type = other RNA
gaggaaataagagagaaaagaagagtaagaagaaatataagaccccggcgccgccaccatggatcacgacgccggaggaagcgcacccgccagacccatgctgcctccgcagcccgaccttcccggacgggaggccatcctgcggcggttccctctggacctgcggaccctgctgcaggctatcggcgctgccgctacacggatcgacacccgggccatcgaccagttcttcggcagccagatcagcaacaccgagatgtacatcatgtacgccatggccatccggcaagccatcggcagcggaagcggcagcggaagcggaagcagagatcaggccaagtggcggctgcaaaccttagccgctggctggcccatgggctaccaggcctacagcagctggatgtacagctacaccgaccaccagaccactcccaccttcgtgcacctgcaggccacactcggctgcactggcggtagacggtgccacgtgaccttcagcgccggcaccttcaagctgcctcggtgtacacccggcgaccgtcagtggctgtacgtgcagagcagcgtgggcaacatcgtgcagagctgcaatccccggtacagcatcttcttcgactacatggccatccaccggagcctgaccaagatctgggaggaggtgctgactcccgaccagcgggtgagcttcatggagttcctgggcttcctgcagcggaccgacctgagctacatcaagagcttcgtgagcgacgccctgggcaccaccagcatccagacgccctggatcgacgacaaccccagcaccgagacagcccaggcctggaacgccggctttctaggtggaggcgcctacggaatcgacctgctgcggaccgagggcgagcacgtcgagggcgctacgggtgagacacgggaggagagcgaggacaccgagagcgacggcgacgacgaggacctgccctgcatcgtgagcagaggcggcggctccggcagcggcagcggcatcttcattggcggcctccaccgtctgctgctgatgggcagcggcagcggcaccgagcagggcaaggaggtgctggagaaggcccggggcagcacctacggaaccccacggcctcctgtgcccaagcccaggcccgaagtgccccagagcgacgagacagccaccagccacggcagtgcccaggtgcccgagcctccaaccatccatctggccgcacagggcatggcctatcccctgcacgagcagcacggcatggcaccctgtcccgtggcccaggctcctcctactcccctgccacccgtgagccctggcgaccaactccccggggtgttcagcgacggcagggtggcttgcgctccagtaccggcccctgcaggacctatcgttcggccgtgggagccaagcctgacccaggcagcgggccagtgataataggctggagcctcggtggcctagcttcttgccccttgggcctccccccagcccctcctccccttcctgcacccgtacccccgtggtctttgaataaagtctgagtgggcggc

SEQ ID NO: 35 - RNA, 1548 nt, synthetic construct
  source 1..1548: mol_type = other RNA
gaggaaataagagagaaaagaagagtaagaagaaatataagaccccggcgccgccaccatggatcacgacgccggaggaagcgcacccgccagacccatgctgcctccgcagcccgaccttcccggacgggaggccatcctgcggcggttccctctggacctgcggaccctgctgcaggctatcggcgctgccgctacacggatcgacacccgggccatcgaccagttcttcggcagccagatcagcaacaccgagatgtacatcatgtacgccatggccatccggcaagccatcggcagcggaagcggcagcggaagcggaagcagagatcaggccaagtggcggctgcaaaccttagccgctggctggcccatgggctaccaggcctacagcagctggatgtacagctacaccgaccaccagaccactcccaccttcgtgcacctgcaggccacactcggctgcactggcggtagacggtgccacgtgaccttcagcgccggcaccttcaagctgcctcggtgtacacccggcgaccgtcagtggctgtacgtgcagagcagcgtgggcaacatcgtgcagagctgcaatccccggtacagcatcttcttcgactacatggccatccaccggagcctgaccaagatctgggaggaggtgctgactcccgaccagcgggtgagcttcatggagttcctgggcttcctgcagcggaccgacctgagctacatcaagagcttcgtgagcgacgccctgggcaccaccagcatccagacgccctggatcgacgacaaccccagcaccgagacagcccaggcctggaacgccggctttctaggtggaggcgcctacggaatcgacctgctgcggaccgagggcgagcacgtcgagggcgctacgggtgagacacgggaggagagcgaggacaccgagagcgacggcgacgacgaggacctgccctgcatcgtgagcagaggcggcggctccggcagcagacctcccatcttcattcgccggctccaccgtctgctgctgatgggcagcggcagcggcaccgagcagggcaaggaggtgctggagaaggcccggggcagcacctacggaaccccacggcctcctgtgcccaagcccaggcccgaagtgccccagagcgacgagacagccaccagccacggcagtgcccaggtgcccgagcctccaaccatccatctggccgcacagggcatggcctatcccctgcacgagcagcacggcatggcaccctgtcccgtggcccaggctcctcctactcccctgccacccgtgagccctggcgaccaactccccggggtgttcagcgacggcagggtggcttgcgctccagtaccggcccctgcaggacctatcgttcggccgtgggagccaagcctgacccaggcagcgggccagtgataataggctggagcctcggtggcctagcttcttgccccttgggcctccccccagcccctcctccccttcctgcacccgtacccccgtggtctttgaataaagtctgagtgggcggc

SEQ ID NO: 36 - protein, 15 aa, synthetic construct
GGSGGSGGSGGSGGG

SEQ ID NO: 37 - protein, 8 aa, synthetic construct
GGGSGGGS

SEQ ID NO: 38 - protein, 7 aa, synthetic construct
GGGSGGG

SEQ ID NO: 39 - protein, 6 aa, synthetic construct
GGGSGG

SEQ ID NO: 40 - protein, 4 aa, synthetic construct
GGGS

SEQ ID NO: 41 - protein, 20 aa, synthetic construct
GGGSGGGSGGGSGGGSGGGS

SEQ ID NO: 42 - protein, 4 aa, synthetic construct
GSGG

SEQ ID NO: 43 - protein, 20 aa, synthetic construct
GSGGGSGGGSGGGSGGGSGG

SEQ ID NO: 44 - RNA, 11 nt, synthetic construct
  source 1..11: mol_type = other RNA
gggatcctacc

SEQ ID NO: 45 - RNA, 21 nt, synthetic construct
  source 1..21: mol_type = other RNA
gagattgagtgtagtgactag

SEQ ID NO: 46 - RNA, 18 nt, synthetic construct
  source 1..18: mol_type = other RNA
gagattgagtgtagtgac

SEQ ID NO: 47 - RNA, 16 nt, synthetic construct
  source 1..16: mol_type = other RNA
gagattgagtgtagtg

SEQ ID NO: 48 - RNA, 15 nt, synthetic construct
  source 1..15: mol_type = other RNA
gattgagactacggg

SEQ ID NO: 49 - RNA, 13 nt, synthetic construct
  source 1..13: mol_type = other RNA
catagacactacg

SEQ ID NO: 50 - protein, 20 aa, Homo sapiens
METPAQLLFLLLLWLPDTTG

SEQ ID NO: 51 - protein, 18 aa, Homo sapiens
MDWTWILFLVAAATRVHS

SEQ ID NO: 52 - protein, 24 aa, Japanese encephalitis virus
MLGSNSGQRVVFTILLLLVAPAYS

SEQ ID NO: 53 - protein, 17 aa, Vesiculovirus indiana
MKCLLYLAFLFIGVNCA

SEQ ID NO: 54 - protein, 15 aa, Japanese encephalitis virus
MWLVSLAIVTACAGA

SEQ ID NO: 55 - RNA, 47 nt, synthetic construct
  source 1..47: mol_type = other RNA
gggaaataagagagaaaagaagagtaagaagaaatataagagccacc

SEQ ID NO: 56 - RNA, 57 nt, synthetic construct
  source 1..57: mol_type = other RNA
gggaaataagagagaaaagaagagtaagaagaaatataagaccccggcgccgccacc

SEQ ID NO: 57 - RNA, 75 nt, synthetic construct
  source 1..75: mol_type = other RNA
ggaaatcgcaaaatttgctcttcgcgttagatttcttttagttttctcgcaactagcaagctttttgttctcgcc

SEQ ID NO: 58 - RNA, 203 nt, synthetic construct
  source 1..203: mol_type = other RNA
ggaaatccccacaaccgcctcatatccaggctcaagaatagagctcagtgttttgttgtttaatcattccgacgtgttttgcgatattcgcgcaaagcagccagtcgcgcgcttgcttttaagtagagttgtttttccacccgtttgccaggcatctttaatttaacatatttttatttttcaggctaacctacgccgccacc

SEQ ID NO: 59 - RNA, 76 nt, synthetic construct
  source 1..76: mol_type = other RNA
ggaaataagagagaaaagaagagtaagaagaaatataagatctccctgagcttcagggagccccggcgccgccacc

SEQ ID NO: 60 - RNA, 89 nt, synthetic construct
  source 1..89: mol_type = other RNA
ggaaaccccccacccccgtaagagagaaaagaagagtaagaagaaatataagatctccctgagcttcagggagccccggcgccgccacc

SEQ ID NO: 61 - RNA, 59 nt, synthetic construct
  source 1..59: mol_type = other RNA
ggagaacttccgcttccgttggcgcaagcgctttcattttttctgctaccgtgactaag

SEQ ID NO: 62 - RNA, 46 nt, synthetic construct
  source 1..46: mol_type = other RNA
ggaaataagagagaaaagaagagtaagaagaaatataagagccacc

SEQ ID NO: 63 - RNA, 56 nt, synthetic construct
  source 1..56: mol_type = other RNA
ggaaataagagagaaaagaagagtaagaagaaatataagaccccggcgccgccacc

SEQ ID NO: 64 - RNA, 81 nt, synthetic construct
  source 1..81: mol_type = other RNA
ggaaatcgcaaaatttgctcttcgcgttagatttcttttagttttctcgcaactagcaagctttttgttctcgccgccgcc

SEQ ID NO: 65 - RNA, 81 nt, synthetic construct
  source 1..81: mol_type = other RNA
ggaaatcgcaaaattttcttttcgcgttagatttcttttagttttctttcaactagcaagctttttgttctcgccgccgcc

SEQ ID NO: 66 - RNA, 83 nt, synthetic construct
  source 1..83: mol_type = other RNA
  misc_feature 14..18: note = may be absent
  misc_feature 19: note = may be absent
  misc_feature 22: note = may be absent
  misc_feature 23..27: note = may be absent
  misc_feature 81..83: note = may be absent
ggaaatcgcaaaatttttgctctttttcgcgttagatttcttttagttttctykcaactagcaagctttttgttctcgccrcc

SEQ ID NO: 67 - RNA, 79 nt, synthetic construct
  source 1..79: mol_type = other RNA
ggaaaattttagcctggaacgttagataactgtcctgttgtctttatatacttggtccccaagtagtttgtcttccaaa

SEQ ID NO: 68 - RNA, 79 nt, synthetic construct
  source 1..79: mol_type = other RNA
ggaaactttatttagtgttactttattttctgtttatttgtgtttcttcagtgggtttgttctaatttccttggccgcc

SEQ ID NO: 69 - RNA, 79 nt, synthetic construct
  source 1..79: mol_type = other RNA
ggaaaatctgtattaggttggcgtgttctttggtcggttgttagtattgttgttgattcgtttgtggtcggttgccgcc

SEQ ID NO: 70 - RNA, 79 nt, synthetic construct
  source 1..79: mol_type = other RNA
ggaaaattattaacatcttggtattctcgataaccattcgttggattttattgtattcgtagtttgggttcctgccgcc

SEQ ID NO: 71 - RNA, 79 nt, synthetic construct
  source 1..79: mol_type = other RNA
ggaaattattattatttctagctacaatttatcattgtattattttagctattcatcattatttacttggtgatcaaca

SEQ ID NO: 72 - RNA, 79 nt, synthetic construct
  source 1..79: mol_type = other RNA
ggaaataggttgttaaccaagttcaagcctaataagcttggattctggtgacttgcttcaccgttggcgggcaccgatc

SEQ ID NO: 73 - RNA, 74 nt, synthetic construct
  source 1..74: mol_type = other RNA
ggaaatcgtagagagtcgtacttagtacatatcgactatcggtggacaccatcaagattataaaccaggccaga

SEQ ID NO: 74 - RNA, 79 nt, synthetic construct
  source 1..79: mol_type = other RNA
ggaaacccgcccaagcgaccccaacatatcagcagttgcccaatcccaactcccaacacaatccccaagcaacgccgcc

SEQ ID NO: 75 - RNA, 79 nt, synthetic construct
  source 1..79: mol_type = other RNA
ggaaagcgattgaaggcgtcttttcaactactcgattaaggttgggtatcgtcgtgggacttggaaatttgttgtttcc

SEQ ID NO: 76 - RNA, 79 nt, synthetic construct
  source 1..79: mol_type = other RNA
ggaaactaatcgaaataaaagagccccgtactcttttatttctattaggttaggagccttagcatttgtatcttaggta

SEQ ID NO: 77 - RNA, 79 nt, synthetic construct
  source 1..79: mol_type = other RNA
ggaaatgtgatttccagcaacttcttttgaatatattgaattcctaattcaaagcgaacaaatctacaagccatatacc

SEQ ID NO: 78 - RNA, 74 nt, synthetic construct
  source 1..74: mol_type = other RNA
ggaaatcgtagagagtcgtacttacgtggtcgccattgcatagcgcgcgaaagcaacaggaacaagaacgcgcc

SEQ ID NO: 79 - RNA, 74 nt, synthetic construct
  source 1..74: mol_type = other RNA
ggaaatcgtagagagtcgtacttagaataaacagagtcgggtcgacttgtctctgatactacgacgtcacaatc

SEQ ID NO: 80 - RNA, 79 nt, synthetic construct
  source 1..79: mol_type = other RNA
ggaaaatttgccttcggagttgcgtatcctgaactgcccagcctcctgatatacaactgttccgcttattcgggccgcc

SEQ ID NO: 81 - RNA, 79 nt, synthetic construct
  source 1..79: mol_type = other RNA
ggaaatctgagcaggaatcctttgtgcattgaagactttagattcctctctgcggtagacgtgcacttataagtatttg

SEQ ID NO: 82 - RNA, 79 nt, synthetic construct
  source 1..79: mol_type = other RNA
ggaaagcgattgaaggcgtcttttcaactactcgattaaggttgggtatcgtcgtgggacttggaaatttgttgccacc

SEQ ID NO: 83 - RNA, 104 nt, synthetic construct
  source 1..104: mol_type = other RNA
ggaaatttttttttgatattataagagtttttttttgatattaagaaaatttttttttgatattagaagagtaagaagaaatataagaccccggcgccgccacc

SEQ ID NO: 84 - RNA, 57 nt, synthetic construct
  source 1..57: mol_type = other RNA
ggaaataagagagaaaagaagagtaagaagaaatataagagccaaaaaaaaaaaacc

SEQ ID NO: 85 - RNA, 76 nt, synthetic construct
  source 1..76: mol_type = other RNA
ggaaatctccctgagcttcagggagtaagagagaaaagaagagtaagaagaaatataagaccccggcgccgccacc

SEQ ID NO: 86 - RNA, 36 nt, synthetic construct
  source 1..36: mol_type = other RNA
ggactcactatttgttttcgcgcccagttgcaaaaa

SEQ ID NO: 87 - RNA, 119 nt, synthetic construct
  source 1..119: mol_type = other RNA
tgataataggctggagcctcggtggccatgcttcttgccccttgggcctccccccagcccctcctccccttcctgcacccgtacccccgtggtctttgaataaagtctgagtgggcggc

SEQ ID NO: 88 - RNA, 119 nt, synthetic construct
  source 1..119: mol_type = other RNA
taaagctccccgggggcctcggtggcctagcttcttgccccttgggcctccccccagcccctcctccccttcctgcacccgtacccccgtggtctttgaataaagtctgagtgggcggc

SEQ ID NO: 89 - RNA, 119 nt, synthetic construct
  source 1..119: mol_type = other RNA
taagtctaagctggagcctcctgagagacctgtgtgaactattgagaagatcggaacagctccttactctgaggaagttggtacccccgtggtctttgaataaagtctgagtgggcggc

SEQ ID NO: 90 - RNA, 185 nt, synthetic construct
  source 1..185: mol_type = other RNA
taaagctccccggggcaaacaccattgtcacactccagcctcggtggcctagcttcttgccccttgggcccaaacaccattgtcacactccatccccccagcccctcctccccttcctgcacccgtaccccccaaacaccattgtcacactccagtggtctttgaataaagtctgagtgggcggc

SEQ ID NO: 91 - RNA, 170 nt, synthetic construct
  source 1..170: mol_type = other RNA
taaagctccccggggtccataaagtaggaaacactacagctggagcctcggtggcctagcttcttgccccttgggcccaaacaccattgtcacactccatccccccagcccctcctccccttcctgcacccgtacccccgtggtctttgaataaagtctgagtgggcggc

SEQ ID NO: 92 - RNA, 141 nt, synthetic construct
  source 1..141: mol_type = other RNA
taaagctccccgggggcctcggtggcctagcttcttgccccttgggcctccccccagcccctcctccccttcctgcacccgtaccccccaaacaccattgtcacactccagtggtctttgaataaagtctgagtgggcggc

SEQ ID NO: 93 - RNA, 185 nt, synthetic construct
  source 1..185: mol_type = other RNA
taagcccctccggggcaaacaccattgtcacactccagcctcggtggcctagcttcttgccccttgggcccaaacaccattgtcacactccatccccccagcccctcctccccttcctgcacccgtaccccccaaacaccattgtcacactccagtggtctttgaataaagtctgagtgggcggc

SEQ ID NO: 94 - RNA, 187 nt, synthetic construct
  source 1..187: mol_type = other RNA
taagcccctccggggtccataaagtaggaaacactacagcctcggtggcctagcttcttgccccttgggcctccataaagtaggaaacactacatccccccagcccctcctccccttcctgcacccgtaccccccgcattattactcacggtacgagtggtctttgaataaagtctgagtgggcggc

SEQ ID NO: 95 - RNA, 170 nt, synthetic construct
  source 1..170: mol_type = other RNA
taagcccctccggggtccataaagtaggaaacactacagctggagcctcggtggcctagcttcttgccccttgggcccaaacaccattgtcacactccatccccccagcccctcctccccttcctgcacccgtacccccgtggtctttgaataaagtctgagtgggcggc

SEQ ID NO: 96 - RNA, 141 nt, synthetic construct
  source 1..141: mol_type = other RNA
taagcccctccgggggcctcggtggcctagcttcttgccccttgggcctccccccagcccctcctccccttcctgcacccgtaccccccaaacaccattgtcacactccagtggtctttgaataaagtctgagtgggcggc

SEQ ID NO: 97 - protein, 9 aa, synthetic construct
RPPIFIRRL

SEQ ID NO: 98 - protein, 9 aa, synthetic construct
  VARIANT 9: note = may be Leucine or Isoleucine
FLRGRAYGX

SEQ ID NO: 99 - protein, 4 aa, synthetic construct
KRKR

SEQ ID NO: 100 - protein, 10 aa, synthetic construct
RDRRRNPASR

SEQ ID NO: 101 - protein, 7 aa, synthetic construct
PKVKRPP

SEQ ID NO: 102 - protein, 4 aa, synthetic construct
PKVK

SEQ ID NO: 103 - protein, 5 aa, synthetic construct
RAGKR

SEQ ID NO: 104 - protein, 4 aa, synthetic construct
GSGS

SEQ ID NO: 105 - protein, 10 aa, synthetic construct
GSGSGSGSGS

SEQ ID NO: 106 - protein, 7 aa, synthetic construct
GSGSGSG

SEQ ID NO: 107 - protein, 5 aa, synthetic construct
GSGSG